(19) United States
(12) Patent Application Publication
(10) Pub. No.: US 2018/0100858 A1
(43) Pub. Date: Apr. 12, 2018

(54) PROTEIN BIOMARKER PANELS FOR DETECTING COLORECTAL CANCER AND ADVANCED ADENOMA

(71) Applicant: Applied Proteomics, Inc., San Diego, CA (US)

(72) Inventors: John Blume, Bellingham, WA (US); Athit Kao, San Marcos, CA (US); Roslyn Dillon, Cardiff, CA (US); Lisa Croner, San Diego, CA (US); Bruce Wilcox, Harrisonburg, VA (US)

(21) Appl. No.: 15/414,456

(22) Filed: Jan. 24, 2017

Related U.S. Application Data
(60) Provisional application No. 62/405,771, filed on Oct. 7, 2016.

(51) Int. Cl. G01N 33/574

(52) U.S. Cl. CPC G01N 33/57419; G01N 2800/60; G01N 2333/705; G01N 2333/948; G01N 2333/91215; G01N 2333/4716

(57) ABSTRACT

Disclosed herein are panels related to the diagnosis or recognition of advanced adenoma and colorectal cancer in a subject. The disclosed panels and related methods are used to predict or assess colon tumor status in a patient. They can be used to determine nature of tumor, recurrence, or patient response to treatments. Some embodiments of the methods include generating a report for clinical management.

### RELATED APPLICATIONS

The present application claims the benefit of priority to U.S. Provisional Application Ser. No. 62/405,771, filed Oct. 7, 2016, which is hereby incorporated by reference in its entirety.

### BACKGROUND

Colorectal cancer is a leading cause of cancer-related deaths in the United States with over 142,820 diagnosed cases and over 50,000 deaths in 2013. According to a 2011 study, there are an estimated 1.2 million diagnoses per year and 600,000 deaths worldwide.

Colorectal cancer (CRC) results from uncontrolled cell growth in the lower gastrointestinal tract, such as the colon, rectum or appendix. CRC can develop from a colon polyp. A colon polyp typically comprises a benign clump of cells that forms on the lining of the large intestine or rectum. While many colon polyps are non-malignant, a polyp can develop into an adenoma. Colorectal adenomas can then grow into advanced colorectal adenomas, which can then develop into CRC.

The risk of developing CRC increases with age. Ninety percent of new cases and 93% of deaths occur in people age 50 and older. During their 60s, men have a 10-fold increased risk of developing CRC compared to their 40s. Regular screening allows for the removal of advanced colorectal adenomas or precancerous polyps and detection of early stage cancer, which is the key factor in the effective treatment of the disease.

The survival rate for patients diagnosed with CRC is highly dependent on when it is caught. CRC usually progresses through four stages, defined as Stage I through Stage IV. Stages I and II are local stages, during which aberrant cell growth is confined to the colon or rectum. Stage III is a regional stage, meaning the cancer has spread to the surrounding tissue but remains local. Stage IV is distal and indicates that the cancer has spread throughout the other organs of the body, most commonly the liver or lungs. It is estimated that the five-year survival rate is over 90% for those patients who were diagnosed with Stage I CRC, compared to 13% for a Stage IV diagnosis. If caught early, CRC is typically treated by surgical removal of the cancer. After the cancer spreads, surgical removal of the cancer is typically followed by chemotherapy.

CRC is one of the most preventable cancers given its typically slow progression from early stages to metastatic disease and available tools for its diagnosis.

It is also one of the least prevented cancers. This is largely due to poor compliance with available CRC screening approaches. Current screening approaches involve either stool sample analysis or direct observation via a colonoscopy or sigmoidoscopy, each of which has a low compliance rate. As a result, CRC is often detected only after progressing past the point at which treatment success rates have declined substantially.

Colonoscopy and sigmoidoscopy remain the gold standard for detecting colon cancer. However, the highly invasive nature and the expense of these exams contribute to low acceptance from the population. Furthermore, such highly invasive procedures expose subjects to risk of complications such as infection.

The most common non-invasive test for colorectal cancer is the fecal occult blood test (“FOBT”). Unfortunately, in addition to its high false-positive rate, the sensitivity of the FOBT remains around 50% and may have less sensitivity for detection of early stage CRC. Numerous serum markers, such as carcinoembryonic antigen (“CEA”), carbohydrate antigen 19-9, and lipid-associated sialic acid, have been investigated in colorectal cancer. However, their low sensitivity has induced the American Society of Clinical Oncology to state that none can be recommended for screening and diagnosis, and that their use should be limited to post-surgery surveillance.

Because of the significantly increased chance of survival if CRC is detected early in the disease progression, CRC is one of three cancers for which the American Cancer Society, or ACS, recommends routine screening (breast and cervical cancer are the others). In the United States, screening for CRC is currently recommended by the ACS and the U.S. Preventative Services Task Force, or USPSTF, for all men and women aged 50-75 using fecal occult blood testing, or FOBT, which is a fecal test, or one of two procedures: colonoscopy or sigmoidoscopy. Despite the benefits of routine screening on improving five-year survival rates if CRC is diagnosed early, the rate of screening compliance is low due in part to the limitations of existing solutions.

CRC often develops from pre-cancerous adenomas in the lower gastrointestinal tract, such as the colon, rectum or appendix. Thus advanced adenoma (AA) detection is a valuable tool for the early detection of CRC. Although not all AA develops into CRC, the detection of AA in an individual is a valuable tool for identifying and addressing mis-dividing cell clusters either prior to or early in their development into CRC, when the condition is most easily treated.

### SUMMARY

Provided herein are noninvasive methods of assessing a CRC status in an individual, for example using a blood sample of an individual. Some such methods comprise the steps of obtaining a circulating blood sample from the individual; obtaining a biomarker panel level for a biomarker panel comprising a list of proteins in the sample comprising C9, CEA, DPP4, MIF, ORM1, PKM, SAA, and TFRC, and also including individual age and gender as biomarkers to comprise panel information from said individual, and using said panel information to make a CRC health assessment. Some approaches comprise comparing said panel information from said individual to a reference panel information set corresponding to a known colorectal cancer status, such as at least one of no CRC, stage I CRC, Stage II CRC, stage III CRC, stage IV CRC, and more generally early CRC, advanced CRC; and categorizing said individual as having said colorectal cancer status if said individual's reference panel information does not differ significantly from said reference panel information set. Some approaches comprise using panel levels in an algorithm to obtain a panel score, and comparing the panel score to that of panel scores for at least one reference panel information set score corresponding to a known colorectal cancer status, such as at least one of no CRC, stage I CRC, Stage II CRC, stage III CRC, stage IV CRC, and more generally early CRC, advanced CRC; and categorizing said individual as having said colorectal cancer status if said individual's reference panel information does not differ significantly from said reference panel information set. Some approaches comprise using ratios of selected biomarkers relative to one another in an algorithm to obtain a panel score, and comparing the panel score to that of panel scores for at least one reference panel information set score corresponding to a known colorectal cancer status, such as at least one of no CRC, stage I CRC, Stage II CRC, stage III CRC, stage IV CRC, and more generally early CRC, advanced CRC; and categorizing said individual as having said colorectal cancer status if said individual's reference panel information does not differ significantly from said reference panel information set.

Some approaches comprise comparing said panel information from said individual to a reference panel information set corresponding to a known colorectal cancer status, such as at least one of no CRC, stage I CRC, Stage II CRC, stage III CRC, stage IV CRC, and more generally early CRC, advanced CRC; and categorizing said individual as having a CRC status different from said reference panel if said individual's reference panel information differs significantly from said reference panel information set. Some approaches comprise using panel levels in an algorithm to obtain a panel score, and comparing the panel score to that of panel scores for at least one reference panel information set score corresponding to a known colorectal cancer status, such as at least one of no CRC, stage I CRC, Stage II CRC, stage III CRC, stage IV CRC, and more generally early CRC, advanced CRC; and categorizing said individual as not having said colorectal cancer status if said individual's reference panel information differs significantly from said reference panel information set. Some approaches comprise using ratios of selected biomarkers relative to one another in an algorithm to obtain a panel score, and comparing the panel score to that of panel scores for at least one reference panel information set score corresponding to a known colorectal cancer status, such as at least one of no CRC, stage I CRC, Stage II CRC, stage III CRC, stage IV CRC, and more generally early CRC, advanced CRC; and categorizing said individual as not having said colorectal cancer status if said individual's reference panel information differs significantly from said reference panel information set.

Some CRC panels disclosed herein demonstrate a Validation Area Under curve (AUC), a parameter of panel test success, of at least 0.83, such as 0.83, 0.84, 0.85, 0.86, 0.87, 0.88, 0.89, 0.90. or greater than 0.90. If a No Call rate of 0% is adopted, in some cases one observes a CRC AUC of 0.83 or about 0.83, and a Validation Sensitivity of 0.80 or about 0.80 and a validation specificity of 0.71 or about 0.71. If a No Call rate of 12.3% or about 12.3% is adopted, in some cases one observes a CRC AUC of 0.85 or about 0.85, and a Validation Sensitivity of 0.80 or about 0.80 and a validation specificity of 0.76 or about 0.76. If a No Call rate of 18.2% or about 18.2% is adopted, in some cases one observes a CRC AUC of 0.85 or about 0.85, and a Validation Sensitivity of 0.82 or about 0.82 and a validation specificity of 0.78 or about 0.78. If a No Call rate of 23.2% or about 23.2% is adopted, in some cases one observes a CRC AUC of 0.86 or about 0.86, and a Validation Sensitivity of 0.80 or about 0.80 and a validation specificity of 0.83 or about 0.83.

Also provided herein are noninvasive methods of assessing an advanced adenoma status in an individual, for example using a blood sample of an individual. Some such methods comprise the steps of obtaining a circulating blood sample from the individual; obtaining a biomarker panel level for a biomarker panel comprising a list of proteins in the sample comprising CLU, CTSD, DPP4, GDF15, GSN, MIF, PKM, SERPINA1, SERPINA3, TFRC, and TIMP1, and obtaining the age of the individual as biomarkers to comprise panel information from said individual, and using said panel information to make a CRC health assessment. Some approaches comprise comparing said panel information from said individual to a reference panel information set corresponding to a known AA status; and categorizing said individual as having said AA status if said individual's reference panel information does not differ significantly from said reference panel information set. Some approaches comprise using panel levels in an algorithm to obtain a panel score, and comparing the panel score to that of panel scores for at least one reference panel information set score corresponding to a known AA status; and categorizing said individual as having said AA status if said individual's reference panel information does not differ significantly from said reference panel information set. Some approaches comprise using ratios of selected biomarkers relative to one another in an algorithm to obtain a panel score, and comparing the panel score to that of panel scores for at least one reference panel information set score corresponding to a known AA status; and categorizing said individual as having said AA status if said individual's reference panel information does not differ significantly from said reference panel information set.

Some approaches comprise comparing said panel information from said individual to a reference panel information set corresponding to a known AA status; and categorizing said individual as having an AA status different from said reference panel if said individual's reference panel information differs significantly from said reference panel information set. Some approaches comprise using panel levels in an algorithm to obtain a panel score, and comparing the panel score to that of panel scores for at least one reference panel information set score corresponding to a known AA status; and categorizing said individual as not having said AA status if said individual's reference panel information differs significantly from said reference panel information set. Some approaches comprise using ratios of selected biomarkers relative to one another in an algorithm to obtain a panel score, and comparing the panel score to that of panel scores for at least one reference panel information set score corresponding to a known AA status; and categorizing said individual as not having said AA status if said individual's reference panel information differs significantly from said reference panel information set.

Some AA panels disclosed herein demonstrate a Validation Area Under curve (AUC), a parameter of panel test success, of at least 0.69, such as 0.69, 0.70, 0.71, 0.72, 0.73, 0.74, 0.75, 0.80, 0.85, or greater than 0.85. If a No Call rate of 0% is adopted, in some cases one observes an AA AUC of 0.69 or about 0.69, and a Validation Sensitivity of 0.44 or about 0.44 and a validation specificity of 0.80 or about 0.80. If a No Call rate of 8.5% or about 8.5% is adopted, in some cases one observes a CRC AUC of 0.69 or about 0.69, and a Validation Sensitivity of 0.47 or about 0.47 and a validation specificity of 0.80 or about 0.80.

In light of the above and the disclosure herein, provided herein are methods, compositions, kits, computer readable media, and systems for the diagnosis and/or treatment of at least one of advanced colorectal adenoma and colorectal cancer. Through the methods and compositions provided herein, a sample is taken from an individual. In some cases the individual presents no symptoms of colorectal cancer, or advanced adenoma, or both colorectal cancer and adenoma. Some individuals are tested as part of routine health observation or monitoring. Alternately, some individuals are tested in relation to presenting at least one symptom of a colorectal health issue such as colorectal cancer, or advanced adenoma, or both colorectal cancer and adenoma. In some cases the individual is identified as being at risk of colorectal cancer, or advanced adenoma, or both colorectal cancer and adenoma. The sample is assayed to determine the accumulation levels of a panel of markers such as proteins, or proteins and age, or proteins and gender, or proteins and age and gender, for example a panel of markers comprising or consisting of the markers in panels disclosed herein. In many cases the panels comprise proteins that individually are known to play a role in indicating the presence of advanced colorectal adenoma or colorectal cancer, while in other cases the panels comprise a protein or proteins not know to correlate with advanced colorectal adenoma or colorectal cancer. However, in all cases the identification and accumulation of markers into a panel results in a level of specificity, sensitivity or specificity and sensitivity that substantially surpasses that of individual markers or smaller or less accurate sets of markers.

Additionally, methods, panels and other tests disclosed herein substantially surpass the sensitivity, specificity, or sensitivity and specificity of many commercially available tests, in particular many currently available blood-based tests. Methods, panels and other tests disclosed herein have the further benefit of being easily executed, such that an individual in need of gastrointestinal health evaluation test results is much more likely to have this test performed, rather than collecting a stool sample or having an invasive procedure such as a colonoscopy, for example. Panel accumulation levels are measured in a number of ways in various embodiments, for example through an antibody florescence binding assay or an ELISA assay, through mass spectroscopy analysis, through detection of florescence of an antibody set, or through alternate approaches to protein accumulation level quantification.

Panel accumulation levels are assessed through a number of approaches consistent with the disclosure herein. For example panel accumulation levels are compared to a positive control or negative control standard comprising at least one and up to 10, 100, or more than 100 standards of known colorectal health status, or to a model of advanced colorectal adenoma or colorectal cancer accumulation levels or of healthy accumulation levels, such that a prediction is made regarding an assayed individual's health status. Alternately or in combination, panel results are compared to a machine learning or other model trained on or built upon data obtained from known positive or known negative patient samples. In some cases, a panel assay result is accompanied by a recommendation regarding an intervention or an alternate verification of the panel assay results.

Accordingly, provided herein are biomarker panels and assays useful for the diagnosis and/or treatment of at least one of advanced colorectal adenoma and colorectal cancer.

Also provided herein are kits, comprising a computer readable medium described herein, and instructions for use of the computer readable medium.

A number of treatment regimens are contemplated herein and known to one of skill in the art, such as chemotherapy, administration of a biologic therapeutic agent, and surgical intervention such as low anterior resection or abdominoperineal resection, or ostomy.

### BRIEF DESCRIPTION OF THE DRAWINGS

FIG. 1 illustrates an AUC curve for a lead CRC panel having 0% No Calls.

FIG. 2 illustrates an AUC curve for a lead CRC panel having 15% No Calls.

FIG. 3 illustrates an AUC curve for a lead CRC panel having 20% No Calls.

FIG. 4 illustrates an AUC curve for a lead CRC panel having 25% No Calls.

FIG. 5 illustrates an AUC curve for a lead AA panel having 0% No Calls.

FIG. 6 illustrates an AUC curve for a lead AA panel having 10% No Calls.

FIG. 7 depicts discovery AUCs from randomly generated CRC panels (columns), as compared to the thin vertical line indicating the AUC for CRC panels as disclosed herein.

FIG. 8 depicts discovery AUCs from randomly generated AA panels (columns), as compared to the thin vertical line indicating the AUC for CRC panels as disclosed herein.

FIG. 9A depicts a correlation between biomarker level and overall model score for a first subset of CRC panel members.

FIG. 9B depicts a correlation between biomarker level and overall model score for a second subset of CRC panel members.

FIG. 9C depicts a correlation between biomarker level and overall model score for a third subset of CRC panel members.

FIG. 10 depicts a computer system consistent with the disclosure herein.

### DETAILED DESCRIPTION

Provided herein are biomarker panels, methods, compositions, kits, and systems for the non-invasive assessment of colorectal health, for example through the detection of at least one of advanced colorectal adenoma (“AA”) and colorectal cancer (“CRC”). Biomarker panels, methods, compositions, kits, and systems described herein are used to determine a likelihood that a subject has a colorectal condition such as at least one of an advanced colorectal adenoma and CRC through the noninvasive assay of a sample taken from circulating blood circulating blood. Some such biomarker panels are used noninvasively to detect a colorectal health issue such as colorectal cancer with a sensitivity of as much as 81% or greater, and a specificity of as much as 78% or greater. An exemplary CRC biomarker panel comprises the markers C9, CEA, DPP4, MIF, ORM1, PKM, SAA, and TFRC, and the non-protein biomarkers of age and gender of the individual providing the sample. Some such biomarker panels are used noninvasively to detect a colorectal health issue such as an advanced adenoma with a sensitivity of as much as 50% or greater, and a specificity of as much as 80% or greater. An exemplary biomarker panel relevant to advanced adenoma assessment comprises the markers CLU, CTSD, DPP4, GDF15, GSN, MIF, PKM, SERPINA1, SERPINA3, TFRC, and TIMP1, and also comprises obtaining the age of the individual.

Biomarker panels as disclosed herein share a property that sensitive, specific conclusions regarding an individual's colorectal health are made using protein level information derived from circulating blood, alone or in combination with other information such as an individual's age, gender, health history or other characteristics. A benefit of the present biomarker panels is that they provide a sensitive, specific colorectal health assessment using conveniently, noninvasively obtained samples. There is no need to rely upon data obtained from an intrusive abdominal assay such as a colonoscopy or a sigmoidoscopy, or from stool sample material. As a result compliance rates are substantially higher, and colorectal health issues are more easily recognized early in their progression, so that they may be more efficiently treated. Ultimately, the effect of this benefit is measured in lives saved, and is substantial.

Biomarker panels as disclosed herein are selected such that their predictive value as panels is substantially greater than the predictive value of their individual members. Panel members generally do not co-vary with one another, such that panel members provide independent contributions to the panel's overall health signal. Accordingly, a panel is able to substantially outperform the performance of any individual constituent indicative of an individual's colorectal health status, such that a commercially and medicinally relevant degree of confidence (such as sensitivity, specificity or sensitivity and specificity) is obtained. Thus, in the panels as disclosed herein, multiple panel members indicative of a health issue provide a much stronger signal than is found, for example in a panel wherein two or more members rise or fall in strict concert such that the signal derived therefrom is effectively a single signal, repeated twice. Accordingly, panels as disclosed herein are robust to variation in single constituent measurements. For example because panel members vary independently of one another, panels herein often indicate a health risk despite the fact that one or more than one individual members of the panel would not indicate that the health risk is present if measured alone. In some cases, panels herein indicate a health risk at a significant level of confidence despite the fact that no individual panel member indicates the health risk at a significant level of confidence on its own. In some cases, panels herein indicate a health risk at a significant level of confidence despite the fact that at least one individual member indicates at a significant level of confidence that the health risk is not present.

Biomarkers consistent with the panels herein comprise biological molecules that circulate in the bloodstream of an individual, such as proteins. Readily available information including demographic information such as individual's age or gender is also included in some cases. Physiological information including weight, height, body mass index, as well as other easily measured or obtained information is also eligible as a marker. In particular, some panels herein rely upon age, gender, or age and gender as biomarkers.

Common to many biomarkers herein is the ease with which they are assayed in an individual. Biomarkers herein are readily obtained by a blood draw from an artery or vein of an individual, or are obtained via interview or by simple biometric analysis. A benefit of the ease with which biomarkers herein are obtained is that invasive assays such as colonoscopy or sigmoidoscopy are not required for biomarker measurement. Similarly, stool samples are not required for biomarker determination. As a result, panel information as disclosed herein is often readily obtained through a blood draw in combination with a visit to a doctor's office. Compliance rates are accordingly substantially higher than are compliance rates for colorectal health assays involving stool samples or invasive procedures.

Exemplary panels disclosed herein comprise circulating proteins or fragments thereof that are recognizably or uniquely mapped to their parent protein, and in some cases comprise a readily obtained biomarker such as an individual's age.

### Panel Constituents

Some biomarker panels comprise some or all of the protein markers recited herein, subsets thereof or listed markers in combination with additional markers or biological parameters. A lead biomarker panel relevant to colorectal cancer assessment comprises at least 4 markers, up to the full list, alone or in combination with additional markers, said list selected from the following: C9, CEA, DPP4, MIF, ORM1, PKM, SAA, and TFRC, and also including age and gender as biomarkers. A lead biomarker panel relevant to advanced adenoma assessment comprises markers selected from the following: CLU, CTSD, DPP4, GDF15, GSN, MIF, PKM, SERPINA1, SERPINA3, TFRC, and TIMP1, and also including age of the individual as a biomarker. A lead biomarker panel, or a combination of biomarker panels having combined colorectal cancer and advanced adenoma assessment capabilities comprises biomarkers such as C9, CEA, ORM1, PKM, SAA, CLU, CTSD, DPP4, GDF15, GSN, MIF, SERPINA1, SERPINA3, TFRC, and TIMP1, and age and gender as biomarker, or a subset thereof optionally having at least one individual marker excluded or replaced with one or more markers.

Often, it is convenient or efficient to combine a CRC biomarker panel and an advanced adenoma panel into a single kit or a single biomarker panel. In these cases, one sees a kit comprising eleven biomarkers, or a subset or larger set thereof, including C9, CEA, ORM1, PKM, SAA, CLU, CTSD, DPP4, GDF15, GSN, MIF, SERPINA1, SERPINA3, TFRC, and TIMP1, of which C9, CEA, DPP4, MIF, ORM1, PKM, SAA, and TFRC or a subset or larger group comprising these markers is informative as to colorectal cancer status; CLU, CTSD, DPP4, GDF15, GSN, MIF, PKM, SERPINA1, SERPINA3, TFRC, and TIMP1, or a subset or larger group comprising these markers is informative as to advanced adenoma status; and C9, CEA, CLU, CTSD, DPP4, GDF15, GSN, MIF, ORM1, PKM, SAA, SERPINA1, SERPINA3, TFRC, and TIMP1, if included, is informative as to both colorectal cancer status and advanced adenoma status, particularly in combination with information regarding patient age and gender. Alternate and variant colorectal cancer biomarker panels are listed below.

Much like the panel discussed above, these panels, or subsets or additions, are used alone or in combination with the above-mentioned advanced adenoma panel, optionally using markers such as CLU, CTSD, DPP4, GDF15, GSN, MIF, PKM, SERPINA1, SERPINA3, TFRC, and TIMP1, to be indicative of advanced adenoma. An exemplary biomarker panel comprises at least 4 markers, up to the full list, alone or in combination with additional markers, said list selected from the following: C9, CEA, DPP4, MIF, ORM1, PKM, SAA, and TFRC, and also including individual age and gender.

Accordingly, disclosed herein are colorectal health assessment panels comprising the biomarkers mentioned above. Panels comprise at least 3, 4, 5, 6, 7, 8, 9, 10, 11, 12, o more than 12 of the biomarkers mentioned herein.

Similarly, disclosed herein are colorectal health assessment panels consisting of the biomarkers mentioned above. Panels comprise at least 3, 4, 5, 6, 7, 8, 9, 10, 11, 12, o more than 12 of the biomarkers mentioned herein.

### Biomarkers

In some cases, biomarker panels described herein comprise at least three biomarkers. The biomarkers are be selected from the group of identifiable polypeptides or fragments of the 17 biomarkers listed in Table 1. Any of the biomarkers described herein can be protein biomarkers. Furthermore, the group of biomarkers in this example can in some cases additionally comprise polypeptides with the characteristics found in Table 1.

Exemplary protein biomarkers and, when available, their human amino acid sequences, are listed in Table 1, below. Protein biomarkers comprise full length molecules of the polypeptide sequences of Table 1, as well as uniquely identifiable fragments of the polypeptide sequences of Table 1. Markers can be but do not need to be full length to be informative. In many cases, so long as a fragment is uniquely identifiable as being derived from or representing a polypeptide of Table 1, it is informative for purposes herein.

TABLE 1
Biomarkers and corresponding Descriptors

| No./Protein Name/ | |
|---|---|
| Protein Symbol and | Protein Sequence (N- to C-terminal single letter amino |
| Synonyms/Uniprot ID | acid sequence) or other Descriptor of Biomarker |
| No. 1/Alpha/1-acid | MALSWVLTVLSLLPLLEAQIPLCANLVPVPITNATLDQITGKWFYIASAFRNEE |
| glycoprotein 1/ | YNKSVQEIQATFFYFTPNKTEDTIFLREYQTRQDQCIYNTTYLNVQRENGTISR |
| A1AG1/A1AG/ORM1/ | YVGGQEHFAHLLILRDTKTYMLAFDVNDEKNWGLSVYADKPETTKEQLGEFYEA |
| P02763 | LDCLRIPKSDVVYTDWKKDKCEPLEKQHEKERKQEEGES |
| No. 2/Alpha-1 | MPSSVSWGILLLAGLCCLVPVSLAEDPQGDAAQKTDTSHHDQDHPTFNKITPNL |
| Antitrypsin/A1AT, | AEFAFSLYRQALHQSNSTNIFFSPVSIATAFAMLSLGTKADTHDEILEGLNFNL |
| PI, SERPINA 1/ | TEIPEAQIHEGFQELLRTLNQPDSQLQLTTGNGLFLSEGLKLVDKFLEDVKKLY |
| P01009 | HSEAFTVNFGDTEEAKKQINDYVEKGTQGKIVDLVKELDRDTVFALVNYIFFKG |
| | KWERPFEVKDTEEEDFHVDQVTTVKVPMMKRLGMFNIQHCKKLSSWVLLMKYLG |
| | NATAIFFLPDEGKLQHLENELTHDIITKFLENEDRRSASLHLPKLSITGTYDLK |
| | SVLGQLGITKVFSNGADLSGVTEEAPLKLSKAVHKAVLTIDEKGTEAAGAMFLE |
| | AIPMSIPPEVKFNKPFVFLMIEQNTKSPLFMGKVVNPTQK |
| No. 3/Alpha-1/ | MERMLPLLALGLLAAGFCPAVLCHPNSPLDEENLTQENQDRGTHVDLGLASANV |
| Antichymotrypsin/ | DFAFSLYKQLVLKAPDKNVIFSPLSISTALAFLSLGAHNTTLTEILKGLKFNLT |
| AACT, SERPINA 3/ | ETSEAEIHQSFQHLLRTLNQSSDELQLSMGNAMFVKEQLSLLDRFTEDAKRLYG |
| P01011 | SEAFATDFQDSAAAKKLINDYVKNGTRGKITDLIKDLDSQTMMVLVNYIFFKAK |
| | WEMPFDPQDTHQSRFYLSKKKWVMVPMMSLHHLTIPYFRDEELSCTVVELKYTG |
| | NASALFILPDQDKMEEVEAMLLPETLKRWRDSLEFREIGELYLPKFSISRDYNL |
| | NDILLQLGIEEAFTSKADLSGITGARNLAVSQVVHKAVLDVFEEGTEASAATAV |
| | KITLLSALVETRTIVRFNRPFLMIIVPTDTQNIFFMSKVTNPKQA |
| No. 4/Cathepsin D/ | MQPSSLLPLALCLLAAPASALVRIPLHKFTSIRRTMSEVGGSVEDLIAKGPVSK |
| CATD, CTSD, CPSD/ | YSQAVPAVTEGPIPEVLKNYMDAQYYGEIGIGTPPQCFTVVFDTGSSNLWVPSI |
| P07339 | HCKLLDIACWIHHKYNSDKSSTYVKNGTSFDIHYGSGSLSGYLSQDTVSVPCQS |
| | ASSASALGGVKVERQVFGEATKQPGITFIAAKFDGILGMAYPRISVNNVLPVFD |
| | NLMQQKLVDQNIFSFYLSRDPDAQPGGELMLGGTDSKYYKGSLSYLNVTRKAYW |
| | QVHLDQVEVASGLTLCKEGCEAIVDTGTSLMVGPVDEVRELQKAIGAVPLIQGE |
| | YMIPCEKVSTLPAITLKLGGKGYKLSPEDYTLKVSQAGKTLCLSGFMGMDIPPP |
| | SGPLWILGDVFIGRYYTVFDRDNNRVGFAEAARL |
| No. 5/Carcinoembry- | MESPSAPPHRWCIPWQRLLLTASLLTFWNPPTTAKLTIESTPFNVAEGKEVLLL |
| onic antigen- | VHNLPQHLFGYSWYKGERVDGNRQIIGYVIGTQQATPGPAYSGREIIYPNASLL |
| related cell | IQNIIQNDTGFYTLHVIKSDLVNEEATGQFRVYPELPKPSISSNNSKPVEDKDA |
| adhesion molecule | VAFTCEPETQDATYLWWVNNQSLPVSPRLQLSNGNRTLTLFNVTRNDTASYKCE |
| 3/CEA CAM5 (CEA)/ | TQNPVSARRSDSVILNVLYGPDAPTISPLNTSYRSGENLNLSCHAASNPPAQYS |
| P06731 | WFVNGTFQQSTQELFIPNITVNNSGSYTCQAHNSDTGLNRTTVTTITVYAEPPK |
| | PFITSNNSNPVEDEDAVALTCEPEIQNTTYLWWVNNQSLPVSPRLQLSNDNRTL |
| | TLLSVTRNDVGPYECGIQNKLSVDHSDPVILNVLYGPDDPTISPSYTYYRPGVN |
| | LSLSCHAASNPPAQYSWLIDGNIQQHTQELFISNITEKNSGLYTCQANNSASGH |
| | SRTTVKTITVSAELPKPSISSNNSKPVEDKDAVAFTCEPEAQNTTYLWWVNGQS |
| | LPVSPRLQLSNGNRTLTLFNVTRNDARAYVCGIQNSVSANRSDPVTLDVLYGPD |
| | TPIISPPDSSYLSGANLNLSCHSASNPSPQYSWRINGIPQQGTQVLFIAKITPN |
| | NNGTYACFVSNLATGRNNSIVKSITVSASGTSPGLSAGATVGIMIGVLVGVALI |
| No. 6/Clusterin/ | MMKTLLLFVGLLLTWESGQVLGDQTVSDNELQEMSNQGSKYVNKEIQNAVNGVK |
| CLUS, CLU, APOJ, | QIKTLIEKTNEERKTLLSNLEEAKKKEDALNETRESETKLKELPGVCNETMMAL |
| CLI, KUB1/P10909 | WEECKPCLKQTCMKFYARVCRSGSGLVGRQLEEFLNQSSPFYFWMNGDRIDSLL |
| | ENDRQQTHMLDVMQDHRSRASSIIDELFQDRFFTREPQDTYHYLPFSLPHRRPH |
| | FFFPKSRIVRSLMPFSPYEPLNFHAMFQPFLEMIHEAQQAMDIHFHSPAFQHPP |
| | TEFIREGDDDRTVCREIRHNSTGCLRMKDQCDKCREILSVDCSTNNPSQAKLRR |
| | ELDESLQVAERLTRKYNELLKSYQWKMLNTSSLLEQLNEQFNWVSRLANLTQGE |
| | DQYYLRVTTVASHTSDSDVPSGVTEVVVKLFDSDPITVTVPVEVSRKNPKFMET |
| | VAEKALQEYRKKHREE |
| No. 7/ | MSACRSFAVAICILEISILTAQYTTSYDPELTESSGSASHIDCRMSPWSEWSQC |
| Complement C9/C9, | DPCLRQMFRSRSIEVFGQFNGKRCTDAVGDRRQCVPTEPCEDAEDDCGNDFQCS |
| CO9/P02748 | TGRCIKMRLRCNGDNDCGDFSDEDDCESEPRPPCRDRVVEESELARTAGYGINI |
| | LGMDPLSTPFDNEFYNGLCNRDRDGNTLTYYRRPWNVASLIYETKGEKNFRTEH |
| | YEEQIEAFKSIIQEKTSNFNAAISLKFTPTETNKAEQCCEETASSISLHGKGSF |
| | RFSYSKNETYQLFLSYSSKKEKMFLHVKGEIHLGRFVMRNRDVVLTTTFVDDIK |
| | ALPTTYEKGEYFAFLETYGTHYSSSGSLGGLYELIYVLDKASMKRKGVELDKIK |
| | RCLGYHLDVSLAFSEISVGAEFNKDDCVKRGEGRAVNITSENLIDDVVSLIRGG |
| | TRKYAFELKEKLLRGTVIDVTDFVNWASSINDAPVLISQKLSPIYNLVPVKMKN |
| | AHLKKQNLERAIEDYINEFSVRKCHTCQNGGTVILMDGKCLCACPFKFEGIACE |
| | ISKQKISEGLPALEFPNEK |
| No. 8/Dipeptidyl | MKTPWKVLLGLLGAAALVTIITVPVVLLNKGTDDATADSRKTYTLTDYLKNTYR |
| peptidase 4/DPP4, | LKLYSLRWISDHEYLYKQENNILVFNAEYGNSVFLENSTFDEFGHSINDYSISP |
| DPPIV, ADCP2, | DGQFILLEYNYVKQWRHSYTASYDIYDLNKRQLITEERIPNNTQWVTWSPVGHK |
| CD26/P27487 | LAYVWNNDIYVKIEPNLPSYRITWTGKEDIIYNGITDWVYEEEVFSAYSALWWS |
| | PNGTFLAYAQFNDTEVPLIEYSFYSDESLQYPKTVRVPYPKAGAVNPTVKFFVV |
| | NTDSLSSVTNATSIQITAPASMLIGDHYLCDVTWATQERISLQWLRRIQNYSVM |
| | DICDYDESSGRWNCLVARQHIEMSTTGWVGRFRPSEPHFTLDGNSFYKIISNEE |
| | GYRHICYFQIDKKTDCTFITKGTWEVIGIEALTSDYLYYISNEYKGMPGGRNLY |
| | KIQLSDYTKVTCLSCELNPERCQYYSVSFSKEAKYYQLRCSGPGLPLYTLHSSV |
| | NDKGLRVLEDNSALDKMLQNVQMPSKKLDFIILNETKFWYQMILPPHFDKSKKY |
| | PLLLDVYAGPCSQKADTVFRLNWATYLASTENIIVASFDGRGSGYQGDKIMHAI |
| | NRRLGTFEVEDQIEAARQFSKMGFVDNKRIAIWGWSYGGTVTSMVLGSGSGVFK |
| | CGIAVAPVSRWEYYDSVYTERYMGLPTPEDNLDHYRNSTVMSRAENFKQVEYLL |
| | IHGTADDNVHFQQSAQISKALVDVGVDFQAMWYTDEDHGIASSTAHQHIYTHMS |
| | HFIKQCFSLP |
| No. 9/Gelsolin/ | MAPHRPAPALLCALSLALCALSLPVRAATASRGASQAGAPQGRVPEARPNSMVV |
| GELS, GSN/P06396 | EHPEFLKAGKEPGLQIWRVEKFDLVPVPTNLYGDFFTGDAYVILKTVQLRNGNL |
| | QYDLHYWLGNECSQDESGAAAIFTVQLDDYLNGRAVQHREVQGFESATFLGYFK |
| | SGLKYKKGGVASGFKHVVPNEVVVQRLFQVKGRRVVRATEVPVSWESFNNGDCF |
| | ILDLGNNIHQWCGSNSNRYERLKATQVSKGIRDNERSGRARVHVSEEGTEPEAM |
| | LQVLGPKPALPAGTEDTAKEDAANRKLAKLYKVSNGAGTMSVLVADENPFAQGA |
| | LKSEDCFILDHGKDGKIFVWKGKQANTEERKAALKTASDFITKMDYPKQTQVSV |
| | LPEGGETPLFKQFFKNWRDPDQTDGLGLSYLSSHIANCERVPFDAATLHTSTAM |
| | AAQHGMDDDGTGQKQIWRIEGSNKVPVDPATYGQFYGGDSYIILYNYRHGGRQG |
| | QIIYNWQGAQSTQDEVAASIALTAQLDEELGGTPVQSRVVQGKEPAHLMSLFGG |
| | KPMIIYKGGTSREGGQTAPASTRLFQVRANSAGATRAVEVLPKAGALNSNDAFV |
| | LKTPSAAYLWVGTGASEAEKTGAQELLRVLRAQPVQVAEGSEPDGFWEALGGKA |
| | AYRTSPRLKDKKMDAHPPRLFACSNKIGRFVIEEVPGELMQEDLATDDVMLLDT |
| | WDQVFVWVGKDSQEEEKTEALTSAKRYIETDPANRDRRTPITVVKQGFEPPSFV |
| | GWFLGWDDDYWSVDPLDRAMAELAA |
| No. 10/Macrophage | MPMFIVNTNVPRASVPDGFLSELTQQLAQATGKPPQYIAVHVVPDQLMAFGGSS |
| migration | EPCALCSLHSIGKIGGAQNRSYSKLLCGLLAERLRISPDRVYINYYDMNAANVG |
| inhibitory factor/ | WNNSTFA |
| MIF, GLIF, MMIF/ | |
| P14174 | |
| No. 11/Pyruvate | MSKPHSEAGTAFIQTQQLHAAMADTFLEHMCRLDIDSPPITARNTGIICTIGPA |
| kinase/PKM, OIP3, | SRSVETLKEMIKSGMNVARLNFSHGTHEYHAETIKNVRTATESGASDPILYRPV |
| PK2, PK3, PKM2/ | AVALDTKGPEIRTGLIKGSGTAEVELKKGATLKITLDNAYMEKCDENILWLDYK |
| P14618 | NICKVVEVGSKIYVDDGLISLQVKQKGADFLVTEVENGGSLGSKKGVNLPGAAV |
| | DLPAVSEKDIQDLKFGVEQDVDMVFASFIRKASDVHEVRKVLGEKGKNIKIISK |
| | IENHEGVRRFDEILEASDGIMVARGDLGIEIPAEKVFLAQKMMIGRCNRAGKPV |
| | ICATQMLESMIKKPRPTRAEGSDVANAVLDGADCIMLSGETAKGDYPLEAVRMQ |
| | HLIAREAEAAIYHLQLFEELRRLAPITSDPTEATAVGAVEASFKCCSGAIIVLT |
| | KSGRSAHQVARYRPRAPIIAVTRNPQTARQAHLYRGIFPVLCKDPVQEAWAEDV |
| | DLRVNFAMNVGKARGFFKKGDVVIVLTGWRPGSGFTNTMRVVPVP |
| No. 12/“SAA” Serum | >SAA1 |
| amyloid A-1 | MKLLTGLVFCSLVLGVSSRSFFSFLGEAFDGARDMWRAYSDMREANYIGSDKYF |
| protein/Serum | HARGNYDAAKRGPGGVWAAEAISDARENIQRFFGHGAEDSLADQAANEWGRSGK |
| Amyloid A-2 | DPNHFRPAGLPEKY |
| protein/SAA1. SAA2, | >SAA2 |
| SAA1/2/SAA2/4 | MKLLTGLVFCSLVLSVSSRSFFSFLGEAFDGARDMWRAYSDMREANYIGSDKYF |
| P0DJI8/P0DJI9. | HARGNYDAAKRGPGGAWAAEVISNARENIQRLTGRGAEDSLADQAANKWGRSGR |
| | DPNHFRPAGLPEKY |
| | Note that unlike the other markers, marker ‘SAA’ |
| | represents either or both of two closely related SAA |
| | proteins listed above. The proteins share 93% identity |
| | over their common 122 residue length. An ‘SAA’ |
| | measurement variously refers to SAA1, SAA2, or a |
| | combined measurement of SAA1 and SAA2. |
| No. 13/ | MAPFEPLASGILLLLWLIAPSRACTCVPPHPQTAFCNSDLVIRAKFVGTPEVNQ |
| Metalloproteinase | TTLYQRYEIKMTKNYKGFQALGDAADIRFVYTPAMESVCGYFHRSHNRSEEFLI |
| inhibitor 1/TIMP1, | AGKLQDGLLHITTCSFVAPWNSLSLAQRRGFTKTYTVGCEECTVFPCLSIPCKL |
| CLGI/P01033 | QSGTHCLWTDQLLQGSEKGFQSRHLACLPREPGLCTWQSLRSQIA |
| No. 14/Transferrin | MMDQARSAFSNLFGGEPLSYTRFSLARQVDGDNSHVEMKLAVDEEENADNNTKA |
| Receptor Protein 1/ | NVTKPKRCSGSICYGTIAVIVFFLIGFMIGYLGYCKGVEPKTECERLAGTESPV |
| TFRC/P02786 | REEPGEDFPAARRLYWDDLKRKLSEKLDSTDFTGTIKLLNENSYVPREAGSQKD |
| | ENLALYVENQFREFKLSKVWRDQHFVKIQVKDSAQNSVIIVDKNGRLVYLVENP |
| | GGYVAYSKAATVTGKLVHANFGTKKDFEDLYTPVNGSIVIVRAGKITFAEKVAN |
| | AESLNAIGVLIYMDQTKFPIVNAELSFFGHAHLGTGDPYTPGFPSFNHTQFPPS |
| | RSSGLPNIPVQTISRAAAEKLFGNMEGDCPSDWKTDSTCRMVTSESKNVKLTVS |
| | NVLKEIKILNIFGVIKGFVEPDHYVVVGAQRDAWGPGAAKSGVGTALLLKLAQM |
| | FSDMVLKDGFQPSRIIFASWSAGDFGSVGATEWLEGYLSSLHLKAFTYINLDKA |
| | VLGTSNFKVSASPLLYTLIEKTMQNVKHPVTGQFLYQDSNWASKVEKLTLDNAA |
| | FPFLAYSGIPAVSFCFCEDTDYPYLGTTMDTYKELIERIPELNKVARAAAEVAG |
| | QGVIKLTHDVELNLDYERYNSQLLSFVRDLNQYRADIKEMGLSLQWLYSARGDF |
| | FRATSRLTTDFGNAEKTDRFVMKKLNDRVMRVEYHFLSPYVSPKESPFRHVFWG |
| | SGSHTLPALLENLKLRKQNNGAFNETLFRNQLALATWTIQGAANALSGDVWDID |
| | NEF |
| No. 15/Growth/ | MPGQELRTVNGSQMLLVLLVLSWLPHGGALSLAEASRASFPGPSELHSEDSRFR |
| differentiation | ELRKRYEDLLTRLRANQSWEDSNTDLVPAPAVRILTPEVRLGSGGHLHLRISRA |
| factor 15/GDF15, | ALPEGLPEASRLHRALFRLSPTASRSWDVTRPLRRQLSLARPQAPALHLRLSPP |
| MIC1, PDF, PLAB, | PSQSDQLLAESSSARPQLELHLRPQAARGRRRARARNGDHCPLGPGRCCRLHTV |
| PTGFB/Q99988 | RASLEDLGWADWVLSPREVQVTMCIGACPSQFRAANMHAQIKTSLHRLKPDTVP |
| | APCCVPASYNPMVLIQKTDTGVSLQTYDDLLAKDCHCI |
| No. 16 | Patient Age |
| No. 17 | Patient Gender |

Biomarkers contemplated herein also include polypeptides having an amino acid sequence identical to a listed marker of Table 1 over a span of 8 residues, 9, residues, 10 residues, 20 residues, 50 residues, or alternately 5%, 10%, 20%, 30%, 40%, 50%, 60%, 70% 80% 90%, 95% or greater than 95% of the sequence of the biomarker. Variant or alternative forms of the biomarker include for example polypeptides encoded by any splice-variants of transcripts encoding the disclosed biomarkers. In certain cases the modified forms, fragments, or their corresponding RNA or DNA, may exhibit better discriminatory power in diagnosis than the full-length protein.

Biomarkers contemplated herein also include truncated forms or polypeptide fragments of any of the proteins described herein. Truncated forms or polypeptide fragments of a protein can include N-terminally deleted or truncated forms and C-terminally deleted or truncated forms. Truncated forms or fragments of a protein can include fragments arising by any mechanism, such as, without limitation, by alternative translation, exo- and/or endo-proteolysis and/or degradation, for example, by physical, chemical and/or enzymatic proteolysis. Without limitation, a biomarker may comprise a truncated or fragment of a protein, polypeptide or peptide may represent about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or 100% of the amino acid sequence of the protein.

Without limitation, a truncated or fragment of a protein may include a sequence of about 5-20 consecutive amino acids, or about 10-50 consecutive amino acids, or about 20-100 consecutive amino acids, or about 30-150 consecutive amino acids, or about 50-500 consecutive amino acid residues of the corresponding full length protein.

In some instances, a fragment is N-terminally and/or C-terminally truncated by between 1 and about 20 amino acids, such as, for example, by between 1 and about 15 amino acids, or by between 1 and about 10 amino acids, or by between 1 and about 5 amino acids, compared to the corresponding mature, full-length protein or its soluble or plasma circulating form.

Any protein biomarker of the present disclosure such as a peptide, polypeptide or protein and fragments thereof may also encompass modified forms of said marker, peptide, polypeptide or protein and fragments such as bearing post-expression modifications including but not limited to, modifications such as phosphorylation, glycosylation, lipidation, methylation, selenocystine modification, cysteinylation, sulphonation, glutathionylation, acetylation, oxidation of methionine to methionine sulphoxide or methionine sulphone, and the like.

In some instances, a fragmented protein is N-terminally and/or C-terminally truncated. Such fragmented protein can comprise one or more, or all transitional ions of the N-terminally (a, b, c-ion) and/or C-terminally (x, y, z-ion) truncated protein or peptide. Exemplary human markers, nucleic acids, proteins or polypeptides as taught herein are as annotated under NCBI Genbank (accessible at the website ncbi.nlm.nih.gov) or Swissprot/Uniprot (accessible at the website uniprot.org) accession numbers. In some instances said sequences are of precursors (for example, preproteins) of the of markers, nucleic acids, proteins or polypeptides as taught herein and may include parts which are processed away from mature molecules. In some instances although only one or more isoforms is disclosed, all isoforms of the sequences are intended.

Antibodies for the detection of the biomarkers listed herein are commercially available. A partial list of sources for reagents useful for the assay of biomarkers herein is presented in Table 2 below.

TABLE 2
Reagent Sources

| Abbrev. | ELISA Kit Vendor | Assay Reference | Reference Vendor | Plasma Dilution |
|---|---|---|---|---|
| A1AT | Genway Biotech, San Diego, CA | Native protein | MyBiosource, San Diego, CA | 1:240,000 |
| A1AG1 | R&D Systems, Minneapolis, MN | Native protein | BioVendor, Asheville, NC | 1:20,000 |
| AACT | Genway Biotech, San Diego, CA | Native protein | MyBiosource, San Diego, CA | 1:10,000 |
| ANXA1 | Cloud Clone, Wuhan, PRC | Recombinant protein | Origene, Rockville, MD | 1:8,000 |
| APOA1 | Cusabio, Wuhan, PRC | Native protein | MyBiosource, San Diego, CA | 1:800 |
| CRP | BioVendor, Asheville, NC | Recombinant protein | R&D Systems, Minneapolis, MN | 1:1,000 |
| CAH1 | Cloud Clone, Wuhan, PRC | Recombinant protein | MyBiosource, San Diego, CA | 1:32 |
| CEA | IBL International, Toronto, ON | Native protein | Origene, Rockville, MD | 1:1 |
| CATD | AbCam, Cambridge, MA | Native protein | Novus Biologicals, Littleton, CA | 1:250 |
| CLUS | BioVendor, Asheville, NC | Native protein | MyBiosource, San Diego, CA | 1:3,000 |
| CO3 | Abnova, Taipei, Teawan | Native protein | MyBiosource, San Diego, CA | 1:250 |
| CO9 | AssayPro, St. Charles, MO | Native protein | MyBiosource, San Diego, CA | 1:20,000 |
| DPP4 | Cloud Clone, Wuhan, PRC | Native protein | BioVendor, Asheville, NC | 1:2,000 |
| FGB | Cloud Clone, Wuhan, PRC | Recombinant protein | Antibodies Online, Atlanta, GA | 1:8,000 |
| FIBG | Cloud Clone, Wuhan, PRC | Native protein | MyBiosource, San Diego, CA | 1:8,000 |
| GELS | Cloud Clone, Wuhan, PRC | Recombinant protein | Origene, Rockville, MD | 1:100 |
| GARS | Cloud Clone, Wuhan, PRC | Recombinant protein | Novus Biologicals, Littleton, CA | 1:40 |
| GDF15 | R&D Systems, Minneapolis, MN | Native protein | Abcam, Cambridge, MA | 1:8 |
| HPT | AssayPro, St. Charles, MO | Recombinant protein | Origene, Rockville, MD | 1:2,000 |
| MIF | R&D Systems, Minneapolis, MN | Recombinant protein | MyBiosource, San Diego, CA | 1:10 |
| OSTP | R&D Systems, Minneapolis, MN | Recombinant protein | Origene, Rockville, MD | 1:20 |
| PSGL | IBL America, Minneapolis, MN | Recombinant protein | Life Technologies, Camarillo, CA | 1:30 |
| PRDX1 | Cloud Clone, Wuhan, PRC | Recombinant protein | MyBiosource, San Diego, CA | 1:100 |
| SBP1 | Cloud Clone, Wuhan, PRC | Recombinant protein | Origene, Rockville, MD | 1:16 |
| SEPR | R&D Systems, Minneapolis, MN | Recombinant protein | Origene, Rockville, MD | 1:40 |
| SAA1 | Life Technologies, Camarillo, CA | Recombinant protein | Origene, Rockville, MD | 1:240 |
| TIMP1 | R&D Systems, Minneapolis, MN | Recombinant protein | Life Technologies, Camarillo, CA | 1:100 |
| TFRC | Cloud Clone, Wuhan, PRC | Native protein | MyBiosource, San Diego, CA | 1:250 |
| TFF3 | R&D Systems, Minneapolis, MN | Recombinant protein | R&D Systems, Minneapolis, MN | 1:50 |
| PKM2 | ScheBo, Giessen, GER | Recombinant protein | Origene, Rockville, MD | 1:100 |

For a given biomarker panel recited herein, variant biomarker panels differing in one or more than one constituent are also contemplated. Thus, turning to a lead CRC panel C9, CEA, DPP4, MIF, ORM1, PKM, SAA, and TFRC, and also including individual age and gender, as an example, a number of related panels are disclosed. For this and other panels disclosed herein, variants are contemplated comprising at least 8, at least 7, at least 6, at least 5, at least 4, at least 3, or at least 2 of the biomarker constituents of a recited biomarker panel.

Exemplary CRC panels consistent with the disclosure herein are listed in Table 3. Also disclosed are panels comprising the markers listed in entries of Table 3.

TABLE 3
CRC biomarker panel constituents

| Ref | CRC Protein Biomarker | Demographics | Features |
|---|---|---|---|
| 1 | C9, CEA, DPP4, MIF, ORM1, PKM, SAA, TFRC | Age and Gender | 10 |
| 2 | C9, CEA, DPP4, MIF, ORM1, PKM, SAA | Age and Gender | 9 |
| 3 | C9, CEA, DPP4, MIF, ORM1, PKM, TFRC | Age and Gender | 9 |
| 4 | C9, CEA, DPP4, MIF, ORM1, SAA, TFRC | Age and Gender | 9 |
| 5 | C9, CEA, DPP4, MIF, PKM, SAA, TFRC | Age and Gender | 9 |
| 6 | C9, CEA, DPP4, ORM1, PKM, SAA, TFRC | Age and Gender | 9 |
| 7 | C9, CEA, MIF, ORM1, PKM, SAA, TFRC | Age and Gender | 9 |
| 8 | C9, DPP4, MIF, ORM1, PKM, SAA, TFRC | Age and Gender | 9 |
| 9 | CEA, DPP4, MIF, ORM1, PKM, SAA, TFRC | Age and Gender | 9 |
| 10 | C9, CEA, DPP4, MIF, ORM1, PKM, SAA, TFRC | Age | 9 |
| 11 | C9, CEA, DPP4, MIF, ORM1, PKM, SAA, TFRC | Gender | 9 |
| 12 | C9, CEA, DPP4, MIF, ORM1, PKM | Age and Gender | 8 |
| 13 | C9, CEA, DPP4, MIF, ORM1, SAA | Age and Gender | 8 |
| 14 | C9, CEA, DPP4, MIF, ORM1, TFRC | Age and Gender | 8 |
| 15 | C9, CEA, DPP4, MIF, PKM, SAA | Age and Gender | 8 |
| 16 | C9, CEA, DPP4, MIF, PKM, TFRC | Age and Gender | 8 |
| 17 | C9, CEA, DPP4, MIF, SAA, TFRC | Age and Gender | 8 |
| 18 | C9, CEA, DPP4, ORM1, PKM, SAA | Age and Gender | 8 |
| 19 | C9, CEA, DPP4, ORM1, PKM, TFRC | Age and Gender | 8 |
| 20 | C9, CEA, DPP4, ORM1, SAA, TFRC | Age and Gender | 8 |
| 21 | C9, CEA, DPP4, PKM, SAA, TFRC | Age and Gender | 8 |
| 22 | C9, CEA, MIF, ORM1, PKM, SAA | Age and Gender | 8 |
| 23 | C9, CEA, MIF, ORM1, PKM, TFRC | Age and Gender | 8 |
| 24 | C9, CEA, MIF, ORM1, SAA, TFRC | Age and Gender | 8 |
| 25 | C9, CEA, MIF, PKM, SAA, TFRC | Age and Gender | 8 |
| 26 | C9, CEA, ORM1, PKM, SAA, TFRC | Age and Gender | 8 |
| 27 | C9, DPP4, MIF, ORM1, PKM, SAA | Age and Gender | 8 |
| 28 | C9, DPP4, MIF, ORM1, PKM, TFRC | Age and Gender | 8 |
| 29 | C9, DPP4, MIF, ORM1, SAA, TFRC | Age and Gender | 8 |
| 30 | C9, DPP4, MIF, PKM, SAA, TFRC | Age and Gender | 8 |
| 31 | C9, DPP4, ORM1, PKM, SAA, TFRC | Age and Gender | 8 |
| 32 | C9, MIF, ORM1, PKM, SAA, TFRC | Age and Gender | 8 |
| 33 | CEA, DPP4, MIF, ORM1, PKM, SAA | Age and Gender | 8 |
| 34 | CEA, DPP4, MIF, ORM1, PKM, TFRC | Age and Gender | 8 |
| 35 | CEA, DPP4, MIF, ORM1, SAA, TFRC | Age and Gender | 8 |
| 36 | CEA, DPP4, MIF, PKM, SAA, TFRC | Age and Gender | 8 |
| 37 | CEA, DPP4, ORM1, PKM, SAA, TFRC | Age and Gender | 8 |
| 38 | CEA, MIF, ORM1, PKM, SAA, TFRC | Age and Gender | 8 |
| 39 | DPP4, MIF, ORM1, PKM, SAA, TFRC | Age and Gender | 8 |
| 40 | C9, CEA, DPP4, MIF, ORM1, PKM, SAA | Age | 8 |
| 41 | C9, CEA, DPP4, MIF, ORM1, PKM, TFRC | Age | 8 |
| 42 | C9, CEA, DPP4, MIF, ORM1, SAA, TFRC | Age | 8 |
| 43 | C9, CEA, DPP4, MIF, PKM, SAA, TFRC | Age | 8 |
| 44 | C9, CEA, DPP4, ORM1, PKM, SAA, TFRC | Age | 8 |
| 45 | C9, CEA, MIF, ORM1, PKM, SAA, TFRC | Age | 8 |
| 46 | C9, DPP4, MIF, ORM1, PKM, SAA, TFRC | Age | 8 |
| 47 | CEA, DPP4, MIF, ORM1, PKM, SAA, TFRC | Age | 8 |
| 48 | C9, CEA, DPP4, MIF, ORM1, PKM, SAA | Gender | 8 |
| 49 | C9, CEA, DPP4, MIF, ORM1, PKM, TFRC | Gender | 8 |
| 50 | C9, CEA, DPP4, MIF, ORM1, SAA, TFRC | Gender | 8 |
| 51 | C9, CEA, DPP4, MIF, PKM, SAA, TFRC | Gender | 8 |
| 52 | C9, CEA, DPP4, ORM1, PKM, SAA, TFRC | Gender | 8 |
| 53 | C9, CEA, MIF, ORM1, PKM, SAA, TFRC | Gender | 8 |
| 54 | C9, DPP4, MIF, ORM1, PKM, SAA, TFRC | Gender | 8 |
| 55 | CEA, DPP4, MIF, ORM1, PKM, SAA, TFRC | Gender | 8 |
| 56 | C9, CEA, DPP4, MIF, ORM1, PKM, SAA, TFRC | NONE | 8 |
| 57 | C9, CEA, DPP4, MIF, ORM1 | Age and Gender | 7 |
| 58 | C9, CEA, DPP4, MIF, PKM | Age and Gender | 7 |
| 59 | C9, CEA, DPP4, MIF, SAA | Age and Gender | 7 |
| 60 | C9, CEA, DPP4, MIF, TFRC | Age and Gender | 7 |
| 61 | C9, CEA, DPP4, ORM1, PKM | Age and Gender | 7 |
| 62 | C9, CEA, DPP4, ORM1, SAA | Age and Gender | 7 |
| 63 | C9, CEA, DPP4, ORM1, TFRC | Age and Gender | 7 |
| 64 | C9, CEA, DPP4, PKM, SAA | Age and Gender | 7 |
| 65 | C9, CEA, DPP4, PKM, TFRC | Age and Gender | 7 |
| 66 | C9, CEA, DPP4, SAA, TFRC | Age and Gender | 7 |
| 67 | C9, CEA, MIF, ORM1, PKM | Age and Gender | 7 |
| 68 | C9, CEA, MIF, ORM1, SAA | Age and Gender | 7 |
| 69 | C9, CEA, MIF, ORM1, TFRC | Age and Gender | 7 |
| 70 | C9, CEA, MIF, PKM, SAA | Age and Gender | 7 |
| 71 | C9, CEA, MIF, PKM, TFRC | Age and Gender | 7 |
| 72 | C9, CEA, MIF, SAA, TFRC | Age and Gender | 7 |
| 73 | C9, CEA, ORM1, PKM, SAA | Age and Gender | 7 |
| 74 | C9, CEA, ORM1, PKM, TFRC | Age and Gender | 7 |
| 75 | C9, CEA, ORM1, SAA, TFRC | Age and Gender | 7 |
| 76 | C9, CEA, PKM, SAA, TFRC | Age and Gender | 7 |
| 77 | C9, DPP4, MIF, ORM1, PKM | Age and Gender | 7 |
| 78 | C9, DPP4, MIF, ORM1, SAA | Age and Gender | 7 |
| 79 | C9, DPP4, MIF, ORM1, TFRC | Age and Gender | 7 |
| 80 | C9, DPP4, MIF, PKM, SAA | Age and Gender | 7 |
| 81 | C9, DPP4, MIF, PKM, TFRC | Age and Gender | 7 |
| 82 | C9, DPP4, MIF, SAA, TFRC | Age and Gender | 7 |
| 83 | C9, DPP4, ORM1, PKM, SAA | Age and Gender | 7 |
| 84 | C9, DPP4, ORM1, PKM, TFRC | Age and Gender | 7 |
| 85 | C9, DPP4, ORM1, SAA, TFRC | Age and Gender | 7 |
| 86 | C9, DPP4, PKM, SAA, TFRC | Age and Gender | 7 |
| 87 | C9, MIF, ORM1, PKM, SAA | Age and Gender | 7 |
| 88 | C9, MIF, ORM1, PKM, TFRC | Age and Gender | 7 |
| 89 | C9, MIF, ORM1, SAA, TFRC | Age and Gender | 7 |
| 90 | C9, MIF, PKM, SAA, TFRC | Age and Gender | 7 |
| 91 | C9, ORM1, PKM, SAA, TFRC | Age and Gender | 7 |
| 92 | CEA, DPP4, MIF, ORM1, PKM | Age and Gender | 7 |
| 93 | CEA, DPP4, MIF, ORM1, SAA | Age and Gender | 7 |
| 94 | CEA, DPP4, MIF, ORM1, TFRC | Age and Gender | 7 |
| 95 | CEA, DPP4, MIF, PKM, SAA | Age and Gender | 7 |
| 96 | CEA, DPP4, MIF, PKM, TFRC | Age and Gender | 7 |
| 97 | CEA, DPP4, MIF, SAA, TFRC | Age and Gender | 7 |
| 98 | CEA, DPP4, ORM1, PKM, SAA | Age and Gender | 7 |
| 99 | CEA, DPP4, ORM1, PKM, TFRC | Age and Gender | 7 |
| 100 | CEA, DPP4, ORM1, SAA, TFRC | Age and Gender | 7 |
| 101 | CEA, DPP4, PKM, SAA, TFRC | Age and Gender | 7 |
| 102 | CEA, MIF, ORM1, PKM, SAA | Age and Gender | 7 |
| 103 | CEA, MIF, ORM1, PKM, TFRC | Age and Gender | 7 |
| 104 | CEA, MIF, ORM1, SAA, TFRC | Age and Gender | 7 |
| 105 | CEA, MIF, PKM, SAA, TFRC | Age and Gender | 7 |
| 106 | CEA, ORM1, PKM, SAA, TFRC | Age and Gender | 7 |
| 107 | DPP4, MIF, ORM1, PKM, SAA | Age and Gender | 7 |
| 108 | DPP4, MIF, ORM1, PKM, TFRC | Age and Gender | 7 |
| 109 | DPP4, MIF, ORM1, SAA, TFRC | Age and Gender | 7 |
| 110 | DPP4, MIF, PKM, SAA, TFRC | Age and Gender | 7 |
| 111 | DPP4, ORM1, PKM, SAA, TFRC | Age and Gender | 7 |
| 112 | MIF, ORM1, PKM, SAA, TFRC | Age and Gender | 7 |
| 113 | C9, CEA, DPP4, MIF, ORM1, PKM | Age | 7 |
| 114 | C9, CEA, DPP4, MIF, ORM1, SAA | Age | 7 |
| 115 | C9, CEA, DPP4, MIF, ORM1, TFRC | Age | 7 |
| 116 | C9, CEA, DPP4, MIF, PKM, SAA | Age | 7 |
| 117 | C9, CEA, DPP4, MIF, PKM, TFRC | Age | 7 |
| 118 | C9, CEA, DPP4, MIF, SAA, TFRC | Age | 7 |
| 119 | C9, CEA, DPP4, ORM1, PKM, SAA | Age | 7 |
| 120 | C9, CEA, DPP4, ORM1, PKM, TFRC | Age | 7 |
| 121 | C9, CEA, DPP4, ORM1, SAA, TFRC | Age | 7 |
| 122 | C9, CEA, DPP4, PKM, SAA, TFRC | Age | 7 |
| 123 | C9, CEA, MIF, ORM1, PKM, SAA | Age | 7 |
| 124 | C9, CEA, MIF, ORM1, PKM, TFRC | Age | 7 |
| 125 | C9, CEA, MIF, ORM1, SAA, TFRC | Age | 7 |
| 126 | C9, CEA, MIF, PKM, SAA, TFRC | Age | 7 |
| 127 | C9, CEA, ORM1, PKM, SAA, TFRC | Age | 7 |
| 128 | C9, DPP4, MIF, ORM1, PKM, SAA | Age | 7 |
| 129 | C9, DPP4, MIF, ORM1, PKM, TFRC | Age | 7 |
| 130 | C9, DPP4, MIF, ORM1, SAA, TFRC | Age | 7 |
| 131 | C9, DPP4, MIF, PKM, SAA, TFRC | Age | 7 |
| 132 | C9, DPP4, ORM1, PKM, SAA, TFRC | Age | 7 |
| 133 | C9, MIF, ORM1, PKM, SAA, TFRC | Age | 7 |
| 134 | CEA, DPP4, MIF, ORM1, PKM, SAA | Age | 7 |
| 135 | CEA, DPP4, MIF, ORM1, PKM, TFRC | Age | 7 |
| 136 | CEA, DPP4, MIF, ORM1, SAA, TFRC | Age | 7 |
| 137 | CEA, DPP4, MIF, PKM, SAA, TFRC | Age | 7 |
| 138 | CEA, DPP4, ORM1, PKM, SAA, TFRC | Age | 7 |
| 139 | CEA, MIF, ORM1, PKM, SAA, TFRC | Age | 7 |
| 140 | DPP4, MIF, ORM1, PKM, SAA, TFRC | Age | 7 |
| 141 | C9, CEA, DPP4, MIF, ORM1, PKM | Gender | 7 |
| 142 | C9, CEA, DPP4, MIF, ORM1, SAA | Gender | 7 |
| 143 | C9, CEA, DPP4, MIF, ORM1, TFRC | Gender | 7 |
| 144 | C9, CEA, DPP4, MIF, PKM, SAA | Gender | 7 |
| 145 | C9, CEA, DPP4, MIF, PKM, TFRC | Gender | 7 |
| 146 | C9, CEA, DPP4, MIF, SAA, TFRC | Gender | 7 |
| 147 | C9, CEA, DPP4, ORM1, PKM, SAA | Gender | 7 |
| 148 | C9, CEA, DPP4, ORM1, PKM, TFRC | Gender | 7 |
| 149 | C9, CEA, DPP4, ORM1, SAA, TFRC | Gender | 7 |
| 150 | C9, CEA, DPP4, PKM, SAA, TFRC | Gender | 7 |
| 151 | C9, CEA, MIF, ORM1, PKM, SAA | Gender | 7 |
| 152 | C9, CEA, MIF, ORM1, PKM, TFRC | Gender | 7 |
| 153 | C9, CEA, MIF, ORM1, SAA, TFRC | Gender | 7 |
| 154 | C9, CEA, MIF, PKM, SAA, TFRC | Gender | 7 |
| 155 | C9, CEA, ORM1, PKM, SAA, TFRC | Gender | 7 |
| 156 | C9, DPP4, MIF, ORM1, PKM, SAA | Gender | 7 |
| 157 | C9, DPP4, MIF, ORM1, PKM, TFRC | Gender | 7 |
| 158 | C9, DPP4, MIF, ORM1, SAA, TFRC | Gender | 7 |
| 159 | C9, DPP4, MIF, PKM, SAA, TFRC | Gender | 7 |
| 160 | C9, DPP4, ORM1, PKM, SAA, TFRC | Gender | 7 |
| 161 | C9, MIF, ORM1, PKM, SAA, TFRC | Gender | 7 |
| 162 | CEA, DPP4, MIF, ORM1, PKM, SAA | Gender | 7 |
| 163 | CEA, DPP4, MIF, ORM1, PKM, TFRC | Gender | 7 |
| 164 | CEA, DPP4, MIF, ORM1, SAA, TFRC | Gender | 7 |
| 165 | CEA, DPP4, MIF, PKM, SAA, TFRC | Gender | 7 |
| 166 | CEA, DPP4, ORM1, PKM, SAA, TFRC | Gender | 7 |
| 167 | CEA, MIF, ORM1, PKM, SAA, TFRC | Gender | 7 |
| 168 | DPP4, MIF, ORM1, PKM, SAA, TFRC | Gender | 7 |
| 169 | C9, CEA, DPP4, MIF, ORM1, PKM, SAA | NONE | 7 |
| 170 | C9, CEA, DPP4, MIF, ORM1, PKM, TFRC | NONE | 7 |
| 171 | C9, CEA, DPP4, MIF, ORM1, SAA, TFRC | NONE | 7 |
| 172 | C9, CEA, DPP4, MIF, PKM, SAA, TFRC | NONE | 7 |
| 173 | C9, CEA, DPP4, ORM1, PKM, SAA, TFRC | NONE | 7 |
| 174 | C9, CEA, MIF, ORM1, PKM, SAA, TFRC | NONE | 7 |
| 175 | C9, DPP4, MIF, ORM1, PKM, SAA, TFRC | NONE | 7 |
| 176 | CEA, DPP4, MIF, ORM1, PKM, SAA, TFRC | NONE | 7 |
| 177 | C9, CEA, DPP4, MIF | Age and Gender | 6 |
| 178 | C9, CEA, DPP4, ORM1 | Age and Gender | 6 |
| 179 | C9, CEA, DPP4, PKM | Age and Gender | 6 |
| 180 | C9, CEA, DPP4, SAA | Age and Gender | 6 |
| 181 | C9, CEA, DPP4, TFRC | Age and Gender | 6 |
| 182 | C9, CEA, MIF, ORM1 | Age and Gender | 6 |
| 183 | C9, CEA, MIF, PKM | Age and Gender | 6 |
| 184 | C9, CEA, MIF, SAA | Age and Gender | 6 |
| 185 | C9, CEA, MIF, TFRC | Age and Gender | 6 |
| 186 | C9, CEA, ORM1, PKM | Age and Gender | 6 |
| 187 | C9, CEA, ORM1, SAA | Age and Gender | 6 |
| 188 | C9, CEA, ORM1, TFRC | Age and Gender | 6 |
| 189 | C9, CEA, PKM, SAA | Age and Gender | 6 |
| 190 | C9, CEA, PKM, TFRC | Age and Gender | 6 |
| 191 | C9, CEA, SAA, TFRC | Age and Gender | 6 |
| 192 | C9, DPP4, MIF, ORM1 | Age and Gender | 6 |
| 193 | C9, DPP4, MIF, PKM | Age and Gender | 6 |
| 194 | C9, DPP4, MIF, SAA | Age and Gender | 6 |
| 195 | C9, DPP4, MIF, TFRC | Age and Gender | 6 |
| 196 | C9, DPP4, ORM1, PKM | Age and Gender | 6 |
| 197 | C9, DPP4, ORM1, SAA | Age and Gender | 6 |
| 198 | C9, DPP4, ORM1, TFRC | Age and Gender | 6 |
| 199 | C9, DPP4, PKM, SAA | Age and Gender | 6 |
| 200 | C9, DPP4, PKM, TFRC | Age and Gender | 6 |
| 201 | C9, DPP4, SAA, TFRC | Age and Gender | 6 |
| 202 | C9, MIF, ORM1, PKM | Age and Gender | 6 |
| 203 | C9, MIF, ORM1, SAA | Age and Gender | 6 |
| 204 | C9, MIF, ORM1, TFRC | Age and Gender | 6 |
| 205 | C9, MIF, PKM, SAA | Age and Gender | 6 |
| 206 | C9, MIF, PKM, TFRC | Age and Gender | 6 |
| 207 | C9, MIF, SAA, TFRC | Age and Gender | 6 |
| 208 | C9, ORM1, PKM, SAA | Age and Gender | 6 |
| 209 | C9, ORM1, PKM, TFRC | Age and Gender | 6 |
| 210 | C9, ORM1, SAA, TFRC | Age and Gender | 6 |
| 211 | C9, PKM, SAA, TFRC | Age and Gender | 6 |
| 212 | CEA, DPP4, MIF, ORM1 | Age and Gender | 6 |
| 213 | CEA, DPP4, MIF, PKM | Age and Gender | 6 |
| 214 | CEA, DPP4, MIF, SAA | Age and Gender | 6 |
| 215 | CEA, DPP4, MIF, TFRC | Age and Gender | 6 |
| 216 | CEA, DPP4, ORM1, PKM | Age and Gender | 6 |
| 217 | CEA, DPP4, ORM1, SAA | Age and Gender | 6 |
| 218 | CEA, DPP4, ORM1, TFRC | Age and Gender | 6 |
| 219 | CEA, DPP4, PKM, SAA | Age and Gender | 6 |
| 220 | CEA, DPP4, PKM, TFRC | Age and Gender | 6 |
| 221 | CEA, DPP4, SAA, TFRC | Age and Gender | 6 |
| 222 | CEA, MIF, ORM1, PKM | Age and Gender | 6 |
| 223 | CEA, MIF, ORM1, SAA | Age and Gender | 6 |
| 224 | CEA, MIF, ORM1, TFRC | Age and Gender | 6 |
| 225 | CEA, MIF, PKM, SAA | Age and Gender | 6 |
| 226 | CEA, MIF, PKM, TFRC | Age and Gender | 6 |
| 227 | CEA, MIF, SAA, TFRC | Age and Gender | 6 |
| 228 | CEA, ORM1, PKM, SAA | Age and Gender | 6 |
| 229 | CEA, ORM1, PKM, TFRC | Age and Gender | 6 |
| 230 | CEA, ORM1, SAA, TFRC | Age and Gender | 6 |
| 231 | CEA, PKM, SAA, TFRC | Age and Gender | 6 |
| 232 | DPP4, MIF, ORM1, PKM | Age and Gender | 6 |
| 233 | DPP4, MIF, ORM1, SAA | Age and Gender | 6 |
| 234 | DPP4, MIF, ORM1, TFRC | Age and Gender | 6 |
| 235 | DPP4, MIF, PKM, SAA | Age and Gender | 6 |
| 236 | DPP4, MIF, PKM, TFRC | Age and Gender | 6 |
| 237 | DPP4, MIF, SAA, TFRC | Age and Gender | 6 |
| 238 | DPP4, ORM1, PKM, SAA | Age and Gender | 6 |
| 239 | DPP4, ORM1, PKM, TFRC | Age and Gender | 6 |
| 240 | DPP4, ORM1, SAA, TFRC | Age and Gender | 6 |
| 241 | DPP4, PKM, SAA, TFRC | Age and Gender | 6 |
| 242 | MIF, ORM1, PKM, SAA | Age and Gender | 6 |
| 243 | MIF, ORM1, PKM, TFRC | Age and Gender | 6 |
| 244 | MIF, ORM1, SAA, TFRC | Age and Gender | 6 |
| 245 | MIF, PKM, SAA, TFRC | Age and Gender | 6 |
| 246 | ORM1, PKM, SAA, TFRC | Age and Gender | 6 |
| 247 | C9, CEA, DPP4, MIF, ORM1 | Age | 6 |
| 248 | C9, CEA, DPP4, MIF, PKM | Age | 6 |
| 249 | C9, CEA, DPP4, MIF, SAA | Age | 6 |
| 250 | C9, CEA, DPP4, MIF, TFRC | Age | 6 |
| 251 | C9, CEA, DPP4, ORM1, PKM | Age | 6 |
| 252 | C9, CEA, DPP4, ORM1, SAA | Age | 6 |
| 253 | C9, CEA, DPP4, ORM1, TFRC | Age | 6 |
| 254 | C9, CEA, DPP4, PKM, SAA | Age | 6 |
| 255 | C9, CEA, DPP4, PKM, TFRC | Age | 6 |
| 256 | C9, CEA, DPP4, SAA, TFRC | Age | 6 |
| 257 | C9, CEA, MIF, ORM1, PKM | Age | 6 |
| 258 | C9, CEA, MIF, ORM1, SAA | Age | 6 |
| 259 | C9, CEA, MIF, ORM1, TFRC | Age | 6 |
| 260 | C9, CEA, MIF, PKM, SAA | Age | 6 |
| 261 | C9, CEA, MIF, PKM, TFRC | Age | 6 |
| 262 | C9, CEA, MIF, SAA, TFRC | Age | 6 |
| 263 | C9, CEA, ORM1, PKM, SAA | Age | 6 |
| 264 | C9, CEA, ORM1, PKM, TFRC | Age | 6 |
| 265 | C9, CEA, ORM1, SAA, TFRC | Age | 6 |
| 266 | C9, CEA, PKM, SAA, TFRC | Age | 6 |
| 267 | C9, DPP4, MIF, ORM1, PKM | Age | 6 |
| 268 | C9, DPP4, MIF, ORM1, SAA | Age | 6 |
| 269 | C9, DPP4, MIF, ORM1, TFRC | Age | 6 |
| 270 | C9, DPP4, MIF, PKM, SAA | Age | 6 |
| 271 | C9, DPP4, MIF, PKM, TFRC | Age | 6 |
| 272 | C9, DPP4, MIF, SAA, TFRC | Age | 6 |
| 273 | C9, DPP4, ORM1, PKM, SAA | Age | 6 |
| 274 | C9, DPP4, ORM1, PKM, TFRC | Age | 6 |
| 275 | C9, DPP4, ORM1, SAA, TFRC | Age | 6 |
| 276 | C9, DPP4, PKM, SAA, TFRC | Age | 6 |
| 277 | C9, MIF, ORM1, PKM, SAA | Age | 6 |
| 278 | C9, MIF, ORM1, PKM, TFRC | Age | 6 |
| 279 | C9, MIF, ORM1, SAA, TFRC | Age | 6 |
| 280 | C9, MIF, PKM, SAA, TFRC | Age | 6 |
| 281 | C9, ORM1, PKM, SAA, TFRC | Age | 6 |
| 282 | CEA, DPP4, MIF, ORM1, PKM | Age | 6 |
| 283 | CEA, DPP4, MIF, ORM1, SAA | Age | 6 |
| 284 | CEA, DPP4, MIF, ORM1, TFRC | Age | 6 |
| 285 | CEA, DPP4, MIF, PKM, SAA | Age | 6 |
| 286 | CEA, DPP4, MIF, PKM, TFRC | Age | 6 |
| 287 | CEA, DPP4, MIF, SAA, TFRC | Age | 6 |
| 288 | CEA, DPP4, ORM1, PKM, SAA | Age | 6 |
| 289 | CEA, DPP4, ORM1, PKM, TFRC | Age | 6 |
| 290 | CEA, DPP4, ORM1, SAA, TFRC | Age | 6 |
| 291 | CEA, DPP4, PKM, SAA, TFRC | Age | 6 |
| 292 | CEA, MIF, ORM1, PKM, SAA | Age | 6 |
| 293 | CEA, MIF, ORM1, PKM, TFRC | Age | 6 |
| 294 | CEA, MIF, ORM1, SAA, TFRC | Age | 6 |
| 295 | CEA, MIF, PKM, SAA, TFRC | Age | 6 |
| 296 | CEA, ORM1, PKM, SAA, TFRC | Age | 6 |
| 297 | DPP4, MIF, ORM1, PKM, SAA | Age | 6 |
| 298 | DPP4, MIF, ORM1, PKM, TFRC | Age | 6 |
| 299 | DPP4, MIF, ORM1, SAA, TFRC | Age | 6 |
| 300 | DPP4, MIF, PKM, SAA, TFRC | Age | 6 |
| 301 | DPP4, ORM1, PKM, SAA, TFRC | Age | 6 |
| 302 | MIF, ORM1, PKM, SAA, TFRC | Age | 6 |
| 303 | C9, CEA, DPP4, MIF, ORM1 | Gender | 6 |
| 304 | C9, CEA, DPP4, MIF, PKM | Gender | 6 |
| 305 | C9, CEA, DPP4, MIF, SAA | Gender | 6 |
| 306 | C9, CEA, DPP4, MIF, TFRC | Gender | 6 |
| 307 | C9, CEA, DPP4, ORM1, PKM | Gender | 6 |
| 308 | C9, CEA, DPP4, ORM1, SAA | Gender | 6 |
| 309 | C9, CEA, DPP4, ORM1, TFRC | Gender | 6 |
| 310 | C9, CEA, DPP4, PKM, SAA | Gender | 6 |
| 311 | C9, CEA, DPP4, PKM, TFRC | Gender | 6 |
| 312 | C9, CEA, DPP4, SAA, TFRC | Gender | 6 |
| 313 | C9, CEA, MIF, ORM1, PKM | Gender | 6 |
| 314 | C9, CEA, MIF, ORM1, SAA | Gender | 6 |
| 315 | C9, CEA, MIF, ORM1, TFRC | Gender | 6 |
| 316 | C9, CEA, MIF, PKM, SAA | Gender | 6 |
| 317 | C9, CEA, MIF, PKM, TFRC | Gender | 6 |
| 318 | C9, CEA, MIF, SAA, TFRC | Gender | 6 |
| 319 | C9, CEA, ORM1, PKM, SAA | Gender | 6 |
| 320 | C9, CEA, ORM1, PKM, TFRC | Gender | 6 |
| 321 | C9, CEA, ORM1, SAA, TFRC | Gender | 6 |
| 322 | C9, CEA, PKM, SAA, TFRC | Gender | 6 |
| 323 | C9, DPP4, MIF, ORM1, PKM | Gender | 6 |
| 324 | C9, DPP4, MIF, ORM1, SAA | Gender | 6 |
| 325 | C9, DPP4, MIF, ORM1, TFRC | Gender | 6 |
| 326 | C9, DPP4, MIF, PKM, SAA | Gender | 6 |
| 327 | C9, DPP4, MIF, PKM, TFRC | Gender | 6 |
| 328 | C9, DPP4, MIF, SAA, TFRC | Gender | 6 |
| 329 | C9, DPP4, ORM1, PKM, SAA | Gender | 6 |
| 330 | C9, DPP4, ORM1, PKM, TFRC | Gender | 6 |
| 331 | C9, DPP4, ORM1, SAA, TFRC | Gender | 6 |
| 332 | C9, DPP4, PKM, SAA, TFRC | Gender | 6 |
| 333 | C9, MIF, ORM1, PKM, SAA | Gender | 6 |
| 334 | C9, MIF, ORM1, PKM, TFRC | Gender | 6 |
| 335 | C9, MIF, ORM1, SAA, TFRC | Gender | 6 |
| 336 | C9, MIF, PKM, SAA, TFRC | Gender | 6 |
| 337 | C9, ORM1, PKM, SAA, TFRC | Gender | 6 |
| 338 | CEA, DPP4, MIF, ORM1, PKM | Gender | 6 |
| 339 | CEA, DPP4, MIF, ORM1, SAA | Gender | 6 |
| 340 | CEA, DPP4, MIF, ORM1, TFRC | Gender | 6 |
| 341 | CEA, DPP4, MIF, PKM, SAA | Gender | 6 |
| 342 | CEA, DPP4, MIF, PKM, TFRC | Gender | 6 |
| 343 | CEA, DPP4, MIF, SAA, TFRC | Gender | 6 |
| 344 | CEA, DPP4, ORM1, PKM, SAA | Gender | 6 |
| 345 | CEA, DPP4, ORM1, PKM, TFRC | Gender | 6 |
| 346 | CEA, DPP4, ORM1, SAA, TFRC | Gender | 6 |
| 347 | CEA, DPP4, PKM, SAA, TFRC | Gender | 6 |
| 348 | CEA, MIF, ORM1, PKM, SAA | Gender | 6 |
| 349 | CEA, MIF, ORM1, PKM, TFRC | Gender | 6 |
| 350 | CEA, MIF, ORM1, SAA, TFRC | Gender | 6 |
| 351 | CEA, MIF, PKM, SAA, TFRC | Gender | 6 |
| 352 | CEA, ORM1, PKM, SAA, TFRC | Gender | 6 |
| 353 | DPP4, MIF, ORM1, PKM, SAA | Gender | 6 |
| 354 | DPP4, MIF, ORM1, PKM, TFRC | Gender | 6 |
| 355 | DPP4, MIF, ORM1, SAA, TFRC | Gender | 6 |
| 356 | DPP4, MIF, PKM, SAA, TFRC | Gender | 6 |
| 357 | DPP4, ORM1, PKM, SAA, TFRC | Gender | 6 |
| 358 | MIF, ORM1, PKM, SAA, TFRC | Gender | 6 |
| 359 | C9, CEA, DPP4, MIF, ORM1, PKM | NONE | 6 |
| 360 | C9, CEA, DPP4, MIF, ORM1, SAA | NONE | 6 |
| 361 | C9, CEA, DPP4, MIF, ORM1, TFRC | NONE | 6 |
| 362 | C9, CEA, DPP4, MIF, PKM, SAA | NONE | 6 |
| 363 | C9, CEA, DPP4, MIF, PKM, TFRC | NONE | 6 |
| 364 | C9, CEA, DPP4, MIF, SAA, TFRC | NONE | 6 |
| 365 | C9, CEA, DPP4, ORM1, PKM, SAA | NONE | 6 |
| 366 | C9, CEA, DPP4, ORM1, PKM, TFRC | NONE | 6 |
| 367 | C9, CEA, DPP4, ORM1, SAA, TFRC | NONE | 6 |
| 368 | C9, CEA, DPP4, PKM, SAA, TFRC | NONE | 6 |
| 369 | C9, CEA, MIF, ORM1, PKM, SAA | NONE | 6 |
| 370 | C9, CEA, MIF, ORM1, PKM, TFRC | NONE | 6 |
| 371 | C9, CEA, MIF, ORM1, SAA, TFRC | NONE | 6 |
| 372 | C9, CEA, MIF, PKM, SAA, TFRC | NONE | 6 |
| 373 | C9, CEA, ORM1, PKM, SAA, TFRC | NONE | 6 |
| 374 | C9, DPP4, MIF, ORM1, PKM, SAA | NONE | 6 |
| 375 | C9, DPP4, MIF, ORM1, PKM, TFRC | NONE | 6 |
| 376 | C9, DPP4, MIF, ORM1, SAA, TFRC | NONE | 6 |
| 377 | C9, DPP4, MIF, PKM, SAA, TFRC | NONE | 6 |
| 378 | C9, DPP4, ORM1, PKM, SAA, TFRC | NONE | 6 |
| 379 | C9, MIF, ORM1, PKM, SAA, TFRC | NONE | 6 |
| 380 | CEA, DPP4, MIF, ORM1, PKM, SAA | NONE | 6 |
| 381 | CEA, DPP4, MIF, ORM1, PKM, TFRC | NONE | 6 |
| 382 | CEA, DPP4, MIF, ORM1, SAA, TFRC | NONE | 6 |
| 383 | CEA, DPP4, MIF, PKM, SAA, TFRC | NONE | 6 |
| 384 | CEA, DPP4, ORM1, PKM, SAA, TFRC | NONE | 6 |
| 385 | CEA, MIF, ORM1, PKM, SAA, TFRC | NONE | 6 |
| 386 | DPP4, MIF, ORM1, PKM, SAA, TFRC | NONE | 6 |
| 387 | C9, CEA, DPP4 | Age and Gender | 5 |
| 388 | C9, CEA, MIF | Age and Gender | 5 |
| 389 | C9, CEA, ORM1 | Age and Gender | 5 |
| 390 | C9, CEA, PKM | Age and Gender | 5 |
| 391 | C9, CEA, SAA | Age and Gender | 5 |
| 392 | C9, CEA, TFRC | Age and Gender | 5 |
| 393 | C9, DPP4, MIF | Age and Gender | 5 |
| 394 | C9, DPP4, ORM1 | Age and Gender | 5 |
| 395 | C9, DPP4, PKM | Age and Gender | 5 |
| 396 | C9, DPP4, SAA | Age and Gender | 5 |
| 397 | C9, DPP4, TFRC | Age and Gender | 5 |
| 398 | C9, MIF, ORM1 | Age and Gender | 5 |
| 399 | C9, MIF, PKM | Age and Gender | 5 |
| 400 | C9, MIF, SAA | Age and Gender | 5 |
| 401 | C9, MIF, TFRC | Age and Gender | 5 |
| 402 | C9, ORM1, PKM | Age and Gender | 5 |
| 403 | C9, ORM1, SAA | Age and Gender | 5 |
| 404 | C9, ORM1, TFRC | Age and Gender | 5 |
| 405 | C9, PKM, SAA | Age and Gender | 5 |
| 406 | C9, PKM, TFRC | Age and Gender | 5 |
| 407 | C9, SAA, TFRC | Age and Gender | 5 |
| 408 | CEA, DPP4, MIF | Age and Gender | 5 |
| 409 | CEA, DPP4, ORM1 | Age and Gender | 5 |
| 410 | CEA, DPP4, PKM | Age and Gender | 5 |
| 411 | CEA, DPP4, SAA | Age and Gender | 5 |
| 412 | CEA, DPP4, TFRC | Age and Gender | 5 |
| 413 | CEA, MIF, ORM1 | Age and Gender | 5 |
| 414 | CEA, MIF, PKM | Age and Gender | 5 |
| 415 | CEA, MIF, SAA | Age and Gender | 5 |
| 416 | CEA, MIF, TFRC | Age and Gender | 5 |
| 417 | CEA, ORM1, PKM | Age and Gender | 5 |
| 418 | CEA, ORM1, SAA | Age and Gender | 5 |
| 419 | CEA, ORM1, TFRC | Age and Gender | 5 |
| 420 | CEA, PKM, SAA | Age and Gender | 5 |
| 421 | CEA, PKM, TFRC | Age and Gender | 5 |
| 422 | CEA, SAA, TFRC | Age and Gender | 5 |
| 423 | DPP4, MIF, ORM1 | Age and Gender | 5 |
| 424 | DPP4, MIF, PKM | Age and Gender | 5 |
| 425 | DPP4, MIF, SAA | Age and Gender | 5 |
| 426 | DPP4, MIF, TFRC | Age and Gender | 5 |
| 427 | DPP4, ORM1, PKM | Age and Gender | 5 |
| 428 | DPP4, ORM1, SAA | Age and Gender | 5 |
| 429 | DPP4, ORM1, TFRC | Age and Gender | 5 |
| 430 | DPP4, PKM, SAA | Age and Gender | 5 |
| 431 | DPP4, PKM, TFRC | Age and Gender | 5 |
| 432 | DPP4, SAA, TFRC | Age and Gender | 5 |
| 433 | MIF, ORM1, PKM | Age and Gender | 5 |
| 434 | MIF, ORM1, SAA | Age and Gender | 5 |
| 435 | MIF, ORM1, TFRC | Age and Gender | 5 |
| 436 | MIF, PKM, SAA | Age and Gender | 5 |
| 437 | MIF, PKM, TFRC | Age and Gender | 5 |
| 438 | MIF, SAA, TFRC | Age and Gender | 5 |
| 439 | ORM1, PKM, SAA | Age and Gender | 5 |
| 440 | ORM1, PKM, TFRC | Age and Gender | 5 |
| 441 | ORM1, SAA, TFRC | Age and Gender | 5 |
| 442 | PKM, SAA, TFRC | Age and Gender | 5 |
| 443 | C9, CEA, DPP4, MIF | Age | 5 |
| 444 | C9, CEA, DPP4, ORM1 | Age | 5 |
| 445 | C9, CEA, DPP4, PKM | Age | 5 |
| 446 | C9, CEA, DPP4, SAA | Age | 5 |
| 447 | C9, CEA, DPP4, TFRC | Age | 5 |
| 448 | C9, CEA, MIF, ORM1 | Age | 5 |
| 449 | C9, CEA, MIF, PKM | Age | 5 |
| 450 | C9, CEA, MIF, SAA | Age | 5 |
| 451 | C9, CEA, MIF, TFRC | Age | 5 |
| 452 | C9, CEA, ORM1, PKM | Age | 5 |
| 453 | C9, CEA, ORM1, SAA | Age | 5 |
| 454 | C9, CEA, ORM1, TFRC | Age | 5 |
| 455 | C9, CEA, PKM, SAA | Age | 5 |
| 456 | C9, CEA, PKM, TFRC | Age | 5 |
| 457 | C9, CEA, SAA, TFRC | Age | 5 |
| 458 | C9, DPP4, MIF, ORM1 | Age | 5 |
| 459 | C9, DPP4, MIF, PKM | Age | 5 |
| 460 | C9, DPP4, MIF, SAA | Age | 5 |
| 461 | C9, DPP4, MIF, TFRC | Age | 5 |
| 462 | C9, DPP4, ORM1, PKM | Age | 5 |
| 463 | C9, DPP4, ORM1, SAA | Age | 5 |
| 464 | C9, DPP4, ORM1, TFRC | Age | 5 |
| 465 | C9, DPP4, PKM, SAA | Age | 5 |
| 466 | C9, DPP4, PKM, TFRC | Age | 5 |
| 467 | C9, DPP4, SAA, TFRC | Age | 5 |
| 468 | C9, MIF, ORM1, PKM | Age | 5 |
| 469 | C9, MIF, ORM1, SAA | Age | 5 |
| 470 | C9, MIF, ORM1, TFRC | Age | 5 |
| 471 | C9, MIF, PKM, SAA | Age | 5 |
| 472 | C9, MIF, PKM, TFRC | Age | 5 |
| 473 | C9, MIF, SAA, TFRC | Age | 5 |
| 474 | C9, ORM1, PKM, SAA | Age | 5 |
| 475 | C9, ORM1, PKM, TFRC | Age | 5 |
| 476 | C9, ORM1, SAA, TFRC | Age | 5 |
| 477 | C9, PKM, SAA, TFRC | Age | 5 |
| 478 | CEA, DPP4, MIF, ORM1 | Age | 5 |
| 479 | CEA, DPP4, MIF, PKM | Age | 5 |
| 480 | CEA, DPP4, MIF, SAA | Age | 5 |
| 481 | CEA, DPP4, MIF, TFRC | Age | 5 |
| 482 | CEA, DPP4, ORM1, PKM | Age | 5 |
| 483 | CEA, DPP4, ORM1, SAA | Age | 5 |
| 484 | CEA, DPP4, ORM1, TFRC | Age | 5 |
| 485 | CEA, DPP4, PKM, SAA | Age | 5 |
| 486 | CEA, DPP4, PKM, TFRC | Age | 5 |
| 487 | CEA, DPP4, SAA, TFRC | Age | 5 |
| 488 | CEA, MIF, ORM1, PKM | Age | 5 |
| 489 | CEA, MIF, ORM1, SAA | Age | 5 |
| 490 | CEA, MIF, ORM1, TFRC | Age | 5 |
| 491 | CEA, MIF, PKM, SAA | Age | 5 |
| 492 | CEA, MIF, PKM, TFRC | Age | 5 |
| 493 | CEA, MIF, SAA, TFRC | Age | 5 |
| 494 | CEA, ORM1, PKM, SAA | Age | 5 |
| 495 | CEA, ORM1, PKM, TFRC | Age | 5 |
| 496 | CEA, ORM1, SAA, TFRC | Age | 5 |
| 497 | CEA, PKM, SAA, TFRC | Age | 5 |
| 498 | DPP4, MIF, ORM1, PKM | Age | 5 |
| 499 | DPP4, MIF, ORM1, SAA | Age | 5 |
| 500 | DPP4, MIF, ORM1, TFRC | Age | 5 |
| 501 | DPP4, MIF, PKM, SAA | Age | 5 |
| 502 | DPP4, MIF, PKM, TFRC | Age | 5 |
| 503 | DPP4, MIF, SAA, TFRC | Age | 5 |
| 504 | DPP4, ORM1, PKM, SAA | Age | 5 |
| 505 | DPP4, ORM1, PKM, TFRC | Age | 5 |
| 506 | DPP4, ORM1, SAA, TFRC | Age | 5 |
| 507 | DPP4, PKM, SAA, TFRC | Age | 5 |
| 508 | MIF, ORM1, PKM, SAA | Age | 5 |
| 509 | MIF, ORM1, PKM, TFRC | Age | 5 |
| 510 | MIF, ORM1, SAA, TFRC | Age | 5 |
| 511 | MIF, PKM, SAA, TFRC | Age | 5 |
| 512 | ORM1, PKM, SAA, TFRC | Age | 5 |
| 513 | C9, CEA, DPP4, MIF | Gender | 5 |
| 514 | C9, CEA, DPP4, ORM1 | Gender | 5 |
| 515 | C9, CEA, DPP4, PKM | Gender | 5 |
| 516 | C9, CEA, DPP4, SAA | Gender | 5 |
| 517 | C9, CEA, DPP4, TFRC | Gender | 5 |
| 518 | C9, CEA, MIF, ORM1 | Gender | 5 |
| 519 | C9, CEA, MIF, PKM | Gender | 5 |
| 520 | C9, CEA, MIF, SAA | Gender | 5 |
| 521 | C9, CEA, MIF, TFRC | Gender | 5 |
| 522 | C9, CEA, ORM1, PKM | Gender | 5 |
| 523 | C9, CEA, ORM1, SAA | Gender | 5 |
| 524 | C9, CEA, ORM1, TFRC | Gender | 5 |
| 525 | C9, CEA, PKM, SAA | Gender | 5 |
| 526 | C9, CEA, PKM, TFRC | Gender | 5 |
| 527 | C9, CEA, SAA, TFRC | Gender | 5 |
| 528 | C9, DPP4, MIF, ORM1 | Gender | 5 |
| 529 | C9, DPP4, MIF, PKM | Gender | 5 |
| 530 | C9, DPP4, MIF, SAA | Gender | 5 |
| 531 | C9, DPP4, MIF, TFRC | Gender | 5 |
| 532 | C9, DPP4, ORM1, PKM | Gender | 5 |
| 533 | C9, DPP4, ORM1, SAA | Gender | 5 |
| 534 | C9, DPP4, ORM1, TFRC | Gender | 5 |
| 535 | C9, DPP4, PKM, SAA | Gender | 5 |
| 536 | C9, DPP4, PKM, TFRC | Gender | 5 |
| 537 | C9, DPP4, SAA, TFRC | Gender | 5 |
| 538 | C9, MIF, ORM1, PKM | Gender | 5 |
| 539 | C9, MIF, ORM1, SAA | Gender | 5 |
| 540 | C9, MIF, ORM1, TFRC | Gender | 5 |
| 541 | C9, MIF, PKM, SAA | Gender | 5 |
| 542 | C9, MIF, PKM, TFRC | Gender | 5 |
| 543 | C9, MIF, SAA, TFRC | Gender | 5 |
| 544 | C9, ORM1, PKM, SAA | Gender | 5 |
| 545 | C9, ORM1, PKM, TFRC | Gender | 5 |
| 546 | C9, ORM1, SAA, TFRC | Gender | 5 |
| 547 | C9, PKM, SAA, TFRC | Gender | 5 |
| 548 | CEA, DPP4, MIF, ORM1 | Gender | 5 |
| 549 | CEA, DPP4, MIF, PKM | Gender | 5 |
| 550 | CEA, DPP4, MIF, SAA | Gender | 5 |
| 551 | CEA, DPP4, MIF, TFRC | Gender | 5 |
| 552 | CEA, DPP4, ORM1, PKM | Gender | 5 |
| 553 | CEA, DPP4, ORM1, SAA | Gender | 5 |
| 554 | CEA, DPP4, ORM1, TFRC | Gender | 5 |
| 555 | CEA, DPP4, PKM, SAA | Gender | 5 |
| 556 | CEA, DPP4, PKM, TFRC | Gender | 5 |
| 557 | CEA, DPP4, SAA, TFRC | Gender | 5 |
| 558 | CEA, MIF, ORM1, PKM | Gender | 5 |
| 559 | CEA, MIF, ORM1, SAA | Gender | 5 |
| 560 | CEA, MIF, ORM1, TFRC | Gender | 5 |
| 561 | CEA, MIF, PKM, SAA | Gender | 5 |
| 562 | CEA, MIF, PKM, TFRC | Gender | 5 |
| 563 | CEA, MIF, SAA, TFRC | Gender | 5 |
| 564 | CEA, ORM1, PKM, SAA | Gender | 5 |
| 565 | CEA, ORM1, PKM, TFRC | Gender | 5 |
| 566 | CEA, ORM1, SAA, TFRC | Gender | 5 |
| 567 | CEA, PKM, SAA, TFRC | Gender | 5 |
| 568 | DPP4, MIF, ORM1, PKM | Gender | 5 |
| 569 | DPP4, MIF, ORM1, SAA | Gender | 5 |
| 570 | DPP4, MIF, ORM1, TFRC | Gender | 5 |
| 571 | DPP4, MIF, PKM, SAA | Gender | 5 |
| 572 | DPP4, MIF, PKM, TFRC | Gender | 5 |
| 573 | DPP4, MIF, SAA, TFRC | Gender | 5 |
| 574 | DPP4, ORM1, PKM, SAA | Gender | 5 |
| 575 | DPP4, ORM1, PKM, TFRC | Gender | 5 |
| 576 | DPP4, ORM1, SAA, TFRC | Gender | 5 |
| 577 | DPP4, PKM, SAA, TFRC | Gender | 5 |
| 578 | MIF, ORM1, PKM, SAA | Gender | 5 |
| 579 | MIF, ORM1, PKM, TFRC | Gender | 5 |
| 580 | MIF, ORM1, SAA, TFRC | Gender | 5 |
| 581 | MIF, PKM, SAA, TFRC | Gender | 5 |
| 582 | ORM1, PKM, SAA, TFRC | Gender | 5 |
| 583 | C9, CEA, DPP4, MIF, ORM1 | NONE | 5 |
| 584 | C9, CEA, DPP4, MIF, PKM | NONE | 5 |
| 585 | C9, CEA, DPP4, MIF, SAA | NONE | 5 |
| 586 | C9, CEA, DPP4, MIF, TFRC | NONE | 5 |
| 587 | C9, CEA, DPP4, ORM1, PKM | NONE | 5 |
| 588 | C9, CEA, DPP4, ORM1, SAA | NONE | 5 |
| 589 | C9, CEA, DPP4, ORM1, TFRC | NONE | 5 |
| 590 | C9, CEA, DPP4, PKM, SAA | NONE | 5 |
| 591 | C9, CEA, DPP4, PKM, TFRC | NONE | 5 |
| 592 | C9, CEA, DPP4, SAA, TFRC | NONE | 5 |
| 593 | C9, CEA, MIF, ORM1, PKM | NONE | 5 |
| 594 | C9, CEA, MIF, ORM1, SAA | NONE | 5 |
| 595 | C9, CEA, MIF, ORM1, TFRC | NONE | 5 |
| 596 | C9, CEA, MIF, PKM, SAA | NONE | 5 |
| 597 | C9, CEA, MIF, PKM, TFRC | NONE | 5 |
| 598 | C9, CEA, MIF, SAA, TFRC | NONE | 5 |
| 599 | C9, CEA, ORM1, PKM, SAA | NONE | 5 |
| 600 | C9, CEA, ORM1, PKM, TFRC | NONE | 5 |
| 601 | C9, CEA, ORM1, SAA, TFRC | NONE | 5 |
| 602 | C9, CEA, PKM, SAA, TFRC | NONE | 5 |
| 603 | C9, DPP4, MIF, ORM1, PKM | NONE | 5 |
| 604 | C9, DPP4, MIF, ORM1, SAA | NONE | 5 |
| 605 | C9, DPP4, MIF, ORM1, TFRC | NONE | 5 |
| 606 | C9, DPP4, MIF, PKM, SAA | NONE | 5 |
| 607 | C9, DPP4, MIF, PKM, TFRC | NONE | 5 |
| 608 | C9, DPP4, MIF, SAA, TFRC | NONE | 5 |
| 609 | C9, DPP4, ORM1, PKM, SAA | NONE | 5 |
| 610 | C9, DPP4, ORM1, PKM, TFRC | NONE | 5 |
| 611 | C9, DPP4, ORM1, SAA, TFRC | NONE | 5 |
| 612 | C9, DPP4, PKM, SAA, TFRC | NONE | 5 |
| 613 | C9, MIF, ORM1, PKM, SAA | NONE | 5 |
| 614 | C9, MIF, ORM1, PKM, TFRC | NONE | 5 |
| 615 | C9, MIF, ORM1, SAA, TFRC | NONE | 5 |
| 616 | C9, MIF, PKM, SAA, TFRC | NONE | 5 |
| 617 | C9, ORM1, PKM, SAA, TFRC | NONE | 5 |
| 618 | CEA, DPP4, MIF, ORM1, PKM | NONE | 5 |
| 619 | CEA, DPP4, MIF, ORM1, SAA | NONE | 5 |
| 620 | CEA, DPP4, MIF, ORM1, TFRC | NONE | 5 |
| 621 | CEA, DPP4, MIF, PKM, SAA | NONE | 5 |
| 622 | CEA, DPP4, MIF, PKM, TFRC | NONE | 5 |
| 623 | CEA, DPP4, MIF, SAA, TFRC | NONE | 5 |
| 624 | CEA, DPP4, ORM1, PKM, SAA | NONE | 5 |
| 625 | CEA, DPP4, ORM1, PKM, TFRC | NONE | 5 |
| 626 | CEA, DPP4, ORM1, SAA, TFRC | NONE | 5 |
| 627 | CEA, DPP4, PKM, SAA, TFRC | NONE | 5 |
| 628 | CEA, MIF, ORM1, PKM, SAA | NONE | 5 |
| 629 | CEA, MIF, ORM1, PKM, TFRC | NONE | 5 |
| 630 | CEA, MIF, ORM1, SAA, TFRC | NONE | 5 |
| 631 | CEA, MIF, PKM, SAA, TFRC | NONE | 5 |
| 632 | CEA, ORM1, PKM, SAA, TFRC | NONE | 5 |
| 633 | DPP4, MIF, ORM1, PKM, SAA | NONE | 5 |
| 634 | DPP4, MIF, ORM1, PKM, TFRC | NONE | 5 |
| 635 | DPP4, MIF, ORM1, SAA, TFRC | NONE | 5 |
| 636 | DPP4, MIF, PKM, SAA, TFRC | NONE | 5 |
| 637 | DPP4, ORM1, PKM, SAA, TFRC | NONE | 5 |
| 638 | MIF, ORM1, PKM, SAA, TFRC | NONE | 5 |
| 639 | C9, CEA | Age and Gender | 4 |
| 640 | C9, DPP4 | Age and Gender | 4 |
| 641 | C9, MIF | Age and Gender | 4 |
| 642 | C9, ORM1 | Age and Gender | 4 |
| 643 | C9, PKM | Age and Gender | 4 |
| 644 | C9, SAA | Age and Gender | 4 |
| 645 | C9, TFRC | Age and Gender | 4 |
| 646 | CEA, DPP4 | Age and Gender | 4 |
| 647 | CEA, MIF | Age and Gender | 4 |
| 648 | CEA, ORM1 | Age and Gender | 4 |
| 649 | CEA, PKM | Age and Gender | 4 |
| 650 | CEA, SAA | Age and Gender | 4 |
| 651 | CEA, TFRC | Age and Gender | 4 |
| 652 | DPP4, MIF | Age and Gender | 4 |
| 653 | DPP4, ORM1 | Age and Gender | 4 |
| 654 | DPP4, PKM | Age and Gender | 4 |
| 655 | DPP4, SAA | Age and Gender | 4 |
| 656 | DPP4, TFRC | Age and Gender | 4 |
| 657 | MIF, ORM1 | Age and Gender | 4 |
| 658 | MIF, PKM | Age and Gender | 4 |
| 659 | MIF, SAA | Age and Gender | 4 |
| 660 | MIF, TFRC | Age and Gender | 4 |
| 661 | ORM1, PKM | Age and Gender | 4 |
| 662 | ORM1, SAA | Age and Gender | 4 |
| 663 | ORM1, TFRC | Age and Gender | 4 |
| 664 | PKM, SAA | Age and Gender | 4 |
| 665 | PKM, TFRC | Age and Gender | 4 |
| 666 | SAA, TFRC | Age and Gender | 4 |
| 667 | C9, CEA, DPP4 | Age | 4 |
| 668 | C9, CEA, MIF | Age | 4 |
| 669 | C9, CEA, ORM1 | Age | 4 |
| 670 | C9, CEA, PKM | Age | 4 |
| 671 | C9, CEA, SAA | Age | 4 |
| 672 | C9, CEA, TFRC | Age | 4 |
| 673 | C9, DPP4, MIF | Age | 4 |
| 674 | C9, DPP4, ORM1 | Age | 4 |
| 675 | C9, DPP4, PKM | Age | 4 |
| 676 | C9, DPP4, SAA | Age | 4 |
| 677 | C9, DPP4, TFRC | Age | 4 |
| 678 | C9, MIF, ORM1 | Age | 4 |
| 679 | C9, MIF, PKM | Age | 4 |
| 680 | C9, MIF, SAA | Age | 4 |
| 681 | C9, MIF, TFRC | Age | 4 |
| 682 | C9, ORM1, PKM | Age | 4 |
| 683 | C9, ORM1, SAA | Age | 4 |
| 684 | C9, ORM1, TFRC | Age | 4 |
| 685 | C9, PKM, SAA | Age | 4 |
| 686 | C9, PKM, TFRC | Age | 4 |
| 687 | C9, SAA, TFRC | Age | 4 |
| 688 | CEA, DPP4, MIF | Age | 4 |
| 689 | CEA, DPP4, ORM1 | Age | 4 |
| 690 | CEA, DPP4, PKM | Age | 4 |
| 691 | CEA, DPP4, SAA | Age | 4 |
| 692 | CEA, DPP4, TFRC | Age | 4 |
| 693 | CEA, MIF, ORM1 | Age | 4 |
| 694 | CEA, MIF, PKM | Age | 4 |
| 695 | CEA, MIF, SAA | Age | 4 |
| 696 | CEA, MIF, TFRC | Age | 4 |
| 697 | CEA, ORM1, PKM | Age | 4 |
| 698 | CEA, ORM1, SAA | Age | 4 |
| 699 | CEA, ORM1, TFRC | Age | 4 |
| 700 | CEA, PKM, SAA | Age | 4 |
| 701 | CEA, PKM, TFRC | Age | 4 |
| 702 | CEA, SAA, TFRC | Age | 4 |
| 703 | DPP4, MIF, ORM1 | Age | 4 |
| 704 | DPP4, MIF, PKM | Age | 4 |
| 705 | DPP4, MIF, SAA | Age | 4 |
| 706 | DPP4, MIF, TFRC | Age | 4 |
| 707 | DPP4, ORM1, PKM | Age | 4 |
| 708 | DPP4, ORM1, SAA | Age | 4 |
| 709 | DPP4, ORM1, TFRC | Age | 4 |
| 710 | DPP4, PKM, SAA | Age | 4 |
| 711 | DPP4, PKM, TFRC | Age | 4 |
| 712 | DPP4, SAA, TFRC | Age | 4 |
| 713 | MIF, ORM1, PKM | Age | 4 |
| 714 | MIF, ORM1, SAA | Age | 4 |
| 715 | MIF, ORM1, TFRC | Age | 4 |
| 716 | MIF, PKM, SAA | Age | 4 |
| 717 | MIF, PKM, TFRC | Age | 4 |
| 718 | MIF, SAA, TFRC | Age | 4 |
| 719 | ORM1, PKM, SAA | Age | 4 |
| 720 | ORM1, PKM, TFRC | Age | 4 |
| 721 | ORM1, SAA, TFRC | Age | 4 |
| 722 | PKM, SAA, TFRC | Age | 4 |
| 723 | C9, CEA, DPP4 | Gender | 4 |
| 724 | C9, CEA, MIF | Gender | 4 |
| 725 | C9, CEA, ORM1 | Gender | 4 |
| 726 | C9, CEA, PKM | Gender | 4 |
| 727 | C9, CEA, SAA | Gender | 4 |
| 728 | C9, CEA, TFRC | Gender | 4 |
| 729 | C9, DPP4, MIF | Gender | 4 |
| 730 | C9, DPP4, ORM1 | Gender | 4 |
| 731 | C9, DPP4, PKM | Gender | 4 |
| 732 | C9, DPP4, SAA | Gender | 4 |
| 733 | C9, DPP4, TFRC | Gender | 4 |
| 734 | C9, MIF, ORM1 | Gender | 4 |
| 735 | C9, MIF, PKM | Gender | 4 |
| 736 | C9, MIF, SAA | Gender | 4 |
| 737 | C9, MIF, TFRC | Gender | 4 |
| 738 | C9, ORM1, PKM | Gender | 4 |
| 739 | C9, ORM1, SAA | Gender | 4 |
| 740 | C9, ORM1, TFRC | Gender | 4 |
| 741 | C9, PKM, SAA | Gender | 4 |
| 742 | C9, PKM, TFRC | Gender | 4 |
| 743 | C9, SAA, TFRC | Gender | 4 |
| 744 | CEA, DPP4, MIF | Gender | 4 |
| 745 | CEA, DPP4, ORM1 | Gender | 4 |
| 746 | CEA, DPP4, PKM | Gender | 4 |
| 747 | CEA, DPP4, SAA | Gender | 4 |
| 748 | CEA, DPP4, TFRC | Gender | 4 |
| 749 | CEA, MIF, ORM1 | Gender | 4 |
| 750 | CEA, MIF, PKM | Gender | 4 |
| 751 | CEA, MIF, SAA | Gender | 4 |
| 752 | CEA, MIF, TFRC | Gender | 4 |
| 753 | CEA, ORM1, PKM | Gender | 4 |
| 754 | CEA, ORM1, SAA | Gender | 4 |
| 755 | CEA, ORM1, TFRC | Gender | 4 |
| 756 | CEA, PKM, SAA | Gender | 4 |
| 757 | CEA, PKM, TFRC | Gender | 4 |
| 758 | CEA, SAA, TFRC | Gender | 4 |
| 759 | DPP4, MIF, ORM1 | Gender | 4 |
| 760 | DPP4, MIF, PKM | Gender | 4 |
| 761 | DPP4, MIF, SAA | Gender | 4 |
| 762 | DPP4, MIF, TFRC | Gender | 4 |
| 763 | DPP4, ORM1, PKM | Gender | 4 |
| 764 | DPP4, ORM1, SAA | Gender | 4 |
| 765 | DPP4, ORM1, TFRC | Gender | 4 |
| 766 | DPP4, PKM, SAA | Gender | 4 |
| 767 | DPP4, PKM, TFRC | Gender | 4 |
| 768 | DPP4, SAA, TFRC | Gender | 4 |
| 769 | MIF, ORM1, PKM | Gender | 4 |
| 770 | MIF, ORM1, SAA | Gender | 4 |
| 771 | MIF, ORM1, TFRC | Gender | 4 |
| 772 | MIF, PKM, SAA | Gender | 4 |
| 773 | MIF, PKM, TFRC | Gender | 4 |
| 774 | MIF, SAA, TFRC | Gender | 4 |
| 775 | ORM1, PKM, SAA | Gender | 4 |
| 776 | ORM1, PKM, TFRC | Gender | 4 |
| 777 | ORM1, SAA, TFRC | Gender | 4 |
| 778 | PKM, SAA, TFRC | Gender | 4 |
| 779 | C9, CEA, DPP4, MIF | NONE | 4 |
| 780 | C9, CEA, DPP4, ORM1 | NONE | 4 |
| 781 | C9, CEA, DPP4, PKM | NONE | 4 |
| 782 | C9, CEA, DPP4, SAA | NONE | 4 |
| 783 | C9, CEA, DPP4, TFRC | NONE | 4 |
| 784 | C9, CEA, MIF, ORM1 | NONE | 4 |
| 785 | C9, CEA, MIF, PKM | NONE | 4 |
| 786 | C9, CEA, MIF, SAA | NONE | 4 |
| 787 | C9, CEA, MIF, TFRC | NONE | 4 |
| 788 | C9, CEA, ORM1, PKM | NONE | 4 |
| 789 | C9, CEA, ORM1, SAA | NONE | 4 |
| 790 | C9, CEA, ORM1, TFRC | NONE | 4 |
| 791 | C9, CEA, PKM, SAA | NONE | 4 |
| 792 | C9, CEA, PKM, TFRC | NONE | 4 |
| 793 | C9, CEA, SAA, TFRC | NONE | 4 |
| 794 | C9, DPP4, MIF, ORM1 | NONE | 4 |
| 795 | C9, DPP4, MIF, PKM | NONE | 4 |
| 796 | C9, DPP4, MIF, SAA | NONE | 4 |
| 797 | C9, DPP4, MIF, TFRC | NONE | 4 |
| 798 | C9, DPP4, ORM1, PKM | NONE | 4 |
| 799 | C9, DPP4, ORM1, SAA | NONE | 4 |
| 800 | C9, DPP4, ORM1, TFRC | NONE | 4 |
| 801 | C9, DPP4, PKM, SAA | NONE | 4 |
| 802 | C9, DPP4, PKM, TFRC | NONE | 4 |
| 803 | C9, DPP4, SAA, TFRC | NONE | 4 |
| 804 | C9, MIF, ORM1, PKM | NONE | 4 |
| 805 | C9, MIF, ORM1, SAA | NONE | 4 |
| 806 | C9, MIF, ORM1, TFRC | NONE | 4 |
| 807 | C9, MIF, PKM, SAA | NONE | 4 |
| 808 | C9, MIF, PKM, TFRC | NONE | 4 |
| 809 | C9, MIF, SAA, TFRC | NONE | 4 |
| 810 | C9, ORM1, PKM, SAA | NONE | 4 |
| 811 | C9, ORM1, PKM, TFRC | NONE | 4 |
| 812 | C9, ORM1, SAA, TFRC | NONE | 4 |
| 813 | C9, PKM, SAA, TFRC | NONE | 4 |
| 814 | CEA, DPP4, MIF, ORM1 | NONE | 4 |
| 815 | CEA, DPP4, MIF, PKM | NONE | 4 |
| 816 | CEA, DPP4, MIF, SAA | NONE | 4 |
| 817 | CEA, DPP4, MIF, TFRC | NONE | 4 |
| 818 | CEA, DPP4, ORM1, PKM | NONE | 4 |
| 819 | CEA, DPP4, ORM1, SAA | NONE | 4 |
| 820 | CEA, DPP4, ORM1, TFRC | NONE | 4 |
| 821 | CEA, DPP4, PKM, SAA | NONE | 4 |
| 822 | CEA, DPP4, PKM, TFRC | NONE | 4 |
| 823 | CEA, DPP4, SAA, TFRC | NONE | 4 |
| 824 | CEA, MIF, ORM1, PKM | NONE | 4 |
| 825 | CEA, MIF, ORM1, SAA | NONE | 4 |
| 826 | CEA, MIF, ORM1, TFRC | NONE | 4 |
| 827 | CEA, MIF, PKM, SAA | NONE | 4 |
| 828 | CEA, MIF, PKM, TFRC | NONE | 4 |
| 829 | CEA, MIF, SAA, TFRC | NONE | 4 |
| 830 | CEA, ORM1, PKM, SAA | NONE | 4 |
| 831 | CEA, ORM1, PKM, TFRC | NONE | 4 |
| 832 | CEA, ORM1, SAA, TFRC | NONE | 4 |
| 833 | CEA, PKM, SAA, TFRC | NONE | 4 |
| 834 | DPP4, MIF, ORM1, PKM | NONE | 4 |
| 835 | DPP4, MIF, ORM1, SAA | NONE | 4 |
| 836 | DPP4, MIF, ORM1, TFRC | NONE | 4 |
| 837 | DPP4, MIF, PKM, SAA | NONE | 4 |
| 838 | DPP4, MIF, PKM, TFRC | NONE | 4 |
| 839 | DPP4, MIF, SAA, TFRC | NONE | 4 |
| 840 | DPP4, ORM1, PKM, SAA | NONE | 4 |
| 841 | DPP4, ORM1, PKM, TFRC | NONE | 4 |
| 842 | DPP4, ORM1, SAA, TFRC | NONE | 4 |
| 843 | DPP4, PKM, SAA, TFRC | NONE | 4 |
| 844 | MIF, ORM1, PKM, SAA | NONE | 4 |
| 845 | MIF, ORM1, PKM, TFRC | NONE | 4 |
| 846 | MIF, ORM1, SAA, TFRC | NONE | 4 |
| 847 | MIF, PKM, SAA, TFRC | NONE | 4 |
| 848 | ORM1, PKM, SAA, TFRC | NONE | 4 |
| 849 | C9 | Age and Gender | 3 |
| 850 | CEA | Age and Gender | 3 |
| 851 | DPP4 | Age and Gender | 3 |
| 852 | MIF | Age and Gender | 3 |
| 853 | ORM1 | Age and Gender | 3 |
| 854 | PKM | Age and Gender | 3 |
| 855 | SAA | Age and Gender | 3 |
| 856 | TFRC | Age and Gender | 3 |
| 857 | C9, CEA | Age | 3 |
| 858 | C9, DPP4 | Age | 3 |
| 859 | C9, MIF | Age | 3 |
| 860 | C9, ORM1 | Age | 3 |
| 861 | C9, PKM | Age | 3 |
| 862 | C9, SAA | Age | 3 |
| 863 | C9, TFRC | Age | 3 |
| 864 | CEA, DPP4 | Age | 3 |
| 865 | CEA, MIF | Age | 3 |
| 866 | CEA, ORM1 | Age | 3 |
| 867 | CEA, PKM | Age | 3 |
| 868 | CEA, SAA | Age | 3 |
| 869 | CEA, TFRC | Age | 3 |
| 870 | DPP4, MIF | Age | 3 |
| 871 | DPP4, ORM1 | Age | 3 |
| 872 | DPP4, PKM | Age | 3 |
| 873 | DPP4, SAA | Age | 3 |
| 874 | DPP4, TFRC | Age | 3 |
| 875 | MIF, ORM1 | Age | 3 |
| 876 | MIF, PKM | Age | 3 |
| 877 | MIF, SAA | Age | 3 |
| 878 | MIF, TFRC | Age | 3 |
| 879 | ORM1, PKM | Age | 3 |
| 880 | ORM1, SAA | Age | 3 |
| 881 | ORM1, TFRC | Age | 3 |
| 882 | PKM, SAA | Age | 3 |
| 883 | PKM, TFRC | Age | 3 |
| 884 | SAA, TFRC | Age | 3 |
| 885 | C9, CEA | Gender | 3 |
| 886 | C9, DPP4 | Gender | 3 |
| 887 | C9, MIF | Gender | 3 |
| 888 | C9, ORM1 | Gender | 3 |
| 889 | C9, PKM | Gender | 3 |
| 890 | C9, SAA | Gender | 3 |
| 891 | C9, TFRC | Gender | 3 |
| 892 | CEA, DPP4 | Gender | 3 |
| 893 | CEA, MIF | Gender | 3 |
| 894 | CEA, ORM1 | Gender | 3 |
| 895 | CEA, PKM | Gender | 3 |
| 896 | CEA, SAA | Gender | 3 |
| 897 | CEA, TFRC | Gender | 3 |
| 898 | DPP4, MIF | Gender | 3 |
| 899 | DPP4, ORM1 | Gender | 3 |
| 900 | DPP4, PKM | Gender | 3 |
| 901 | DPP4, SAA | Gender | 3 |
| 902 | DPP4, TFRC | Gender | 3 |
| 903 | MIF, ORM1 | Gender | 3 |
| 904 | MIF, PKM | Gender | 3 |
| 905 | MIF, SAA | Gender | 3 |
| 906 | MIF, TFRC | Gender | 3 |
| 907 | ORM1, PKM | Gender | 3 |
| 908 | ORM1, SAA | Gender | 3 |
| 909 | ORM1, TFRC | Gender | 3 |
| 910 | PKM, SAA | Gender | 3 |
| 911 | PKM, TFRC | Gender | 3 |
| 912 | SAA, TFRC | Gender | 3 |
| 913 | C9, CEA, DPP4 | NONE | 3 |
| 914 | C9, CEA, MIF | NONE | 3 |
| 915 | C9, CEA, ORM1 | NONE | 3 |
| 916 | C9, CEA, PKM | NONE | 3 |
| 917 | C9, CEA, SAA | NONE | 3 |
| 918 | C9, CEA, TFRC | NONE | 3 |
| 919 | C9, DPP4, MIF | NONE | 3 |
| 920 | C9, DPP4, ORM1 | NONE | 3 |
| 921 | C9, DPP4, PKM | NONE | 3 |
| 922 | C9, DPP4, SAA | NONE | 3 |
| 923 | C9, DPP4, TFRC | NONE | 3 |
| 924 | C9, MIF, ORM1 | NONE | 3 |
| 925 | C9, MIF, PKM | NONE | 3 |
| 926 | C9, MIF, SAA | NONE | 3 |
| 927 | C9, MIF, TFRC | NONE | 3 |
| 928 | C9, ORM1, PKM | NONE | 3 |
| 929 | C9, ORM1, SAA | NONE | 3 |
| 930 | C9, ORM1, TFRC | NONE | 3 |
| 931 | C9, PKM, SAA | NONE | 3 |
| 932 | C9, PKM, TFRC | NONE | 3 |
| 933 | C9, SAA, TFRC | NONE | 3 |
| 934 | CEA, DPP4, MIF | NONE | 3 |
| 935 | CEA, DPP4, ORM1 | NONE | 3 |
| 936 | CEA, DPP4, PKM | NONE | 3 |
| 937 | CEA, DPP4, SAA | NONE | 3 |
| 938 | CEA, DPP4, TFRC | NONE | 3 |
| 939 | CEA, MIF, ORM1 | NONE | 3 |
| 940 | CEA, MIF, PKM | NONE | 3 |
| 941 | CEA, MIF, SAA | NONE | 3 |
| 942 | CEA, MIF, TFRC | NONE | 3 |
| 943 | CEA, ORM1, PKM | NONE | 3 |
| 944 | CEA, ORM1, SAA | NONE | 3 |
| 945 | CEA, ORM1, TFRC | NONE | 3 |
| 946 | CEA, PKM, SAA | NONE | 3 |
| 947 | CEA, PKM, TFRC | NONE | 3 |
| 948 | CEA, SAA, TFRC | NONE | 3 |
| 949 | DPP4, MIF, ORM1 | NONE | 3 |
| 950 | DPP4, MIF, PKM | NONE | 3 |
| 951 | DPP4, MIF, SAA | NONE | 3 |
| 952 | DPP4, MIF, TFRC | NONE | 3 |
| 953 | DPP4, ORM1, PKM | NONE | 3 |
| 954 | DPP4, ORM1, SAA | NONE | 3 |
| 955 | DPP4, ORM1, TFRC | NONE | 3 |
| 956 | DPP4, PKM, SAA | NONE | 3 |
| 957 | DPP4, PKM, TFRC | NONE | 3 |
| 958 | DPP4, SAA, TFRC | NONE | 3 |
| 959 | MIF, ORM1, PKM | NONE | 3 |
| 960 | MIF, ORM1, SAA | NONE | 3 |
| 961 | MIF, ORM1, TFRC | NONE | 3 |
| 962 | MIF, PKM, SAA | NONE | 3 |
| 963 | MIF, PKM, TFRC | NONE | 3 |
| 964 | MIF, SAA, TFRC | NONE | 3 |
| 965 | ORM1, PKM, SAA | NONE | 3 |
| 966 | ORM1, PKM, TFRC | NONE | 3 |
| 967 | ORM1, SAA, TFRC | NONE | 3 |
| 968 | PKM, SAA, TFRC | NONE | 3 |

Additional exemplary CRC panels consistent with the disclosure herein are listed in Table 4. Also disclosed are panels comprising the markers listed in entries of Table 4. In some cases, the panels listed in Table 4 can be used as alternatives to panels listed in Table 3 above. Table 4 also includes the Area Under Curve values “AUC”, sensitivity “Sens” and specificity “Spec” values corresponding to each panel.

TABLE 4
CRC biomarker panel constituents

| | | | | | Sens/ |
|---|---|---|---|---|---|
| Ref | CRC Protein Biomarker | Demographics | Features | AUC | Spec |
| 1 | ORM1, SERPINA1, SERPINA3, | Age and Gender | 14 | 84 | 80/71 |
| | CTSD, CEA, CLU, C9, DPP4, GSN, | | | | |
| | MIF, PKM, TIMP1 | | | | |
| 2 | ORM1, SERPINA1, SERPINA3, | Age | 12 | 84 | 80/71 |
| | CEA, CLU, C9, DPP4, GDF15, GSN, MIF, PKM | | | | |
| 3 | ORM1, SERPINA1, SERPINA3, | Age | 13 | 83 | 80/71 |
| | CEA, CLU, DPP4, GDF15, | | | | |
| | GSN, MIF, PKM, SAA, TFRC | | | | |
| 4 | ORM1, SERPINA1, SERPINA3, | Age | 11 | 84 | 80/71 |
| | CEA, C9, DPP4, GDF15, GSN, MIF, PKM | | | | |
| 5 | ORM1, SERPINA1, SERPINA3, | Age | 11 | 84 | 80/74 |
| | CEA, C9, GDF15, GSN, MIF, PKM, TFRC | | | | |
| 6 | ORM1, SERPINA1, SERPINA3, | Age | 11 | 82 | 80/71 |
| | CEA, C9, GDF15, GSN, PKM, SAA, TIMP1 | | | | |
| 7 | ORM1, SERPINA1, SERPINA3, | Age | 12 | 83 | 80/71 |
| | CEA, DPP4, GDF15, | | | | |
| | GSN, MIF, PKM, SAA, TFRC | | | | |
| 8 | ORM1, SERPINA1, SERPINA3, | Age | 11 | 83 | 80/71 |
| | CEA, DPP4, GDF15, MIF, PKM, SAA, TFRC | | | | |
| 9 | ORM1, SERPINA1, CEA, CLU, DPP4, GSN, | Age and Gender | 13 | 83 | 80/71 |
| | MIF, PKM, SAA, TFRC, TIMP1 | | | | |
| 10 | ORM1, SERPINA1, CEA, C9, DPP4, GDF15, | Age | 10 | 84 | 80/71 |
| | GSN, MIF, PKM | | | | |
| 11 | ORM1, SERPINA1, CEA, DPP4, GSN, | Age and Gender | 12 | 83 | 80/71 |
| | MIF, PKM, SAA, TFRC, TIMP1 | | | | |
| 12 | ORM1, SERPINA 1, C9, DPP4, GDF15, GSN, | Age and Gender | 12 | 81 | 80/69 |
| | MIF, PKM, SAA, TFRC | | | | |
| 13 | ORM1, SERPINA1, C9, DPP4, GDF15, | Age | 11 | 81 | 80/69 |
| | GSN, MIF, PKM2, SAA, TFRC | | | | |
| 14 | ORM1, SERPINA1, C9, GDF15, | Age | 9 | 81 | 80/69 |
| | GSN, MIF, PKM, TFRC | | | | |
| 15 | ORM1, SERPINA3, | Age and Gender | 13 | 84 | 80/71 |
| | CTSD, CEA, CLU, C9, DPP4, GSN, | | | | |
| | MIF, PKM, TIMP1 | | | | |
| 16 | ORM1, SERPINA3, CTSD, CEA, C9, DPP4, | Age and Gender | 12 | 84 | 80/71 |
| | GSN, MIF, PKM, TIMP1 | | | | |
| 17 | ORM1, SERPINA3, CEA, C9, GDF15, | Age | 10 | 84 | 80/74 |
| | GSN, MIF, PKM, TFRC | | | | |
| 18 | ORM1, CEA, CLU, C9, DPP4, GSN, | Age and Gender | 11 | 82 | 80/69 |
| | MIF, SAA, TFRC | | | | |
| 19 | ORM1, CEA, CLU, C9, DPP4, GSN, MIF, TFRC | Age and Gender | 10 | 82 | 80/69 |

Exemplary AA panels consistent with the disclosure herein are listed in Table 5. Also disclosed are panels comprising the markers listed in entries of Table 5.

TABLE 5
AA biomarker panel constituents

| Ref | AA Protein Biomarkers | Demo |
|---|---|---|
| 1 | SERPINA1, SERPINA3, CTSD, CLU, DPP4, GDF15, GSN, MIF, PKM, TIMP1, TFRC | Age |
| 2 | SERPINA1, SERPINA3, CTSD, CLU, DPP4, GDF15, GSN, MIF, PKM, TIMP1 | Age |
| 3 | SERPINA1, SERPINA3, CTSD, CLU, DPP4, GDF15, GSN, MIF, PKM, TFRC | Age |
| 4 | SERPINA1, SERPINA3, CTSD, CLU, DPP4, GDF15, GSN, MIF, TIMP1, TFRC | Age |
| 5 | SERPINA1, SERPINA3, CTSD, CLU, DPP4, GDF15, GSN, PKM, TIMP1, TFRC | Age |
| 6 | SERPINA1, SERPINA3, CTSD, CLU, DPP4, GDF15, MIF, PKM, TIMP1, TFRC | Age |
| 7 | SERPINA1, SERPINA3, CTSD, CLU, DPP4, GSN, MIF, PKM, TIMP1, TFRC | Age |
| 8 | SERPINA1, SERPINA3, CTSD, CLU, GDF15, GSN, MIF, PKM, TIMP1, TFRC | Age |
| 9 | SERPINA1, SERPINA3, CTSD, DPP4, GDF15, GSN, MIF, PKM, TIMP1, TFRC | Age |
| 10 | SERPINA1, SERPINA3, CLU, DPP4, GDF15, GSN, MIF, PKM, TIMP1, TFRC | Age |
| 11 | SERPINA1, CTSD, CLU, DPP4, GDF15, GSN, MIF, PKM, TIMP1, TFRC | Age |
| 12 | SERPINA3, CTSD, CLU, DPP4, GDF15, GSN, MIF, PKM, TIMP1, TFRC | Age |
| 13 | SERPINA1, SERPINA3, CTSD, CLU, DPP4, GDF15, GSN, MIF, PKM, TIMP1, TFRC | NONE |
| 14 | SERPINA1, SERPINA3, CTSD, CLU, DPP4, GDF15, GSN, MIF, PKM | Age |
| 15 | SERPINA1, SERPINA3, CTSD, CLU, DPP4, GDF15, GSN, MIF, TIMP1 | Age |
| 16 | SERPINA1, SERPINA3, CTSD, CLU, DPP4, GDF15, GSN, MIF, TFRC | Age |
| 17 | SERPINA1, SERPINA3, CTSD, CLU, DPP4, GDF15, GSN, PKM, TIMP1 | Age |
| 18 | SERPINA1, SERPINA3, CTSD, CLU, DPP4, GDF15, GSN, PKM, TFRC | Age |
| 19 | SERPINA1, SERPINA3, CTSD, CLU, DPP4, GDF15, GSN, TIMP1, TFRC | Age |
| 20 | SERPINA1, SERPINA3, CTSD, CLU, DPP4, GDF15, MIF, PKM, TIMP1 | Age |
| 21 | SERPINA1, SERPINA3, CTSD, CLU, DPP4, GDF15, MIF, PKM, TFRC | Age |
| 22 | SERPINA1, SERPINA3, CTSD, CLU, DPP4, GDF15, MIF, TIMP1, TFRC | Age |
| 23 | SERPINA1, SERPINA3, CTSD, CLU, DPP4, GDF15, PKM, TIMP1, TFRC | Age |
| 24 | SERPINA1, SERPINA3, CTSD, CLU, DPP4, GSN, MIF, PKM, TIMP1 | Age |
| 25 | SERPINA1, SERPINA3, CTSD, CLU, DPP4, GSN, MIF, PKM, TFRC | Age |
| 26 | SERPINA1, SERPINA3, CTSD, CLU, DPP4, GSN, MIF, TIMP1, TFRC | Age |
| 27 | SERPINA1, SERPINA3, CTSD, CLU, DPP4, GSN, PKM, TIMP1, TFRC | Age |
| 28 | SERPINA1, SERPINA3, CTSD, CLU, DPP4, MIF, PKM, TIMP1, TFRC | Age |
| 29 | SERPINA1, SERPINA3, CTSD, CLU, GDF15, GSN, MIF, PKM, TIMP1 | Age |
| 30 | SERPINA1, SERPINA3, CTSD, CLU, GDF15, GSN, MIF, PKM, TFRC | Age |
| 31 | SERPINA1, SERPINA3, CTSD, CLU, GDF15, GSN, MIF, TIMP1, TFRC | Age |
| 32 | SERPINA1, SERPINA3, CTSD, CLU, GDF15, GSN, PKM, TIMP1, TFRC | Age |
| 33 | SERPINA1, SERPINA3, CTSD, CLU, GDF15, MIF, PKM, TIMP1, TFRC | Age |
| 34 | SERPINA1, SERPINA3, CTSD, CLU, GSN, MIF, PKM, TIMP1, TFRC | Age |
| 35 | SERPINA1, SERPINA3, CTSD, DPP4, GDF15, GSN, MIF, PKM, TIMP1 | Age |
| 36 | SERPINA1, SERPINA3, CTSD, DPP4, GDF15, GSN, MIF, PKM, TFRC | Age |
| 37 | SERPINA1, SERPINA3, CTSD, DPP4, GDF15, GSN, MIF, TIMP1, TFRC | Age |
| 38 | SERPINA1, SERPINA3, CTSD, DPP4, GDF15, GSN, PKM, TIMP1, TFRC | Age |
| 39 | SERPINA1, SERPINA3, CTSD, DPP4, GDF15, MIF, PKM, TIMP1, TFRC | Age |
| 40 | SERPINA1, SERPINA3, CTSD, DPP4, GSN, MIF, PKM, TIMP1, TFRC | Age |
| 41 | SERPINA1, SERPINA3, CTSD, GDF15, GSN, MIF, PKM, TIMP1, TFRC | Age |
| 42 | SERPINA1, SERPINA3, CLU, DPP4, GDF15, GSN, MIF, PKM, TIMP1 | Age |
| 43 | SERPINA1, SERPINA3, CLU, DPP4, GDF15, GSN, MIF, PKM, TFRC | Age |
| 44 | SERPINA1, SERPINA3, CLU, DPP4, GDF15, GSN, MIF, TIMP1, TFRC | Age |
| 45 | SERPINA1, SERPINA3, CLU, DPP4, GDF15, GSN, PKM, TIMP1, TFRC | Age |
| 46 | SERPINA1, SERPINA3, CLU, DPP4, GDF15, MIF, PKM, TIMP1, TFRC | Age |
| 47 | SERPINA1, SERPINA3, CLU, DPP4, GSN, MIF, PKM, TIMP1, TFRC | Age |
| 48 | SERPINA1, SERPINA3, CLU, GDF15, GSN, MIF, PKM, TIMP1, TFRC | Age |
| 49 | SERPINA1, SERPINA3, DPP4, GDF15, GSN, MIF, PKM, TIMP1, TFRC | Age |
| 50 | SERPINA1, CTSD, CLU, DPP4, GDF15, GSN, MIF, PKM, TIMP1 | Age |
| 51 | SERPINA1, CTSD, CLU, DPP4, GDF15, GSN, MIF, PKM, TFRC | Age |
| 52 | SERPINA1, CTSD, CLU, DPP4, GDF15, GSN, MIF, TIMP1, TFRC | Age |
| 53 | SERPINA1, CTSD, CLU, DPP4, GDF15, GSN, PKM, TIMP1, TFRC | Age |
| 54 | SERPINA1, CTSD, CLU, DPP4, GDF15, MIF, PKM, TIMP1, TFRC | Age |
| 55 | SERPINA1, CTSD, CLU, DPP4, GSN, MIF, PKM, TIMP1, TFRC | Age |
| 56 | SERPINA1, CTSD, CLU, GDF15, GSN, MIF, PKM, TIMP1, TFRC | Age |
| 57 | SERPINA1, CTSD, DPP4, GDF15, GSN, MIF, PKM, TIMP1, TFRC | Age |
| 58 | SERPINA1, CLU, DPP4, GDF15, GSN, MIF, PKM, TIMP1, TFRC | Age |
| 59 | SERPINA3, CTSD, CLU, DPP4, GDF15, GSN, MIF, PKM, TIMP1 | Age |
| 60 | SERPINA3, CTSD, CLU, DPP4, GDF15, GSN, MIF, PKM, TFRC | Age |
| 61 | SERPINA3, CTSD, CLU, DPP4, GDF15, GSN, MIF, TIMP1, TFRC | Age |
| 62 | SERPINA3, CTSD, CLU, DPP4, GDF15, GSN, PKM, TIMP1, TFRC | Age |
| 63 | SERPINA3, CTSD, CLU, DPP4, GDF15, MIF, PKM, TIMP1, TFRC | Age |
| 64 | SERPINA3, CTSD, CLU, DPP4, GSN, MIF, PKM, TIMP1, TFRC | Age |
| 65 | SERPINA3, CTSD, CLU, GDF15, GSN, MIF, PKM, TIMP1, TFRC | Age |
| 66 | SERPINA3, CTSD, DPP4, GDF15, GSN, MIF, PKM, TIMP1, TFRC | Age |
| 67 | SERPINA3, CLU, DPP4, GDF15, GSN, MIF, PKM, TIMP1, TFRC | Age |
| 68 | CTSD, CLU, DPP4, GDF15, GSN, MIF, PKM, TIMP1, TFRC | Age |
| 69 | SERPINA1, SERPINA3, CTSD, CLU, DPP4, GDF15, GSN, MIF, PKM, TIMP1 | NONE |
| 70 | SERPINA1, SERPINA3, CTSD, CLU, DPP4, GDF15, GSN, MIF, PKM, TFRC | NONE |
| 71 | SERPINA1, SERPINA3, CTSD, CLU, DPP4, GDF15, GSN, MIF, TIMP1, TFRC | NONE |
| 72 | SERPINA1, SERPINA3, CTSD, CLU, DPP4, GDF15, GSN, PKM, TIMP1, TFRC | NONE |
| 73 | SERPINA1, SERPINA3, CTSD, CLU, DPP4, GDF15, MIF, PKM, TIMP1, TFRC | NONE |
| 74 | SERPINA1, SERPINA3, CTSD, CLU, DPP4, GSN, MIF, PKM, TIMP1, TFRC | NONE |
| 75 | SERPINA1, SERPINA3, CTSD, CLU, GDF15, GSN, MIF, PKM, TIMP1, TFRC | NONE |
| 76 | SERPINA1, SERPINA3, CTSD, DPP4, GDF15, GSN, MIF, PKM, TIMP1, TFRC | NONE |
| 77 | SERPINA1, SERPINA3, CLU, DPP4, GDF15, GSN, MIF, PKM, TIMP1, TFRC | NONE |
| 78 | SERPINA1, CTSD, CLU, DPP4, GDF15, GSN, MIF, PKM, TIMP1, TFRC | NONE |
| 79 | SERPINA3, CTSD, CLU, DPP4, GDF15, GSN, MIF, PKM, TIMP1, TFRC | NONE |
| 80 | SERPINA1, SERPINA3, CTSD, CLU, DPP4, GDF15, GSN, MIF | Age |
| 81 | SERPINA1, SERPINA3, CTSD, CLU, DPP4, GDF15, GSN, PKM | Age |
| 82 | SERPINA1, SERPINA3, CTSD, CLU, DPP4, GDF15, GSN, TIMP1 | Age |
| 83 | SERPINA1, SERPINA3, CTSD, CLU, DPP4, GDF15, GSN, TFRC | Age |
| 84 | SERPINA1, SERPINA3, CTSD, CLU, DPP4, GDF15, MIF, PKM | Age |
| 85 | SERPINA1, SERPINA3, CTSD, CLU, DPP4, GDF15, MIF, TIMP1 | Age |
| 86 | SERPINA1, SERPINA3, CTSD, CLU, DPP4, GDF15, MIF, TFRC | Age |
| 87 | SERPINA1, SERPINA3, CTSD, CLU, DPP4, GDF15, PKM, TIMP1 | Age |
| 88 | SERPINA1, SERPINA3, CTSD, CLU, DPP4, GDF15, PKM, TFRC | Age |
| 89 | SERPINA1, SERPINA3, CTSD, CLU, DPP4, GDF15, TIMP1, TFRC | Age |
| 90 | SERPINA1, SERPINA3, CTSD, CLU, DPP4, GSN, MIF, PKM | Age |
| 91 | SERPINA1, SERPINA3, CTSD, CLU, DPP4, GSN, MIF, TIMP1 | Age |
| 92 | SERPINA1, SERPINA3, CTSD, CLU, DPP4, GSN, MIF, TFRC | Age |
| 93 | SERPINA1, SERPINA3, CTSD, CLU, DPP4, GSN, PKM, TIMP1 | Age |
| 94 | SERPINA1, SERPINA3, CTSD, CLU, DPP4, GSN, PKM, TFRC | Age |
| 95 | SERPINA1, SERPINA3, CTSD, CLU, DPP4, GSN, TIMP1, TFRC | Age |
| 96 | SERPINA1, SERPINA3, CTSD, CLU, DPP4, MIF, PKM, TIMP1 | Age |
| 97 | SERPINA1, SERPINA3, CTSD, CLU, DPP4, MIF, PKM, TFRC | Age |
| 98 | SERPINA1, SERPINA3, CTSD, CLU, DPP4, MIF, TIMP1, TFRC | Age |
| 99 | SERPINA1, SERPINA3, CTSD, CLU, DPP4, PKM, TIMP1, TFRC | Age |
| 100 | SERPINA1, SERPINA3, CTSD, CLU, GDF15, GSN, MIF, PKM | Age |
| 101 | SERPINA1, SERPINA3, CTSD, CLU, GDF15, GSN, MIF, TIMP1 | Age |
| 102 | SERPINA1, SERPINA3, CTSD, CLU, GDF15, GSN, MIF, TFRC | Age |
| 103 | SERPINA1, SERPINA3, CTSD, CLU, GDF15, GSN, PKM, TIMP1 | Age |
| 104 | SERPINA1, SERPINA3, CTSD, CLU, GDF15, GSN, PKM, TFRC | Age |
| 105 | SERPINA1, SERPINA3, CTSD, CLU, GDF15, GSN, TIMP1, TFRC | Age |
| 106 | SERPINA1, SERPINA3, CTSD, CLU, GDF15, MIF, PKM, TIMP1 | Age |
| 107 | SERPINA1, SERPINA3, CTSD, CLU, GDF15, MIF, PKM, TFRC | Age |
| 108 | SERPINA1, SERPINA3, CTSD, CLU, GDF15, MIF, TIMP1, TFRC | Age |
| 109 | SERPINA1, SERPINA3, CTSD, CLU, GDF15, PKM, TIMP1, TFRC | Age |
| 110 | SERPINA1, SERPINA3, CTSD, CLU, GSN, MIF, PKM, TIMP1 | Age |
| 111 | SERPINA1, SERPINA3, CTSD, CLU, GSN, MIF, PKM, TFRC | Age |
| 112 | SERPINA1, SERPINA3, CTSD, CLU, GSN, MIF, TIMP1, TFRC | Age |
| 113 | SERPINA1, SERPINA3, CTSD, CLU, GSN, PKM, TIMP1, TFRC | Age |
| 114 | SERPINA1, SERPINA3, CTSD, CLU, MIF, PKM, TIMP1, TFRC | Age |
| 115 | SERPINA1, SERPINA3, CTSD, DPP4, GDF15, GSN, MIF, PKM | Age |
| 116 | SERPINA1, SERPINA3, CTSD, DPP4, GDF15, GSN, MIF, TIMP1 | Age |
| 117 | SERPINA1, SERPINA3, CTSD, DPP4, GDF15, GSN, MIF, TFRC | Age |
| 118 | SERPINA1, SERPINA3, CTSD, DPP4, GDF15, GSN, PKM, TIMP1 | Age |
| 119 | SERPINA1, SERPINA3, CTSD, DPP4, GDF15, GSN, PKM, TFRC | Age |
| 120 | SERPINA1, SERPINA3, CTSD, DPP4, GDF15, GSN, TIMP1, TFRC | Age |
| 121 | SERPINA1, SERPINA3, CTSD, DPP4, GDF15, MIF, PKM, TIMP1 | Age |
| 122 | SERPINA1, SERPINA3, CTSD, DPP4, GDF15, MIF, PKM, TFRC | Age |
| 123 | SERPINA1, SERPINA3, CTSD, DPP4, GDF15, MIF, TIMP1, TFRC | Age |
| 124 | SERPINA1, SERPINA3, CTSD, DPP4, GDF15, PKM, TIMP1, TFRC | Age |
| 125 | SERPINA1, SERPINA3, CTSD, DPP4, GSN, MIF, PKM, TIMP1 | Age |
| 126 | SERPINA1, SERPINA3, CTSD, DPP4, GSN, MIF, PKM, TFRC | Age |
| 127 | SERPINA1, SERPINA3, CTSD, DPP4, GSN, MIF, TIMP1, TFRC | Age |
| 128 | SERPINA1, SERPINA3, CTSD, DPP4, GSN, PKM, TIMP1, TFRC | Age |
| 129 | SERPINA1, SERPINA3, CTSD, DPP4, MIF, PKM, TIMP1, TFRC | Age |
| 130 | SERPINA1, SERPINA3, CTSD, GDF15, GSN, MIF, PKM, TIMP1 | Age |
| 131 | SERPINA1, SERPINA3, CTSD, GDF15, GSN, MIF, PKM, TFRC | Age |
| 132 | SERPINA1, SERPINA3, CTSD, GDF15, GSN, MIF, TIMP1, TFRC | Age |
| 133 | SERPINA1, SERPINA3, CTSD, GDF15, GSN, PKM, TIMP1, TFRC | Age |
| 134 | SERPINA1, SERPINA3, CTSD, GDF15, MIF, PKM, TIMP1, TFRC | Age |
| 135 | SERPINA1, SERPINA3, CTSD, GSN, MIF, PKM, TIMP1, TFRC | Age |
| 136 | SERPINA1, SERPINA3, CLU, DPP4, GDF15, GSN, MIF, PKM | Age |
| 137 | SERPINA1, SERPINA3, CLU, DPP4, GDF15, GSN, MIF, TIMP1 | Age |
| 138 | SERPINA1, SERPINA3, CLU, DPP4, GDF15, GSN, MIF, TFRC | Age |
| 139 | SERPINA1, SERPINA3, CLU, DPP4, GDF15, GSN, PKM, TIMP1 | Age |
| 140 | SERPINA1, SERPINA3, CLU, DPP4, GDF15, GSN, PKM, TFRC | Age |
| 141 | SERPINA1, SERPINA3, CLU, DPP4, GDF15, GSN, TIMP1, TFRC | Age |
| 142 | SERPINA1, SERPINA3, CLU, DPP4, GDF15, MIF, PKM, TIMP1 | Age |
| 143 | SERPINA1, SERPINA3, CLU, DPP4, GDF15, MIF, PKM, TFRC | Age |
| 144 | SERPINA1, SERPINA3, CLU, DPP4, GDF15, MIF, TIMP1, TFRC | Age |
| 145 | SERPINA1, SERPINA3, CLU, DPP4, GDF15, PKM, TIMP1, TFRC | Age |
| 146 | SERPINA1, SERPINA3, CLU, DPP4, GSN, MIF, PKM, TIMP1 | Age |
| 147 | SERPINA1, SERPINA3, CLU, DPP4, GSN, MIF, PKM, TFRC | Age |
| 148 | SERPINA1, SERPINA3, CLU, DPP4, GSN, MIF, TIMP1, TFRC | Age |
| 149 | SERPINA1, SERPINA3, CLU, DPP4, GSN, PKM, TIMP1, TFRC | Age |
| 150 | SERPINA1, SERPINA3, CLU, DPP4, MIF, PKM, TIMP1, TFRC | Age |
| 151 | SERPINA1, SERPINA3, CLU, GDF15, GSN, MIF, PKM, TIMP1 | Age |
| 152 | SERPINA1, SERPINA3, CLU, GDF15, GSN, MIF, PKM, TFRC | Age |
| 153 | SERPINA1, SERPINA3, CLU, GDF15, GSN, MIF, TIMP1, TFRC | Age |
| 154 | SERPINA1, SERPINA3, CLU, GDF15, GSN, PKM, TIMP1, TFRC | Age |
| 155 | SERPINA1, SERPINA3, CLU, GDF15, MIF, PKM, TIMP1, TFRC | Age |
| 156 | SERPINA1, SERPINA3, CLU, GSN, MIF, PKM, TIMP1, TFRC | Age |
| 157 | SERPINA1, SERPINA3, DPP4, GDF15, GSN, MIF, PKM, TIMP1 | Age |
| 158 | SERPINA1, SERPINA3, DPP4, GDF15, GSN, MIF, PKM, TFRC | Age |
| 159 | SERPINA1, SERPINA3, DPP4, GDF15, GSN, MIF, TIMP1, TFRC | Age |
| 160 | SERPINA1, SERPINA3, DPP4, GDF15, GSN, PKM, TIMP1, TFRC | Age |
| 161 | SERPINA1, SERPINA3, DPP4, GDF15, MIF, PKM, TIMP1, TFRC | Age |
| 162 | SERPINA1, SERPINA3, DPP4, GSN, MIF, PKM, TIMP1, TFRC | Age |
| 163 | SERPINA1, SERPINA3, GDF15, GSN, MIF, PKM, TIMP1, TFRC | Age |
| 164 | SERPINA1, CTSD, CLU, DPP4, GDF15, GSN, MIF, PKM | Age |
| 165 | SERPINA1, CTSD, CLU, DPP4, GDF15, GSN, MIF, TIMP1 | Age |
| 166 | SERPINA1, CTSD, CLU, DPP4, GDF15, GSN, MIF, TFRC | Age |
| 167 | SERPINA1, CTSD, CLU, DPP4, GDF15, GSN, PKM, TIMP1 | Age |
| 168 | SERPINA1, CTSD, CLU, DPP4, GDF15, GSN, PKM, TFRC | Age |
| 169 | SERPINA1, CTSD, CLU, DPP4, GDF15, GSN, TIMP1, TFRC | Age |
| 170 | SERPINA1, CTSD, CLU, DPP4, GDF15, MIF, PKM, TIMP1 | Age |
| 171 | SERPINA1, CTSD, CLU, DPP4, GDF15, MIF, PKM, TFRC | Age |
| 172 | SERPINA1, CTSD, CLU, DPP4, GDF15, MIF, TIMP1, TFRC | Age |
| 173 | SERPINA1, CTSD, CLU, DPP4, GDF15, PKM, TIMP1, TFRC | Age |
| 174 | SERPINA1, CTSD, CLU, DPP4, GSN, MIF, PKM, TIMP1 | Age |
| 175 | SERPINA1, CTSD, CLU, DPP4, GSN, MIF, PKM, TFRC | Age |
| 176 | SERPINA1, CTSD, CLU, DPP4, GSN, MIF, TIMP1, TFRC | Age |
| 177 | SERPINA1, CTSD, CLU, DPP4, GSN, PKM, TIMP1, TFRC | Age |
| 178 | SERPINA1, CTSD, CLU, DPP4, MIF, PKM, TIMP1, TFRC | Age |
| 179 | SERPINA1, CTSD, CLU, GDF15, GSN, MIF, PKM, TIMP1 | Age |
| 180 | SERPINA1, CTSD, CLU, GDF15, GSN, MIF, PKM, TFRC | Age |
| 181 | SERPINA1, CTSD, CLU, GDF15, GSN, MIF, TIMP1, TFRC | Age |
| 182 | SERPINA1, CTSD, CLU, GDF15, GSN, PKM, TIMP1, TFRC | Age |
| 183 | SERPINA1, CTSD, CLU, GDF15, MIF, PKM, TIMP1, TFRC | Age |
| 184 | SERPINA1, CTSD, CLU, GSN, MIF, PKM, TIMP1, TFRC | Age |
| 185 | SERPINA1, CTSD, DPP4, GDF15, GSN, MIF, PKM, TIMP1 | Age |
| 186 | SERPINA1, CTSD, DPP4, GDF15, GSN, MIF, PKM, TFRC | Age |
| 187 | SERPINA1, CTSD, DPP4, GDF15, GSN, MIF, TIMP1, TFRC | Age |
| 188 | SERPINA1, CTSD, DPP4, GDF15, GSN, PKM, TIMP1, TFRC | Age |
| 189 | SERPINA1, CTSD, DPP4, GDF15, MIF, PKM, TIMP1, TFRC | Age |
| 190 | SERPINA1, CTSD, DPP4, GSN, MIF, PKM, TIMP1, TFRC | Age |
| 191 | SERPINA1, CTSD, GDF15, GSN, MIF, PKM, TIMP1, TFRC | Age |
| 192 | SERPINA1, CLU, DPP4, GDF15, GSN, MIF, PKM, TIMP1 | Age |
| 193 | SERPINA1, CLU, DPP4, GDF15, GSN, MIF, PKM, TFRC | Age |
| 194 | SERPINA1, CLU, DPP4, GDF15, GSN, MIF, TIMP1, TFRC | Age |
| 195 | SERPINA1, CLU, DPP4, GDF15, GSN, PKM, TIMP1, TFRC | Age |
| 196 | SERPINA1, CLU, DPP4, GDF15, MIF, PKM, TIMP1, TFRC | Age |
| 197 | SERPINA1, CLU, DPP4, GSN, MIF, PKM, TIMP1, TFRC | Age |
| 198 | SERPINA1, CLU, GDF15, GSN, MIF, PKM, TIMP1, TFRC | Age |
| 199 | SERPINA1, DPP4, GDF15, GSN, MIF, PKM, TIMP1, TFRC | Age |
| 200 | SERPINA3, CTSD, CLU, DPP4, GDF15, GSN, MIF, PKM | Age |
| 201 | SERPINA3, CTSD, CLU, DPP4, GDF15, GSN, MIF, TIMP1 | Age |
| 202 | SERPINA3, CTSD, CLU, DPP4, GDF15, GSN, MIF, TFRC | Age |
| 203 | SERPINA3, CTSD, CLU, DPP4, GDF15, GSN, PKM, TIMP1 | Age |
| 204 | SERPINA3, CTSD, CLU, DPP4, GDF15, GSN, PKM, TFRC | Age |
| 205 | SERPINA3, CTSD, CLU, DPP4, GDF15, GSN, TIMP1, TFRC | Age |
| 206 | SERPINA3, CTSD, CLU, DPP4, GDF15, MIF, PKM, TIMP1 | Age |
| 207 | SERPINA3, CTSD, CLU, DPP4, GDF15, MIF, PKM, TFRC | Age |
| 208 | SERPINA3, CTSD, CLU, DPP4, GDF15, MIF, TIMP1, TFRC | Age |
| 209 | SERPINA3, CTSD, CLU, DPP4, GDF15, PKM, TIMP1, TFRC | Age |
| 210 | SERPINA3, CTSD, CLU, DPP4, GSN, MIF, PKM, TIMP1 | Age |
| 211 | SERPINA3, CTSD, CLU, DPP4, GSN, MIF, PKM, TFRC | Age |
| 212 | SERPINA3, CTSD, CLU, DPP4, GSN, MIF, TIMP1, TFRC | Age |
| 213 | SERPINA3, CTSD, CLU, DPP4, GSN, PKM, TIMP1, TFRC | Age |
| 214 | SERPINA3, CTSD, CLU, DPP4, MIF, PKM, TIMP1, TFRC | Age |
| 215 | SERPINA3, CTSD, CLU, GDF15, GSN, MIF, PKM, TIMP1 | Age |
| 216 | SERPINA3, CTSD, CLU, GDF15, GSN, MIF, PKM, TFRC | Age |
| 217 | SERPINA3, CTSD, CLU, GDF15, GSN, MIF, TIMP1, TFRC | Age |
| 218 | SERPINA3, CTSD, CLU, GDF15, GSN, PKM, TIMP1, TFRC | Age |
| 219 | SERPINA3, CTSD, CLU, GDF15, MIF, PKM, TIMP1, TFRC | Age |
| 220 | SERPINA3, CTSD, CLU, GSN, MIF, PKM, TIMP1, TFRC | Age |
| 221 | SERPINA3, CTSD, DPP4, GDF15, GSN, MIF, PKM, TIMP1 | Age |
| 222 | SERPINA3, CTSD, DPP4, GDF15, GSN, MIF, PKM, TFRC | Age |
| 223 | SERPINA3, CTSD, DPP4, GDF15, GSN, MIF, TIMP1, TFRC | Age |
| 224 | SERPINA3, CTSD, DPP4, GDF15, GSN, PKM, TIMP1, TFRC | Age |
| 225 | SERPINA3, CTSD, DPP4, GDF15, MIF, PKM, TIMP1, TFRC | Age |
| 226 | SERPINA3, CTSD, DPP4, GSN, MIF, PKM, TIMP1, TFRC | Age |
| 227 | SERPINA3, CTSD, GDF15, GSN, MIF, PKM, TIMP1, TFRC | Age |
| 228 | SERPINA3, CLU, DPP4, GDF15, GSN, MIF, PKM, TIMP1 | Age |
| 229 | SERPINA3, CLU, DPP4, GDF15, GSN, MIF, PKM, TFRC | Age |
| 230 | SERPINA3, CLU, DPP4, GDF15, GSN, MIF, TIMP1, TFRC | Age |
| 231 | SERPINA3, CLU, DPP4, GDF15, GSN, PKM, TIMP1, TFRC | Age |
| 232 | SERPINA3, CLU, DPP4, GDF15, MIF, PKM, TIMP1, TFRC | Age |
| 233 | SERPINA3, CLU, DPP4, GSN, MIF, PKM, TIMP1, TFRC | Age |
| 234 | SERPINA3, CLU, GDF15, GSN, MIF, PKM, TIMP1, TFRC | Age |
| 235 | SERPINA3, DPP4, GDF15, GSN, MIF, PKM, TIMP1, TFRC | Age |
| 236 | CTSD, CLU, DPP4, GDF15, GSN, MIF, PKM, TIMP1 | Age |
| 237 | CTSD, CLU, DPP4, GDF15, GSN, MIF, PKM, TFRC | Age |
| 238 | CTSD, CLU, DPP4, GDF15, GSN, MIF, TIMP1, TFRC | Age |
| 239 | CTSD, CLU, DPP4, GDF15, GSN, PKM, TIMP1, TFRC | Age |
| 240 | CTSD, CLU, DPP4, GDF15, MIF, PKM, TIMP1, TFRC | Age |
| 241 | CTSD, CLU, DPP4, GSN, MIF, PKM, TIMP1, TFRC | Age |
| 242 | CTSD, CLU, GDF15, GSN, MIF, PKM, TIMP1, TFRC | Age |
| 243 | CTSD, DPP4, GDF15, GSN, MIF, PKM, TIMP1, TFRC | Age |
| 244 | CLU, DPP4, GDF15, GSN, MIF, PKM, TIMP1, TFRC | Age |
| 245 | SERPINA1, SERPINA3, CTSD, CLU, DPP4, GDF15, GSN, MIF, PKM | NONE |
| 246 | SERPINA1, SERPINA3, CTSD, CLU, DPP4, GDF15, GSN, MIF, TIMP1 | NONE |
| 247 | SERPINA1, SERPINA3, CTSD, CLU, DPP4, GDF15, GSN, MIF, TFRC | NONE |
| 248 | SERPINA1, SERPINA3, CTSD, CLU, DPP4, GDF15, GSN, PKM, TIMP1 | NONE |
| 249 | SERPINA1, SERPINA3, CTSD, CLU, DPP4, GDF15, GSN, PKM, TFRC | NONE |
| 250 | SERPINA1, SERPINA3, CTSD, CLU, DPP4, GDF15, GSN, TIMP1, TFRC | NONE |
| 251 | SERPINA1, SERPINA3, CTSD, CLU, DPP4, GDF15, MIF, PKM, TIMP1 | NONE |
| 252 | SERPINA1, SERPINA3, CTSD, CLU, DPP4, GDF15, MIF, PKM, TFRC | NONE |
| 253 | SERPINA1, SERPINA3, CTSD, CLU, DPP4, GDF15, MIF, TIMP1, TFRC | NONE |
| 254 | SERPINA1, SERPINA3, CTSD, CLU, DPP4, GDF15, PKM, TIMP1, TFRC | NONE |
| 255 | SERPINA1, SERPINA3, CTSD, CLU, DPP4, GSN, MIF, PKM, TIMP1 | NONE |
| 256 | SERPINA1, SERPINA3, CTSD, CLU, DPP4, GSN, MIF, PKM, TFRC | NONE |
| 257 | SERPINA1, SERPINA3, CTSD, CLU, DPP4, GSN, MIF, TIMP1, TFRC | NONE |
| 258 | SERPINA1, SERPINA3, CTSD, CLU, DPP4, GSN, PKM, TIMP1, TFRC | NONE |
| 259 | SERPINA1, SERPINA3, CTSD, CLU, DPP4, MIF, PKM, TIMP1, TFRC | NONE |
| 260 | SERPINA1, SERPINA3, CTSD, CLU, GDF15, GSN, MIF, PKM, TIMP1 | NONE |
| 261 | SERPINA1, SERPINA3, CTSD, CLU, GDF15, GSN, MIF, PKM, TFRC | NONE |
| 262 | SERPINA1, SERPINA3, CTSD, CLU, GDF15, GSN, MIF, TIMP1, TFRC | NONE |
| 263 | SERPINA1, SERPINA3, CTSD, CLU, GDF15, GSN, PKM, TIMP1, TFRC | NONE |
| 264 | SERPINA1, SERPINA3, CTSD, CLU, GDF15, MIF, PKM, TIMP1, TFRC | NONE |
| 265 | SERPINA1, SERPINA3, CTSD, CLU, GSN, MIF, PKM, TIMP1, TFRC | NONE |
| 266 | SERPINA1, SERPINA3, CTSD, DPP4, GDF15, GSN, MIF, PKM, TIMP1 | NONE |
| 267 | SERPINA1, SERPINA3, CTSD, DPP4, GDF15, GSN, MIF, PKM, TFRC | NONE |
| 268 | SERPINA1, SERPINA3, CTSD, DPP4, GDF15, GSN, MIF, TIMP1, TFRC | NONE |
| 269 | SERPINA1, SERPINA3, CTSD, DPP4, GDF15, GSN, PKM, TIMP1, TFRC | NONE |
| 270 | SERPINA1, SERPINA3, CTSD, DPP4, GDF15, MIF, PKM, TIMP1, TFRC | NONE |
| 271 | SERPINA1, SERPINA3, CTSD, DPP4, GSN, MIF, PKM, TIMP1, TFRC | NONE |
| 272 | SERPINA1, SERPINA3, CTSD, GDF15, GSN, MIF, PKM, TIMP1, TFRC | NONE |
| 273 | SERPINA1, SERPINA3, CLU, DPP4, GDF15, GSN, MIF, PKM, TIMP1 | NONE |
| 274 | SERPINA1, SERPINA3, CLU, DPP4, GDF15, GSN, MIF, PKM, TFRC | NONE |
| 275 | SERPINA1, SERPINA3, CLU, DPP4, GDF15, GSN, MIF, TIMP1, TFRC | NONE |
| 276 | SERPINA1, SERPINA3, CLU, DPP4, GDF15, GSN, PKM, TIMP1, TFRC | NONE |
| 277 | SERPINA1, SERPINA3, CLU, DPP4, GDF15, MIF, PKM, TIMP1, TFRC | NONE |
| 278 | SERPINA1, SERPINA3, CLU, DPP4, GSN, MIF, PKM, TIMP1, TFRC | NONE |
| 279 | SERPINA1, SERPINA3, CLU, GDF15, GSN, MIF, PKM, TIMP1, TFRC | NONE |
| 280 | SERPINA1, SERPINA3, DPP4, GDF15, GSN, MIF, PKM, TIMP1, TFRC | NONE |
| 281 | SERPINA1, CTSD, CLU, DPP4, GDF15, GSN, MIF, PKM, TIMP1 | NONE |
| 282 | SERPINA1, CTSD, CLU, DPP4, GDF15, GSN, MIF, PKM, TFRC | NONE |
| 283 | SERPINA1, CTSD, CLU, DPP4, GDF15, GSN, MIF, TIMP1, TFRC | NONE |
| 284 | SERPINA1, CTSD, CLU, DPP4, GDF15, GSN, PKM, TIMP1, TFRC | NONE |
| 285 | SERPINA1, CTSD, CLU, DPP4, GDF15, MIF, PKM, TIMP1, TFRC | NONE |
| 286 | SERPINA1, CTSD, CLU, DPP4, GSN, MIF, PKM, TIMP1, TFRC | NONE |
| 287 | SERPINA1, CTSD, CLU, GDF15, GSN, MIF, PKM, TIMP1, TFRC | NONE |
| 288 | SERPINA1, CTSD, DPP4, GDF15, GSN, MIF, PKM, TIMP1, TFRC | NONE |
| 289 | SERPINA1, CLU, DPP4, GDF15, GSN, MIF, PKM, TIMP1, TFRC | NONE |
| 290 | SERPINA3, CTSD, CLU, DPP4, GDF15, GSN, MIF, PKM, TIMP1 | NONE |
| 291 | SERPINA3, CTSD, CLU, DPP4, GDF15, GSN, MIF, PKM, TFRC | NONE |
| 292 | SERPINA3, CTSD, CLU, DPP4, GDF15, GSN, MIF, TIMP1, TFRC | NONE |
| 293 | SERPINA3, CTSD, CLU, DPP4, GDF15, GSN, PKM, TIMP1, TFRC | NONE |
| 294 | SERPINA3, CTSD, CLU, DPP4, GDF15, MIF, PKM, TIMP1, TFRC | NONE |
| 295 | SERPINA3, CTSD, CLU, DPP4, GSN, MIF, PKM, TIMP1, TFRC | NONE |
| 296 | SERPINA3, CTSD, CLU, GDF15, GSN, MIF, PKM, TIMP1, TFRC | NONE |
| 297 | SERPINA3, CTSD, DPP4, GDF15, GSN, MIF, PKM, TIMP1, TFRC | NONE |
| 298 | SERPINA3, CLU, DPP4, GDF15, GSN, MIF, PKM, TIMP1, TFRC | NONE |
| 299 | CTSD, CLU, DPP4, GDF15, GSN, MIF, PKM, TIMP1, TFRC | NONE |
| 300 | SERPINA1, SERPINA3, CTSD, CLU, DPP4, GDF15, GSN | Age |
| 301 | SERPINA1, SERPINA3, CTSD, CLU, DPP4, GDF15, MIF | Age |
| 302 | SERPINA1, SERPINA3, CTSD, CLU, DPP4, GDF15, PKM | Age |
| 303 | SERPINA1, SERPINA3, CTSD, CLU, DPP4, GDF15, TIMP1 | Age |
| 304 | SERPINA1, SERPINA3, CTSD, CLU, DPP4, GDF15, TFRC | Age |
| 305 | SERPINA1, SERPINA3, CTSD, CLU, DPP4, GSN, MIF | Age |
| 306 | SERPINA1, SERPINA3, CTSD, CLU, DPP4, GSN, PKM | Age |
| 307 | SERPINA1, SERPINA3, CTSD, CLU, DPP4, GSN, TIMP1 | Age |
| 308 | SERPINA1, SERPINA3, CTSD, CLU, DPP4, GSN, TFRC | Age |
| 309 | SERPINA1, SERPINA3, CTSD, CLU, DPP4, MIF, PKM | Age |
| 310 | SERPINA1, SERPINA3, CTSD, CLU, DPP4, MIF, TIMP1 | Age |
| 311 | SERPINA1, SERPINA3, CTSD, CLU, DPP4, MIF, TFRC | Age |
| 312 | SERPINA1, SERPINA3, CTSD, CLU, DPP4, PKM, TIMP1 | Age |
| 313 | SERPINA1, SERPINA3, CTSD, CLU, DPP4, PKM, TFRC | Age |
| 314 | SERPINA1, SERPINA3, CTSD, CLU, DPP4, TIMP1, TFRC | Age |
| 315 | SERPINA1, SERPINA3, CTSD, CLU, GDF15, GSN, MIF | Age |
| 316 | SERPINA1, SERPINA3, CTSD, CLU, GDF15, GSN, PKM | Age |
| 317 | SERPINA1, SERPINA3, CTSD, CLU, GDF15, GSN, TIMP1 | Age |
| 318 | SERPINA1, SERPINA3, CTSD, CLU, GDF15, GSN, TFRC | Age |
| 319 | SERPINA1, SERPINA3, CTSD, CLU, GDF15, MIF, PKM | Age |
| 320 | SERPINA1, SERPINA3, CTSD, CLU, GDF15, MIF, TIMP1 | Age |
| 321 | SERPINA1, SERPINA3, CTSD, CLU, GDF15, MIF, TFRC | Age |
| 322 | SERPINA1, SERPINA3, CTSD, CLU, GDF15, PKM, TIMP1 | Age |
| 323 | SERPINA1, SERPINA3, CTSD, CLU, GDF15, PKM, TFRC | Age |
| 324 | SERPINA1, SERPINA3, CTSD, CLU, GDF15, TIMP1, TFRC | Age |
| 325 | SERPINA1, SERPINA3, CTSD, CLU, GSN, MIF, PKM | Age |
| 326 | SERPINA1, SERPINA3, CTSD, CLU, GSN, MIF, TIMP1 | Age |
| 327 | SERPINA1, SERPINA3, CTSD, CLU, GSN, MIF, TFRC | Age |
| 328 | SERPINA1, SERPINA3, CTSD, CLU, GSN, PKM, TIMP1 | Age |
| 329 | SERPINA1, SERPINA3, CTSD, CLU, GSN, PKM, TFRC | Age |
| 330 | SERPINA1, SERPINA3, CTSD, CLU, GSN, TIMP1, TFRC | Age |
| 331 | SERPINA1, SERPINA3, CTSD, CLU, MIF, PKM, TIMP1 | Age |
| 332 | SERPINA1, SERPINA3, CTSD, CLU, MIF, PKM, TFRC | Age |
| 333 | SERPINA1, SERPINA3, CTSD, CLU, MIF, TIMP1, TFRC | Age |
| 334 | SERPINA1, SERPINA3, CTSD, CLU, PKM, TIMP1, TFRC | Age |
| 335 | SERPINA1, SERPINA3, CTSD, DPP4, GDF15, GSN, MIF | Age |
| 336 | SERPINA1, SERPINA3, CTSD, DPP4, GDF15, GSN, PKM | Age |
| 337 | SERPINA1, SERPINA3, CTSD, DPP4, GDF15, GSN, TIMP1 | Age |
| 338 | SERPINA1, SERPINA3, CTSD, DPP4, GDF15, GSN, TFRC | Age |
| 339 | SERPINA1, SERPINA3, CTSD, DPP4, GDF15, MIF, PKM | Age |
| 340 | SERPINA1, SERPINA3, CTSD, DPP4, GDF15, MIF, TIMP1 | Age |
| 341 | SERPINA1, SERPINA3, CTSD, DPP4, GDF15, MIF, TFRC | Age |
| 342 | SERPINA1, SERPINA3, CTSD, DPP4, GDF15, PKM, TIMP1 | Age |
| 343 | SERPINA1, SERPINA3, CTSD, DPP4, GDF15, PKM, TFRC | Age |
| 344 | SERPINA1, SERPINA3, CTSD, DPP4, GDF15, TIMP1, TFRC | Age |
| 345 | SERPINA1, SERPINA3, CTSD, DPP4, GSN, MIF, PKM | Age |
| 346 | SERPINA1, SERPINA3, CTSD, DPP4, GSN, MIF, TIMP1 | Age |
| 347 | SERPINA1, SERPINA3, CTSD, DPP4, GSN, MIF, TFRC | Age |
| 348 | SERPINA1, SERPINA3, CTSD, DPP4, GSN, PKM, TIMP1 | Age |
| 349 | SERPINA1, SERPINA3, CTSD, DPP4, GSN, PKM, TFRC | Age |
| 350 | SERPINA1, SERPINA3, CTSD, DPP4, GSN, TIMP1, TFRC | Age |
| 351 | SERPINA1, SERPINA3, CTSD, DPP4, MIF, PKM, TIMP1 | Age |
| 352 | SERPINA1, SERPINA3, CTSD, DPP4, MIF, PKM, TFRC | Age |
| 353 | SERPINA1, SERPINA3, CTSD, DPP4, MIF, TIMP1, TFRC | Age |
| 354 | SERPINA1, SERPINA3, CTSD, DPP4, PKM, TIMP1, TFRC | Age |
| 355 | SERPINA1, SERPINA3, CTSD, GDF15, GSN, MIF, PKM | Age |
| 356 | SERPINA1, SERPINA3, CTSD, GDF15, GSN, MIF, TIMP1 | Age |
| 357 | SERPINA1, SERPINA3, CTSD, GDF15, GSN, MIF, TFRC | Age |
| 358 | SERPINA1, SERPINA3, CTSD, GDF15, GSN, PKM, TIMP1 | Age |
| 359 | SERPINA1, SERPINA3, CTSD, GDF15, GSN, PKM, TFRC | Age |
| 360 | SERPINA1, SERPINA3, CTSD, GDF15, GSN, TIMP1, TFRC | Age |
| 361 | SERPINA1, SERPINA3, CTSD, GDF15, MIF, PKM, TIMP1 | Age |
| 362 | SERPINA1, SERPINA3, CTSD, GDF15, MIF, PKM, TFRC | Age |
| 363 | SERPINA1, SERPINA3, CTSD, GDF15, MIF, TIMP1, TFRC | Age |
| 364 | SERPINA1, SERPINA3, CTSD, GDF15, PKM, TIMP1, TFRC | Age |
| 365 | SERPINA1, SERPINA3, CTSD, GSN, MIF, PKM, TIMP1 | Age |
| 366 | SERPINA1, SERPINA3, CTSD, GSN, MIF, PKM, TFRC | Age |
| 367 | SERPINA1, SERPINA3, CTSD, GSN, MIF, TIMP1, TFRC | Age |
| 368 | SERPINA1, SERPINA3, CTSD, GSN, PKM, TIMP1, TFRC | Age |
| 369 | SERPINA1, SERPINA3, CTSD, MIF, PKM, TIMP1, TFRC | Age |
| 370 | SERPINA1, SERPINA3, CLU, DPP4, GDF15, GSN, MIF | Age |
| 371 | SERPINA1, SERPINA3, CLU, DPP4, GDF15, GSN, PKM | Age |
| 372 | SERPINA1, SERPINA3, CLU, DPP4, GDF15, GSN, TIMP1 | Age |
| 373 | SERPINA1, SERPINA3, CLU, DPP4, GDF15, GSN, TFRC | Age |
| 374 | SERPINA1, SERPINA3, CLU, DPP4, GDF15, MIF, PKM | Age |
| 375 | SERPINA1, SERPINA3, CLU, DPP4, GDF15, MIF, TIMP1 | Age |
| 376 | SERPINA1, SERPINA3, CLU, DPP4, GDF15, MIF, TFRC | Age |
| 377 | SERPINA1, SERPINA3, CLU, DPP4, GDF15, PKM, TIMP1 | Age |
| 378 | SERPINA1, SERPINA3, CLU, DPP4, GDF15, PKM, TFRC | Age |
| 379 | SERPINA1, SERPINA3, CLU, DPP4, GDF15, TIMP1, TFRC | Age |
| 380 | SERPINA1, SERPINA3, CLU, DPP4, GSN, MIF, PKM | Age |
| 381 | SERPINA1, SERPINA3, CLU, DPP4, GSN, MIF, TIMP1 | Age |
| 382 | SERPINA1, SERPINA3, CLU, DPP4, GSN, MIF, TFRC | Age |
| 383 | SERPINA1, SERPINA3, CLU, DPP4, GSN, PKM, TIMP1 | Age |
| 384 | SERPINA1, SERPINA3, CLU, DPP4, GSN, PKM, TFRC | Age |
| 385 | SERPINA1, SERPINA3, CLU, DPP4, GSN, TIMP1, TFRC | Age |
| 386 | SERPINA1, SERPINA3, CLU, DPP4, MIF, PKM, TIMP1 | Age |
| 387 | SERPINA1, SERPINA3, CLU, DPP4, MIF, PKM, TFRC | Age |
| 388 | SERPINA1, SERPINA3, CLU, DPP4, MIF, TIMP1, TFRC | Age |
| 389 | SERPINA1, SERPINA3, CLU, DPP4, PKM, TIMP1, TFRC | Age |
| 390 | SERPINA1, SERPINA3, CLU, GDF15, GSN, MIF, PKM | Age |
| 391 | SERPINA1, SERPINA3, CLU, GDF15, GSN, MIF, TIMP1 | Age |
| 392 | SERPINA1, SERPINA3, CLU, GDF15, GSN, MIF, TFRC | Age |
| 393 | SERPINA1, SERPINA3, CLU, GDF15, GSN, PKM, TIMP1 | Age |
| 394 | SERPINA1, SERPINA3, CLU, GDF15, GSN, PKM, TFRC | Age |
| 395 | SERPINA1, SERPINA3, CLU, GDF15, GSN, TIMP1, TFRC | Age |
| 396 | SERPINA1, SERPINA3, CLU, GDF15, MIF, PKM, TIMP1 | Age |
| 397 | SERPINA1, SERPINA3, CLU, GDF15, MIF, PKM, TFRC | Age |
| 398 | SERPINA1, SERPINA3, CLU, GDF15, MIF, TIMP1, TFRC | Age |
| 399 | SERPINA1, SERPINA3, CLU, GDF15, PKM, TIMP1, TFRC | Age |
| 400 | SERPINA1, SERPINA3, CLU, GSN, MIF, PKM, TIMP1 | Age |
| 401 | SERPINA1, SERPINA3, CLU, GSN, MIF, PKM, TFRC | Age |
| 402 | SERPINA1, SERPINA3, CLU, GSN, MIF, TIMP1, TFRC | Age |
| 403 | SERPINA1, SERPINA3, CLU, GSN, PKM, TIMP1, TFRC | Age |
| 404 | SERPINA1, SERPINA3, CLU, MIF, PKM, TIMP1, TFRC | Age |
| 405 | SERPINA1, SERPINA3, DPP4, GDF15, GSN, MIF, PKM | Age |
| 406 | SERPINA1, SERPINA3, DPP4, GDF15, GSN, MIF, TIMP1 | Age |
| 407 | SERPINA1, SERPINA3, DPP4, GDF15, GSN, MIF, TFRC | Age |
| 408 | SERPINA1, SERPINA3, DPP4, GDF15, GSN, PKM, TIMP1 | Age |
| 409 | SERPINA1, SERPINA3, DPP4, GDF15, GSN, PKM, TFRC | Age |
| 410 | SERPINA1, SERPINA3, DPP4, GDF15, GSN, TIMP1, TFRC | Age |
| 411 | SERPINA1, SERPINA3, DPP4, GDF15, MIF, PKM, TIMP1 | Age |
| 412 | SERPINA1, SERPINA3, DPP4, GDF15, MIF, PKM, TFRC | Age |
| 413 | SERPINA1, SERPINA3, DPP4, GDF15, MIF, TIMP1, TFRC | Age |
| 414 | SERPINA1, SERPINA3, DPP4, GDF15, PKM, TIMP1, TFRC | Age |
| 415 | SERPINA1, SERPINA3, DPP4, GSN, MIF, PKM, TIMP1 | Age |
| 416 | SERPINA1, SERPINA3, DPP4, GSN, MIF, PKM, TFRC | Age |
| 417 | SERPINA1, SERPINA3, DPP4, GSN, MIF, TIMP1, TFRC | Age |
| 418 | SERPINA1, SERPINA3, DPP4, GSN, PKM, TIMP1, TFRC | Age |
| 419 | SERPINA1, SERPINA3, DPP4, MIF, PKM, TIMP1, TFRC | Age |
| 420 | SERPINA1, SERPINA3, GDF15, GSN, MIF, PKM, TIMP1 | Age |
| 421 | SERPINA1, SERPINA3, GDF15, GSN, MIF, PKM, TFRC | Age |
| 422 | SERPINA1, SERPINA3, GDF15, GSN, MIF, TIMP1, TFRC | Age |
| 423 | SERPINA1, SERPINA3, GDF15, GSN, PKM, TIMP1, TFRC | Age |
| 424 | SERPINA1, SERPINA3, GDF15, MIF, PKM, TIMP1, TFRC | Age |
| 425 | SERPINA1, SERPINA3, GSN, MIF, PKM, TIMP1, TFRC | Age |
| 426 | SERPINA1, CTSD, CLU, DPP4, GDF15, GSN, MIF | Age |
| 427 | SERPINA1, CTSD, CLU, DPP4, GDF15, GSN, PKM | Age |
| 428 | SERPINA1, CTSD, CLU, DPP4, GDF15, GSN, TIMP1 | Age |
| 429 | SERPINA1, CTSD, CLU, DPP4, GDF15, GSN, TFRC | Age |
| 430 | SERPINA1, CTSD, CLU, DPP4, GDF15, MIF, PKM | Age |
| 431 | SERPINA1, CTSD, CLU, DPP4, GDF15, MIF, TIMP1 | Age |
| 432 | SERPINA1, CTSD, CLU, DPP4, GDF15, MIF, TFRC | Age |
| 433 | SERPINA1, CTSD, CLU, DPP4, GDF15, PKM, TIMP1 | Age |
| 434 | SERPINA1, CTSD, CLU, DPP4, GDF15, PKM, TFRC | Age |
| 435 | SERPINA1, CTSD, CLU, DPP4, GDF15, TIMP1, TFRC | Age |
| 436 | SERPINA1, CTSD, CLU, DPP4, GSN, MIF, PKM | Age |
| 437 | SERPINA1, CTSD, CLU, DPP4, GSN, MIF, TIMP1 | Age |
| 438 | SERPINA1, CTSD, CLU, DPP4, GSN, MIF, TFRC | Age |
| 439 | SERPINA1, CTSD, CLU, DPP4, GSN, PKM, TIMP1 | Age |
| 440 | SERPINA1, CTSD, CLU, DPP4, GSN, PKM, TFRC | Age |
| 441 | SERPINA1, CTSD, CLU, DPP4, GSN, TIMP1, TFRC | Age |
| 442 | SERPINA1, CTSD, CLU, DPP4, MIF, PKM, TIMP1 | Age |
| 443 | SERPINA1, CTSD, CLU, DPP4, MIF, PKM, TFRC | Age |
| 444 | SERPINA1, CTSD, CLU, DPP4, MIF, TIMP1, TFRC | Age |
| 445 | SERPINA1, CTSD, CLU, DPP4, PKM, TIMP1, TFRC | Age |
| 446 | SERPINA1, CTSD, CLU, GDF15, GSN, MIF, PKM | Age |
| 447 | SERPINA1, CTSD, CLU, GDF15, GSN, MIF, TIMP1 | Age |
| 448 | SERPINA1, CTSD, CLU, GDF15, GSN, MIF, TFRC | Age |
| 449 | SERPINA1, CTSD, CLU, GDF15, GSN, PKM, TIMP1 | Age |
| 450 | SERPINA1, CTSD, CLU, GDF15, GSN, PKM, TFRC | Age |
| 451 | SERPINA1, CTSD, CLU, GDF15, GSN, TIMP1, TFRC | Age |
| 452 | SERPINA1, CTSD, CLU, GDF15, MIF, PKM, TIMP1 | Age |
| 453 | SERPINA1, CTSD, CLU, GDF15, MIF, PKM, TFRC | Age |
| 454 | SERPINA1, CTSD, CLU, GDF15, MIF, TIMP1, TFRC | Age |
| 455 | SERPINA1, CTSD, CLU, GDF15, PKM, TIMP1, TFRC | Age |
| 456 | SERPINA1, CTSD, CLU, GSN, MIF, PKM, TIMP1 | Age |
| 457 | SERPINA1, CTSD, CLU, GSN, MIF, PKM, TFRC | Age |
| 458 | SERPINA1, CTSD, CLU, GSN, MIF, TIMP1, TFRC | Age |
| 459 | SERPINA1, CTSD, CLU, GSN, PKM, TIMP1, TFRC | Age |
| 460 | SERPINA1, CTSD, CLU, MIF, PKM, TIMP1, TFRC | Age |
| 461 | SERPINA1, CTSD, DPP4, GDF15, GSN, MIF, PKM | Age |
| 462 | SERPINA1, CTSD, DPP4, GDF15, GSN, MIF, TIMP1 | Age |
| 463 | SERPINA1, CTSD, DPP4, GDF15, GSN, MIF, TFRC | Age |
| 464 | SERPINA1, CTSD, DPP4, GDF15, GSN, PKM, TIMP1 | Age |
| 465 | SERPINA1, CTSD, DPP4, GDF15, GSN, PKM, TFRC | Age |
| 466 | SERPINA1, CTSD, DPP4, GDF15, GSN, TIMP1, TFRC | Age |
| 467 | SERPINA1, CTSD, DPP4, GDF15, MIF, PKM, TIMP1 | Age |
| 468 | SERPINA1, CTSD, DPP4, GDF15, MIF, PKM, TFRC | Age |
| 469 | SERPINA1, CTSD, DPP4, GDF15, MIF, TIMP1, TFRC | Age |
| 470 | SERPINA1, CTSD, DPP4, GDF15, PKM, TIMP1, TFRC | Age |
| 471 | SERPINA1, CTSD, DPP4, GSN, MIF, PKM, TIMP1 | Age |
| 472 | SERPINA1, CTSD, DPP4, GSN, MIF, PKM, TFRC | Age |
| 473 | SERPINA1, CTSD, DPP4, GSN, MIF, TIMP1, TFRC | Age |
| 474 | SERPINA1, CTSD, DPP4, GSN, PKM, TIMP1, TFRC | Age |
| 475 | SERPINA1, CTSD, DPP4, MIF, PKM, TIMP1, TFRC | Age |
| 476 | SERPINA1, CTSD, GDF15, GSN, MIF, PKM, TIMP1 | Age |
| 477 | SERPINA1, CTSD, GDF15, GSN, MIF, PKM, TFRC | Age |
| 478 | SERPINA1, CTSD, GDF15, GSN, MIF, TIMP1, TFRC | Age |
| 479 | SERPINA1, CTSD, GDF15, GSN, PKM, TIMP1, TFRC | Age |
| 480 | SERPINA1, CTSD, GDF15, MIF, PKM, TIMP1, TFRC | Age |
| 481 | SERPINA1, CTSD, GSN, MIF, PKM, TIMP1, TFRC | Age |
| 482 | SERPINA1, CLU, DPP4, GDF15, GSN, MIF, PKM | Age |
| 483 | SERPINA1, CLU, DPP4, GDF15, GSN, MIF, TIMP1 | Age |
| 484 | SERPINA1, CLU, DPP4, GDF15, GSN, MIF, TFRC | Age |
| 485 | SERPINA1, CLU, DPP4, GDF15, GSN, PKM, TIMP1 | Age |
| 486 | SERPINA1, CLU, DPP4, GDF15, GSN, PKM, TFRC | Age |
| 487 | SERPINA1, CLU, DPP4, GDF15, GSN, TIMP1, TFRC | Age |
| 488 | SERPINA1, CLU, DPP4, GDF15, MIF, PKM, TIMP1 | Age |
| 489 | SERPINA1, CLU, DPP4, GDF15, MIF, PKM, TFRC | Age |
| 490 | SERPINA1, CLU, DPP4, GDF15, MIF, TIMP1, TFRC | Age |
| 491 | SERPINA1, CLU, DPP4, GDF15, PKM, TIMP1, TFRC | Age |
| 492 | SERPINA1, CLU, DPP4, GSN, MIF, PKM, TIMP1 | Age |
| 493 | SERPINA1, CLU, DPP4, GSN, MIF, PKM, TFRC | Age |
| 494 | SERPINA1, CLU, DPP4, GSN, MIF, TIMP1, TFRC | Age |
| 495 | SERPINA1, CLU, DPP4, GSN, PKM, TIMP1, TFRC | Age |
| 496 | SERPINA1, CLU, DPP4, MIF, PKM, TIMP1, TFRC | Age |
| 497 | SERPINA1, CLU, GDF15, GSN, MIF, PKM, TIMP1 | Age |
| 498 | SERPINA1, CLU, GDF15, GSN, MIF, PKM, TFRC | Age |
| 499 | SERPINA1, CLU, GDF15, GSN, MIF, TIMP1, TFRC | Age |
| 500 | SERPINA1, CLU, GDF15, GSN, PKM, TIMP1, TFRC | Age |
| 501 | SERPINA1, CLU, GDF15, MIF, PKM, TIMP1, TFRC | Age |
| 502 | SERPINA1, CLU, GSN, MIF, PKM, TIMP1, TFRC | Age |
| 503 | SERPINA1, DPP4, GDF15, GSN, MIF, PKM, TIMP1 | Age |
| 504 | SERPINA1, DPP4, GDF15, GSN, MIF, PKM, TFRC | Age |
| 505 | SERPINA1, DPP4, GDF15, GSN, MIF, TIMP1, TFRC | Age |
| 506 | SERPINA1, DPP4, GDF15, GSN, PKM, TIMP1, TFRC | Age |
| 507 | SERPINA1, DPP4, GDF15, MIF, PKM, TIMP1, TFRC | Age |
| 508 | SERPINA1, DPP4, GSN, MIF, PKM, TIMP1, TFRC | Age |
| 509 | SERPINA1, GDF15, GSN, MIF, PKM, TIMP1, TFRC | Age |
| 510 | SERPINA3, CTSD, CLU, DPP4, GDF15, GSN, MIF | Age |
| 511 | SERPINA3, CTSD, CLU, DPP4, GDF15, GSN, PKM | Age |
| 512 | SERPINA3, CTSD, CLU, DPP4, GDF15, GSN, TIMP1 | Age |
| 513 | SERPINA3, CTSD, CLU, DPP4, GDF15, GSN, TFRC | Age |
| 514 | SERPINA3, CTSD, CLU, DPP4, GDF15, MIF, PKM | Age |
| 515 | SERPINA3, CTSD, CLU, DPP4, GDF15, MIF, TIMP1 | Age |
| 516 | SERPINA3, CTSD, CLU, DPP4, GDF15, MIF, TFRC | Age |
| 517 | SERPINA3, CTSD, CLU, DPP4, GDF15, PKM, TIMP1 | Age |
| 518 | SERPINA3, CTSD, CLU, DPP4, GDF15, PKM, TFRC | Age |
| 519 | SERPINA3, CTSD, CLU, DPP4, GDF15, TIMP1, TFRC | Age |
| 520 | SERPINA3, CTSD, CLU, DPP4, GSN, MIF, PKM | Age |
| 521 | SERPINA3, CTSD, CLU, DPP4, GSN, MIF, TIMP1 | Age |
| 522 | SERPINA3, CTSD, CLU, DPP4, GSN, MIF, TFRC | Age |
| 523 | SERPINA3, CTSD, CLU, DPP4, GSN, PKM, TIMP1 | Age |
| 524 | SERPINA3, CTSD, CLU, DPP4, GSN, PKM, TFRC | Age |
| 525 | SERPINA3, CTSD, CLU, DPP4, GSN, TIMP1, TFRC | Age |
| 526 | SERPINA3, CTSD, CLU, DPP4, MIF, PKM, TIMP1 | Age |
| 527 | SERPINA3, CTSD, CLU, DPP4, MIF, PKM, TFRC | Age |
| 528 | SERPINA3, CTSD, CLU, DPP4, MIF, TIMP1, TFRC | Age |
| 529 | SERPINA3, CTSD, CLU, DPP4, PKM, TIMP1, TFRC | Age |
| 530 | SERPINA3, CTSD, CLU, GDF15, GSN, MIF, PKM | Age |
| 531 | SERPINA3, CTSD, CLU, GDF15, GSN, MIF, TIMP1 | Age |
| 532 | SERPINA3, CTSD, CLU, GDF15, GSN, MIF, TFRC | Age |
| 533 | SERPINA3, CTSD, CLU, GDF15, GSN, PKM, TIMP1 | Age |
| 534 | SERPINA3, CTSD, CLU, GDF15, GSN, PKM, TFRC | Age |
| 535 | SERPINA3, CTSD, CLU, GDF15, GSN, TIMP1, TFRC | Age |
| 536 | SERPINA3, CTSD, CLU, GDF15, MIF, PKM, TIMP1 | Age |
| 537 | SERPINA3, CTSD, CLU, GDF15, MIF, PKM, TFRC | Age |
| 538 | SERPINA3, CTSD, CLU, GDF15, MIF, TIMP1, TFRC | Age |
| 539 | SERPINA3, CTSD, CLU, GDF15, PKM, TIMP1, TFRC | Age |
| 540 | SERPINA3, CTSD, CLU, GSN, MIF, PKM, TIMP1 | Age |
| 541 | SERPINA3, CTSD, CLU, GSN, MIF, PKM, TFRC | Age |
| 542 | SERPINA3, CTSD, CLU, GSN, MIF, TIMP1, TFRC | Age |
| 543 | SERPINA3, CTSD, CLU, GSN, PKM, TIMP1, TFRC | Age |
| 544 | SERPINA3, CTSD, CLU, MIF, PKM, TIMP1, TFRC | Age |
| 545 | SERPINA3, CTSD, DPP4, GDF15, GSN, MIF, PKM | Age |
| 546 | SERPINA3, CTSD, DPP4, GDF15, GSN, MIF, TIMP1 | Age |
| 547 | SERPINA3, CTSD, DPP4, GDF15, GSN, MIF, TFRC | Age |
| 548 | SERPINA3, CTSD, DPP4, GDF15, GSN, PKM, TIMP1 | Age |
| 549 | SERPINA3, CTSD, DPP4, GDF15, GSN, PKM, TFRC | Age |
| 550 | SERPINA3, CTSD, DPP4, GDF15, GSN, TIMP1, TFRC | Age |
| 551 | SERPINA3, CTSD, DPP4, GDF15, MIF, PKM, TIMP1 | Age |
| 552 | SERPINA3, CTSD, DPP4, GDF15, MIF, PKM, TFRC | Age |
| 553 | SERPINA3, CTSD, DPP4, GDF15, MIF, TIMP1, TFRC | Age |
| 554 | SERPINA3, CTSD, DPP4, GDF15, PKM, TIMP1, TFRC | Age |
| 555 | SERPINA3, CTSD, DPP4, GSN, MIF, PKM, TIMP1 | Age |
| 556 | SERPINA3, CTSD, DPP4, GSN, MIF, PKM, TFRC | Age |
| 557 | SERPINA3, CTSD, DPP4, GSN, MIF, TIMP1, TFRC | Age |
| 558 | SERPINA3, CTSD, DPP4, GSN, PKM, TIMP1, TFRC | Age |
| 559 | SERPINA3, CTSD, DPP4, MIF, PKM, TIMP1, TFRC | Age |
| 560 | SERPINA3, CTSD, GDF15, GSN, MIF, PKM, TIMP1 | Age |
| 561 | SERPINA3, CTSD, GDF15, GSN, MIF, PKM, TFRC | Age |
| 562 | SERPINA3, CTSD, GDF15, GSN, MIF, TIMP1, TFRC | Age |
| 563 | SERPINA3, CTSD, GDF15, GSN, PKM, TIMP1, TFRC | Age |
| 564 | SERPINA3, CTSD, GDF15, MIF, PKM, TIMP1, TFRC | Age |
| 565 | SERPINA3, CTSD, GSN, MIF, PKM, TIMP1, TFRC | Age |
| 566 | SERPINA3, CLU, DPP4, GDF15, GSN, MIF, PKM | Age |
| 567 | SERPINA3, CLU, DPP4, GDF15, GSN, MIF, TIMP1 | Age |
| 568 | SERPINA3, CLU, DPP4, GDF15, GSN, MIF, TFRC | Age |
| 569 | SERPINA3, CLU, DPP4, GDF15, GSN, PKM, TIMP1 | Age |
| 570 | SERPINA3, CLU, DPP4, GDF15, GSN, PKM, TFRC | Age |
| 571 | SERPINA3, CLU, DPP4, GDF15, GSN, TIMP1, TFRC | Age |
| 572 | SERPINA3, CLU, DPP4, GDF15, MIF, PKM, TIMP1 | Age |
| 573 | SERPINA3, CLU, DPP4, GDF15, MIF, PKM, TFRC | Age |
| 574 | SERPINA3, CLU, DPP4, GDF15, MIF, TIMP1, TFRC | Age |
| 575 | SERPINA3, CLU, DPP4, GDF15, PKM, TIMP1, TFRC | Age |
| 576 | SERPINA3, CLU, DPP4, GSN, MIF, PKM, TIMP1 | Age |
| 577 | SERPINA3, CLU, DPP4, GSN, MIF, PKM, TFRC | Age |
| 578 | SERPINA3, CLU, DPP4, GSN, MIF, TIMP1, TFRC | Age |
| 579 | SERPINA3, CLU, DPP4, GSN, PKM, TIMP1, TFRC | Age |
| 580 | SERPINA3, CLU, DPP4, MIF, PKM, TIMP1, TFRC | Age |
| 581 | SERPINA3, CLU, GDF15, GSN, MIF, PKM, TIMP1 | Age |
| 582 | SERPINA3, CLU, GDF15, GSN, MIF, PKM, TFRC | Age |
| 583 | SERPINA3, CLU, GDF15, GSN, MIF, TIMP1, TFRC | Age |
| 584 | SERPINA3, CLU, GDF15, GSN, PKM, TIMP1, TFRC | Age |
| 585 | SERPINA3, CLU, GDF15, MIF, PKM, TIMP1, TFRC | Age |
| 586 | SERPINA3, CLU, GSN, MIF, PKM, TIMP1, TFRC | Age |
| 587 | SERPINA3, DPP4, GDF15, GSN, MIF, PKM, TIMP1 | Age |
| 588 | SERPINA3, DPP4, GDF15, GSN, MIF, PKM, TFRC | Age |
| 589 | SERPINA3, DPP4, GDF15, GSN, MIF, TIMP1, TFRC | Age |
| 590 | SERPINA3, DPP4, GDF15, GSN, PKM, TIMP1, TFRC | Age |
| 591 | SERPINA3, DPP4, GDF15, MIF, PKM, TIMP1, TFRC | Age |
| 592 | SERPINA3, DPP4, GSN, MIF, PKM, TIMP1, TFRC | Age |
| 593 | SERPINA3, GDF15, GSN, MIF, PKM, TIMP1, TFRC | Age |
| 594 | CTSD, CLU, DPP4, GDF15, GSN, MIF, PKM | Age |
| 595 | CTSD, CLU, DPP4, GDF15, GSN, MIF, TIMP1 | Age |
| 596 | CTSD, CLU, DPP4, GDF15, GSN, MIF, TFRC | Age |
| 597 | CTSD, CLU, DPP4, GDF15, GSN, PKM, TIMP1 | Age |
| 598 | CTSD, CLU, DPP4, GDF15, GSN, PKM, TFRC | Age |
| 599 | CTSD, CLU, DPP4, GDF15, GSN, TIMP1, TFRC | Age |
| 600 | CTSD, CLU, DPP4, GDF15, MIF, PKM, TIMP1 | Age |
| 601 | CTSD, CLU, DPP4, GDF15, MIF, PKM, TFRC | Age |
| 602 | CTSD, CLU, DPP4, GDF15, MIF, TIMP1, TFRC | Age |
| 603 | CTSD, CLU, DPP4, GDF15, PKM, TIMP1, TFRC | Age |
| 604 | CTSD, CLU, DPP4, GSN, MIF, PKM, TIMP1 | Age |
| 605 | CTSD, CLU, DPP4, GSN, MIF, PKM, TFRC | Age |
| 606 | CTSD, CLU, DPP4, GSN, MIF, TIMP1, TFRC | Age |
| 607 | CTSD, CLU, DPP4, GSN, PKM, TIMP1, TFRC | Age |
| 608 | CTSD, CLU, DPP4, MIF, PKM, TIMP1, TFRC | Age |
| 609 | CTSD, CLU, GDF15, GSN, MIF, PKM, TIMP1 | Age |
| 610 | CTSD, CLU, GDF15, GSN, MIF, PKM, TFRC | Age |
| 611 | CTSD, CLU, GDF15, GSN, MIF, TIMP1, TFRC | Age |
| 612 | CTSD, CLU, GDF15, GSN, PKM, TIMP1, TFRC | Age |
| 613 | CTSD, CLU, GDF15, MIF, PKM, TIMP1, TFRC | Age |
| 614 | CTSD, CLU, GSN, MIF, PKM, TIMP1, TFRC | Age |
| 615 | CTSD, DPP4, GDF15, GSN, MIF, PKM, TIMP1 | Age |
| 616 | CTSD, DPP4, GDF15, GSN, MIF, PKM, TFRC | Age |
| 617 | CTSD, DPP4, GDF15, GSN, MIF, TIMP1, TFRC | Age |
| 618 | CTSD, DPP4, GDF15, GSN, PKM, TIMP1, TFRC | Age |
| 619 | CTSD, DPP4, GDF15, MIF, PKM, TIMP1, TFRC | Age |
| 620 | CTSD, DPP4, GSN, MIF, PKM, TIMP1, TFRC | Age |
| 621 | CTSD, GDF15, GSN, MIF, PKM, TIMP1, TFRC | Age |
| 622 | CLU, DPP4, GDF15, GSN, MIF, PKM, TIMP1 | Age |
| 623 | CLU, DPP4, GDF15, GSN, MIF, PKM, TFRC | Age |
| 624 | CLU, DPP4, GDF15, GSN, MIF, TIMP1, TFRC | Age |
| 625 | CLU, DPP4, GDF15, GSN, PKM, TIMP1, TFRC | Age |
| 626 | CLU, DPP4, GDF15, MIF, PKM, TIMP1, TFRC | Age |
| 627 | CLU, DPP4, GSN, MIF, PKM, TIMP1, TFRC | Age |
| 628 | CLU, GDF15, GSN, MIF, PKM, TIMP1, TFRC | Age |
| 629 | DPP4, GDF15, GSN, MIF, PKM, TIMP1, TFRC | Age |
| 630 | SERPINA1, SERPINA3, CTSD, CLU, DPP4, GDF15, GSN, MIF | NONE |
| 631 | SERPINA1, SERPINA3, CTSD, CLU, DPP4, GDF15, GSN, PKM | NONE |
| 632 | SERPINA1, SERPINA3, CTSD, CLU, DPP4, GDF15, GSN, TIMP1 | NONE |
| 633 | SERPINA1, SERPINA3, CTSD, CLU, DPP4, GDF15, GSN, TFRC | NONE |
| 634 | SERPINA1, SERPINA3, CTSD, CLU, DPP4, GDF15, MIF, PKM | NONE |
| 635 | SERPINA1, SERPINA3, CTSD, CLU, DPP4, GDF15, MIF, TIMP1 | NONE |
| 636 | SERPINA1, SERPINA3, CTSD, CLU, DPP4, GDF15, MIF, TFRC | NONE |
| 637 | SERPINA1, SERPINA3, CTSD, CLU, DPP4, GDF15, PKM, TIMP1 | NONE |
| 638 | SERPINA1, SERPINA3, CTSD, CLU, DPP4, GDF15, PKM, TFRC | NONE |
| 639 | SERPINA1, SERPINA3, CTSD, CLU, DPP4, GDF15, TIMP1, TFRC | NONE |
| 640 | SERPINA1, SERPINA3, CTSD, CLU, DPP4, GSN, MIF, PKM | NONE |
| 641 | SERPINA1, SERPINA3, CTSD, CLU, DPP4, GSN, MIF, TIMP1 | NONE |
| 642 | SERPINA1, SERPINA3, CTSD, CLU, DPP4, GSN, MIF, TFRC | NONE |
| 643 | SERPINA1, SERPINA3, CTSD, CLU, DPP4, GSN, PKM, TIMP1 | NONE |
| 644 | SERPINA1, SERPINA3, CTSD, CLU, DPP4, GSN, PKM, TFRC | NONE |
| 645 | SERPINA1, SERPINA3, CTSD, CLU, DPP4, GSN, TIMP1, TFRC | NONE |
| 646 | SERPINA1, SERPINA3, CTSD, CLU, DPP4, MIF, PKM, TIMP1 | NONE |
| 647 | SERPINA1, SERPINA3, CTSD, CLU, DPP4, MIF, PKM, TFRC | NONE |
| 648 | SERPINA1, SERPINA3, CTSD, CLU, DPP4, MIF, TIMP1, TFRC | NONE |
| 649 | SERPINA1, SERPINA3, CTSD, CLU, DPP4, PKM, TIMP1, TFRC | NONE |
| 650 | SERPINA1, SERPINA3, CTSD, CLU, GDF15, GSN, MIF, PKM | NONE |
| 651 | SERPINA1, SERPINA3, CTSD, CLU, GDF15, GSN, MIF, TIMP1 | NONE |
| 652 | SERPINA1, SERPINA3, CTSD, CLU, GDF15, GSN, MIF, TFRC | NONE |
| 653 | SERPINA1, SERPINA3, CTSD, CLU, GDF15, GSN, PKM, TIMP1 | NONE |
| 654 | SERPINA1, SERPINA3, CTSD, CLU, GDF15, GSN, PKM, TFRC | NONE |
| 655 | SERPINA1, SERPINA3, CTSD, CLU, GDF15, GSN, TIMP1, TFRC | NONE |
| 656 | SERPINA1, SERPINA3, CTSD, CLU, GDF15, MIF, PKM, TIMP1 | NONE |
| 657 | SERPINA1, SERPINA3, CTSD, CLU, GDF15, MIF, PKM, TFRC | NONE |
| 658 | SERPINA1, SERPINA3, CTSD, CLU, GDF15, MIF, TIMP1, TFRC | NONE |
| 659 | SERPINA1, SERPINA3, CTSD, CLU, GDF15, PKM, TIMP1, TFRC | NONE |
| 660 | SERPINA1, SERPINA3, CTSD, CLU, GSN, MIF, PKM, TIMP1 | NONE |
| 661 | SERPINA1, SERPINA3, CTSD, CLU, GSN, MIF, PKM, TFRC | NONE |
| 662 | SERPINA1, SERPINA3, CTSD, CLU, GSN, MIF, TIMP1, TFRC | NONE |
| 663 | SERPINA1, SERPINA3, CTSD, CLU, GSN, PKM, TIMP1, TFRC | NONE |
| 664 | SERPINA1, SERPINA3, CTSD, CLU, MIF, PKM, TIMP1, TFRC | NONE |
| 665 | SERPINA1, SERPINA3, CTSD, DPP4, GDF15, GSN, MIF, PKM | NONE |
| 666 | SERPINA1, SERPINA3, CTSD, DPP4, GDF15, GSN, MIF, TIMP1 | NONE |
| 667 | SERPINA1, SERPINA3, CTSD, DPP4, GDF15, GSN, MIF, TFRC | NONE |
| 668 | SERPINA1, SERPINA3, CTSD, DPP4, GDF15, GSN, PKM, TIMP1 | NONE |
| 669 | SERPINA1, SERPINA3, CTSD, DPP4, GDF15, GSN, PKM, TFRC | NONE |
| 670 | SERPINA1, SERPINA3, CTSD, DPP4, GDF15, GSN, TIMP1, TFRC | NONE |
| 671 | SERPINA1, SERPINA3, CTSD, DPP4, GDF15, MIF, PKM, TIMP1 | NONE |
| 672 | SERPINA1, SERPINA3, CTSD, DPP4, GDF15, MIF, PKM, TFRC | NONE |
| 673 | SERPINA1, SERPINA3, CTSD, DPP4, GDF15, MIF, TIMP1, TFRC | NONE |
| 674 | SERPINA1, SERPINA3, CTSD, DPP4, GDF15, PKM, TIMP1, TFRC | NONE |
| 675 | SERPINA1, SERPINA3, CTSD, DPP4, GSN, MIF, PKM, TIMP1 | NONE |
| 676 | SERPINA1, SERPINA3, CTSD, DPP4, GSN, MIF, PKM, TFRC | NONE |
| 677 | SERPINA1, SERPINA3, CTSD, DPP4, GSN, MIF, TIMP1, TFRC | NONE |
| 678 | SERPINA1, SERPINA3, CTSD, DPP4, GSN, PKM, TIMP1, TFRC | NONE |
| 679 | SERPINA1, SERPINA3, CTSD, DPP4, MIF, PKM, TIMP1, TFRC | NONE |
| 680 | SERPINA1, SERPINA3, CTSD, GDF15, GSN, MIF, PKM, TIMP1 | NONE |
| 681 | SERPINA1, SERPINA3, CTSD, GDF15, GSN, MIF, PKM, TFRC | NONE |
| 682 | SERPINA1, SERPINA3, CTSD, GDF15, GSN, MIF, TIMP1, TFRC | NONE |
| 683 | SERPINA1, SERPINA3, CTSD, GDF15, GSN, PKM, TIMP1, TFRC | NONE |
| 684 | SERPINA1, SERPINA3, CTSD, GDF15, MIF, PKM, TIMP1, TFRC | NONE |
| 685 | SERPINA1, SERPINA3, CTSD, GSN, MIF, PKM, TIMP1, TFRC | NONE |
| 686 | SERPINA1, SERPINA3, CLU, DPP4, GDF15, GSN, MIF, PKM | NONE |
| 687 | SERPINA1, SERPINA3, CLU, DPP4, GDF15, GSN, MIF, TIMP1 | NONE |
| 688 | SERPINA1, SERPINA3, CLU, DPP4, GDF15, GSN, MIF, TFRC | NONE |
| 689 | SERPINA1, SERPINA3, CLU, DPP4, GDF15, GSN, PKM, TIMP1 | NONE |
| 690 | SERPINA1, SERPINA3, CLU, DPP4, GDF15, GSN, PKM, TFRC | NONE |
| 691 | SERPINA1, SERPINA3, CLU, DPP4, GDF15, GSN, TIMP1, TFRC | NONE |
| 692 | SERPINA1, SERPINA3, CLU, DPP4, GDF15, MIF, PKM, TIMP1 | NONE |
| 693 | SERPINA1, SERPINA3, CLU, DPP4, GDF15, MIF, PKM, TFRC | NONE |
| 694 | SERPINA1, SERPINA3, CLU, DPP4, GDF15, MIF, TIMP1, TFRC | NONE |
| 695 | SERPINA1, SERPINA3, CLU, DPP4, GDF15, PKM, TIMP1, TFRC | NONE |
| 696 | SERPINA1, SERPINA3, CLU, DPP4, GSN, MIF, PKM, TIMP1 | NONE |
| 697 | SERPINA1, SERPINA3, CLU, DPP4, GSN, MIF, PKM, TFRC | NONE |
| 698 | SERPINA1, SERPINA3, CLU, DPP4, GSN, MIF, TIMP1, TFRC | NONE |
| 699 | SERPINA1, SERPINA3, CLU, DPP4, GSN, PKM, TIMP1, TFRC | NONE |
| 700 | SERPINA1, SERPINA3, CLU, DPP4, MIF, PKM, TIMP1, TFRC | NONE |
| 701 | SERPINA1, SERPINA3, CLU, GDF15, GSN, MIF, PKM, TIMP1 | NONE |
| 702 | SERPINA1, SERPINA3, CLU, GDF15, GSN, MIF, PKM, TFRC | NONE |
| 703 | SERPINA1, SERPINA3, CLU, GDF15, GSN, MIF, TIMP1, TFRC | NONE |
| 704 | SERPINA1, SERPINA3, CLU, GDF15, GSN, PKM, TIMP1, TFRC | NONE |
| 705 | SERPINA1, SERPINA3, CLU, GDF15, MIF, PKM, TIMP1, TFRC | NONE |
| 706 | SERPINA1, SERPINA3, CLU, GSN, MIF, PKM, TIMP1, TFRC | NONE |
| 707 | SERPINA1, SERPINA3, DPP4, GDF15, GSN, MIF, PKM, TIMP1 | NONE |
| 708 | SERPINA1, SERPINA3, DPP4, GDF15, GSN, MIF, PKM, TFRC | NONE |
| 709 | SERPINA1, SERPINA3, DPP4, GDF15, GSN, MIF, TIMP1, TFRC | NONE |
| 710 | SERPINA1, SERPINA3, DPP4, GDF15, GSN, PKM, TIMP1, TFRC | NONE |
| 711 | SERPINA1, SERPINA3, DPP4, GDF15, MIF, PKM, TIMP1, TFRC | NONE |
| 712 | SERPINA1, SERPINA3, DPP4, GSN, MIF, PKM, TIMP1, TFRC | NONE |
| 713 | SERPINA1, SERPINA3, GDF15, GSN, MIF, PKM, TIMP1, TFRC | NONE |
| 714 | SERPINA1, CTSD, CLU, DPP4, GDF15, GSN, MIF, PKM | NONE |
| 715 | SERPINA1, CTSD, CLU, DPP4, GDF15, GSN, MIF, TIMP1 | NONE |
| 716 | SERPINA1, CTSD, CLU, DPP4, GDF15, GSN, MIF, TFRC | NONE |
| 717 | SERPINA1, CTSD, CLU, DPP4, GDF15, GSN, PKM, TIMP1 | NONE |
| 718 | SERPINA1, CTSD, CLU, DPP4, GDF15, GSN, PKM, TFRC | NONE |
| 719 | SERPINA1, CTSD, CLU, DPP4, GDF15, GSN, TIMP1, TFRC | NONE |
| 720 | SERPINA1, CTSD, CLU, DPP4, GDF15, MIF, PKM, TIMP1 | NONE |
| 721 | SERPINA1, CTSD, CLU, DPP4, GDF15, MIF, PKM, TFRC | NONE |
| 722 | SERPINA1, CTSD, CLU, DPP4, GDF15, MIF, TIMP1, TFRC | NONE |
| 723 | SERPINA1, CTSD, CLU, DPP4, GDF15, PKM, TIMP1, TFRC | NONE |
| 724 | SERPINA1, CTSD, CLU, DPP4, GSN, MIF, PKM, TIMP1 | NONE |
| 725 | SERPINA1, CTSD, CLU, DPP4, GSN, MIF, PKM, TFRC | NONE |
| 726 | SERPINA1, CTSD, CLU, DPP4, GSN, MIF, TIMP1, TFRC | NONE |
| 727 | SERPINA1, CTSD, CLU, DPP4, GSN, PKM, TIMP1, TFRC | NONE |
| 728 | SERPINA1, CTSD, CLU, DPP4, MIF, PKM, TIMP1, TFRC | NONE |
| 729 | SERPINA1, CTSD, CLU, GDF15, GSN, MIF, PKM, TIMP1 | NONE |
| 730 | SERPINA1, CTSD, CLU, GDF15, GSN, MIF, PKM, TFRC | NONE |
| 731 | SERPINA1, CTSD, CLU, GDF15, GSN, MIF, TIMP1, TFRC | NONE |
| 732 | SERPINA1, CTSD, CLU, GDF15, GSN, PKM, TIMP1, TFRC | NONE |
| 733 | SERPINA1, CTSD, CLU, GDF15, MIF, PKM, TIMP1, TFRC | NONE |
| 734 | SERPINA1, CTSD, CLU, GSN, MIF, PKM, TIMP1, TFRC | NONE |
| 735 | SERPINA1, CTSD, DPP4, GDF15, GSN, MIF, PKM, TIMP1 | NONE |
| 736 | SERPINA1, CTSD, DPP4, GDF15, GSN, MIF, PKM, TFRC | NONE |
| 737 | SERPINA1, CTSD, DPP4, GDF15, GSN, MIF, TIMP1, TFRC | NONE |
| 738 | SERPINA1, CTSD, DPP4, GDF15, GSN, PKM, TIMP1, TFRC | NONE |
| 739 | SERPINA1, CTSD, DPP4, GDF15, MIF, PKM, TIMP1, TFRC | NONE |
| 740 | SERPINA1, CTSD, DPP4, GSN, MIF, PKM, TIMP1, TFRC | NONE |
| 741 | SERPINA1, CTSD, GDF15, GSN, MIF, PKM, TIMP1, TFRC | NONE |
| 742 | SERPINA1, CLU, DPP4, GDF15, GSN, MIF, PKM, TIMP1 | NONE |
| 743 | SERPINA1, CLU, DPP4, GDF15, GSN, MIF, PKM, TFRC | NONE |
| 744 | SERPINA1, CLU, DPP4, GDF15, GSN, MIF, TIMP1, TFRC | NONE |
| 745 | SERPINA1, CLU, DPP4, GDF15, GSN, PKM, TIMP1, TFRC | NONE |
| 746 | SERPINA1, CLU, DPP4, GDF15, MIF, PKM, TIMP1, TFRC | NONE |
| 747 | SERPINA1, CLU, DPP4, GSN, MIF, PKM, TIMP1, TFRC | NONE |
| 748 | SERPINA1, CLU, GDF15, GSN, MIF, PKM, TIMP1, TFRC | NONE |
| 749 | SERPINA1, DPP4, GDF15, GSN, MIF, PKM, TIMP1, TFRC | NONE |
| 750 | SERPINA3, CTSD, CLU, DPP4, GDF15, GSN, MIF, PKM | NONE |
| 751 | SERPINA3, CTSD, CLU, DPP4, GDF15, GSN, MIF, TIMP1 | NONE |
| 752 | SERPINA3, CTSD, CLU, DPP4, GDF15, GSN, MIF, TFRC | NONE |
| 753 | SERPINA3, CTSD, CLU, DPP4, GDF15, GSN, PKM, TIMP1 | NONE |
| 754 | SERPINA3, CTSD, CLU, DPP4, GDF15, GSN, PKM, TFRC | NONE |
| 755 | SERPINA3, CTSD, CLU, DPP4, GDF15, GSN, TIMP1, TFRC | NONE |
| 756 | SERPINA3, CTSD, CLU, DPP4, GDF15, MIF, PKM, TIMP1 | NONE |
| 757 | SERPINA3, CTSD, CLU, DPP4, GDF15, MIF, PKM, TFRC | NONE |
| 758 | SERPINA3, CTSD, CLU, DPP4, GDF15, MIF, TIMP1, TFRC | NONE |
| 759 | SERPINA3, CTSD, CLU, DPP4, GDF15, PKM, TIMP1, TFRC | NONE |
| 760 | SERPINA3, CTSD, CLU, DPP4, GSN, MIF, PKM, TIMP1 | NONE |
| 761 | SERPINA3, CTSD, CLU, DPP4, GSN, MIF, PKM, TFRC | NONE |
| 762 | SERPINA3, CTSD, CLU, DPP4, GSN, MIF, TIMP1, TFRC | NONE |
| 763 | SERPINA3, CTSD, CLU, DPP4, GSN, PKM, TIMP1, TFRC | NONE |
| 764 | SERPINA3, CTSD, CLU, DPP4, MIF, PKM, TIMP1, TFRC | NONE |
| 765 | SERPINA3, CTSD, CLU, GDF15, GSN, MIF, PKM, TIMP1 | NONE |
| 766 | SERPINA3, CTSD, CLU, GDF15, GSN, MIF, PKM, TFRC | NONE |
| 767 | SERPINA3, CTSD, CLU, GDF15, GSN, MIF, TIMP1, TFRC | NONE |
| 768 | SERPINA3, CTSD, CLU, GDF15, GSN, PKM, TIMP1, TFRC | NONE |
| 769 | SERPINA3, CTSD, CLU, GDF15, MIF, PKM, TIMP1, TFRC | NONE |
| 770 | SERPINA3, CTSD, CLU, GSN, MIF, PKM, TIMP1, TFRC | NONE |
| 771 | SERPINA3, CTSD, DPP4, GDF15, GSN, MIF, PKM, TIMP1 | NONE |
| 772 | SERPINA3, CTSD, DPP4, GDF15, GSN, MIF, PKM, TFRC | NONE |
| 773 | SERPINA3, CTSD, DPP4, GDF15, GSN, MIF, TIMP1, TFRC | NONE |
| 774 | SERPINA3, CTSD, DPP4, GDF15, GSN, PKM, TIMP1, TFRC | NONE |
| 775 | SERPINA3, CTSD, DPP4, GDF15, MIF, PKM, TIMP1, TFRC | NONE |
| 776 | SERPINA3, CTSD, DPP4, GSN, MIF, PKM, TIMP1, TFRC | NONE |
| 777 | SERPINA3, CTSD, GDF15, GSN, MIF, PKM, TIMP1, TFRC | NONE |
| 778 | SERPINA3, CLU, DPP4, GDF15, GSN, MIF, PKM, TIMP1 | NONE |
| 779 | SERPINA3, CLU, DPP4, GDF15, GSN, MIF, PKM, TFRC | NONE |
| 780 | SERPINA3, CLU, DPP4, GDF15, GSN, MIF, TIMP1, TFRC | NONE |
| 781 | SERPINA3, CLU, DPP4, GDF15, GSN, PKM, TIMP1, TFRC | NONE |
| 782 | SERPINA3, CLU, DPP4, GDF15, MIF, PKM, TIMP1, TFRC | NONE |
| 783 | SERPINA3, CLU, DPP4, GSN, MIF, PKM, TIMP1, TFRC | NONE |
| 784 | SERPINA3, CLU, GDF15, GSN, MIF, PKM, TIMP1, TFRC | NONE |
| 785 | SERPINA3, DPP4, GDF15, GSN, MIF, PKM, TIMP1, TFRC | NONE |
| 786 | CTSD, CLU, DPP4, GDF15, GSN, MIF, PKM, TIMP1 | NONE |
| 787 | CTSD, CLU, DPP4, GDF15, GSN, MIF, PKM, TFRC | NONE |
| 788 | CTSD, CLU, DPP4, GDF15, GSN, MIF, TIMP1, TFRC | NONE |
| 789 | CTSD, CLU, DPP4, GDF15, GSN, PKM, TIMP1, TFRC | NONE |
| 790 | CTSD, CLU, DPP4, GDF15, MIF, PKM, TIMP1, TFRC | NONE |
| 791 | CTSD, CLU, DPP4, GSN, MIF, PKM, TIMP1, TFRC | NONE |
| 792 | CTSD, CLU, GDF15, GSN, MIF, PKM, TIMP1, TFRC | NONE |
| 793 | CTSD, DPP4, GDF15, GSN, MIF, PKM, TIMP1, TFRC | NONE |
| 794 | CLU, DPP4, GDF15, GSN, MIF, PKM, TIMP1, TFRC | NONE |
| 795 | SERPINA1, SERPINA3, CTSD, CLU, DPP4, GDF15 | Age |
| 796 | SERPINA1, SERPINA3, CTSD, CLU, DPP4, GSN | Age |
| 797 | SERPINA1, SERPINA3, CTSD, CLU, DPP4, MIF | Age |
| 798 | SERPINA1, SERPINA3, CTSD, CLU, DPP4, PKM | Age |
| 799 | SERPINA1, SERPINA3, CTSD, CLU, DPP4, TIMP1 | Age |
| 800 | SERPINA1, SERPINA3, CTSD, CLU, DPP4, TFRC | Age |
| 801 | SERPINA1, SERPINA3, CTSD, CLU, GDF15, GSN | Age |
| 802 | SERPINA1, SERPINA3, CTSD, CLU, GDF15, MIF | Age |
| 803 | SERPINA1, SERPINA3, CTSD, CLU, GDF15, PKM | Age |
| 804 | SERPINA1, SERPINA3, CTSD, CLU, GDF15, TIMP1 | Age |
| 805 | SERPINA1, SERPINA3, CTSD, CLU, GDF15, TFRC | Age |
| 806 | SERPINA1, SERPINA3, CTSD, CLU, GSN, MIF | Age |
| 807 | SERPINA1, SERPINA3, CTSD, CLU, GSN, PKM | Age |
| 808 | SERPINA1, SERPINA3, CTSD, CLU, GSN, TIMP1 | Age |
| 809 | SERPINA1, SERPINA3, CTSD, CLU, GSN, TFRC | Age |
| 810 | SERPINA1, SERPINA3, CTSD, CLU, MIF, PKM | Age |
| 811 | SERPINA1, SERPINA3, CTSD, CLU, MIF, TIMP1 | Age |
| 812 | SERPINA1, SERPINA3, CTSD, CLU, MIF, TFRC | Age |
| 813 | SERPINA1, SERPINA3, CTSD, CLU, PKM, TIMP1 | Age |
| 814 | SERPINA1, SERPINA3, CTSD, CLU, PKM, TFRC | Age |
| 815 | SERPINA1, SERPINA3, CTSD, CLU, TIMP1, TFRC | Age |
| 816 | SERPINA1, SERPINA3, CTSD, DPP4, GDF15, GSN | Age |
| 817 | SERPINA1, SERPINA3, CTSD, DPP4, GDF15, MIF | Age |
| 818 | SERPINA1, SERPINA3, CTSD, DPP4, GDF15, PKM | Age |
| 819 | SERPINA1, SERPINA3, CTSD, DPP4, GDF15, TIMP1 | Age |
| 820 | SERPINA1, SERPINA3, CTSD, DPP4, GDF15, TFRC | Age |
| 821 | SERPINA1, SERPINA3, CTSD, DPP4, GSN, MIF | Age |
| 822 | SERPINA1, SERPINA3, CTSD, DPP4, GSN, PKM | Age |
| 823 | SERPINA1, SERPINA3, CTSD, DPP4, GSN, TIMP1 | Age |
| 824 | SERPINA1, SERPINA3, CTSD, DPP4, GSN, TFRC | Age |
| 825 | SERPINA1, SERPINA3, CTSD, DPP4, MIF, PKM | Age |
| 826 | SERPINA1, SERPINA3, CTSD, DPP4, MIF, TIMP1 | Age |
| 827 | SERPINA1, SERPINA3, CTSD, DPP4, MIF, TFRC | Age |
| 828 | SERPINA1, SERPINA3, CTSD, DPP4, PKM, TIMP1 | Age |
| 829 | SERPINA1, SERPINA3, CTSD, DPP4, PKM, TFRC | Age |
| 830 | SERPINA1, SERPINA3, CTSD, DPP4, TIMP1, TFRC | Age |
| 831 | SERPINA1, SERPINA3, CTSD, GDF15, GSN, MIF | Age |
| 832 | SERPINA1, SERPINA3, CTSD, GDF15, GSN, PKM | Age |
| 833 | SERPINA1, SERPINA3, CTSD, GDF15, GSN, TIMP1 | Age |
| 834 | SERPINA1, SERPINA3, CTSD, GDF15, GSN, TFRC | Age |
| 835 | SERPINA1, SERPINA3, CTSD, GDF15, MIF, PKM | Age |
| 836 | SERPINA1, SERPINA3, CTSD, GDF15, MIF, TIMP1 | Age |
| 837 | SERPINA1, SERPINA3, CTSD, GDF15, MIF, TFRC | Age |
| 838 | SERPINA1, SERPINA3, CTSD, GDF15, PKM, TIMP1 | Age |
| 839 | SERPINA1, SERPINA3, CTSD, GDF15, PKM, TFRC | Age |
| 840 | SERPINA1, SERPINA3, CTSD, GDF15, TIMP1, TFRC | Age |
| 841 | SERPINA1, SERPINA3, CTSD, GSN, MIF, PKM | Age |
| 842 | SERPINA1, SERPINA3, CTSD, GSN, MIF, TIMP1 | Age |
| 843 | SERPINA1, SERPINA3, CTSD, GSN, MIF, TFRC | Age |
| 844 | SERPINA1, SERPINA3, CTSD, GSN, PKM, TIMP1 | Age |
| 845 | SERPINA1, SERPINA3, CTSD, GSN, PKM, TFRC | Age |
| 846 | SERPINA1, SERPINA3, CTSD, GSN, TIMP1, TFRC | Age |
| 847 | SERPINA1, SERPINA3, CTSD, MIF, PKM, TIMP1 | Age |
| 848 | SERPINA1, SERPINA3, CTSD, MIF, PKM, TFRC | Age |
| 849 | SERPINA1, SERPINA3, CTSD, MIF, TIMP1, TFRC | Age |
| 850 | SERPINA1, SERPINA3, CTSD, PKM, TIMP1, TFRC | Age |
| 851 | SERPINA1, SERPINA3, CLU, DPP4, GDF15, GSN | Age |
| 852 | SERPINA1, SERPINA3, CLU, DPP4, GDF15, MIF | Age |
| 853 | SERPINA1, SERPINA3, CLU, DPP4, GDF15, PKM | Age |
| 854 | SERPINA1, SERPINA3, CLU, DPP4, GDF15, TIMP1 | Age |
| 855 | SERPINA1, SERPINA3, CLU, DPP4, GDF15, TFRC | Age |
| 856 | SERPINA1, SERPINA3, CLU, DPP4, GSN, MIF | Age |
| 857 | SERPINA1, SERPINA3, CLU, DPP4, GSN, PKM | Age |
| 858 | SERPINA1, SERPINA3, CLU, DPP4, GSN, TIMP1 | Age |
| 859 | SERPINA1, SERPINA3, CLU, DPP4, GSN, TFRC | Age |
| 860 | SERPINA1, SERPINA3, CLU, DPP4, MIF, PKM | Age |
| 861 | SERPINA1, SERPINA3, CLU, DPP4, MIF, TIMP1 | Age |
| 862 | SERPINA1, SERPINA3, CLU, DPP4, MIF, TFRC | Age |
| 863 | SERPINA1, SERPINA3, CLU, DPP4, PKM, TIMP1 | Age |
| 864 | SERPINA1, SERPINA3, CLU, DPP4, PKM, TFRC | Age |
| 865 | SERPINA1, SERPINA3, CLU, DPP4, TIMP1, TFRC | Age |
| 866 | SERPINA1, SERPINA3, CLU, GDF15, GSN, MIF | Age |
| 867 | SERPINA1, SERPINA3, CLU, GDF15, GSN, PKM | Age |
| 868 | SERPINA1, SERPINA3, CLU, GDF15, GSN, TIMP1 | Age |
| 869 | SERPINA1, SERPINA3, CLU, GDF15, GSN, TFRC | Age |
| 870 | SERPINA1, SERPINA3, CLU, GDF15, MIF, PKM | Age |
| 871 | SERPINA1, SERPINA3, CLU, GDF15, MIF, TIMP1 | Age |
| 872 | SERPINA1, SERPINA3, CLU, GDF15, MIF, TFRC | Age |
| 873 | SERPINA1, SERPINA3, CLU, GDF15, PKM, TIMP1 | Age |
| 874 | SERPINA1, SERPINA3, CLU, GDF15, PKM, TFRC | Age |
| 875 | SERPINA1, SERPINA3, CLU, GDF15, TIMP1, TFRC | Age |
| 876 | SERPINA1, SERPINA3, CLU, GSN, MIF, PKM | Age |
| 877 | SERPINA1, SERPINA3, CLU, GSN, MIF, TIMP1 | Age |
| 878 | SERPINA1, SERPINA3, CLU, GSN, MIF, TFRC | Age |
| 879 | SERPINA1, SERPINA3, CLU, GSN, PKM, TIMP1 | Age |
| 880 | SERPINA1, SERPINA3, CLU, GSN, PKM, TFRC | Age |
| 881 | SERPINA1, SERPINA3, CLU, GSN, TIMP1, TFRC | Age |
| 882 | SERPINA1, SERPINA3, CLU, MIF, PKM, TIMP1 | Age |
| 883 | SERPINA1, SERPINA3, CLU, MIF, PKM, TFRC | Age |
| 884 | SERPINA1, SERPINA3, CLU, MIF, TIMP1, TFRC | Age |
| 885 | SERPINA1, SERPINA3, CLU, PKM, TIMP1, TFRC | Age |
| 886 | SERPINA1, SERPINA3, DPP4, GDF15, GSN, MIF | Age |
| 887 | SERPINA1, SERPINA3, DPP4, GDF15, GSN, PKM | Age |
| 888 | SERPINA1, SERPINA3, DPP4, GDF15, GSN, TIMP1 | Age |
| 889 | SERPINA1, SERPINA3, DPP4, GDF15, GSN, TFRC | Age |
| 890 | SERPINA1, SERPINA3, DPP4, GDF15, MIF, PKM | Age |
| 891 | SERPINA1, SERPINA3, DPP4, GDF15, MIF, TIMP1 | Age |
| 892 | SERPINA1, SERPINA3, DPP4, GDF15, MIF, TFRC | Age |
| 893 | SERPINA1, SERPINA3, DPP4, GDF15, PKM, TIMP1 | Age |
| 894 | SERPINA1, SERPINA3, DPP4, GDF15, PKM, TFRC | Age |
| 895 | SERPINA1, SERPINA3, DPP4, GDF15, TIMP1, TFRC | Age |
| 896 | SERPINA1, SERPINA3, DPP4, GSN, MIF, PKM | Age |
| 897 | SERPINA1, SERPINA3, DPP4, GSN, MIF, TIMP1 | Age |
| 898 | SERPINA1, SERPINA3, DPP4, GSN, MIF, TFRC | Age |
| 899 | SERPINA1, SERPINA3, DPP4, GSN, PKM, TIMP1 | Age |
| 900 | SERPINA1, SERPINA3, DPP4, GSN, PKM, TFRC | Age |
| 901 | SERPINA1, SERPINA3, DPP4, GSN, TIMP1, TFRC | Age |
| 902 | SERPINA1, SERPINA3, DPP4, MIF, PKM, TIMP1 | Age |
| 903 | SERPINA1, SERPINA3, DPP4, MIF, PKM, TFRC | Age |
| 904 | SERPINA1, SERPINA3, DPP4, MIF, TIMP1, TFRC | Age |
| 905 | SERPINA1, SERPINA3, DPP4, PKM, TIMP1, TFRC | Age |
| 906 | SERPINA1, SERPINA3, GDF15, GSN, MIF, PKM | Age |
| 907 | SERPINA1, SERPINA3, GDF15, GSN, MIF, TIMP1 | Age |
| 908 | SERPINA1, SERPINA3, GDF15, GSN, MIF, TFRC | Age |
| 909 | SERPINA1, SERPINA3, GDF15, GSN, PKM, TIMP1 | Age |
| 910 | SERPINA1, SERPINA3, GDF15, GSN, PKM, TFRC | Age |
| 911 | SERPINA1, SERPINA3, GDF15, GSN, TIMP1, TFRC | Age |
| 912 | SERPINA1, SERPINA3, GDF15, MIF, PKM, TIMP1 | Age |
| 913 | SERPINA1, SERPINA3, GDF15, MIF, PKM, TFRC | Age |
| 914 | SERPINA1, SERPINA3, GDF15, MIF, TIMP1, TFRC | Age |
| 915 | SERPINA1, SERPINA3, GDF15, PKM, TIMP1, TFRC | Age |
| 916 | SERPINA1, SERPINA3, GSN, MIF, PKM, TIMP1 | Age |
| 917 | SERPINA1, SERPINA3, GSN, MIF, PKM, TFRC | Age |
| 918 | SERPINA1, SERPINA3, GSN, MIF, TIMP1, TFRC | Age |
| 919 | SERPINA1, SERPINA3, GSN, PKM, TIMP1, TFRC | Age |
| 920 | SERPINA1, SERPINA3, MIF, PKM, TIMP1, TFRC | Age |
| 921 | SERPINA1, CTSD, CLU, DPP4, GDF15, GSN | Age |
| 922 | SERPINA1, CTSD, CLU, DPP4, GDF15, MIF | Age |
| 923 | SERPINA1, CTSD, CLU, DPP4, GDF15, PKM | Age |
| 924 | SERPINA1, CTSD, CLU, DPP4, GDF15, TIMP1 | Age |
| 925 | SERPINA1, CTSD, CLU, DPP4, GDF15, TFRC | Age |
| 926 | SERPINA1, CTSD, CLU, DPP4, GSN, MIF | Age |
| 927 | SERPINA1, CTSD, CLU, DPP4, GSN, PKM | Age |
| 928 | SERPINA1, CTSD, CLU, DPP4, GSN, TIMP1 | Age |
| 929 | SERPINA1, CTSD, CLU, DPP4, GSN, TFRC | Age |
| 930 | SERPINA1, CTSD, CLU, DPP4, MIF, PKM | Age |
| 931 | SERPINA1, CTSD, CLU, DPP4, MIF, TIMP1 | Age |
| 932 | SERPINA1, CTSD, CLU, DPP4, MIF, TFRC | Age |
| 933 | SERPINA1, CTSD, CLU, DPP4, PKM, TIMP1 | Age |
| 934 | SERPINA1, CTSD, CLU, DPP4, PKM, TFRC | Age |
| 935 | SERPINA1, CTSD, CLU, DPP4, TIMP1, TFRC | Age |
| 936 | SERPINA1, CTSD, CLU, GDF15, GSN, MIF | Age |
| 937 | SERPINA1, CTSD, CLU, GDF15, GSN, PKM | Age |
| 938 | SERPINA1, CTSD, CLU, GDF15, GSN, TIMP1 | Age |
| 939 | SERPINA1, CTSD, CLU, GDF15, GSN, TFRC | Age |
| 940 | SERPINA1, CTSD, CLU, GDF15, MIF, PKM | Age |
| 941 | SERPINA1, CTSD, CLU, GDF15, MIF, TIMP1 | Age |
| 942 | SERPINA1, CTSD, CLU, GDF15, MIF, TFRC | Age |
| 943 | SERPINA1, CTSD, CLU, GDF15, PKM, TIMP1 | Age |
| 944 | SERPINA1, CTSD, CLU, GDF15, PKM, TFRC | Age |
| 945 | SERPINA1, CTSD, CLU, GDF15, TIMP1, TFRC | Age |
| 946 | SERPINA1, CTSD, CLU, GSN, MIF, PKM | Age |
| 947 | SERPINA1, CTSD, CLU, GSN, MIF, TIMP1 | Age |
| 948 | SERPINA1, CTSD, CLU, GSN, MIF, TFRC | Age |
| 949 | SERPINA1, CTSD, CLU, GSN, PKM, TIMP1 | Age |
| 950 | SERPINA1, CTSD, CLU, GSN, PKM, TFRC | Age |
| 951 | SERPINA1, CTSD, CLU, GSN, TIMP1, TFRC | Age |
| 952 | SERPINA1, CTSD, CLU, MIF, PKM, TIMP1 | Age |
| 953 | SERPINA1, CTSD, CLU, MIF, PKM, TFRC | Age |
| 954 | SERPINA1, CTSD, CLU, MIF, TIMP1, TFRC | Age |
| 955 | SERPINA1, CTSD, CLU, PKM, TIMP1, TFRC | Age |
| 956 | SERPINA1, CTSD, DPP4, GDF15, GSN, MIF | Age |
| 957 | SERPINA1, CTSD, DPP4, GDF15, GSN, PKM | Age |
| 958 | SERPINA1, CTSD, DPP4, GDF15, GSN, TIMP1 | Age |
| 959 | SERPINA1, CTSD, DPP4, GDF15, GSN, TFRC | Age |
| 960 | SERPINA1, CTSD, DPP4, GDF15, MIF, PKM | Age |
| 961 | SERPINA1, CTSD, DPP4, GDF15, MIF, TIMP1 | Age |
| 962 | SERPINA1, CTSD, DPP4, GDF15, MIF, TFRC | Age |
| 963 | SERPINA1, CTSD, DPP4, GDF15, PKM, TIMP1 | Age |
| 964 | SERPINA1, CTSD, DPP4, GDF15, PKM, TFRC | Age |
| 965 | SERPINA1, CTSD, DPP4, GDF15, TIMP1, TFRC | Age |
| 966 | SERPINA1, CTSD, DPP4, GSN, MIF, PKM | Age |
| 967 | SERPINA1, CTSD, DPP4, GSN, MIF, TIMP1 | Age |
| 968 | SERPINA1, CTSD, DPP4, GSN, MIF, TFRC | Age |
| 969 | SERPINA1, CTSD, DPP4, GSN, PKM, TIMP1 | Age |
| 970 | SERPINA1, CTSD, DPP4, GSN, PKM, TFRC | Age |
| 971 | SERPINA1, CTSD, DPP4, GSN, TIMP1, TFRC | Age |
| 972 | SERPINA1, CTSD, DPP4, MIF, PKM, TIMP1 | Age |
| 973 | SERPINA1, CTSD, DPP4, MIF, PKM, TFRC | Age |
| 974 | SERPINA1, CTSD, DPP4, MIF, TIMP1, TFRC | Age |
| 975 | SERPINA1, CTSD, DPP4, PKM, TIMP1, TFRC | Age |
| 976 | SERPINA1, CTSD, GDF15, GSN, MIF, PKM | Age |
| 977 | SERPINA1, CTSD, GDF15, GSN, MIF, TIMP1 | Age |
| 978 | SERPINA1, CTSD, GDF15, GSN, MIF, TFRC | Age |
| 979 | SERPINA1, CTSD, GDF15, GSN, PKM, TIMP1 | Age |
| 980 | SERPINA1, CTSD, GDF15, GSN, PKM, TFRC | Age |
| 981 | SERPINA1, CTSD, GDF15, GSN, TIMP1, TFRC | Age |
| 982 | SERPINA1, CTSD, GDF15, MIF, PKM, TIMP1 | Age |
| 983 | SERPINA1, CTSD, GDF15, MIF, PKM, TFRC | Age |
| 984 | SERPINA1, CTSD, GDF15, MIF, TIMP1, TFRC | Age |
| 985 | SERPINA1, CTSD, GDF15, PKM, TIMP1, TFRC | Age |
| 986 | SERPINA1, CTSD, GSN, MIF, PKM, TIMP1 | Age |
| 987 | SERPINA1, CTSD, GSN, MIF, PKM, TFRC | Age |
| 988 | SERPINA1, CTSD, GSN, MIF, TIMP1, TFRC | Age |
| 989 | SERPINA1, CTSD, GSN, PKM, TIMP1, TFRC | Age |
| 990 | SERPINA1, CTSD, MIF, PKM, TIMP1, TFRC | Age |
| 991 | SERPINA1, CLU, DPP4, GDF15, GSN, MIF | Age |
| 992 | SERPINA1, CLU, DPP4, GDF15, GSN, PKM | Age |
| 993 | SERPINA1, CLU, DPP4, GDF15, GSN, TIMP1 | Age |
| 994 | SERPINA1, CLU, DPP4, GDF15, GSN, TFRC | Age |
| 995 | SERPINA1, CLU, DPP4, GDF15, MIF, PKM | Age |
| 996 | SERPINA1, CLU, DPP4, GDF15, MIF, TIMP1 | Age |
| 997 | SERPINA1, CLU, DPP4, GDF15, MIF, TFRC | Age |
| 998 | SERPINA1, CLU, DPP4, GDF15, PKM, TIMP1 | Age |
| 999 | SERPINA1, CLU, DPP4, GDF15, PKM, TFRC | Age |
| 1000 | SERPINA1, CLU, DPP4, GDF15, TIMP1, TFRC | Age |
| 1001 | SERPINA1, CLU, DPP4, GSN, MIF, PKM | Age |
| 1002 | SERPINA1, CLU, DPP4, GSN, MIF, TIMP1 | Age |
| 1003 | SERPINA1, CLU, DPP4, GSN, MIF, TFRC | Age |
| 1004 | SERPINA1, CLU, DPP4, GSN, PKM, TIMP1 | Age |
| 1005 | SERPINA1, CLU, DPP4, GSN, PKM, TFRC | Age |
| 1006 | SERPINA1, CLU, DPP4, GSN, TIMP1, TFRC | Age |
| 1007 | SERPINA1, CLU, DPP4, MIF, PKM, TIMP1 | Age |
| 1008 | SERPINA1, CLU, DPP4, MIF, PKM, TFRC | Age |
| 1009 | SERPINA1, CLU, DPP4, MIF, TIMP1, TFRC | Age |
| 1010 | SERPINA1, CLU, DPP4, PKM, TIMP1, TFRC | Age |
| 1011 | SERPINA1, CLU, GDF15, GSN, MIF, PKM | Age |
| 1012 | SERPINA1, CLU, GDF15, GSN, MIF, TIMP1 | Age |
| 1013 | SERPINA1, CLU, GDF15, GSN, MIF, TFRC | Age |
| 1014 | SERPINA1, CLU, GDF15, GSN, PKM, TIMP1 | Age |
| 1015 | SERPINA1, CLU, GDF15, GSN, PKM, TFRC | Age |
| 1016 | SERPINA1, CLU, GDF15, GSN, TIMP1, TFRC | Age |
| 1017 | SERPINA1, CLU, GDF15, MIF, PKM, TIMP1 | Age |
| 1018 | SERPINA1, CLU, GDF15, MIF, PKM, TFRC | Age |
| 1019 | SERPINA1, CLU, GDF15, MIF, TIMP1, TFRC | Age |
| 1020 | SERPINA1, CLU, GDF15, PKM, TIMP1, TFRC | Age |
| 1021 | SERPINA1, CLU, GSN, MIF, PKM, TIMP1 | Age |
| 1022 | SERPINA1, CLU, GSN, MIF, PKM, TFRC | Age |
| 1023 | SERPINA1, CLU, GSN, MIF, TIMP1, TFRC | Age |
| 1024 | SERPINA1, CLU, GSN, PKM, TIMP1, TFRC | Age |
| 1025 | SERPINA1, CLU, MIF, PKM, TIMP1, TFRC | Age |
| 1026 | SERPINA1, DPP4, GDF15, GSN, MIF, PKM | Age |
| 1027 | SERPINA1, DPP4, GDF15, GSN, MIF, TIMP1 | Age |
| 1028 | SERPINA1, DPP4, GDF15, GSN, MIF, TFRC | Age |
| 1029 | SERPINA1, DPP4, GDF15, GSN, PKM, TIMP1 | Age |
| 1030 | SERPINA1, DPP4, GDF15, GSN, PKM, TFRC | Age |
| 1031 | SERPINA1, DPP4, GDF15, GSN, TIMP1, TFRC | Age |
| 1032 | SERPINA1, DPP4, GDF15, MIF, PKM, TIMP1 | Age |
| 1033 | SERPINA1, DPP4, GDF15, MIF, PKM, TFRC | Age |
| 1034 | SERPINA1, DPP4, GDF15, MIF, TIMP1, TFRC | Age |
| 1035 | SERPINA1, DPP4, GDF15, PKM, TIMP1, TFRC | Age |
| 1036 | SERPINA1, DPP4, GSN, MIF, PKM, TIMP1 | Age |
| 1037 | SERPINA1, DPP4, GSN, MIF, PKM, TFRC | Age |
| 1038 | SERPINA1, DPP4, GSN, MIF, TIMP1, TFRC | Age |
| 1039 | SERPINA1, DPP4, GSN, PKM, TIMP1, TFRC | Age |
| 1040 | SERPINA1, DPP4, MIF, PKM, TIMP1, TFRC | Age |
| 1041 | SERPINA1, GDF15, GSN, MIF, PKM, TIMP1 | Age |
| 1042 | SERPINA1, GDF15, GSN, MIF, PKM, TFRC | Age |
| 1043 | SERPINA1, GDF15, GSN, MIF, TIMP1, TFRC | Age |
| 1044 | SERPINA1, GDF15, GSN, PKM, TIMP1, TFRC | Age |
| 1045 | SERPINA1, GDF15, MIF, PKM, TIMP1, TFRC | Age |
| 1046 | SERPINA1, GSN, MIF, PKM, TIMP1, TFRC | Age |
| 1047 | SERPINA3, CTSD, CLU, DPP4, GDF15, GSN | Age |
| 1048 | SERPINA3, CTSD, CLU, DPP4, GDF15, MIF | Age |
| 1049 | SERPINA3, CTSD, CLU, DPP4, GDF15, PKM | Age |
| 1050 | SERPINA3, CTSD, CLU, DPP4, GDF15, TIMP1 | Age |
| 1051 | SERPINA3, CTSD, CLU, DPP4, GDF15, TFRC | Age |
| 1052 | SERPINA3, CTSD, CLU, DPP4, GSN, MIF | Age |
| 1053 | SERPINA3, CTSD, CLU, DPP4, GSN, PKM | Age |
| 1054 | SERPINA3, CTSD, CLU, DPP4, GSN, TIMP1 | Age |
| 1055 | SERPINA3, CTSD, CLU, DPP4, GSN, TFRC | Age |
| 1056 | SERPINA3, CTSD, CLU, DPP4, MIF, PKM | Age |
| 1057 | SERPINA3, CTSD, CLU, DPP4, MIF, TIMP1 | Age |
| 1058 | SERPINA3, CTSD, CLU, DPP4, MIF, TFRC | Age |
| 1059 | SERPINA3, CTSD, CLU, DPP4, PKM, TIMP1 | Age |
| 1060 | SERPINA3, CTSD, CLU, DPP4, PKM, TFRC | Age |
| 1061 | SERPINA3, CTSD, CLU, DPP4, TIMP1, TFRC | Age |
| 1062 | SERPINA3, CTSD, CLU, GDF15, GSN, MIF | Age |
| 1063 | SERPINA3, CTSD, CLU, GDF15, GSN, PKM | Age |
| 1064 | SERPINA3, CTSD, CLU, GDF15, GSN, TIMP1 | Age |
| 1065 | SERPINA3, CTSD, CLU, GDF15, GSN, TFRC | Age |
| 1066 | SERPINA3, CTSD, CLU, GDF15, MIF, PKM | Age |
| 1067 | SERPINA3, CTSD, CLU, GDF15, MIF, TIMP1 | Age |
| 1068 | SERPINA3, CTSD, CLU, GDF15, MIF, TFRC | Age |
| 1069 | SERPINA3, CTSD, CLU, GDF15, PKM, TIMP1 | Age |
| 1070 | SERPINA3, CTSD, CLU, GDF15, PKM, TFRC | Age |
| 1071 | SERPINA3, CTSD, CLU, GDF15, TIMP1, TFRC | Age |
| 1072 | SERPINA3, CTSD, CLU, GSN, MIF, PKM | Age |
| 1073 | SERPINA3, CTSD, CLU, GSN, MIF, TIMP1 | Age |
| 1074 | SERPINA3, CTSD, CLU, GSN, MIF, TFRC | Age |
| 1075 | SERPINA3, CTSD, CLU, GSN, PKM, TIMP1 | Age |
| 1076 | SERPINA3, CTSD, CLU, GSN, PKM, TFRC | Age |
| 1077 | SERPINA3, CTSD, CLU, GSN, TIMP1, TFRC | Age |
| 1078 | SERPINA3, CTSD, CLU, MIF, PKM, TIMP1 | Age |
| 1079 | SERPINA3, CTSD, CLU, MIF, PKM, TFRC | Age |
| 1080 | SERPINA3, CTSD, CLU, MIF, TIMP1, TFRC | Age |
| 1081 | SERPINA3, CTSD, CLU, PKM, TIMP1, TFRC | Age |
| 1082 | SERPINA3, CTSD, DPP4, GDF15, GSN, MIF | Age |
| 1083 | SERPINA3, CTSD, DPP4, GDF15, GSN, PKM | Age |
| 1084 | SERPINA3, CTSD, DPP4, GDF15, GSN, TIMP1 | Age |
| 1085 | SERPINA3, CTSD, DPP4, GDF15, GSN, TFRC | Age |
| 1086 | SERPINA3, CTSD, DPP4, GDF15, MIF, PKM | Age |
| 1087 | SERPINA3, CTSD, DPP4, GDF15, MIF, TIMP1 | Age |
| 1088 | SERPINA3, CTSD, DPP4, GDF15, MIF, TFRC | Age |
| 1089 | SERPINA3, CTSD, DPP4, GDF15, PKM, TIMP1 | Age |
| 1090 | SERPINA3, CTSD, DPP4, GDF15, PKM, TFRC | Age |
| 1091 | SERPINA3, CTSD, DPP4, GDF15, TIMP1, TFRC | Age |
| 1092 | SERPINA3, CTSD, DPP4, GSN, MIF, PKM | Age |
| 1093 | SERPINA3, CTSD, DPP4, GSN, MIF, TIMP1 | Age |
| 1094 | SERPINA3, CTSD, DPP4, GSN, MIF, TFRC | Age |
| 1095 | SERPINA3, CTSD, DPP4, GSN, PKM, TIMP1 | Age |
| 1096 | SERPINA3, CTSD, DPP4, GSN, PKM, TFRC | Age |
| 1097 | SERPINA3, CTSD, DPP4, GSN, TIMP1, TFRC | Age |
| 1098 | SERPINA3, CTSD, DPP4, MIF, PKM, TIMP1 | Age |
| 1099 | SERPINA3, CTSD, DPP4, MIF, PKM, TFRC | Age |
| 1100 | SERPINA3, CTSD, DPP4, MIF, TIMP1, TFRC | Age |
| 1101 | SERPINA3, CTSD, DPP4, PKM, TIMP1, TFRC | Age |
| 1102 | SERPINA3, CTSD, GDF15, GSN, MIF, PKM | Age |
| 1103 | SERPINA3, CTSD, GDF15, GSN, MIF, TIMP1 | Age |
| 1104 | SERPINA3, CTSD, GDF15, GSN, MIF, TFRC | Age |
| 1105 | SERPINA3, CTSD, GDF15, GSN, PKM, TIMP1 | Age |
| 1106 | SERPINA3, CTSD, GDF15, GSN, PKM, TFRC | Age |
| 1107 | SERPINA3, CTSD, GDF15, GSN, TIMP1, TFRC | Age |
| 1108 | SERPINA3, CTSD, GDF15, MIF, PKM, TIMP1 | Age |
| 1109 | SERPINA3, CTSD, GDF15, MIF, PKM, TFRC | Age |
| 1110 | SERPINA3, CTSD, GDF15, MIF, TIMP1, TFRC | Age |
| 1111 | SERPINA3, CTSD, GDF15, PKM, TIMP1, TFRC | Age |
| 1112 | SERPINA3, CTSD, GSN, MIF, PKM, TIMP1 | Age |
| 1113 | SERPINA3, CTSD, GSN, MIF, PKM, TFRC | Age |
| 1114 | SERPINA3, CTSD, GSN, MIF, TIMP1, TFRC | Age |
| 1115 | SERPINA3, CTSD, GSN, PKM, TIMP1, TFRC | Age |
| 1116 | SERPINA3, CTSD, MIF, PKM, TIMP1, TFRC | Age |
| 1117 | SERPINA3, CLU, DPP4, GDF15, GSN, MIF | Age |
| 1118 | SERPINA3, CLU, DPP4, GDF15, GSN, PKM | Age |
| 1119 | SERPINA3, CLU, DPP4, GDF15, GSN, TIMP1 | Age |
| 1120 | SERPINA3, CLU, DPP4, GDF15, GSN, TFRC | Age |
| 1121 | SERPINA3, CLU, DPP4, GDF15, MIF, PKM | Age |
| 1122 | SERPINA3, CLU, DPP4, GDF15, MIF, TIMP1 | Age |
| 1123 | SERPINA3, CLU, DPP4, GDF15, MIF, TFRC | Age |
| 1124 | SERPINA3, CLU, DPP4, GDF15, PKM, TIMP1 | Age |
| 1125 | SERPINA3, CLU, DPP4, GDF15, PKM, TFRC | Age |
| 1126 | SERPINA3, CLU, DPP4, GDF15, TIMP1, TFRC | Age |
| 1127 | SERPINA3, CLU, DPP4, GSN, MIF, PKM | Age |
| 1128 | SERPINA3, CLU, DPP4, GSN, MIF, TIMP1 | Age |
| 1129 | SERPINA3, CLU, DPP4, GSN, MIF, TFRC | Age |
| 1130 | SERPINA3, CLU, DPP4, GSN, PKM, TIMP1 | Age |
| 1131 | SERPINA3, CLU, DPP4, GSN, PKM, TFRC | Age |
| 1132 | SERPINA3, CLU, DPP4, GSN, TIMP1, TFRC | Age |
| 1133 | SERPINA3, CLU, DPP4, MIF, PKM, TIMP1 | Age |
| 1134 | SERPINA3, CLU, DPP4, MIF, PKM, TFRC | Age |
| 1135 | SERPINA3, CLU, DPP4, MIF, TIMP1, TFRC | Age |
| 1136 | SERPINA3, CLU, DPP4, PKM, TIMP1, TFRC | Age |
| 1137 | SERPINA3, CLU, GDF15, GSN, MIF, PKM | Age |
| 1138 | SERPINA3, CLU, GDF15, GSN, MIF, TIMP1 | Age |
| 1139 | SERPINA3, CLU, GDF15, GSN, MIF, TFRC | Age |
| 1140 | SERPINA3, CLU, GDF15, GSN, PKM, TIMP1 | Age |
| 1141 | SERPINA3, CLU, GDF15, GSN, PKM, TFRC | Age |
| 1142 | SERPINA3, CLU, GDF15, GSN, TIMP1, TFRC | Age |
| 1143 | SERPINA3, CLU, GDF15, MIF, PKM, TIMP1 | Age |
| 1144 | SERPINA3, CLU, GDF15, MIF, PKM, TFRC | Age |
| 1145 | SERPINA3, CLU, GDF15, MIF, TIMP1, TFRC | Age |
| 1146 | SERPINA3, CLU, GDF15, PKM, TIMP1, TFRC | Age |
| 1147 | SERPINA3, CLU, GSN, MIF, PKM, TIMP1 | Age |
| 1148 | SERPINA3, CLU, GSN, MIF, PKM, TFRC | Age |
| 1149 | SERPINA3, CLU, GSN, MIF, TIMP1, TFRC | Age |
| 1150 | SERPINA3, CLU, GSN, PKM, TIMP1, TFRC | Age |
| 1151 | SERPINA3, CLU, MIF, PKM, TIMP1, TFRC | Age |
| 1152 | SERPINA3, DPP4, GDF15, GSN, MIF, PKM | Age |
| 1153 | SERPINA3, DPP4, GDF15, GSN, MIF, TIMP1 | Age |
| 1154 | SERPINA3, DPP4, GDF15, GSN, MIF, TFRC | Age |
| 1155 | SERPINA3, DPP4, GDF15, GSN, PKM, TIMP1 | Age |
| 1156 | SERPINA3, DPP4, GDF15, GSN, PKM, TFRC | Age |
| 1157 | SERPINA3, DPP4, GDF15, GSN, TIMP1, TFRC | Age |
| 1158 | SERPINA3, DPP4, GDF15, MIF, PKM, TIMP1 | Age |
| 1159 | SERPINA3, DPP4, GDF15, MIF, PKM, TFRC | Age |
| 1160 | SERPINA3, DPP4, GDF15, MIF, TIMP1, TFRC | Age |
| 1161 | SERPINA3, DPP4, GDF15, PKM, TIMP1, TFRC | Age |
| 1162 | SERPINA3, DPP4, GSN, MIF, PKM, TIMP1 | Age |
| 1163 | SERPINA3, DPP4, GSN, MIF, PKM, TFRC | Age |
| 1164 | SERPINA3, DPP4, GSN, MIF, TIMP1, TFRC | Age |
| 1165 | SERPINA3, DPP4, GSN, PKM, TIMP1, TFRC | Age |
| 1166 | SERPINA3, DPP4, MIF, PKM, TIMP1, TFRC | Age |
| 1167 | SERPINA3, GDF15, GSN, MIF, PKM, TIMP1 | Age |
| 1168 | SERPINA3, GDF15, GSN, MIF, PKM, TFRC | Age |
| 1169 | SERPINA3, GDF15, GSN, MIF, TIMP1, TFRC | Age |
| 1170 | SERPINA3, GDF15, GSN, PKM, TIMP1, TFRC | Age |
| 1171 | SERPINA3, GDF15, MIF, PKM, TIMP1, TFRC | Age |
| 1172 | SERPINA3, GSN, MIF, PKM, TIMP1, TFRC | Age |
| 1173 | CTSD, CLU, DPP4, GDF15, GSN, MIF | Age |
| 1174 | CTSD, CLU, DPP4, GDF15, GSN, PKM | Age |
| 1175 | CTSD, CLU, DPP4, GDF15, GSN, TIMP1 | Age |
| 1176 | CTSD, CLU, DPP4, GDF15, GSN, TFRC | Age |
| 1177 | CTSD, CLU, DPP4, GDF15, MIF, PKM | Age |
| 1178 | CTSD, CLU, DPP4, GDF15, MIF, TIMP1 | Age |
| 1179 | CTSD, CLU, DPP4, GDF15, MIF, TFRC | Age |
| 1180 | CTSD, CLU, DPP4, GDF15, PKM, TIMP1 | Age |
| 1181 | CTSD, CLU, DPP4, GDF15, PKM, TFRC | Age |
| 1182 | CTSD, CLU, DPP4, GDF15, TIMP1, TFRC | Age |
| 1183 | CTSD, CLU, DPP4, GSN, MIF, PKM | Age |
| 1184 | CTSD, CLU, DPP4, GSN, MIF, TIMP1 | Age |
| 1185 | CTSD, CLU, DPP4, GSN, MIF, TFRC | Age |
| 1186 | CTSD, CLU, DPP4, GSN, PKM, TIMP1 | Age |
| 1187 | CTSD, CLU, DPP4, GSN, PKM, TFRC | Age |
| 1188 | CTSD, CLU, DPP4, GSN, TIMP1, TFRC | Age |
| 1189 | CTSD, CLU, DPP4, MIF, PKM, TIMP1 | Age |
| 1190 | CTSD, CLU, DPP4, MIF, PKM, TFRC | Age |
| 1191 | CTSD, CLU, DPP4, MIF, TIMP1, TFRC | Age |
| 1192 | CTSD, CLU, DPP4, PKM, TIMP1, TFRC | Age |
| 1193 | CTSD, CLU, GDF15, GSN, MIF, PKM | Age |
| 1194 | CTSD, CLU, GDF15, GSN, MIF, TIMP1 | Age |
| 1195 | CTSD, CLU, GDF15, GSN, MIF, TFRC | Age |
| 1196 | CTSD, CLU, GDF15, GSN, PKM, TIMP1 | Age |
| 1197 | CTSD, CLU, GDF15, GSN, PKM, TFRC | Age |
| 1198 | CTSD, CLU, GDF15, GSN, TIMP1, TFRC | Age |
| 1199 | CTSD, CLU, GDF15, MIF, PKM, TIMP1 | Age |
| 1200 | CTSD, CLU, GDF15, MIF, PKM, TFRC | Age |
| 1201 | CTSD, CLU, GDF15, MIF, TIMP1, TFRC | Age |
| 1202 | CTSD, CLU, GDF15, PKM, TIMP1, TFRC | Age |
| 1203 | CTSD, CLU, GSN, MIF, PKM, TIMP1 | Age |
| 1204 | CTSD, CLU, GSN, MIF, PKM, TFRC | Age |
| 1205 | CTSD, CLU, GSN, MIF, TIMP1, TFRC | Age |
| 1206 | CTSD, CLU, GSN, PKM, TIMP1, TFRC | Age |
| 1207 | CTSD, CLU, MIF, PKM, TIMP1, TFRC | Age |
| 1208 | CTSD, DPP4, GDF15, GSN, MIF, PKM | Age |
| 1209 | CTSD, DPP4, GDF15, GSN, MIF, TIMP1 | Age |
| 1210 | CTSD, DPP4, GDF15, GSN, MIF, TFRC | Age |
| 1211 | CTSD, DPP4, GDF15, GSN, PKM, TIMP1 | Age |
| 1212 | CTSD, DPP4, GDF15, GSN, PKM, TFRC | Age |
| 1213 | CTSD, DPP4, GDF15, GSN, TIMP1, TFRC | Age |
| 1214 | CTSD, DPP4, GDF15, MIF, PKM, TIMP1 | Age |
| 1215 | CTSD, DPP4, GDF15, MIF, PKM, TFRC | Age |
| 1216 | CTSD, DPP4, GDF15, MIF, TIMP1, TFRC | Age |
| 1217 | CTSD, DPP4, GDF15, PKM, TIMP1, TFRC | Age |
| 1218 | CTSD, DPP4, GSN, MIF, PKM, TIMP1 | Age |
| 1219 | CTSD, DPP4, GSN, MIF, PKM, TFRC | Age |
| 1220 | CTSD, DPP4, GSN, MIF, TIMP1, TFRC | Age |
| 1221 | CTSD, DPP4, GSN, PKM, TIMP1, TFRC | Age |
| 1222 | CTSD, DPP4, MIF, PKM, TIMP1, TFRC | Age |
| 1223 | CTSD, GDF15, GSN, MIF, PKM, TIMP1 | Age |
| 1224 | CTSD, GDF15, GSN, MIF, PKM, TFRC | Age |
| 1225 | CTSD, GDF15, GSN, MIF, TIMP1, TFRC | Age |
| 1226 | CTSD, GDF15, GSN, PKM, TIMP1, TFRC | Age |
| 1227 | CTSD, GDF15, MIF, PKM, TIMP1, TFRC | Age |
| 1228 | CTSD, GSN, MIF, PKM, TIMP1, TFRC | Age |
| 1229 | CLU, DPP4, GDF15, GSN, MIF, PKM | Age |
| 1230 | CLU, DPP4, GDF15, GSN, MIF, TIMP1 | Age |
| 1231 | CLU, DPP4, GDF15, GSN, MIF, TFRC | Age |
| 1232 | CLU, DPP4, GDF15, GSN, PKM, TIMP1 | Age |
| 1233 | CLU, DPP4, GDF15, GSN, PKM, TFRC | Age |
| 1234 | CLU, DPP4, GDF15, GSN, TIMP1, TFRC | Age |
| 1235 | CLU, DPP4, GDF15, MIF, PKM, TIMP1 | Age |
| 1236 | CLU, DPP4, GDF15, MIF, PKM, TFRC | Age |
| 1237 | CLU, DPP4, GDF15, MIF, TIMP1, TFRC | Age |
| 1238 | CLU, DPP4, GDF15, PKM, TIMP1, TFRC | Age |
| 1239 | CLU, DPP4, GSN, MIF, PKM, TIMP1 | Age |
| 1240 | CLU, DPP4, GSN, MIF, PKM, TFRC | Age |
| 1241 | CLU, DPP4, GSN, MIF, TIMP1, TFRC | Age |
| 1242 | CLU, DPP4, GSN, PKM, TIMP1, TFRC | Age |
| 1243 | CLU, DPP4, MIF, PKM, TIMP1, TFRC | Age |
| 1244 | CLU, GDF15, GSN, MIF, PKM, TIMP1 | Age |
| 1245 | CLU, GDF15, GSN, MIF, PKM, TFRC | Age |
| 1246 | CLU, GDF15, GSN, MIF, TIMP1, TFRC | Age |
| 1247 | CLU, GDF15, GSN, PKM, TIMP1, TFRC | Age |
| 1248 | CLU, GDF15, MIF, PKM, TIMP1, TFRC | Age |
| 1249 | CLU, GSN, MIF, PKM, TIMP1, TFRC | Age |
| 1250 | DPP4, GDF15, GSN, MIF, PKM, TIMP1 | Age |
| 1251 | DPP4, GDF15, GSN, MIF, PKM, TFRC | Age |
| 1252 | DPP4, GDF15, GSN, MIF, TIMP1, TFRC | Age |
| 1253 | DPP4, GDF15, GSN, PKM, TIMP1, TFRC | Age |
| 1254 | DPP4, GDF15, MIF, PKM, TIMP1, TFRC | Age |
| 1255 | DPP4, GSN, MIF, PKM, TIMP1, TFRC | Age |
| 1256 | GDF15, GSN, MIF, PKM, TIMP1, TFRC | Age |
| 1257 | SERPINA1, SERPINA3, CTSD, CLU, DPP4, GDF15, GSN | NONE |
| 1258 | SERPINA1, SERPINA3, CTSD, CLU, DPP4, GDF15, MIF | NONE |
| 1259 | SERPINA1, SERPINA3, CTSD, CLU, DPP4, GDF15, PKM | NONE |
| 1260 | SERPINA1, SERPINA3, CTSD, CLU, DPP4, GDF15, TIMP1 | NONE |
| 1261 | SERPINA1, SERPINA3, CTSD, CLU, DPP4, GDF15, TFRC | NONE |
| 1262 | SERPINA1, SERPINA3, CTSD, CLU, DPP4, GSN, MIF | NONE |
| 1263 | SERPINA1, SERPINA3, CTSD, CLU, DPP4, GSN, PKM | NONE |
| 1264 | SERPINA1, SERPINA3, CTSD, CLU, DPP4, GSN, TIMP1 | NONE |
| 1265 | SERPINA1, SERPINA3, CTSD, CLU, DPP4, GSN, TFRC | NONE |
| 1266 | SERPINA1, SERPINA3, CTSD, CLU, DPP4, MIF, PKM | NONE |
| 1267 | SERPINA1, SERPINA3, CTSD, CLU, DPP4, MIF, TIMP1 | NONE |
| 1268 | SERPINA1, SERPINA3, CTSD, CLU, DPP4, MIF, TFRC | NONE |
| 1269 | SERPINA1, SERPINA3, CTSD, CLU, DPP4, PKM, TIMP1 | NONE |
| 1270 | SERPINA1, SERPINA3, CTSD, CLU, DPP4, PKM, TFRC | NONE |
| 1271 | SERPINA1, SERPINA3, CTSD, CLU, DPP4, TIMP1, TFRC | NONE |
| 1272 | SERPINA1, SERPINA3, CTSD, CLU, GDF15, GSN, MIF | NONE |
| 1273 | SERPINA1, SERPINA3, CTSD, CLU, GDF15, GSN, PKM | NONE |
| 1274 | SERPINA1, SERPINA3, CTSD, CLU, GDF15, GSN, TIMP1 | NONE |
| 1275 | SERPINA1, SERPINA3, CTSD, CLU, GDF15, GSN, TFRC | NONE |
| 1276 | SERPINA1, SERPINA3, CTSD, CLU, GDF15, MIF, PKM | NONE |
| 1277 | SERPINA1, SERPINA3, CTSD, CLU, GDF15, MIF, TIMP1 | NONE |
| 1278 | SERPINA1, SERPINA3, CTSD, CLU, GDF15, MIF, TFRC | NONE |
| 1279 | SERPINA1, SERPINA3, CTSD, CLU, GDF15, PKM, TIMP1 | NONE |
| 1280 | SERPINA1, SERPINA3, CTSD, CLU, GDF15, PKM, TFRC | NONE |
| 1281 | SERPINA1, SERPINA3, CTSD, CLU, GDF15, TIMP1, TFRC | NONE |
| 1282 | SERPINA1, SERPINA3, CTSD, CLU, GSN, MIF, PKM | NONE |
| 1283 | SERPINA1, SERPINA3, CTSD, CLU, GSN, MIF, TIMP1 | NONE |
| 1284 | SERPINA1, SERPINA3, CTSD, CLU, GSN, MIF, TFRC | NONE |
| 1285 | SERPINA1, SERPINA3, CTSD, CLU, GSN, PKM, TIMP1 | NONE |
| 1286 | SERPINA1, SERPINA3, CTSD, CLU, GSN, PKM, TFRC | NONE |
| 1287 | SERPINA1, SERPINA3, CTSD, CLU, GSN, TIMP1, TFRC | NONE |
| 1288 | SERPINA1, SERPINA3, CTSD, CLU, MIF, PKM, TIMP1 | NONE |
| 1289 | SERPINA1, SERPINA3, CTSD, CLU, MIF, PKM, TFRC | NONE |
| 1290 | SERPINA1, SERPINA3, CTSD, CLU, MIF, TIMP1, TFRC | NONE |
| 1291 | SERPINA1, SERPINA3, CTSD, CLU, PKM, TIMP1, TFRC | NONE |
| 1292 | SERPINA1, SERPINA3, CTSD, DPP4, GDF15, GSN, MIF | NONE |
| 1293 | SERPINA1, SERPINA3, CTSD, DPP4, GDF15, GSN, PKM | NONE |
| 1294 | SERPINA1, SERPINA3, CTSD, DPP4, GDF15, GSN, TIMP1 | NONE |
| 1295 | SERPINA1, SERPINA3, CTSD, DPP4, GDF15, GSN, TFRC | NONE |
| 1296 | SERPINA1, SERPINA3, CTSD, DPP4, GDF15, MIF, PKM | NONE |
| 1297 | SERPINA1, SERPINA3, CTSD, DPP4, GDF15, MIF, TIMP1 | NONE |
| 1298 | SERPINA1, SERPINA3, CTSD, DPP4, GDF15, MIF, TFRC | NONE |
| 1299 | SERPINA1, SERPINA3, CTSD, DPP4, GDF15, PKM, TIMP1 | NONE |
| 1300 | SERPINA1, SERPINA3, CTSD, DPP4, GDF15, PKM, TFRC | NONE |
| 1301 | SERPINA1, SERPINA3, CTSD, DPP4, GDF15, TIMP1, TFRC | NONE |
| 1302 | SERPINA1, SERPINA3, CTSD, DPP4, GSN, MIF, PKM | NONE |
| 1303 | SERPINA1, SERPINA3, CTSD, DPP4, GSN, MIF, TIMP1 | NONE |
| 1304 | SERPINA1, SERPINA3, CTSD, DPP4, GSN, MIF, TFRC | NONE |
| 1305 | SERPINA1, SERPINA3, CTSD, DPP4, GSN, PKM, TIMP1 | NONE |
| 1306 | SERPINA1, SERPINA3, CTSD, DPP4, GSN, PKM, TFRC | NONE |
| 1307 | SERPINA1, SERPINA3, CTSD, DPP4, GSN, TIMP1, TFRC | NONE |
| 1308 | SERPINA1, SERPINA3, CTSD, DPP4, MIF, PKM, TIMP1 | NONE |
| 1309 | SERPINA1, SERPINA3, CTSD, DPP4, MIF, PKM, TFRC | NONE |
| 1310 | SERPINA1, SERPINA3, CTSD, DPP4, MIF, TIMP1, TFRC | NONE |
| 1311 | SERPINA1, SERPINA3, CTSD, DPP4, PKM, TIMP1, TFRC | NONE |
| 1312 | SERPINA1, SERPINA3, CTSD, GDF15, GSN, MIF, PKM | NONE |
| 1313 | SERPINA1, SERPINA3, CTSD, GDF15, GSN, MIF, TIMP1 | NONE |
| 1314 | SERPINA1, SERPINA3, CTSD, GDF15, GSN, MIF, TFRC | NONE |
| 1315 | SERPINA1, SERPINA3, CTSD, GDF15, GSN, PKM, TIMP1 | NONE |
| 1316 | SERPINA1, SERPINA3, CTSD, GDF15, GSN, PKM, TFRC | NONE |
| 1317 | SERPINA1, SERPINA3, CTSD, GDF15, GSN, TIMP1, TFRC | NONE |
| 1318 | SERPINA1, SERPINA3, CTSD, GDF15, MIF, PKM, TIMP1 | NONE |
| 1319 | SERPINA1, SERPINA3, CTSD, GDF15, MIF, PKM, TFRC | NONE |
| 1320 | SERPINA1, SERPINA3, CTSD, GDF15, MIF, TIMP1, TFRC | NONE |
| 1321 | SERPINA1, SERPINA3, CTSD, GDF15, PKM, TIMP1, TFRC | NONE |
| 1322 | SERPINA1, SERPINA3, CTSD, GSN, MIF, PKM, TIMP1 | NONE |
| 1323 | SERPINA1, SERPINA3, CTSD, GSN, MIF, PKM, TFRC | NONE |
| 1324 | SERPINA1, SERPINA3, CTSD, GSN, MIF, TIMP1, TFRC | NONE |
| 1325 | SERPINA1, SERPINA3, CTSD, GSN, PKM, TIMP1, TFRC | NONE |
| 1326 | SERPINA1, SERPINA3, CTSD, MIF, PKM, TIMP1, TFRC | NONE |
| 1327 | SERPINA1, SERPINA3, CLU, DPP4, GDF15, GSN, MIF | NONE |
| 1328 | SERPINA1, SERPINA3, CLU, DPP4, GDF15, GSN, PKM | NONE |
| 1329 | SERPINA1, SERPINA3, CLU, DPP4, GDF15, GSN, TIMP1 | NONE |
| 1330 | SERPINA1, SERPINA3, CLU, DPP4, GDF15, GSN, TFRC | NONE |
| 1331 | SERPINA1, SERPINA3, CLU, DPP4, GDF15, MIF, PKM | NONE |
| 1332 | SERPINA1, SERPINA3, CLU, DPP4, GDF15, MIF, TIMP1 | NONE |
| 1333 | SERPINA1, SERPINA3, CLU, DPP4, GDF15, MIF, TFRC | NONE |
| 1334 | SERPINA1, SERPINA3, CLU, DPP4, GDF15, PKM, TIMP1 | NONE |
| 1335 | SERPINA1, SERPINA3, CLU, DPP4, GDF15, PKM, TFRC | NONE |
| 1336 | SERPINA1, SERPINA3, CLU, DPP4, GDF15, TIMP1, TFRC | NONE |
| 1337 | SERPINA1, SERPINA3, CLU, DPP4, GSN, MIF, PKM | NONE |
| 1338 | SERPINA1, SERPINA3, CLU, DPP4, GSN, MIF, TIMP1 | NONE |
| 1339 | SERPINA1, SERPINA3, CLU, DPP4, GSN, MIF, TFRC | NONE |
| 1340 | SERPINA1, SERPINA3, CLU, DPP4, GSN, PKM, TIMP1 | NONE |
| 1341 | SERPINA1, SERPINA3, CLU, DPP4, GSN, PKM, TFRC | NONE |
| 1342 | SERPINA1, SERPINA3, CLU, DPP4, GSN, TIMP1, TFRC | NONE |
| 1343 | SERPINA1, SERPINA3, CLU, DPP4, MIF, PKM, TIMP1 | NONE |
| 1344 | SERPINA1, SERPINA3, CLU, DPP4, MIF, PKM, TFRC | NONE |
| 1345 | SERPINA1, SERPINA3, CLU, DPP4, MIF, TIMP1, TFRC | NONE |
| 1346 | SERPINA1, SERPINA3, CLU, DPP4, PKM, TIMP1, TFRC | NONE |
| 1347 | SERPINA1, SERPINA3, CLU, GDF15, GSN, MIF, PKM | NONE |
| 1348 | SERPINA1, SERPINA3, CLU, GDF15, GSN, MIF, TIMP1 | NONE |
| 1349 | SERPINA1, SERPINA3, CLU, GDF15, GSN, MIF, TFRC | NONE |
| 1350 | SERPINA1, SERPINA3, CLU, GDF15, GSN, PKM, TIMP1 | NONE |
| 1351 | SERPINA1, SERPINA3, CLU, GDF15, GSN, PKM, TFRC | NONE |
| 1352 | SERPINA1, SERPINA3, CLU, GDF15, GSN, TIMP1, TFRC | NONE |
| 1353 | SERPINA1, SERPINA3, CLU, GDF15, MIF, PKM, TIMP1 | NONE |
| 1354 | SERPINA1, SERPINA3, CLU, GDF15, MIF, PKM, TFRC | NONE |
| 1355 | SERPINA1, SERPINA3, CLU, GDF15, MIF, TIMP1, TFRC | NONE |
| 1356 | SERPINA1, SERPINA3, CLU, GDF15, PKM, TIMP1, TFRC | NONE |
| 1357 | SERPINA1, SERPINA3, CLU, GSN, MIF, PKM, TIMP1 | NONE |
| 1358 | SERPINA1, SERPINA3, CLU, GSN, MIF, PKM, TFRC | NONE |
| 1359 | SERPINA1, SERPINA3, CLU, GSN, MIF, TIMP1, TFRC | NONE |
| 1360 | SERPINA1, SERPINA3, CLU, GSN, PKM, TIMP1, TFRC | NONE |
| 1361 | SERPINA1, SERPINA3, CLU, MIF, PKM, TIMP1, TFRC | NONE |
| 1362 | SERPINA1, SERPINA3, DPP4, GDF15, GSN, MIF, PKM | NONE |
| 1363 | SERPINA1, SERPINA3, DPP4, GDF15, GSN, MIF, TIMP1 | NONE |
| 1364 | SERPINA1, SERPINA3, DPP4, GDF15, GSN, MIF, TFRC | NONE |
| 1365 | SERPINA1, SERPINA3, DPP4, GDF15, GSN, PKM, TIMP1 | NONE |
| 1366 | SERPINA1, SERPINA3, DPP4, GDF15, GSN, PKM, TFRC | NONE |
| 1367 | SERPINA1, SERPINA3, DPP4, GDF15, GSN, TIMP1, TFRC | NONE |
| 1368 | SERPINA1, SERPINA3, DPP4, GDF15, MIF, PKM, TIMP1 | NONE |
| 1369 | SERPINA1, SERPINA3, DPP4, GDF15, MIF, PKM, TFRC | NONE |
| 1370 | SERPINA1, SERPINA3, DPP4, GDF15, MIF, TIMP1, TFRC | NONE |
| 1371 | SERPINA1, SERPINA3, DPP4, GDF15, PKM, TIMP1, TFRC | NONE |
| 1372 | SERPINA1, SERPINA3, DPP4, GSN, MIF, PKM, TIMP1 | NONE |
| 1373 | SERPINA1, SERPINA3, DPP4, GSN, MIF, PKM, TFRC | NONE |
| 1374 | SERPINA1, SERPINA3, DPP4, GSN, MIF, TIMP1, TFRC | NONE |
| 1375 | SERPINA1, SERPINA3, DPP4, GSN, PKM, TIMP1, TFRC | NONE |
| 1376 | SERPINA1, SERPINA3, DPP4, MIF, PKM, TIMP1, TFRC | NONE |
| 1377 | SERPINA1, SERPINA3, GDF15, GSN, MIF, PKM, TIMP1 | NONE |
| 1378 | SERPINA1, SERPINA3, GDF15, GSN, MIF, PKM, TFRC | NONE |
| 1379 | SERPINA1, SERPINA3, GDF15, GSN, MIF, TIMP1, TFRC | NONE |
| 1380 | SERPINA1, SERPINA3, GDF15, GSN, PKM, TIMP1, TFRC | NONE |
| 1381 | SERPINA1, SERPINA3, GDF15, MIF, PKM, TIMP1, TFRC | NONE |
| 1382 | SERPINA1, SERPINA3, GSN, MIF, PKM, TIMP1, TFRC | NONE |
| 1383 | SERPINA1, CTSD, CLU, DPP4, GDF15, GSN, MIF | NONE |
| 1384 | SERPINA1, CTSD, CLU, DPP4, GDF15, GSN, PKM | NONE |
| 1385 | SERPINA1, CTSD, CLU, DPP4, GDF15, GSN, TIMP1 | NONE |
| 1386 | SERPINA1, CTSD, CLU, DPP4, GDF15, GSN, TFRC | NONE |
| 1387 | SERPINA1, CTSD, CLU, DPP4, GDF15, MIF, PKM | NONE |
| 1388 | SERPINA1, CTSD, CLU, DPP4, GDF15, MIF, TIMP1 | NONE |
| 1389 | SERPINA1, CTSD, CLU, DPP4, GDF15, MIF, TFRC | NONE |
| 1390 | SERPINA1, CTSD, CLU, DPP4, GDF15, PKM, TIMP1 | NONE |
| 1391 | SERPINA1, CTSD, CLU, DPP4, GDF15, PKM, TFRC | NONE |
| 1392 | SERPINA1, CTSD, CLU, DPP4, GDF15, TIMP1, TFRC | NONE |
| 1393 | SERPINA1, CTSD, CLU, DPP4, GSN, MIF, PKM | NONE |
| 1394 | SERPINA1, CTSD, CLU, DPP4, GSN, MIF, TIMP1 | NONE |
| 1395 | SERPINA1, CTSD, CLU, DPP4, GSN, MIF, TFRC | NONE |
| 1396 | SERPINA1, CTSD, CLU, DPP4, GSN, PKM, TIMP1 | NONE |
| 1397 | SERPINA1, CTSD, CLU, DPP4, GSN, PKM, TFRC | NONE |
| 1398 | SERPINA1, CTSD, CLU, DPP4, GSN, TIMP1, TFRC | NONE |
| 1399 | SERPINA1, CTSD, CLU, DPP4, MIF, PKM, TIMP1 | NONE |
| 1400 | SERPINA1, CTSD, CLU, DPP4, MIF, PKM, TFRC | NONE |
| 1401 | SERPINA1, CTSD, CLU, DPP4, MIF, TIMP1, TFRC | NONE |
| 1402 | SERPINA1, CTSD, CLU, DPP4, PKM, TIMP1, TFRC | NONE |
| 1403 | SERPINA1, CTSD, CLU, GDF15, GSN, MIF, PKM | NONE |
| 1404 | SERPINA1, CTSD, CLU, GDF15, GSN, MIF, TIMP1 | NONE |
| 1405 | SERPINA1, CTSD, CLU, GDF15, GSN, MIF, TFRC | NONE |
| 1406 | SERPINA1, CTSD, CLU, GDF15, GSN, PKM, TIMP1 | NONE |
| 1407 | SERPINA1, CTSD, CLU, GDF15, GSN, PKM, TFRC | NONE |
| 1408 | SERPINA1, CTSD, CLU, GDF15, GSN, TIMP1, TFRC | NONE |
| 1409 | SERPINA1, CTSD, CLU, GDF15, MIF, PKM, TIMP1 | NONE |
| 1410 | SERPINA1, CTSD, CLU, GDF15, MIF, PKM, TFRC | NONE |
| 1411 | SERPINA1, CTSD, CLU, GDF15, MIF, TIMP1, TFRC | NONE |
| 1412 | SERPINA1, CTSD, CLU, GDF15, PKM, TIMP1, TFRC | NONE |
| 1413 | SERPINA1, CTSD, CLU, GSN, MIF, PKM, TIMP1 | NONE |
| 1414 | SERPINA1, CTSD, CLU, GSN, MIF, PKM, TFRC | NONE |
| 1415 | SERPINA1, CTSD, CLU, GSN, MIF, TIMP1, TFRC | NONE |
| 1416 | SERPINA1, CTSD, CLU, GSN, PKM, TIMP1, TFRC | NONE |
| 1417 | SERPINA1, CTSD, CLU, MIF, PKM, TIMP1, TFRC | NONE |
| 1418 | SERPINA1, CTSD, DPP4, GDF15, GSN, MIF, PKM | NONE |
| 1419 | SERPINA1, CTSD, DPP4, GDF15, GSN, MIF, TIMP1 | NONE |
| 1420 | SERPINA1, CTSD, DPP4, GDF15, GSN, MIF, TFRC | NONE |
| 1421 | SERPINA1, CTSD, DPP4, GDF15, GSN, PKM, TIMP1 | NONE |
| 1422 | SERPINA1, CTSD, DPP4, GDF15, GSN, PKM, TFRC | NONE |
| 1423 | SERPINA1, CTSD, DPP4, GDF15, GSN, TIMP1, TFRC | NONE |
| 1424 | SERPINA1, CTSD, DPP4, GDF15, MIF, PKM, TIMP1 | NONE |
| 1425 | SERPINA1, CTSD, DPP4, GDF15, MIF, PKM, TFRC | NONE |
| 1426 | SERPINA1, CTSD, DPP4, GDF15, MIF, TIMP1, TFRC | NONE |
| 1427 | SERPINA1, CTSD, DPP4, GDF15, PKM, TIMP1, TFRC | NONE |
| 1428 | SERPINA1, CTSD, DPP4, GSN, MIF, PKM, TIMP1 | NONE |
| 1429 | SERPINA1, CTSD, DPP4, GSN, MIF, PKM, TFRC | NONE |
| 1430 | SERPINA1, CTSD, DPP4, GSN, MIF, TIMP1, TFRC | NONE |
| 1431 | SERPINA1, CTSD, DPP4, GSN, PKM, TIMP1, TFRC | NONE |
| 1432 | SERPINA1, CTSD, DPP4, MIF, PKM, TIMP1, TFRC | NONE |
| 1433 | SERPINA1, CTSD, GDF15, GSN, MIF, PKM, TIMP1 | NONE |
| 1434 | SERPINA1, CTSD, GDF15, GSN, MIF, PKM, TFRC | NONE |
| 1435 | SERPINA1, CTSD, GDF15, GSN, MIF, TIMP1, TFRC | NONE |
| 1436 | SERPINA1, CTSD, GDF15, GSN, PKM, TIMP1, TFRC | NONE |
| 1437 | SERPINA1, CTSD, GDF15, MIF, PKM, TIMP1, TFRC | NONE |
| 1438 | SERPINA1, CTSD, GSN, MIF, PKM, TIMP1, TFRC | NONE |
| 1439 | SERPINA1, CLU, DPP4, GDF15, GSN, MIF, PKM | NONE |
| 1440 | SERPINA1, CLU, DPP4, GDF15, GSN, MIF, TIMP1 | NONE |
| 1441 | SERPINA1, CLU, DPP4, GDF15, GSN, MIF, TFRC | NONE |
| 1442 | SERPINA1, CLU, DPP4, GDF15, GSN, PKM, TIMP1 | NONE |
| 1443 | SERPINA1, CLU, DPP4, GDF15, GSN, PKM, TFRC | NONE |
| 1444 | SERPINA1, CLU, DPP4, GDF15, GSN, TIMP1, TFRC | NONE |
| 1445 | SERPINA1, CLU, DPP4, GDF15, MIF, PKM, TIMP1 | NONE |
| 1446 | SERPINA1, CLU, DPP4, GDF15, MIF, PKM, TFRC | NONE |
| 1447 | SERPINA1, CLU, DPP4, GDF15, MIF, TIMP1, TFRC | NONE |
| 1448 | SERPINA1, CLU, DPP4, GDF15, PKM, TIMP1, TFRC | NONE |
| 1449 | SERPINA1, CLU, DPP4, GSN, MIF, PKM, TIMP1 | NONE |
| 1450 | SERPINA1, CLU, DPP4, GSN, MIF, PKM, TFRC | NONE |
| 1451 | SERPINA1, CLU, DPP4, GSN, MIF, TIMP1, TFRC | NONE |
| 1452 | SERPINA1, CLU, DPP4, GSN, PKM, TIMP1, TFRC | NONE |
| 1453 | SERPINA1, CLU, DPP4, MIF, PKM, TIMP1, TFRC | NONE |
| 1454 | SERPINA1, CLU, GDF15, GSN, MIF, PKM, TIMP1 | NONE |
| 1455 | SERPINA1, CLU, GDF15, GSN, MIF, PKM, TFRC | NONE |
| 1456 | SERPINA1, CLU, GDF15, GSN, MIF, TIMP1, TFRC | NONE |
| 1457 | SERPINA1, CLU, GDF15, GSN, PKM, TIMP1, TFRC | NONE |
| 1458 | SERPINA1, CLU, GDF15, MIF, PKM, TIMP1, TFRC | NONE |
| 1459 | SERPINA1, CLU, GSN, MIF, PKM, TIMP1, TFRC | NONE |
| 1460 | SERPINA1, DPP4, GDF15, GSN, MIF, PKM, TIMP1 | NONE |
| 1461 | SERPINA1, DPP4, GDF15, GSN, MIF, PKM, TFRC | NONE |
| 1462 | SERPINA1, DPP4, GDF15, GSN, MIF, TIMP1, TFRC | NONE |
| 1463 | SERPINA1, DPP4, GDF15, GSN, PKM, TIMP1, TFRC | NONE |
| 1464 | SERPINA1, DPP4, GDF15, MIF, PKM, TIMP1, TFRC | NONE |
| 1465 | SERPINA1, DPP4, GSN, MIF, PKM, TIMP1, TFRC | NONE |
| 1466 | SERPINA1, GDF15, GSN, MIF, PKM, TIMP1, TFRC | NONE |
| 1467 | SERPINA3, CTSD, CLU, DPP4, GDF15, GSN, MIF | NONE |
| 1468 | SERPINA3, CTSD, CLU, DPP4, GDF15, GSN, PKM | NONE |
| 1469 | SERPINA3, CTSD, CLU, DPP4, GDF15, GSN, TIMP1 | NONE |
| 1470 | SERPINA3, CTSD, CLU, DPP4, GDF15, GSN, TFRC | NONE |
| 1471 | SERPINA3, CTSD, CLU, DPP4, GDF15, MIF, PKM | NONE |
| 1472 | SERPINA3, CTSD, CLU, DPP4, GDF15, MIF, TIMP1 | NONE |
| 1473 | SERPINA3, CTSD, CLU, DPP4, GDF15, MIF, TFRC | NONE |
| 1474 | SERPINA3, CTSD, CLU, DPP4, GDF15, PKM, TIMP1 | NONE |
| 1475 | SERPINA3, CTSD, CLU, DPP4, GDF15, PKM, TFRC | NONE |
| 1476 | SERPINA3, CTSD, CLU, DPP4, GDF15, TIMP1, TFRC | NONE |
| 1477 | SERPINA3, CTSD, CLU, DPP4, GSN, MIF, PKM | NONE |
| 1478 | SERPINA3, CTSD, CLU, DPP4, GSN, MIF, TIMP1 | NONE |
| 1479 | SERPINA3, CTSD, CLU, DPP4, GSN, MIF, TFRC | NONE |
| 1480 | SERPINA3, CTSD, CLU, DPP4, GSN, PKM, TIMP1 | NONE |
| 1481 | SERPINA3, CTSD, CLU, DPP4, GSN, PKM, TFRC | NONE |
| 1482 | SERPINA3, CTSD, CLU, DPP4, GSN, TIMP1, TFRC | NONE |
| 1483 | SERPINA3, CTSD, CLU, DPP4, MIF, PKM, TIMP1 | NONE |
| 1484 | SERPINA3, CTSD, CLU, DPP4, MIF, PKM, TFRC | NONE |
| 1485 | SERPINA3, CTSD, CLU, DPP4, MIF, TIMP1, TFRC | NONE |
| 1486 | SERPINA3, CTSD, CLU, DPP4, PKM, TIMP1, TFRC | NONE |
| 1487 | SERPINA3, CTSD, CLU, GDF15, GSN, MIF, PKM | NONE |
| 1488 | SERPINA3, CTSD, CLU, GDF15, GSN, MIF, TIMP1 | NONE |
| 1489 | SERPINA3, CTSD, CLU, GDF15, GSN, MIF, TFRC | NONE |
| 1490 | SERPINA3, CTSD, CLU, GDF15, GSN, PKM, TIMP1 | NONE |
| 1491 | SERPINA3, CTSD, CLU, GDF15, GSN, PKM, TFRC | NONE |
| 1492 | SERPINA3, CTSD, CLU, GDF15, GSN, TIMP1, TFRC | NONE |
| 1493 | SERPINA3, CTSD, CLU, GDF15, MIF, PKM, TIMP1 | NONE |
| 1494 | SERPINA3, CTSD, CLU, GDF15, MIF, PKM, TFRC | NONE |
| 1495 | SERPINA3, CTSD, CLU, GDF15, MIF, TIMP1, TFRC | NONE |
| 1496 | SERPINA3, CTSD, CLU, GDF15, PKM, TIMP1, TFRC | NONE |
| 1497 | SERPINA3, CTSD, CLU, GSN, MIF, PKM, TIMP1 | NONE |
| 1498 | SERPINA3, CTSD, CLU, GSN, MIF, PKM, TFRC | NONE |
| 1499 | SERPINA3, CTSD, CLU, GSN, MIF, TIMP1, TFRC | NONE |
| 1500 | SERPINA3, CTSD, CLU, GSN, PKM, TIMP1, TFRC | NONE |
| 1501 | SERPINA3, CTSD, CLU, MIF, PKM, TIMP1, TFRC | NONE |
| 1502 | SERPINA3, CTSD, DPP4, GDF15, GSN, MIF, PKM | NONE |
| 1503 | SERPINA3, CTSD, DPP4, GDF15, GSN, MIF, TIMP1 | NONE |
| 1504 | SERPINA3, CTSD, DPP4, GDF15, GSN, MIF, TFRC | NONE |
| 1505 | SERPINA3, CTSD, DPP4, GDF15, GSN, PKM, TIMP1 | NONE |
| 1506 | SERPINA3, CTSD, DPP4, GDF15, GSN, PKM, TFRC | NONE |
| 1507 | SERPINA3, CTSD, DPP4, GDF15, GSN, TIMP1, TFRC | NONE |
| 1508 | SERPINA3, CTSD, DPP4, GDF15, MIF, PKM, TIMP1 | NONE |
| 1509 | SERPINA3, CTSD, DPP4, GDF15, MIF, PKM, TFRC | NONE |
| 1510 | SERPINA3, CTSD, DPP4, GDF15, MIF, TIMP1, TFRC | NONE |
| 1511 | SERPINA3, CTSD, DPP4, GDF15, PKM, TIMP1, TFRC | NONE |
| 1512 | SERPINA3, CTSD, DPP4, GSN, MIF, PKM, TIMP1 | NONE |
| 1513 | SERPINA3, CTSD, DPP4, GSN, MIF, PKM, TFRC | NONE |
| 1514 | SERPINA3, CTSD, DPP4, GSN, MIF, TIMP1, TFRC | NONE |
| 1515 | SERPINA3, CTSD, DPP4, GSN, PKM, TIMP1, TFRC | NONE |
| 1516 | SERPINA3, CTSD, DPP4, MIF, PKM, TIMP1, TFRC | NONE |
| 1517 | SERPINA3, CTSD, GDF15, GSN, MIF, PKM, TIMP1 | NONE |
| 1518 | SERPINA3, CTSD, GDF15, GSN, MIF, PKM, TFRC | NONE |
| 1519 | SERPINA3, CTSD, GDF15, GSN, MIF, TIMP1, TFRC | NONE |
| 1520 | SERPINA3, CTSD, GDF15, GSN, PKM, TIMP1, TFRC | NONE |
| 1521 | SERPINA3, CTSD, GDF15, MIF, PKM, TIMP1, TFRC | NONE |
| 1522 | SERPINA3, CTSD, GSN, MIF, PKM, TIMP1, TFRC | NONE |
| 1523 | SERPINA3, CLU, DPP4, GDF15, GSN, MIF, PKM | NONE |
| 1524 | SERPINA3, CLU, DPP4, GDF15, GSN, MIF, TIMP1 | NONE |
| 1525 | SERPINA3, CLU, DPP4, GDF15, GSN, MIF, TFRC | NONE |
| 1526 | SERPINA3, CLU, DPP4, GDF15, GSN, PKM, TIMP1 | NONE |
| 1527 | SERPINA3, CLU, DPP4, GDF15, GSN, PKM, TFRC | NONE |
| 1528 | SERPINA3, CLU, DPP4, GDF15, GSN, TIMP1, TFRC | NONE |
| 1529 | SERPINA3, CLU, DPP4, GDF15, MIF, PKM, TIMP1 | NONE |
| 1530 | SERPINA3, CLU, DPP4, GDF15, MIF, PKM, TFRC | NONE |
| 1531 | SERPINA3, CLU, DPP4, GDF15, MIF, TIMP1, TFRC | NONE |
| 1532 | SERPINA3, CLU, DPP4, GDF15, PKM, TIMP1, TFRC | NONE |
| 1533 | SERPINA3, CLU, DPP4, GSN, MIF, PKM, TIMP1 | NONE |
| 1534 | SERPINA3, CLU, DPP4, GSN, MIF, PKM, TFRC | NONE |
| 1535 | SERPINA3, CLU, DPP4, GSN, MIF, TIMP1, TFRC | NONE |
| 1536 | SERPINA3, CLU, DPP4, GSN, PKM, TIMP1, TFRC | NONE |
| 1537 | SERPINA3, CLU, DPP4, MIF, PKM, TIMP1, TFRC | NONE |
| 1538 | SERPINA3, CLU, GDF15, GSN, MIF, PKM, TIMP1 | NONE |
| 1539 | SERPINA3, CLU, GDF15, GSN, MIF, PKM, TFRC | NONE |
| 1540 | SERPINA3, CLU, GDF15, GSN, MIF, TIMP1, TFRC | NONE |
| 1541 | SERPINA3, CLU, GDF15, GSN, PKM, TIMP1, TFRC | NONE |
| 1542 | SERPINA3, CLU, GDF15, MIF, PKM, TIMP1, TFRC | NONE |
| 1543 | SERPINA3, CLU, GSN, MIF, PKM, TIMP1, TFRC | NONE |
| 1544 | SERPINA3, DPP4, GDF15, GSN, MIF, PKM, TIMP1 | NONE |
| 1545 | SERPINA3, DPP4, GDF15, GSN, MIF, PKM, TFRC | NONE |
| 1546 | SERPINA3, DPP4, GDF15, GSN, MIF, TIMP1, TFRC | NONE |
| 1547 | SERPINA3, DPP4, GDF15, GSN, PKM, TIMP1, TFRC | NONE |
| 1548 | SERPINA3, DPP4, GDF15, MIF, PKM, TIMP1, TFRC | NONE |
| 1549 | SERPINA3, DPP4, GSN, MIF, PKM, TIMP1, TFRC | NONE |
| 1550 | SERPINA3, GDF15, GSN, MIF, PKM, TIMP1, TFRC | NONE |
| 1551 | CTSD, CLU, DPP4, GDF15, GSN, MIF, PKM | NONE |
| 1552 | CTSD, CLU, DPP4, GDF15, GSN, MIF, TIMP1 | NONE |
| 1553 | CTSD, CLU, DPP4, GDF15, GSN, MIF, TFRC | NONE |
| 1554 | CTSD, CLU, DPP4, GDF15, GSN, PKM, TIMP1 | NONE |
| 1555 | CTSD, CLU, DPP4, GDF15, GSN, PKM, TFRC | NONE |
| 1556 | CTSD, CLU, DPP4, GDF15, GSN, TIMP1, TFRC | NONE |
| 1557 | CTSD, CLU, DPP4, GDF15, MIF, PKM, TIMP1 | NONE |
| 1558 | CTSD, CLU, DPP4, GDF15, MIF, PKM, TFRC | NONE |
| 1559 | CTSD, CLU, DPP4, GDF15, MIF, TIMP1, TFRC | NONE |
| 1560 | CTSD, CLU, DPP4, GDF15, PKM, TIMP1, TFRC | NONE |
| 1561 | CTSD, CLU, DPP4, GSN, MIF, PKM, TIMP1 | NONE |
| 1562 | CTSD, CLU, DPP4, GSN, MIF, PKM, TFRC | NONE |
| 1563 | CTSD, CLU, DPP4, GSN, MIF, TIMP1, TFRC | NONE |
| 1564 | CTSD, CLU, DPP4, GSN, PKM, TIMP1, TFRC | NONE |
| 1565 | CTSD, CLU, DPP4, MIF, PKM, TIMP1, TFRC | NONE |
| 1566 | CTSD, CLU, GDF15, GSN, MIF, PKM, TIMP1 | NONE |
| 1567 | CTSD, CLU, GDF15, GSN, MIF, PKM, TFRC | NONE |
| 1568 | CTSD, CLU, GDF15, GSN, MIF, TIMP1, TFRC | NONE |
| 1569 | CTSD, CLU, GDF15, GSN, PKM, TIMP1, TFRC | NONE |
| 1570 | CTSD, CLU, GDF15, MIF, PKM, TIMP1, TFRC | NONE |
| 1571 | CTSD, CLU, GSN, MIF, PKM, TIMP1, TFRC | NONE |
| 1572 | CTSD, DPP4, GDF15, GSN, MIF, PKM, TIMP1 | NONE |
| 1573 | CTSD, DPP4, GDF15, GSN, MIF, PKM, TFRC | NONE |
| 1574 | CTSD, DPP4, GDF15, GSN, MIF, TIMP1, TFRC | NONE |
| 1575 | CTSD, DPP4, GDF15, GSN, PKM, TIMP1, TFRC | NONE |
| 1576 | CTSD, DPP4, GDF15, MIF, PKM, TIMP1, TFRC | NONE |
| 1577 | CTSD, DPP4, GSN, MIF, PKM, TIMP1, TFRC | NONE |
| 1578 | CTSD, GDF15, GSN, MIF, PKM, TIMP1, TFRC | NONE |
| 1579 | CLU, DPP4, GDF15, GSN, MIF, PKM, TIMP1 | NONE |
| 1580 | CLU, DPP4, GDF15, GSN, MIF, PKM, TFRC | NONE |
| 1581 | CLU, DPP4, GDF15, GSN, MIF, TIMP1, TFRC | NONE |
| 1582 | CLU, DPP4, GDF15, GSN, PKM, TIMP1, TFRC | NONE |
| 1583 | CLU, DPP4, GDF15, MIF, PKM, TIMP1, TFRC | NONE |
| 1584 | CLU, DPP4, GSN, MIF, PKM, TIMP1, TFRC | NONE |
| 1585 | CLU, GDF15, GSN, MIF, PKM, TIMP1, TFRC | NONE |
| 1586 | DPP4, GDF15, GSN, MIF, PKM, TIMP1, TFRC | NONE |
| 1587 | SERPINA1, SERPINA3, CTSD, CLU, DPP4 | Age |
| 1588 | SERPINA1, SERPINA3, CTSD, CLU, GDF15 | Age |
| 1589 | SERPINA1, SERPINA3, CTSD, CLU, GSN | Age |
| 1590 | SERPINA1, SERPINA3, CTSD, CLU, MIF | Age |
| 1591 | SERPINA1, SERPINA3, CTSD, CLU, PKM | Age |
| 1592 | SERPINA1, SERPINA3, CTSD, CLU, TIMP1 | Age |
| 1593 | SERPINA1, SERPINA3, CTSD, CLU, TFRC | Age |
| 1594 | SERPINA1, SERPINA3, CTSD, DPP4, GDF15 | Age |
| 1595 | SERPINA1, SERPINA3, CTSD, DPP4, GSN | Age |
| 1596 | SERPINA1, SERPINA3, CTSD, DPP4, MIF | Age |
| 1597 | SERPINA1, SERPINA3, CTSD, DPP4, PKM | Age |
| 1598 | SERPINA1, SERPINA3, CTSD, DPP4, TIMP1 | Age |
| 1599 | SERPINA1, SERPINA3, CTSD, DPP4, TFRC | Age |
| 1600 | SERPINA1, SERPINA3, CTSD, GDF15, GSN | Age |
| 1601 | SERPINA1, SERPINA3, CTSD, GDF15, MIF | Age |
| 1602 | SERPINA1, SERPINA3, CTSD, GDF15, PKM | Age |
| 1603 | SERPINA1, SERPINA3, CTSD, GDF15, TIMP1 | Age |
| 1604 | SERPINA1, SERPINA3, CTSD, GDF15, TFRC | Age |
| 1605 | SERPINA1, SERPINA3, CTSD, GSN, MIF | Age |
| 1606 | SERPINA1, SERPINA3, CTSD, GSN, PKM | Age |
| 1607 | SERPINA1, SERPINA3, CTSD, GSN, TIMP1 | Age |
| 1608 | SERPINA1, SERPINA3, CTSD, GSN, TFRC | Age |
| 1609 | SERPINA1, SERPINA3, CTSD, MIF, PKM | Age |
| 1610 | SERPINA1, SERPINA3, CTSD, MIF, TIMP1 | Age |
| 1611 | SERPINA1, SERPINA3, CTSD, MIF, TFRC | Age |
| 1612 | SERPINA1, SERPINA3, CTSD, PKM, TIMP1 | Age |
| 1613 | SERPINA1, SERPINA3, CTSD, PKM, TFRC | Age |
| 1614 | SERPINA1, SERPINA3, CTSD, TIMP1, TFRC | Age |
| 1615 | SERPINA1, SERPINA3, CLU, DPP4, GDF15 | Age |
| 1616 | SERPINA1, SERPINA3, CLU, DPP4, GSN | Age |
| 1617 | SERPINA1, SERPINA3, CLU, DPP4, MIF | Age |
| 1618 | SERPINA1, SERPINA3, CLU, DPP4, PKM | Age |
| 1619 | SERPINA1, SERPINA3, CLU, DPP4, TIMP1 | Age |
| 1620 | SERPINA1, SERPINA3, CLU, DPP4, TFRC | Age |
| 1621 | SERPINA1, SERPINA3, CLU, GDF15, GSN | Age |
| 1622 | SERPINA1, SERPINA3, CLU, GDF15, MIF | Age |
| 1623 | SERPINA1, SERPINA3, CLU, GDF15, PKM | Age |
| 1624 | SERPINA1, SERPINA3, CLU, GDF15, TIMP1 | Age |
| 1625 | SERPINA1, SERPINA3, CLU, GDF15, TFRC | Age |
| 1626 | SERPINA1, SERPINA3, CLU, GSN, MIF | Age |
| 1627 | SERPINA1, SERPINA3, CLU, GSN, PKM | Age |
| 1628 | SERPINA1, SERPINA3, CLU, GSN, TIMP1 | Age |
| 1629 | SERPINA1, SERPINA3, CLU, GSN, TFRC | Age |
| 1630 | SERPINA1, SERPINA3, CLU, MIF, PKM | Age |
| 1631 | SERPINA1, SERPINA3, CLU, MIF, TIMP1 | Age |
| 1632 | SERPINA1, SERPINA3, CLU, MIF, TFRC | Age |
| 1633 | SERPINA1, SERPINA3, CLU, PKM, TIMP1 | Age |
| 1634 | SERPINA1, SERPINA3, CLU, PKM, TFRC | Age |
| 1635 | SERPINA1, SERPINA3, CLU, TIMP1, TFRC | Age |
| 1636 | SERPINA1, SERPINA3, DPP4, GDF15, GSN | Age |
| 1637 | SERPINA1, SERPINA3, DPP4, GDF15, MIF | Age |
| 1638 | SERPINA1, SERPINA3, DPP4, GDF15, PKM | Age |
| 1639 | SERPINA1, SERPINA3, DPP4, GDF15, TIMP1 | Age |
| 1640 | SERPINA1, SERPINA3, DPP4, GDF15, TFRC | Age |
| 1641 | SERPINA1, SERPINA3, DPP4, GSN, MIF | Age |
| 1642 | SERPINA1, SERPINA3, DPP4, GSN, PKM | Age |
| 1643 | SERPINA1, SERPINA3, DPP4, GSN, TIMP1 | Age |
| 1644 | SERPINA1, SERPINA3, DPP4, GSN, TFRC | Age |
| 1645 | SERPINA1, SERPINA3, DPP4, MIF, PKM | Age |
| 1646 | SERPINA1, SERPINA3, DPP4, MIF, TIMP1 | Age |
| 1647 | SERPINA1, SERPINA3, DPP4, MIF, TFRC | Age |
| 1648 | SERPINA1, SERPINA3, DPP4, PKM, TIMP1 | Age |
| 1649 | SERPINA1, SERPINA3, DPP4, PKM, TFRC | Age |
| 1650 | SERPINA1, SERPINA3, DPP4, TIMP1, TFRC | Age |
| 1651 | SERPINA1, SERPINA3, GDF15, GSN, MIF | Age |
| 1652 | SERPINA1, SERPINA3, GDF15, GSN, PKM | Age |
| 1653 | SERPINA1, SERPINA3, GDF15, GSN, TIMP1 | Age |
| 1654 | SERPINA1, SERPINA3, GDF15, GSN, TFRC | Age |
| 1655 | SERPINA1, SERPINA3, GDF15, MIF, PKM | Age |
| 1656 | SERPINA1, SERPINA3, GDF15, MIF, TIMP1 | Age |
| 1657 | SERPINA1, SERPINA3, GDF15, MIF, TFRC | Age |
| 1658 | SERPINA1, SERPINA3, GDF15, PKM, TIMP1 | Age |
| 1659 | SERPINA1, SERPINA3, GDF15, PKM, TFRC | Age |
| 1660 | SERPINA1, SERPINA3, GDF15, TIMP1, TFRC | Age |
| 1661 | SERPINA1, SERPINA3, GSN, MIF, PKM | Age |
| 1662 | SERPINA1, SERPINA3, GSN, MIF, TIMP1 | Age |
| 1663 | SERPINA1, SERPINA3, GSN, MIF, TFRC | Age |
| 1664 | SERPINA1, SERPINA3, GSN, PKM, TIMP1 | Age |
| 1665 | SERPINA1, SERPINA3, GSN, PKM, TFRC | Age |
| 1666 | SERPINA1, SERPINA3, GSN, TIMP1, TFRC | Age |
| 1667 | SERPINA1, SERPINA3, MIF, PKM, TIMP1 | Age |
| 1668 | SERPINA1, SERPINA3, MIF, PKM, TFRC | Age |
| 1669 | SERPINA1, SERPINA3, MIF, TIMP1, TFRC | Age |
| 1670 | SERPINA1, SERPINA3, PKM, TIMP1, TFRC | Age |
| 1671 | SERPINA1, CTSD, CLU, DPP4, GDF15 | Age |
| 1672 | SERPINA1, CTSD, CLU, DPP4, GSN | Age |
| 1673 | SERPINA1, CTSD, CLU, DPP4, MIF | Age |
| 1674 | SERPINA1, CTSD, CLU, DPP4, PKM | Age |
| 1675 | SERPINA1, CTSD, CLU, DPP4, TIMP1 | Age |
| 1676 | SERPINA1, CTSD, CLU, DPP4, TFRC | Age |
| 1677 | SERPINA1, CTSD, CLU, GDF15, GSN | Age |
| 1678 | SERPINA1, CTSD, CLU, GDF15, MIF | Age |
| 1679 | SERPINA1, CTSD, CLU, GDF15, PKM | Age |
| 1680 | SERPINA1, CTSD, CLU, GDF15, TIMP1 | Age |
| 1681 | SERPINA1, CTSD, CLU, GDF15, TFRC | Age |
| 1682 | SERPINA1, CTSD, CLU, GSN, MIF | Age |
| 1683 | SERPINA1, CTSD, CLU, GSN, PKM | Age |
| 1684 | SERPINA1, CTSD, CLU, GSN, TIMP1 | Age |
| 1685 | SERPINA1, CTSD, CLU, GSN, TFRC | Age |
| 1686 | SERPINA1, CTSD, CLU, MIF, PKM | Age |
| 1687 | SERPINA1, CTSD, CLU, MIF, TIMP1 | Age |
| 1688 | SERPINA1, CTSD, CLU, MIF, TFRC | Age |
| 1689 | SERPINA1, CTSD, CLU, PKM, TIMP1 | Age |
| 1690 | SERPINA1, CTSD, CLU, PKM, TFRC | Age |
| 1691 | SERPINA1, CTSD, CLU, TIMP1, TFRC | Age |
| 1692 | SERPINA1, CTSD, DPP4, GDF15, GSN | Age |
| 1693 | SERPINA1, CTSD, DPP4, GDF15, MIF | Age |
| 1694 | SERPINA1, CTSD, DPP4, GDF15, PKM | Age |
| 1695 | SERPINA1, CTSD, DPP4, GDF15, TIMP1 | Age |
| 1696 | SERPINA1, CTSD, DPP4, GDF15, TFRC | Age |
| 1697 | SERPINA1, CTSD, DPP4, GSN, MIF | Age |
| 1698 | SERPINA1, CTSD, DPP4, GSN, PKM | Age |
| 1699 | SERPINA1, CTSD, DPP4, GSN, TIMP1 | Age |
| 1700 | SERPINA1, CTSD, DPP4, GSN, TFRC | Age |
| 1701 | SERPINA1, CTSD, DPP4, MIF, PKM | Age |
| 1702 | SERPINA1, CTSD, DPP4, MIF, TIMP1 | Age |
| 1703 | SERPINA1, CTSD, DPP4, MIF, TFRC | Age |
| 1704 | SERPINA1, CTSD, DPP4, PKM, TIMP1 | Age |
| 1705 | SERPINA1, CTSD, DPP4, PKM, TFRC | Age |
| 1706 | SERPINA1, CTSD, DPP4, TIMP1, TFRC | Age |
| 1707 | SERPINA1, CTSD, GDF15, GSN, MIF | Age |
| 1708 | SERPINA1, CTSD, GDF15, GSN, PKM | Age |
| 1709 | SERPINA1, CTSD, GDF15, GSN, TIMP1 | Age |
| 1710 | SERPINA1, CTSD, GDF15, GSN, TFRC | Age |
| 1711 | SERPINA1, CTSD, GDF15, MIF, PKM | Age |
| 1712 | SERPINA1, CTSD, GDF15, MIF, TIMP1 | Age |
| 1713 | SERPINA1, CTSD, GDF15, MIF, TFRC | Age |
| 1714 | SERPINA1, CTSD, GDF15, PKM, TIMP1 | Age |
| 1715 | SERPINA1, CTSD, GDF15, PKM, TFRC | Age |
| 1716 | SERPINA1, CTSD, GDF15, TIMP1, TFRC | Age |
| 1717 | SERPINA1, CTSD, GSN, MIF, PKM | Age |
| 1718 | SERPINA1, CTSD, GSN, MIF, TIMP1 | Age |
| 1719 | SERPINA1, CTSD, GSN, MIF, TFRC | Age |
| 1720 | SERPINA1, CTSD, GSN, PKM, TIMP1 | Age |
| 1721 | SERPINA1, CTSD, GSN, PKM, TFRC | Age |
| 1722 | SERPINA1, CTSD, GSN, TIMP1, TFRC | Age |
| 1723 | SERPINA1, CTSD, MIF, PKM, TIMP1 | Age |
| 1724 | SERPINA1, CTSD, MIF, PKM, TFRC | Age |
| 1725 | SERPINA1, CTSD, MIF, TIMP1, TFRC | Age |
| 1726 | SERPINA1, CTSD, PKM, TIMP1, TFRC | Age |
| 1727 | SERPINA1, CLU, DPP4, GDF15, GSN | Age |
| 1728 | SERPINA1, CLU, DPP4, GDF15, MIF | Age |
| 1729 | SERPINA1, CLU, DPP4, GDF15, PKM | Age |
| 1730 | SERPINA1, CLU, DPP4, GDF15, TIMP1 | Age |
| 1731 | SERPINA1, CLU, DPP4, GDF15, TFRC | Age |
| 1732 | SERPINA1, CLU, DPP4, GSN, MIF | Age |
| 1733 | SERPINA1, CLU, DPP4, GSN, PKM | Age |
| 1734 | SERPINA1, CLU, DPP4, GSN, TIMP1 | Age |
| 1735 | SERPINA1, CLU, DPP4, GSN, TFRC | Age |
| 1736 | SERPINA1, CLU, DPP4, MIF, PKM | Age |
| 1737 | SERPINA1, CLU, DPP4, MIF, TIMP1 | Age |
| 1738 | SERPINA1, CLU, DPP4, MIF, TFRC | Age |
| 1739 | SERPINA1, CLU, DPP4, PKM, TIMP1 | Age |
| 1740 | SERPINA1, CLU, DPP4, PKM, TFRC | Age |
| 1741 | SERPINA1, CLU, DPP4, TIMP1, TFRC | Age |
| 1742 | SERPINA1, CLU, GDF15, GSN, MIF | Age |
| 1743 | SERPINA1, CLU, GDF15, GSN, PKM | Age |
| 1744 | SERPINA1, CLU, GDF15, GSN, TIMP1 | Age |
| 1745 | SERPINA1, CLU, GDF15, GSN, TFRC | Age |
| 1746 | SERPINA1, CLU, GDF15, MIF, PKM | Age |
| 1747 | SERPINA1, CLU, GDF15, MIF, TIMP1 | Age |
| 1748 | SERPINA1, CLU, GDF15, MIF, TFRC | Age |
| 1749 | SERPINA1, CLU, GDF15, PKM, TIMP1 | Age |
| 1750 | SERPINA1, CLU, GDF15, PKM, TFRC | Age |
| 1751 | SERPINA1, CLU, GDF15, TIMP1, TFRC | Age |
| 1752 | SERPINA1, CLU, GSN, MIF, PKM | Age |
| 1753 | SERPINA1, CLU, GSN, MIF, TIMP1 | Age |
| 1754 | SERPINA1, CLU, GSN, MIF, TFRC | Age |
| 1755 | SERPINA1, CLU, GSN, PKM, TIMP1 | Age |
| 1756 | SERPINA1, CLU, GSN, PKM, TFRC | Age |
| 1757 | SERPINA1, CLU, GSN, TIMP1, TFRC | Age |
| 1758 | SERPINA1, CLU, MIF, PKM, TIMP1 | Age |
| 1759 | SERPINA1, CLU, MIF, PKM, TFRC | Age |
| 1760 | SERPINA1, CLU, MIF, TIMP1, TFRC | Age |
| 1761 | SERPINA1, CLU, PKM, TIMP1, TFRC | Age |
| 1762 | SERPINA1, DPP4, GDF15, GSN, MIF | Age |
| 1763 | SERPINA1, DPP4, GDF15, GSN, PKM | Age |
| 1764 | SERPINA1, DPP4, GDF15, GSN, TIMP1 | Age |
| 1765 | SERPINA1, DPP4, GDF15, GSN, TFRC | Age |
| 1766 | SERPINA1, DPP4, GDF15, MIF, PKM | Age |
| 1767 | SERPINA1, DPP4, GDF15, MIF, TIMP1 | Age |
| 1768 | SERPINA1, DPP4, GDF15, MIF, TFRC | Age |
| 1769 | SERPINA1, DPP4, GDF15, PKM, TIMP1 | Age |
| 1770 | SERPINA1, DPP4, GDF15, PKM, TFRC | Age |
| 1771 | SERPINA1, DPP4, GDF15, TIMP1, TFRC | Age |
| 1772 | SERPINA1, DPP4, GSN, MIF, PKM | Age |
| 1773 | SERPINA1, DPP4, GSN, MIF, TIMP1 | Age |
| 1774 | SERPINA1, DPP4, GSN, MIF, TFRC | Age |
| 1775 | SERPINA1, DPP4, GSN, PKM, TIMP1 | Age |
| 1776 | SERPINA1, DPP4, GSN, PKM, TFRC | Age |
| 1777 | SERPINA1, DPP4, GSN, TIMP1, TFRC | Age |
| 1778 | SERPINA1, DPP4, MIF, PKM, TIMP1 | Age |
| 1779 | SERPINA1, DPP4, MIF, PKM, TFRC | Age |
| 1780 | SERPINA1, DPP4, MIF, TIMP1, TFRC | Age |
| 1781 | SERPINA1, DPP4, PKM, TIMP1, TFRC | Age |
| 1782 | SERPINA1, GDF15, GSN, MIF, PKM | Age |
| 1783 | SERPINA1, GDF15, GSN, MIF, TIMP1 | Age |
| 1784 | SERPINA1, GDF15, GSN, MIF, TFRC | Age |
| 1785 | SERPINA1, GDF15, GSN, PKM, TIMP1 | Age |
| 1786 | SERPINA1, GDF15, GSN, PKM, TFRC | Age |
| 1787 | SERPINA1, GDF15, GSN, TIMP1, TFRC | Age |
| 1788 | SERPINA1, GDF15, MIF, PKM, TIMP1 | Age |
| 1789 | SERPINA1, GDF15, MIF, PKM, TFRC | Age |
| 1790 | SERPINA1, GDF15, MIF, TIMP1, TFRC | Age |
| 1791 | SERPINA1, GDF15, PKM, TIMP1, TFRC | Age |
| 1792 | SERPINA1, GSN, MIF, PKM, TIMP1 | Age |
| 1793 | SERPINA1, GSN, MIF, PKM, TFRC | Age |
| 1794 | SERPINA1, GSN, MIF, TIMP1, TFRC | Age |
| 1795 | SERPINA1, GSN, PKM, TIMP1, TFRC | Age |
| 1796 | SERPINA1, MIF, PKM, TIMP1, TFRC | Age |
| 1797 | SERPINA3, CTSD, CLU, DPP4, GDF15 | Age |
| 1798 | SERPINA3, CTSD, CLU, DPP4, GSN | Age |
| 1799 | SERPINA3, CTSD, CLU, DPP4, MIF | Age |
| 1800 | SERPINA3, CTSD, CLU, DPP4, PKM | Age |
| 1801 | SERPINA3, CTSD, CLU, DPP4, TIMP1 | Age |
| 1802 | SERPINA3, CTSD, CLU, DPP4, TFRC | Age |
| 1803 | SERPINA3, CTSD, CLU, GDF15, GSN | Age |
| 1804 | SERPINA3, CTSD, CLU, GDF15, MIF | Age |
| 1805 | SERPINA3, CTSD, CLU, GDF15, PKM | Age |
| 1806 | SERPINA3, CTSD, CLU, GDF15, TIMP1 | Age |
| 1807 | SERPINA3, CTSD, CLU, GDF15, TFRC | Age |
| 1808 | SERPINA3, CTSD, CLU, GSN, MIF | Age |
| 1809 | SERPINA3, CTSD, CLU, GSN, PKM | Age |
| 1810 | SERPINA3, CTSD, CLU, GSN, TIMP1 | Age |
| 1811 | SERPINA3, CTSD, CLU, GSN, TFRC | Age |
| 1812 | SERPINA3, CTSD, CLU, MIF, PKM | Age |
| 1813 | SERPINA3, CTSD, CLU, MIF, TIMP1 | Age |
| 1814 | SERPINA3, CTSD, CLU, MIF, TFRC | Age |
| 1815 | SERPINA3, CTSD, CLU, PKM, TIMP1 | Age |
| 1816 | SERPINA3, CTSD, CLU, PKM, TFRC | Age |
| 1817 | SERPINA3, CTSD, CLU, TIMP1, TFRC | Age |
| 1818 | SERPINA3, CTSD, DPP4, GDF15, GSN | Age |
| 1819 | SERPINA3, CTSD, DPP4, GDF15, MIF | Age |
| 1820 | SERPINA3, CTSD, DPP4, GDF15, PKM | Age |
| 1821 | SERPINA3, CTSD, DPP4, GDF15, TIMP1 | Age |
| 1822 | SERPINA3, CTSD, DPP4, GDF15, TFRC | Age |
| 1823 | SERPINA3, CTSD, DPP4, GSN, MIF | Age |
| 1824 | SERPINA3, CTSD, DPP4, GSN, PKM | Age |
| 1825 | SERPINA3, CTSD, DPP4, GSN, TIMP1 | Age |
| 1826 | SERPINA3, CTSD, DPP4, GSN, TFRC | Age |
| 1827 | SERPINA3, CTSD, DPP4, MIF, PKM | Age |
| 1828 | SERPINA3, CTSD, DPP4, MIF, TIMP1 | Age |
| 1829 | SERPINA3, CTSD, DPP4, MIF, TFRC | Age |
| 1830 | SERPINA3, CTSD, DPP4, PKM, TIMP1 | Age |
| 1831 | SERPINA3, CTSD, DPP4, PKM, TFRC | Age |
| 1832 | SERPINA3, CTSD, DPP4, TIMP1, TFRC | Age |
| 1833 | SERPINA3, CTSD, GDF15, GSN, MIF | Age |
| 1834 | SERPINA3, CTSD, GDF15, GSN, PKM | Age |
| 1835 | SERPINA3, CTSD, GDF15, GSN, TIMP1 | Age |
| 1836 | SERPINA3, CTSD, GDF15, GSN, TFRC | Age |
| 1837 | SERPINA3, CTSD, GDF15, MIF, PKM | Age |
| 1838 | SERPINA3, CTSD, GDF15, MIF, TIMP1 | Age |
| 1839 | SERPINA3, CTSD, GDF15, MIF, TFRC | Age |
| 1840 | SERPINA3, CTSD, GDF15, PKM, TIMP1 | Age |
| 1841 | SERPINA3, CTSD, GDF15, PKM, TFRC | Age |
| 1842 | SERPINA3, CTSD, GDF15, TIMP1, TFRC | Age |
| 1843 | SERPINA3, CTSD, GSN, MIF, PKM | Age |
| 1844 | SERPINA3, CTSD, GSN, MIF, TIMP1 | Age |
| 1845 | SERPINA3, CTSD, GSN, MIF, TFRC | Age |
| 1846 | SERPINA3, CTSD, GSN, PKM, TIMP1 | Age |
| 1847 | SERPINA3, CTSD, GSN, PKM, TFRC | Age |
| 1848 | SERPINA3, CTSD, GSN, TIMP1, TFRC | Age |
| 1849 | SERPINA3, CTSD, MIF, PKM, TIMP1 | Age |
| 1850 | SERPINA3, CTSD, MIF, PKM, TFRC | Age |
| 1851 | SERPINA3, CTSD, MIF, TIMP1, TFRC | Age |
| 1852 | SERPINA3, CTSD, PKM, TIMP1, TFRC | Age |
| 1853 | SERPINA3, CLU, DPP4, GDF15, GSN | Age |
| 1854 | SERPINA3, CLU, DPP4, GDF15, MIF | Age |
| 1855 | SERPINA3, CLU, DPP4, GDF15, PKM | Age |
| 1856 | SERPINA3, CLU, DPP4, GDF15, TIMP1 | Age |
| 1857 | SERPINA3, CLU, DPP4, GDF15, TFRC | Age |
| 1858 | SERPINA3, CLU, DPP4, GSN, MIF | Age |
| 1859 | SERPINA3, CLU, DPP4, GSN, PKM | Age |
| 1860 | SERPINA3, CLU, DPP4, GSN, TIMP1 | Age |
| 1861 | SERPINA3, CLU, DPP4, GSN, TFRC | Age |
| 1862 | SERPINA3, CLU, DPP4, MIF, PKM | Age |
| 1863 | SERPINA3, CLU, DPP4, MIF, TIMP1 | Age |
| 1864 | SERPINA3, CLU, DPP4, MIF, TFRC | Age |
| 1865 | SERPINA3, CLU, DPP4, PKM, TIMP1 | Age |
| 1866 | SERPINA3, CLU, DPP4, PKM, TFRC | Age |
| 1867 | SERPINA3, CLU, DPP4, TIMP1, TFRC | Age |
| 1868 | SERPINA3, CLU, GDF15, GSN, MIF | Age |
| 1869 | SERPINA3, CLU, GDF15, GSN, PKM | Age |
| 1870 | SERPINA3, CLU, GDF15, GSN, TIMP1 | Age |
| 1871 | SERPINA3, CLU, GDF15, GSN, TFRC | Age |
| 1872 | SERPINA3, CLU, GDF15, MIF, PKM | Age |
| 1873 | SERPINA3, CLU, GDF15, MIF, TIMP1 | Age |
| 1874 | SERPINA3, CLU, GDF15, MIF, TFRC | Age |
| 1875 | SERPINA3, CLU, GDF15, PKM, TIMP1 | Age |
| 1876 | SERPINA3, CLU, GDF15, PKM, TFRC | Age |
| 1877 | SERPINA3, CLU, GDF15, TIMP1, TFRC | Age |
| 1878 | SERPINA3, CLU, GSN, MIF, PKM | Age |
| 1879 | SERPINA3, CLU, GSN, MIF, TIMP1 | Age |
| 1880 | SERPINA3, CLU, GSN, MIF, TFRC | Age |
| 1881 | SERPINA3, CLU, GSN, PKM, TIMP1 | Age |
| 1882 | SERPINA3, CLU, GSN, PKM, TFRC | Age |
| 1883 | SERPINA3, CLU, GSN, TIMP1, TFRC | Age |
| 1884 | SERPINA3, CLU, MIF, PKM, TIMP1 | Age |
| 1885 | SERPINA3, CLU, MIF, PKM, TFRC | Age |
| 1886 | SERPINA3, CLU, MIF, TIMP1, TFRC | Age |
| 1887 | SERPINA3, CLU, PKM, TIMP1, TFRC | Age |
| 1888 | SERPINA3, DPP4, GDF15, GSN, MIF | Age |
| 1889 | SERPINA3, DPP4, GDF15, GSN, PKM | Age |
| 1890 | SERPINA3, DPP4, GDF15, GSN, TIMP1 | Age |
| 1891 | SERPINA3, DPP4, GDF15, GSN, TFRC | Age |
| 1892 | SERPINA3, DPP4, GDF15, MIF, PKM | Age |
| 1893 | SERPINA3, DPP4, GDF15, MIF, TIMP1 | Age |
| 1894 | SERPINA3, DPP4, GDF15, MIF, TFRC | Age |
| 1895 | SERPINA3, DPP4, GDF15, PKM, TIMP1 | Age |
| 1896 | SERPINA3, DPP4, GDF15, PKM, TFRC | Age |
| 1897 | SERPINA3, DPP4, GDF15, TIMP1, TFRC | Age |
| 1898 | SERPINA3, DPP4, GSN, MIF, PKM | Age |
| 1899 | SERPINA3, DPP4, GSN, MIF, TIMP1 | Age |
| 1900 | SERPINA3, DPP4, GSN, MIF, TFRC | Age |
| 1901 | SERPINA3, DPP4, GSN, PKM, TIMP1 | Age |
| 1902 | SERPINA3, DPP4, GSN, PKM, TFRC | Age |
| 1903 | SERPINA3, DPP4, GSN, TIMP1, TFRC | Age |
| 1904 | SERPINA3, DPP4, MIF, PKM, TIMP1 | Age |
| 1905 | SERPINA3, DPP4, MIF, PKM, TFRC | Age |
| 1906 | SERPINA3, DPP4, MIF, TIMP1, TFRC | Age |
| 1907 | SERPINA3, DPP4, PKM, TIMP1, TFRC | Age |
| 1908 | SERPINA3, GDF15, GSN, MIF, PKM | Age |
| 1909 | SERPINA3, GDF15, GSN, MIF, TIMP1 | Age |
| 1910 | SERPINA3, GDF15, GSN, MIF, TFRC | Age |
| 1911 | SERPINA3, GDF15, GSN, PKM, TIMP1 | Age |
| 1912 | SERPINA3, GDF15, GSN, PKM, TFRC | Age |
| 1913 | SERPINA3, GDF15, GSN, TIMP1, TFRC | Age |
| 1914 | SERPINA3, GDF15, MIF, PKM, TIMP1 | Age |
| 1915 | SERPINA3, GDF15, MIF, PKM, TFRC | Age |
| 1916 | SERPINA3, GDF15, MIF, TIMP1, TFRC | Age |
| 1917 | SERPINA3, GDF15, PKM, TIMP1, TFRC | Age |
| 1918 | SERPINA3, GSN, MIF, PKM, TIMP1 | Age |
| 1919 | SERPINA3, GSN, MIF, PKM, TFRC | Age |
| 1920 | SERPINA3, GSN, MIF, TIMP1, TFRC | Age |
| 1921 | SERPINA3, GSN, PKM, TIMP1, TFRC | Age |
| 1922 | SERPINA3, MIF, PKM, TIMP1, TFRC | Age |
| 1923 | CTSD, CLU, DPP4, GDF15, GSN | Age |
| 1924 | CTSD, CLU, DPP4, GDF15, MIF | Age |
| 1925 | CTSD, CLU, DPP4, GDF15, PKM | Age |
| 1926 | CTSD, CLU, DPP4, GDF15, TIMP1 | Age |
| 1927 | CTSD, CLU, DPP4, GDF15, TFRC | Age |
| 1928 | CTSD, CLU, DPP4, GSN, MIF | Age |
| 1929 | CTSD, CLU, DPP4, GSN, PKM | Age |
| 1930 | CTSD, CLU, DPP4, GSN, TIMP1 | Age |
| 1931 | CTSD, CLU, DPP4, GSN, TFRC | Age |
| 1932 | CTSD, CLU, DPP4, MIF, PKM | Age |
| 1933 | CTSD, CLU, DPP4, MIF, TIMP1 | Age |
| 1934 | CTSD, CLU, DPP4, MIF, TFRC | Age |
| 1935 | CTSD, CLU, DPP4, PKM, TIMP1 | Age |
| 1936 | CTSD, CLU, DPP4, PKM, TFRC | Age |
| 1937 | CTSD, CLU, DPP4, TIMP1, TFRC | Age |
| 1938 | CTSD, CLU, GDF15, GSN, MIF | Age |
| 1939 | CTSD, CLU, GDF15, GSN, PKM | Age |
| 1940 | CTSD, CLU, GDF15, GSN, TIMP1 | Age |
| 1941 | CTSD, CLU, GDF15, GSN, TFRC | Age |
| 1942 | CTSD, CLU, GDF15, MIF, PKM | Age |
| 1943 | CTSD, CLU, GDF15, MIF, TIMP1 | Age |
| 1944 | CTSD, CLU, GDF15, MIF, TFRC | Age |
| 1945 | CTSD, CLU, GDF15, PKM, TIMP1 | Age |
| 1946 | CTSD, CLU, GDF15, PKM, TFRC | Age |
| 1947 | CTSD, CLU, GDF15, TIMP1, TFRC | Age |
| 1948 | CTSD, CLU, GSN, MIF, PKM | Age |
| 1949 | CTSD, CLU, GSN, MIF, TIMP1 | Age |
| 1950 | CTSD, CLU, GSN, MIF, TFRC | Age |
| 1951 | CTSD, CLU, GSN, PKM, TIMP1 | Age |
| 1952 | CTSD, CLU, GSN, PKM, TFRC | Age |
| 1953 | CTSD, CLU, GSN, TIMP1, TFRC | Age |
| 1954 | CTSD, CLU, MIF, PKM, TIMP1 | Age |
| 1955 | CTSD, CLU, MIF, PKM, TFRC | Age |
| 1956 | CTSD, CLU, MIF, TIMP1, TFRC | Age |
| 1957 | CTSD, CLU, PKM, TIMP1, TFRC | Age |
| 1958 | CTSD, DPP4, GDF15, GSN, MIF | Age |
| 1959 | CTSD, DPP4, GDF15, GSN, PKM | Age |
| 1960 | CTSD, DPP4, GDF15, GSN, TIMP1 | Age |
| 1961 | CTSD, DPP4, GDF15, GSN, TFRC | Age |
| 1962 | CTSD, DPP4, GDF15, MIF, PKM | Age |
| 1963 | CTSD, DPP4, GDF15, MIF, TIMP1 | Age |
| 1964 | CTSD, DPP4, GDF15, MIF, TFRC | Age |
| 1965 | CTSD, DPP4, GDF15, PKM, TIMP1 | Age |
| 1966 | CTSD, DPP4, GDF15, PKM, TFRC | Age |
| 1967 | CTSD, DPP4, GDF15, TIMP1, TFRC | Age |
| 1968 | CTSD, DPP4, GSN, MIF, PKM | Age |
| 1969 | CTSD, DPP4, GSN, MIF, TIMP1 | Age |
| 1970 | CTSD, DPP4, GSN, MIF, TFRC | Age |
| 1971 | CTSD, DPP4, GSN, PKM, TIMP1 | Age |
| 1972 | CTSD, DPP4, GSN, PKM, TFRC | Age |
| 1973 | CTSD, DPP4, GSN, TIMP1, TFRC | Age |
| 1974 | CTSD, DPP4, MIF, PKM, TIMP1 | Age |
| 1975 | CTSD, DPP4, MIF, PKM, TFRC | Age |
| 1976 | CTSD, DPP4, MIF, TIMP1, TFRC | Age |
| 1977 | CTSD, DPP4, PKM, TIMP1, TFRC | Age |
| 1978 | CTSD, GDF15, GSN, MIF, PKM | Age |
| 1979 | CTSD, GDF15, GSN, MIF, TIMP1 | Age |
| 1980 | CTSD, GDF15, GSN, MIF, TFRC | Age |
| 1981 | CTSD, GDF15, GSN, PKM, TIMP1 | Age |
| 1982 | CTSD, GDF15, GSN, PKM, TFRC | Age |
| 1983 | CTSD, GDF15, GSN, TIMP1, TFRC | Age |
| 1984 | CTSD, GDF15, MIF, PKM, TIMP1 | Age |
| 1985 | CTSD, GDF15, MIF, PKM, TFRC | Age |
| 1986 | CTSD, GDF15, MIF, TIMP1, TFRC | Age |
| 1987 | CTSD, GDF15, PKM, TIMP1, TFRC | Age |
| 1988 | CTSD, GSN, MIF, PKM, TIMP1 | Age |
| 1989 | CTSD, GSN, MIF, PKM, TFRC | Age |
| 1990 | CTSD, GSN, MIF, TIMP1, TFRC | Age |
| 1991 | CTSD, GSN, PKM, TIMP1, TFRC | Age |
| 1992 | CTSD, MIF, PKM, TIMP1, TFRC | Age |
| 1993 | CLU, DPP4, GDF15, GSN, MIF | Age |
| 1994 | CLU, DPP4, GDF15, GSN, PKM | Age |
| 1995 | CLU, DPP4, GDF15, GSN, TIMP1 | Age |
| 1996 | CLU, DPP4, GDF15, GSN, TFRC | Age |
| 1997 | CLU, DPP4, GDF15, MIF, PKM | Age |
| 1998 | CLU, DPP4, GDF15, MIF, TIMP1 | Age |
| 1999 | CLU, DPP4, GDF15, MIF, TFRC | Age |
| 2000 | CLU, DPP4, GDF15, PKM, TIMP1 | Age |
| 2001 | CLU, DPP4, GDF15, PKM, TFRC | Age |
| 2002 | CLU, DPP4, GDF15, TIMP1, TFRC | Age |
| 2003 | CLU, DPP4, GSN, MIF, PKM | Age |
| 2004 | CLU, DPP4, GSN, MIF, TIMP1 | Age |
| 2005 | CLU, DPP4, GSN, MIF, TFRC | Age |
| 2006 | CLU, DPP4, GSN, PKM, TIMP1 | Age |
| 2007 | CLU, DPP4, GSN, PKM, TFRC | Age |
| 2008 | CLU, DPP4, GSN, TIMP1, TFRC | Age |
| 2009 | CLU, DPP4, MIF, PKM, TIMP1 | Age |
| 2010 | CLU, DPP4, MIF, PKM, TFRC | Age |
| 2011 | CLU, DPP4, MIF, TIMP1, TFRC | Age |
| 2012 | CLU, DPP4, PKM, TIMP1, TFRC | Age |
| 2013 | CLU, GDF15, GSN, MIF, PKM | Age |
| 2014 | CLU, GDF15, GSN, MIF, TIMP1 | Age |
| 2015 | CLU, GDF15, GSN, MIF, TFRC | Age |
| 2016 | CLU, GDF15, GSN, PKM, TIMP1 | Age |
| 2017 | CLU, GDF15, GSN, PKM, TFRC | Age |
| 2018 | CLU, GDF15, GSN, TIMP1, TFRC | Age |
| 2019 | CLU, GDF15, MIF, PKM, TIMP1 | Age |
| 2020 | CLU, GDF15, MIF, PKM, TFRC | Age |
| 2021 | CLU, GDF15, MIF, TIMP1, TFRC | Age |
| 2022 | CLU, GDF15, PKM, TIMP1, TFRC | Age |
| 2023 | CLU, GSN, MIF, PKM, TIMP1 | Age |
| 2024 | CLU, GSN, MIF, PKM, TFRC | Age |
| 2025 | CLU, GSN, MIF, TIMP1, TFRC | Age |
| 2026 | CLU, GSN, PKM, TIMP1, TFRC | Age |
| 2027 | CLU, MIF, PKM, TIMP1, TFRC | Age |
| 2028 | DPP4, GDF15, GSN, MIF, PKM | Age |
| 2029 | DPP4, GDF15, GSN, MIF, TIMP1 | Age |
| 2030 | DPP4, GDF15, GSN, MIF, TFRC | Age |
| 2031 | DPP4, GDF15, GSN, PKM, TIMP1 | Age |
| 2032 | DPP4, GDF15, GSN, PKM, TFRC | Age |
| 2033 | DPP4, GDF15, GSN, TIMP1, TFRC | Age |
| 2034 | DPP4, GDF15, MIF, PKM, TIMP1 | Age |
| 2035 | DPP4, GDF15, MIF, PKM, TFRC | Age |
| 2036 | DPP4, GDF15, MIF, TIMP1, TFRC | Age |
| 2037 | DPP4, GDF15, PKM, TIMP1, TFRC | Age |
| 2038 | DPP4, GSN, MIF, PKM, TIMP1 | Age |
| 2039 | DPP4, GSN, MIF, PKM, TFRC | Age |
| 2040 | DPP4, GSN, MIF, TIMP1, TFRC | Age |
| 2041 | DPP4, GSN, PKM, TIMP1, TFRC | Age |
| 2042 | DPP4, MIF, PKM, TIMP1, TFRC | Age |
| 2043 | GDF15, GSN, MIF, PKM, TIMP1 | Age |
| 2044 | GDF15, GSN, MIF, PKM, TFRC | Age |
| 2045 | GDF15, GSN, MIF, TIMP1, TFRC | Age |
| 2046 | GDF15, GSN, PKM, TIMP1, TFRC | Age |
| 2047 | GDF15, MIF, PKM, TIMP1, TFRC | Age |
| 2048 | GSN, MIF, PKM, TIMP1, TFRC | Age |
| 2049 | SERPINA1, SERPINA3, CTSD, CLU, DPP4, GDF15 | NONE |
| 2050 | SERPINA1, SERPINA3, CTSD, CLU, DPP4, GSN | NONE |
| 2051 | SERPINA1, SERPINA3, CTSD, CLU, DPP4, MIF | NONE |
| 2052 | SERPINA1, SERPINA3, CTSD, CLU, DPP4, PKM | NONE |
| 2053 | SERPINA1, SERPINA3, CTSD, CLU, DPP4, TIMP1 | NONE |
| 2054 | SERPINA1, SERPINA3, CTSD, CLU, DPP4, TFRC | NONE |
| 2055 | SERPINA1, SERPINA3, CTSD, CLU, GDF15, GSN | NONE |
| 2056 | SERPINA1, SERPINA3, CTSD, CLU, GDF15, MIF | NONE |
| 2057 | SERPINA1, SERPINA3, CTSD, CLU, GDF15, PKM | NONE |
| 2058 | SERPINA1, SERPINA3, CTSD, CLU, GDF15, TIMP1 | NONE |
| 2059 | SERPINA1, SERPINA3, CTSD, CLU, GDF15, TFRC | NONE |
| 2060 | SERPINA1, SERPINA3, CTSD, CLU, GSN, MIF | NONE |
| 2061 | SERPINA1, SERPINA3, CTSD, CLU, GSN, PKM | NONE |
| 2062 | SERPINA1, SERPINA3, CTSD, CLU, GSN, TIMP1 | NONE |
| 2063 | SERPINA1, SERPINA3, CTSD, CLU, GSN, TFRC | NONE |
| 2064 | SERPINA1, SERPINA3, CTSD, CLU, MIF, PKM | NONE |
| 2065 | SERPINA1, SERPINA3, CTSD, CLU, MIF, TIMP1 | NONE |
| 2066 | SERPINA1, SERPINA3, CTSD, CLU, MIF, TFRC | NONE |
| 2067 | SERPINA1, SERPINA3, CTSD, CLU, PKM, TIMP1 | NONE |
| 2068 | SERPINA1, SERPINA3, CTSD, CLU, PKM, TFRC | NONE |
| 2069 | SERPINA1, SERPINA3, CTSD, CLU, TIMP1, TFRC | NONE |
| 2070 | SERPINA1, SERPINA3, CTSD, DPP4, GDF15, GSN | NONE |
| 2071 | SERPINA1, SERPINA3, CTSD, DPP4, GDF15, MIF | NONE |
| 2072 | SERPINA1, SERPINA3, CTSD, DPP4, GDF15, PKM | NONE |
| 2073 | SERPINA1, SERPINA3, CTSD, DPP4, GDF15, TIMP1 | NONE |
| 2074 | SERPINA1, SERPINA3, CTSD, DPP4, GDF15, TFRC | NONE |
| 2075 | SERPINA1, SERPINA3, CTSD, DPP4, GSN, MIF | NONE |
| 2076 | SERPINA1, SERPINA3, CTSD, DPP4, GSN, PKM | NONE |
| 2077 | SERPINA1, SERPINA3, CTSD, DPP4, GSN, TIMP1 | NONE |
| 2078 | SERPINA1, SERPINA3, CTSD, DPP4, GSN, TFRC | NONE |
| 2079 | SERPINA1, SERPINA3, CTSD, DPP4, MIF, PKM | NONE |
| 2080 | SERPINA1, SERPINA3, CTSD, DPP4, MIF, TIMP1 | NONE |
| 2081 | SERPINA1, SERPINA3, CTSD, DPP4, MIF, TFRC | NONE |
| 2082 | SERPINA1, SERPINA3, CTSD, DPP4, PKM, TIMP1 | NONE |
| 2083 | SERPINA1, SERPINA3, CTSD, DPP4, PKM, TFRC | NONE |
| 2084 | SERPINA1, SERPINA3, CTSD, DPP4, TIMP1, TFRC | NONE |
| 2085 | SERPINA1, SERPINA3, CTSD, GDF15, GSN, MIF | NONE |
| 2086 | SERPINA1, SERPINA3, CTSD, GDF15, GSN, PKM | NONE |
| 2087 | SERPINA1, SERPINA3, CTSD, GDF15, GSN, TIMP1 | NONE |
| 2088 | SERPINA1, SERPINA3, CTSD, GDF15, GSN, TFRC | NONE |
| 2089 | SERPINA1, SERPINA3, CTSD, GDF15, MIF, PKM | NONE |
| 2090 | SERPINA1, SERPINA3, CTSD, GDF15, MIF, TIMP1 | NONE |
| 2091 | SERPINA1, SERPINA3, CTSD, GDF15, MIF, TFRC | NONE |
| 2092 | SERPINA1, SERPINA3, CTSD, GDF15, PKM, TIMP1 | NONE |
| 2093 | SERPINA1, SERPINA3, CTSD, GDF15, PKM, TFRC | NONE |
| 2094 | SERPINA1, SERPINA3, CTSD, GDF15, TIMP1, TFRC | NONE |
| 2095 | SERPINA1, SERPINA3, CTSD, GSN, MIF, PKM | NONE |
| 2096 | SERPINA1, SERPINA3, CTSD, GSN, MIF, TIMP1 | NONE |
| 2097 | SERPINA1, SERPINA3, CTSD, GSN, MIF, TFRC | NONE |
| 2098 | SERPINA1, SERPINA3, CTSD, GSN, PKM, TIMP1 | NONE |
| 2099 | SERPINA1, SERPINA3, CTSD, GSN, PKM, TFRC | NONE |
| 2100 | SERPINA1, SERPINA3, CTSD, GSN, TIMP1, TFRC | NONE |
| 2101 | SERPINA1, SERPINA3, CTSD, MIF, PKM, TIMP1 | NONE |
| 2102 | SERPINA1, SERPINA3, CTSD, MIF, PKM, TFRC | NONE |
| 2103 | SERPINA1, SERPINA3, CTSD, MIF, TIMP1, TFRC | NONE |
| 2104 | SERPINA1, SERPINA3, CTSD, PKM, TIMP1, TFRC | NONE |
| 2105 | SERPINA1, SERPINA3, CLU, DPP4, GDF15, GSN | NONE |
| 2106 | SERPINA1, SERPINA3, CLU, DPP4, GDF15, MIF | NONE |
| 2107 | SERPINA1, SERPINA3, CLU, DPP4, GDF15, PKM | NONE |
| 2108 | SERPINA1, SERPINA3, CLU, DPP4, GDF15, TIMP1 | NONE |
| 2109 | SERPINA1, SERPINA3, CLU, DPP4, GDF15, TFRC | NONE |
| 2110 | SERPINA1, SERPINA3, CLU, DPP4, GSN, MIF | NONE |
| 2111 | SERPINA1, SERPINA3, CLU, DPP4, GSN, PKM | NONE |
| 2112 | SERPINA1, SERPINA3, CLU, DPP4, GSN, TIMP1 | NONE |
| 2113 | SERPINA1, SERPINA3, CLU, DPP4, GSN, TFRC | NONE |
| 2114 | SERPINA1, SERPINA3, CLU, DPP4, MIF, PKM | NONE |
| 2115 | SERPINA1, SERPINA3, CLU, DPP4, MIF, TIMP1 | NONE |
| 2116 | SERPINA1, SERPINA3, CLU, DPP4, MIF, TFRC | NONE |
| 2117 | SERPINA1, SERPINA3, CLU, DPP4, PKM, TIMP1 | NONE |
| 2118 | SERPINA1, SERPINA3, CLU, DPP4, PKM, TFRC | NONE |
| 2119 | SERPINA1, SERPINA3, CLU, DPP4, TIMP1, TFRC | NONE |
| 2120 | SERPINA1, SERPINA3, CLU, GDF15, GSN, MIF | NONE |
| 2121 | SERPINA1, SERPINA3, CLU, GDF15, GSN, PKM | NONE |
| 2122 | SERPINA1, SERPINA3, CLU, GDF15, GSN, TIMP1 | NONE |
| 2123 | SERPINA1, SERPINA3, CLU, GDF15, GSN, TFRC | NONE |
| 2124 | SERPINA1, SERPINA3, CLU, GDF15, MIF, PKM | NONE |
| 2125 | SERPINA1, SERPINA3, CLU, GDF15, MIF, TIMP1 | NONE |
| 2126 | SERPINA1, SERPINA3, CLU, GDF15, MIF, TFRC | NONE |
| 2127 | SERPINA1, SERPINA3, CLU, GDF15, PKM, TIMP1 | NONE |
| 2128 | SERPINA1, SERPINA3, CLU, GDF15, PKM, TFRC | NONE |
| 2129 | SERPINA1, SERPINA3, CLU, GDF15, TIMP1, TFRC | NONE |
| 2130 | SERPINA1, SERPINA3, CLU, GSN, MIF, PKM | NONE |
| 2131 | SERPINA1, SERPINA3, CLU, GSN, MIF, TIMP1 | NONE |
| 2132 | SERPINA1, SERPINA3, CLU, GSN, MIF, TFRC | NONE |
| 2133 | SERPINA1, SERPINA3, CLU, GSN, PKM, TIMP1 | NONE |
| 2134 | SERPINA1, SERPINA3, CLU, GSN, PKM, TFRC | NONE |
| 2135 | SERPINA1, SERPINA3, CLU, GSN, TIMP1, TFRC | NONE |
| 2136 | SERPINA1, SERPINA3, CLU, MIF, PKM, TIMP1 | NONE |
| 2137 | SERPINA1, SERPINA3, CLU, MIF, PKM, TFRC | NONE |
| 2138 | SERPINA1, SERPINA3, CLU, MIF, TIMP1, TFRC | NONE |
| 2139 | SERPINA1, SERPINA3, CLU, PKM, TIMP1, TFRC | NONE |
| 2140 | SERPINA1, SERPINA3, DPP4, GDF15, GSN, MIF | NONE |
| 2141 | SERPINA1, SERPINA3, DPP4, GDF15, GSN, PKM | NONE |
| 2142 | SERPINA1, SERPINA3, DPP4, GDF15, GSN, TIMP1 | NONE |
| 2143 | SERPINA1, SERPINA3, DPP4, GDF15, GSN, TFRC | NONE |
| 2144 | SERPINA1, SERPINA3, DPP4, GDF15, MIF, PKM | NONE |
| 2145 | SERPINA1, SERPINA3, DPP4, GDF15, MIF, TIMP1 | NONE |
| 2146 | SERPINA1, SERPINA3, DPP4, GDF15, MIF, TFRC | NONE |
| 2147 | SERPINA1, SERPINA3, DPP4, GDF15, PKM, TIMP1 | NONE |
| 2148 | SERPINA1, SERPINA3, DPP4, GDF15, PKM, TFRC | NONE |
| 2149 | SERPINA1, SERPINA3, DPP4, GDF15, TIMP1, TFRC | NONE |
| 2150 | SERPINA1, SERPINA3, DPP4, GSN, MIF, PKM | NONE |
| 2151 | SERPINA1, SERPINA3, DPP4, GSN, MIF, TIMP1 | NONE |
| 2152 | SERPINA1, SERPINA3, DPP4, GSN, MIF, TFRC | NONE |
| 2153 | SERPINA1, SERPINA3, DPP4, GSN, PKM, TIMP1 | NONE |
| 2154 | SERPINA1, SERPINA3, DPP4, GSN, PKM, TFRC | NONE |
| 2155 | SERPINA1, SERPINA3, DPP4, GSN, TIMP1, TFRC | NONE |
| 2156 | SERPINA1, SERPINA3, DPP4, MIF, PKM, TIMP1 | NONE |
| 2157 | SERPINA1, SERPINA3, DPP4, MIF, PKM, TFRC | NONE |
| 2158 | SERPINA1, SERPINA3, DPP4, MIF, TIMP1, TFRC | NONE |
| 2159 | SERPINA1, SERPINA3, DPP4, PKM, TIMP1, TFRC | NONE |
| 2160 | SERPINA1, SERPINA3, GDF15, GSN, MIF, PKM | NONE |
| 2161 | SERPINA1, SERPINA3, GDF15, GSN, MIF, TIMP1 | NONE |
| 2162 | SERPINA1, SERPINA3, GDF15, GSN, MIF, TFRC | NONE |
| 2163 | SERPINA1, SERPINA3, GDF15, GSN, PKM, TIMP1 | NONE |
| 2164 | SERPINA1, SERPINA3, GDF15, GSN, PKM, TFRC | NONE |
| 2165 | SERPINA1, SERPINA3, GDF15, GSN, TIMP1, TFRC | NONE |
| 2166 | SERPINA1, SERPINA3, GDF15, MIF, PKM, TIMP1 | NONE |
| 2167 | SERPINA1, SERPINA3, GDF15, MIF, PKM, TFRC | NONE |
| 2168 | SERPINA1, SERPINA3, GDF15, MIF, TIMP1, TFRC | NONE |
| 2169 | SERPINA1, SERPINA3, GDF15, PKM, TIMP1, TFRC | NONE |
| 2170 | SERPINA1, SERPINA3, GSN, MIF, PKM, TIMP1 | NONE |
| 2171 | SERPINA1, SERPINA3, GSN, MIF, PKM, TFRC | NONE |
| 2172 | SERPINA1, SERPINA3, GSN, MIF, TIMP1, TFRC | NONE |
| 2173 | SERPINA1, SERPINA3, GSN, PKM, TIMP1, TFRC | NONE |
| 2174 | SERPINA1, SERPINA3, MIF, PKM, TIMP1, TFRC | NONE |
| 2175 | SERPINA1, CTSD, CLU, DPP4, GDF15, GSN | NONE |
| 2176 | SERPINA1, CTSD, CLU, DPP4, GDF15, MIF | NONE |
| 2177 | SERPINA1, CTSD, CLU, DPP4, GDF15, PKM | NONE |
| 2178 | SERPINA1, CTSD, CLU, DPP4, GDF15, TIMP1 | NONE |
| 2179 | SERPINA1, CTSD, CLU, DPP4, GDF15, TFRC | NONE |
| 2180 | SERPINA1, CTSD, CLU, DPP4, GSN, MIF | NONE |
| 2181 | SERPINA1, CTSD, CLU, DPP4, GSN, PKM | NONE |
| 2182 | SERPINA1, CTSD, CLU, DPP4, GSN, TIMP1 | NONE |
| 2183 | SERPINA1, CTSD, CLU, DPP4, GSN, TFRC | NONE |
| 2184 | SERPINA1, CTSD, CLU, DPP4, MIF, PKM | NONE |
| 2185 | SERPINA1, CTSD, CLU, DPP4, MIF, TIMP1 | NONE |
| 2186 | SERPINA1, CTSD, CLU, DPP4, MIF, TFRC | NONE |
| 2187 | SERPINA1, CTSD, CLU, DPP4, PKM, TIMP1 | NONE |
| 2188 | SERPINA1, CTSD, CLU, DPP4, PKM, TFRC | NONE |
| 2189 | SERPINA1, CTSD, CLU, DPP4, TIMP1, TFRC | NONE |
| 2190 | SERPINA1, CTSD, CLU, GDF15, GSN, MIF | NONE |
| 2191 | SERPINA1, CTSD, CLU, GDF15, GSN, PKM | NONE |
| 2192 | SERPINA1, CTSD, CLU, GDF15, GSN, TIMP1 | NONE |
| 2193 | SERPINA1, CTSD, CLU, GDF15, GSN, TFRC | NONE |
| 2194 | SERPINA1, CTSD, CLU, GDF15, MIF, PKM | NONE |
| 2195 | SERPINA1, CTSD, CLU, GDF15, MIF, TIMP1 | NONE |
| 2196 | SERPINA1, CTSD, CLU, GDF15, MIF, TFRC | NONE |
| 2197 | SERPINA1, CTSD, CLU, GDF15, PKM, TIMP1 | NONE |
| 2198 | SERPINA1, CTSD, CLU, GDF15, PKM, TFRC | NONE |
| 2199 | SERPINA1, CTSD, CLU, GDF15, TIMP1, TFRC | NONE |
| 2200 | SERPINA1, CTSD, CLU, GSN, MIF, PKM | NONE |
| 2201 | SERPINA1, CTSD, CLU, GSN, MIF, TIMP1 | NONE |
| 2202 | SERPINA1, CTSD, CLU, GSN, MIF, TFRC | NONE |
| 2203 | SERPINA1, CTSD, CLU, GSN, PKM, TIMP1 | NONE |
| 2204 | SERPINA1, CTSD, CLU, GSN, PKM, TFRC | NONE |
| 2205 | SERPINA1, CTSD, CLU, GSN, TIMP1, TFRC | NONE |
| 2206 | SERPINA1, CTSD, CLU, MIF, PKM, TIMP1 | NONE |
| 2207 | SERPINA1, CTSD, CLU, MIF, PKM, TFRC | NONE |
| 2208 | SERPINA1, CTSD, CLU, MIF, TIMP1, TFRC | NONE |
| 2209 | SERPINA1, CTSD, CLU, PKM, TIMP1, TFRC | NONE |
| 2210 | SERPINA1, CTSD, DPP4, GDF15, GSN, MIF | NONE |
| 2211 | SERPINA1, CTSD, DPP4, GDF15, GSN, PKM | NONE |
| 2212 | SERPINA1, CTSD, DPP4, GDF15, GSN, TIMP1 | NONE |
| 2213 | SERPINA1, CTSD, DPP4, GDF15, GSN, TFRC | NONE |
| 2214 | SERPINA1, CTSD, DPP4, GDF15, MIF, PKM | NONE |
| 2215 | SERPINA1, CTSD, DPP4, GDF15, MIF, TIMP1 | NONE |
| 2216 | SERPINA1, CTSD, DPP4, GDF15, MIF, TFRC | NONE |
| 2217 | SERPINA1, CTSD, DPP4, GDF15, PKM, TIMP1 | NONE |
| 2218 | SERPINA1, CTSD, DPP4, GDF15, PKM, TFRC | NONE |
| 2219 | SERPINA1, CTSD, DPP4, GDF15, TIMP1, TFRC | NONE |
| 2220 | SERPINA1, CTSD, DPP4, GSN, MIF, PKM | NONE |
| 2221 | SERPINA1, CTSD, DPP4, GSN, MIF, TIMP1 | NONE |
| 2222 | SERPINA1, CTSD, DPP4, GSN, MIF, TFRC | NONE |
| 2223 | SERPINA1, CTSD, DPP4, GSN, PKM, TIMP1 | NONE |
| 2224 | SERPINA1, CTSD, DPP4, GSN, PKM, TFRC | NONE |
| 2225 | SERPINA1, CTSD, DPP4, GSN, TIMP1, TFRC | NONE |
| 2226 | SERPINA1, CTSD, DPP4, MIF, PKM, TIMP1 | NONE |
| 2227 | SERPINA1, CTSD, DPP4, MIF, PKM, TFRC | NONE |
| 2228 | SERPINA1, CTSD, DPP4, MIF, TIMP1, TFRC | NONE |
| 2229 | SERPINA1, CTSD, DPP4, PKM, TIMP1, TFRC | NONE |
| 2230 | SERPINA1, CTSD, GDF15, GSN, MIF, PKM | NONE |
| 2231 | SERPINA1, CTSD, GDF15, GSN, MIF, TIMP1 | NONE |
| 2232 | SERPINA1, CTSD, GDF15, GSN, MIF, TFRC | NONE |
| 2233 | SERPINA1, CTSD, GDF15, GSN, PKM, TIMP1 | NONE |
| 2234 | SERPINA1, CTSD, GDF15, GSN, PKM, TFRC | NONE |
| 2235 | SERPINA1, CTSD, GDF15, GSN, TIMP1, TFRC | NONE |
| 2236 | SERPINA1, CTSD, GDF15, MIF, PKM, TIMP1 | NONE |
| 2237 | SERPINA1, CTSD, GDF15, MIF, PKM, TFRC | NONE |
| 2238 | SERPINA1, CTSD, GDF15, MIF, TIMP1, TFRC | NONE |
| 2239 | SERPINA1, CTSD, GDF15, PKM, TIMP1, TFRC | NONE |
| 2240 | SERPINA1, CTSD, GSN, MIF, PKM, TIMP1 | NONE |
| 2241 | SERPINA1, CTSD, GSN, MIF, PKM, TFRC | NONE |
| 2242 | SERPINA1, CTSD, GSN, MIF, TIMP1, TFRC | NONE |
| 2243 | SERPINA1, CTSD, GSN, PKM, TIMP1, TFRC | NONE |
| 2244 | SERPINA1, CTSD, MIF, PKM, TIMP1, TFRC | NONE |
| 2245 | SERPINA1, CLU, DPP4, GDF15, GSN, MIF | NONE |
| 2246 | SERPINA1, CLU, DPP4, GDF15, GSN, PKM | NONE |
| 2247 | SERPINA1, CLU, DPP4, GDF15, GSN, TIMP1 | NONE |
| 2248 | SERPINA1, CLU, DPP4, GDF15, GSN, TFRC | NONE |
| 2249 | SERPINA1, CLU, DPP4, GDF15, MIF, PKM | NONE |
| 2250 | SERPINA1, CLU, DPP4, GDF15, MIF, TIMP1 | NONE |
| 2251 | SERPINA1, CLU, DPP4, GDF15, MIF, TFRC | NONE |
| 2252 | SERPINA1, CLU, DPP4, GDF15, PKM, TIMP1 | NONE |
| 2253 | SERPINA1, CLU, DPP4, GDF15, PKM, TFRC | NONE |
| 2254 | SERPINA1, CLU, DPP4, GDF15, TIMP1, TFRC | NONE |
| 2255 | SERPINA1, CLU, DPP4, GSN, MIF, PKM | NONE |
| 2256 | SERPINA1, CLU, DPP4, GSN, MIF, TIMP1 | NONE |
| 2257 | SERPINA1, CLU, DPP4, GSN, MIF, TFRC | NONE |
| 2258 | SERPINA1, CLU, DPP4, GSN, PKM, TIMP1 | NONE |
| 2259 | SERPINA1, CLU, DPP4, GSN, PKM, TFRC | NONE |
| 2260 | SERPINA1, CLU, DPP4, GSN, TIMP1, TFRC | NONE |
| 2261 | SERPINA1, CLU, DPP4, MIF, PKM, TIMP1 | NONE |
| 2262 | SERPINA1, CLU, DPP4, MIF, PKM, TFRC | NONE |
| 2263 | SERPINA1, CLU, DPP4, MIF, TIMP1, TFRC | NONE |
| 2264 | SERPINA1, CLU, DPP4, PKM, TIMP1, TFRC | NONE |
| 2265 | SERPINA1, CLU, GDF15, GSN, MIF, PKM | NONE |
| 2266 | SERPINA1, CLU, GDF15, GSN, MIF, TIMP1 | NONE |
| 2267 | SERPINA1, CLU, GDF15, GSN, MIF, TFRC | NONE |
| 2268 | SERPINA1, CLU, GDF15, GSN, PKM, TIMP1 | NONE |
| 2269 | SERPINA1, CLU, GDF15, GSN, PKM, TFRC | NONE |
| 2270 | SERPINA1, CLU, GDF15, GSN, TIMP1, TFRC | NONE |
| 2271 | SERPINA1, CLU, GDF15, MIF, PKM, TIMP1 | NONE |
| 2272 | SERPINA1, CLU, GDF15, MIF, PKM, TFRC | NONE |
| 2273 | SERPINA1, CLU, GDF15, MIF, TIMP1, TFRC | NONE |
| 2274 | SERPINA1, CLU, GDF15, PKM, TIMP1, TFRC | NONE |
| 2275 | SERPINA1, CLU, GSN, MIF, PKM, TIMP1 | NONE |
| 2276 | SERPINA1, CLU, GSN, MIF, PKM, TFRC | NONE |
| 2277 | SERPINA1, CLU, GSN, MIF, TIMP1, TFRC | NONE |
| 2278 | SERPINA1, CLU, GSN, PKM, TIMP1, TFRC | NONE |
| 2279 | SERPINA1, CLU, MIF, PKM, TIMP1, TFRC | NONE |
| 2280 | SERPINA1, DPP4, GDF15, GSN, MIF, PKM | NONE |
| 2281 | SERPINA1, DPP4, GDF15, GSN, MIF, TIMP1 | NONE |
| 2282 | SERPINA1, DPP4, GDF15, GSN, MIF, TFRC | NONE |
| 2283 | SERPINA1, DPP4, GDF15, GSN, PKM, TIMP1 | NONE |
| 2284 | SERPINA1, DPP4, GDF15, GSN, PKM, TFRC | NONE |
| 2285 | SERPINA1, DPP4, GDF15, GSN, TIMP1, TFRC | NONE |
| 2286 | SERPINA1, DPP4, GDF15, MIF, PKM, TIMP1 | NONE |
| 2287 | SERPINA1, DPP4, GDF15, MIF, PKM, TFRC | NONE |
| 2288 | SERPINA1, DPP4, GDF15, MIF, TIMP1, TFRC | NONE |
| 2289 | SERPINA1, DPP4, GDF15, PKM, TIMP1, TFRC | NONE |
| 2290 | SERPINA1, DPP4, GSN, MIF, PKM, TIMP1 | NONE |
| 2291 | SERPINA1, DPP4, GSN, MIF, PKM, TFRC | NONE |
| 2292 | SERPINA1, DPP4, GSN, MIF, TIMP1, TFRC | NONE |
| 2293 | SERPINA1, DPP4, GSN, PKM, TIMP1, TFRC | NONE |
| 2294 | SERPINA1, DPP4, MIF, PKM, TIMP1, TFRC | NONE |
| 2295 | SERPINA1, GDF15, GSN, MIF, PKM, TIMP1 | NONE |
| 2296 | SERPINA1, GDF15, GSN, MIF, PKM, TFRC | NONE |
| 2297 | SERPINA1, GDF15, GSN, MIF, TIMP1, TFRC | NONE |
| 2298 | SERPINA1, GDF15, GSN, PKM, TIMP1, TFRC | NONE |
| 2299 | SERPINA1, GDF15, MIF, PKM, TIMP1, TFRC | NONE |
| 2300 | SERPINA1, GSN, MIF, PKM, TIMP1, TFRC | NONE |
| 2301 | SERPINA3, CTSD, CLU, DPP4, GDF15, GSN | NONE |
| 2302 | SERPINA3, CTSD, CLU, DPP4, GDF15, MIF | NONE |
| 2303 | SERPINA3, CTSD, CLU, DPP4, GDF15, PKM | NONE |
| 2304 | SERPINA3, CTSD, CLU, DPP4, GDF15, TIMP1 | NONE |
| 2305 | SERPINA3, CTSD, CLU, DPP4, GDF15, TFRC | NONE |
| 2306 | SERPINA3, CTSD, CLU, DPP4, GSN, MIF | NONE |
| 2307 | SERPINA3, CTSD, CLU, DPP4, GSN, PKM | NONE |
| 2308 | SERPINA3, CTSD, CLU, DPP4, GSN, TIMP1 | NONE |
| 2309 | SERPINA3, CTSD, CLU, DPP4, GSN, TFRC | NONE |
| 2310 | SERPINA3, CTSD, CLU, DPP4, MIF, PKM | NONE |
| 2311 | SERPINA3, CTSD, CLU, DPP4, MIF, TIMP1 | NONE |
| 2312 | SERPINA3, CTSD, CLU, DPP4, MIF, TFRC | NONE |
| 2313 | SERPINA3, CTSD, CLU, DPP4, PKM, TIMP1 | NONE |
| 2314 | SERPINA3, CTSD, CLU, DPP4, PKM, TFRC | NONE |
| 2315 | SERPINA3, CTSD, CLU, DPP4, TIMP1, TFRC | NONE |
| 2316 | SERPINA3, CTSD, CLU, GDF15, GSN, MIF | NONE |
| 2317 | SERPINA3, CTSD, CLU, GDF15, GSN, PKM | NONE |
| 2318 | SERPINA3, CTSD, CLU, GDF15, GSN, TIMP1 | NONE |
| 2319 | SERPINA3, CTSD, CLU, GDF15, GSN, TFRC | NONE |
| 2320 | SERPINA3, CTSD, CLU, GDF15, MIF, PKM | NONE |
| 2321 | SERPINA3, CTSD, CLU, GDF15, MIF, TIMP1 | NONE |
| 2322 | SERPINA3, CTSD, CLU, GDF15, MIF, TFRC | NONE |
| 2323 | SERPINA3, CTSD, CLU, GDF15, PKM, TIMP1 | NONE |
| 2324 | SERPINA3, CTSD, CLU, GDF15, PKM, TFRC | NONE |
| 2325 | SERPINA3, CTSD, CLU, GDF15, TIMP1, TFRC | NONE |
| 2326 | SERPINA3, CTSD, CLU, GSN, MIF, PKM | NONE |
| 2327 | SERPINA3, CTSD, CLU, GSN, MIF, TIMP1 | NONE |
| 2328 | SERPINA3, CTSD, CLU, GSN, MIF, TFRC | NONE |
| 2329 | SERPINA3, CTSD, CLU, GSN, PKM, TIMP1 | NONE |
| 2330 | SERPINA3, CTSD, CLU, GSN, PKM, TFRC | NONE |
| 2331 | SERPINA3, CTSD, CLU, GSN, TIMP1, TFRC | NONE |
| 2332 | SERPINA3, CTSD, CLU, MIF, PKM, TIMP1 | NONE |
| 2333 | SERPINA3, CTSD, CLU, MIF, PKM, TFRC | NONE |
| 2334 | SERPINA3, CTSD, CLU, MIF, TIMP1, TFRC | NONE |
| 2335 | SERPINA3, CTSD, CLU, PKM, TIMP1, TFRC | NONE |
| 2336 | SERPINA3, CTSD, DPP4, GDF15, GSN, MIF | NONE |
| 2337 | SERPINA3, CTSD, DPP4, GDF15, GSN, PKM | NONE |
| 2338 | SERPINA3, CTSD, DPP4, GDF15, GSN, TIMP1 | NONE |
| 2339 | SERPINA3, CTSD, DPP4, GDF15, GSN, TFRC | NONE |
| 2340 | SERPINA3, CTSD, DPP4, GDF15, MIF, PKM | NONE |
| 2341 | SERPINA3, CTSD, DPP4, GDF15, MIF, TIMP1 | NONE |
| 2342 | SERPINA3, CTSD, DPP4, GDF15, MIF, TFRC | NONE |
| 2343 | SERPINA3, CTSD, DPP4, GDF15, PKM, TIMP1 | NONE |
| 2344 | SERPINA3, CTSD, DPP4, GDF15, PKM, TFRC | NONE |
| 2345 | SERPINA3, CTSD, DPP4, GDF15, TIMP1, TFRC | NONE |
| 2346 | SERPINA3, CTSD, DPP4, GSN, MIF, PKM | NONE |
| 2347 | SERPINA3, CTSD, DPP4, GSN, MIF, TIMP1 | NONE |
| 2348 | SERPINA3, CTSD, DPP4, GSN, MIF, TFRC | NONE |
| 2349 | SERPINA3, CTSD, DPP4, GSN, PKM, TIMP1 | NONE |
| 2350 | SERPINA3, CTSD, DPP4, GSN, PKM, TFRC | NONE |
| 2351 | SERPINA3, CTSD, DPP4, GSN, TIMP1, TFRC | NONE |
| 2352 | SERPINA3, CTSD, DPP4, MIF, PKM, TIMP1 | NONE |
| 2353 | SERPINA3, CTSD, DPP4, MIF, PKM, TFRC | NONE |
| 2354 | SERPINA3, CTSD, DPP4, MIF, TIMP1, TFRC | NONE |
| 2355 | SERPINA3, CTSD, DPP4, PKM, TIMP1, TFRC | NONE |
| 2356 | SERPINA3, CTSD, GDF15, GSN, MIF, PKM | NONE |
| 2357 | SERPINA3, CTSD, GDF15, GSN, MIF, TIMP1 | NONE |
| 2358 | SERPINA3, CTSD, GDF15, GSN, MIF, TFRC | NONE |
| 2359 | SERPINA3, CTSD, GDF15, GSN, PKM, TIMP1 | NONE |
| 2360 | SERPINA3, CTSD, GDF15, GSN, PKM, TFRC | NONE |
| 2361 | SERPINA3, CTSD, GDF15, GSN, TIMP1, TFRC | NONE |
| 2362 | SERPINA3, CTSD, GDF15, MIF, PKM, TIMP1 | NONE |
| 2363 | SERPINA3, CTSD, GDF15, MIF, PKM, TFRC | NONE |
| 2364 | SERPINA3, CTSD, GDF15, MIF, TIMP1, TFRC | NONE |
| 2365 | SERPINA3, CTSD, GDF15, PKM, TIMP1, TFRC | NONE |
| 2366 | SERPINA3, CTSD, GSN, MIF, PKM, TIMP1 | NONE |
| 2367 | SERPINA3, CTSD, GSN, MIF, PKM, TFRC | NONE |
| 2368 | SERPINA3, CTSD, GSN, MIF, TIMP1, TFRC | NONE |
| 2369 | SERPINA3, CTSD, GSN, PKM, TIMP1, TFRC | NONE |
| 2370 | SERPINA3, CTSD, MIF, PKM, TIMP1, TFRC | NONE |
| 2371 | SERPINA3, CLU, DPP4, GDF15, GSN, MIF | NONE |
| 2372 | SERPINA3, CLU, DPP4, GDF15, GSN, PKM | NONE |
| 2373 | SERPINA3, CLU, DPP4, GDF15, GSN, TIMP1 | NONE |
| 2374 | SERPINA3, CLU, DPP4, GDF15, GSN, TFRC | NONE |
| 2375 | SERPINA3, CLU, DPP4, GDF15, MIF, PKM | NONE |
| 2376 | SERPINA3, CLU, DPP4, GDF15, MIF, TIMP1 | NONE |
| 2377 | SERPINA3, CLU, DPP4, GDF15, MIF, TFRC | NONE |
| 2378 | SERPINA3, CLU, DPP4, GDF15, PKM, TIMP1 | NONE |
| 2379 | SERPINA3, CLU, DPP4, GDF15, PKM, TFRC | NONE |
| 2380 | SERPINA3, CLU, DPP4, GDF15, TIMP1, TFRC | NONE |
| 2381 | SERPINA3, CLU, DPP4, GSN, MIF, PKM | NONE |
| 2382 | SERPINA3, CLU, DPP4, GSN, MIF, TIMP1 | NONE |
| 2383 | SERPINA3, CLU, DPP4, GSN, MIF, TFRC | NONE |
| 2384 | SERPINA3, CLU, DPP4, GSN, PKM, TIMP1 | NONE |
| 2385 | SERPINA3, CLU, DPP4, GSN, PKM, TFRC | NONE |
| 2386 | SERPINA3, CLU, DPP4, GSN, TIMP1, TFRC | NONE |
| 2387 | SERPINA3, CLU, DPP4, MIF, PKM, TIMP1 | NONE |
| 2388 | SERPINA3, CLU, DPP4, MIF, PKM, TFRC | NONE |
| 2389 | SERPINA3, CLU, DPP4, MIF, TIMP1, TFRC | NONE |
| 2390 | SERPINA3, CLU, DPP4, PKM, TIMP1, TFRC | NONE |
| 2391 | SERPINA3, CLU, GDF15, GSN, MIF, PKM | NONE |
| 2392 | SERPINA3, CLU, GDF15, GSN, MIF, TIMP1 | NONE |
| 2393 | SERPINA3, CLU, GDF15, GSN, MIF, TFRC | NONE |
| 2394 | SERPINA3, CLU, GDF15, GSN, PKM, TIMP1 | NONE |
| 2395 | SERPINA3, CLU, GDF15, GSN, PKM, TFRC | NONE |
| 2396 | SERPINA3, CLU, GDF15, GSN, TIMP1, TFRC | NONE |
| 2397 | SERPINA3, CLU, GDF15, MIF, PKM, TIMP1 | NONE |
| 2398 | SERPINA3, CLU, GDF15, MIF, PKM, TFRC | NONE |
| 2399 | SERPINA3, CLU, GDF15, MIF, TIMP1, TFRC | NONE |
| 2400 | SERPINA3, CLU, GDF15, PKM, TIMP1, TFRC | NONE |
| 2401 | SERPINA3, CLU, GSN, MIF, PKM, TIMP1 | NONE |
| 2402 | SERPINA3, CLU, GSN, MIF, PKM, TFRC | NONE |
| 2403 | SERPINA3, CLU, GSN, MIF, TIMP1, TFRC | NONE |
| 2404 | SERPINA3, CLU, GSN, PKM, TIMP1, TFRC | NONE |
| 2405 | SERPINA3, CLU, MIF, PKM, TIMP1, TFRC | NONE |
| 2406 | SERPINA3, DPP4, GDF15, GSN, MIF, PKM | NONE |
| 2407 | SERPINA3, DPP4, GDF15, GSN, MIF, TIMP1 | NONE |
| 2408 | SERPINA3, DPP4, GDF15, GSN, MIF, TFRC | NONE |
| 2409 | SERPINA3, DPP4, GDF15, GSN, PKM, TIMP1 | NONE |
| 2410 | SERPINA3, DPP4, GDF15, GSN, PKM, TFRC | NONE |
| 2411 | SERPINA3, DPP4, GDF15, GSN, TIMP1, TFRC | NONE |
| 2412 | SERPINA3, DPP4, GDF15, MIF, PKM, TIMP1 | NONE |
| 2413 | SERPINA3, DPP4, GDF15, MIF, PKM, TFRC | NONE |
| 2414 | SERPINA3, DPP4, GDF15, MIF, TIMP1, TFRC | NONE |
| 2415 | SERPINA3, DPP4, GDF15, PKM, TIMP1, TFRC | NONE |
| 2416 | SERPINA3, DPP4, GSN, MIF, PKM, TIMP1 | NONE |
| 2417 | SERPINA3, DPP4, GSN, MIF, PKM, TFRC | NONE |
| 2418 | SERPINA3, DPP4, GSN, MIF, TIMP1, TFRC | NONE |
| 2419 | SERPINA3, DPP4, GSN, PKM, TIMP1, TFRC | NONE |
| 2420 | SERPINA3, DPP4, MIF, PKM, TIMP1, TFRC | NONE |
| 2421 | SERPINA3, GDF15, GSN, MIF, PKM, TIMP1 | NONE |
| 2422 | SERPINA3, GDF15, GSN, MIF, PKM, TFRC | NONE |
| 2423 | SERPINA3, GDF15, GSN, MIF, TIMP1, TFRC | NONE |
| 2424 | SERPINA3, GDF15, GSN, PKM, TIMP1, TFRC | NONE |
| 2425 | SERPINA3, GDF15, MIF, PKM, TIMP1, TFRC | NONE |
| 2426 | SERPINA3, GSN, MIF, PKM, TIMP1, TFRC | NONE |
| 2427 | CTSD, CLU, DPP4, GDF15, GSN, MIF | NONE |
| 2428 | CTSD, CLU, DPP4, GDF15, GSN, PKM | NONE |
| 2429 | CTSD, CLU, DPP4, GDF15, GSN, TIMP1 | NONE |
| 2430 | CTSD, CLU, DPP4, GDF15, GSN, TFRC | NONE |
| 2431 | CTSD, CLU, DPP4, GDF15, MIF, PKM | NONE |
| 2432 | CTSD, CLU, DPP4, GDF15, MIF, TIMP1 | NONE |
| 2433 | CTSD, CLU, DPP4, GDF15, MIF, TFRC | NONE |
| 2434 | CTSD, CLU, DPP4, GDF15, PKM, TIMP1 | NONE |
| 2435 | CTSD, CLU, DPP4, GDF15, PKM, TFRC | NONE |
| 2436 | CTSD, CLU, DPP4, GDF15, TIMP1, TFRC | NONE |
| 2437 | CTSD, CLU, DPP4, GSN, MIF, PKM | NONE |
| 2438 | CTSD, CLU, DPP4, GSN, MIF, TIMP1 | NONE |
| 2439 | CTSD, CLU, DPP4, GSN, MIF, TFRC | NONE |
| 2440 | CTSD, CLU, DPP4, GSN, PKM, TIMP1 | NONE |
| 2441 | CTSD, CLU, DPP4, GSN, PKM, TFRC | NONE |
| 2442 | CTSD, CLU, DPP4, GSN, TIMP1, TFRC | NONE |
| 2443 | CTSD, CLU, DPP4, MIF, PKM, TIMP1 | NONE |
| 2444 | CTSD, CLU, DPP4, MIF, PKM, TFRC | NONE |
| 2445 | CTSD, CLU, DPP4, MIF, TIMP1, TFRC | NONE |
| 2446 | CTSD, CLU, DPP4, PKM, TIMP1, TFRC | NONE |
| 2447 | CTSD, CLU, GDF15, GSN, MIF, PKM | NONE |
| 2448 | CTSD, CLU, GDF15, GSN, MIF, TIMP1 | NONE |
| 2449 | CTSD, CLU, GDF15, GSN, MIF, TFRC | NONE |
| 2450 | CTSD, CLU, GDF15, GSN, PKM, TIMP1 | NONE |
| 2451 | CTSD, CLU, GDF15, GSN, PKM, TFRC | NONE |
| 2452 | CTSD, CLU, GDF15, GSN, TIMP1, TFRC | NONE |
| 2453 | CTSD, CLU, GDF15, MIF, PKM, TIMP1 | NONE |
| 2454 | CTSD, CLU, GDF15, MIF, PKM, TFRC | NONE |
| 2455 | CTSD, CLU, GDF15, MIF, TIMP1, TFRC | NONE |
| 2456 | CTSD, CLU, GDF15, PKM, TIMP1, TFRC | NONE |
| 2457 | CTSD, CLU, GSN, MIF, PKM, TIMP1 | NONE |
| 2458 | CTSD, CLU, GSN, MIF, PKM, TFRC | NONE |
| 2459 | CTSD, CLU, GSN, MIF, TIMP1, TFRC | NONE |
| 2460 | CTSD, CLU, GSN, PKM, TIMP1, TFRC | NONE |
| 2461 | CTSD, CLU, MIF, PKM, TIMP1, TFRC | NONE |
| 2462 | CTSD, DPP4, GDF15, GSN, MIF, PKM | NONE |
| 2463 | CTSD, DPP4, GDF15, GSN, MIF, TIMP1 | NONE |
| 2464 | CTSD, DPP4, GDF15, GSN, MIF, TFRC | NONE |
| 2465 | CTSD, DPP4, GDF15, GSN, PKM, TIMP1 | NONE |
| 2466 | CTSD, DPP4, GDF15, GSN, PKM, TFRC | NONE |
| 2467 | CTSD, DPP4, GDF15, GSN, TIMP1, TFRC | NONE |
| 2468 | CTSD, DPP4, GDF15, MIF, PKM, TIMP1 | NONE |
| 2469 | CTSD, DPP4, GDF15, MIF, PKM, TFRC | NONE |
| 2470 | CTSD, DPP4, GDF15, MIF, TIMP1, TFRC | NONE |
| 2471 | CTSD, DPP4, GDF15, PKM, TIMP1, TFRC | NONE |
| 2472 | CTSD, DPP4, GSN, MIF, PKM, TIMP1 | NONE |
| 2473 | CTSD, DPP4, GSN, MIF, PKM, TFRC | NONE |
| 2474 | CTSD, DPP4, GSN, MIF, TIMP1, TFRC | NONE |
| 2475 | CTSD, DPP4, GSN, PKM, TIMP1, TFRC | NONE |
| 2476 | CTSD, DPP4, MIF, PKM, TIMP1, TFRC | NONE |
| 2477 | CTSD, GDF15, GSN, MIF, PKM, TIMP1 | NONE |
| 2478 | CTSD, GDF15, GSN, MIF, PKM, TFRC | NONE |
| 2479 | CTSD, GDF15, GSN, MIF, TIMP1, TFRC | NONE |
| 2480 | CTSD, GDF15, GSN, PKM, TIMP1, TFRC | NONE |
| 2481 | CTSD, GDF15, MIF, PKM, TIMP1, TFRC | NONE |
| 2482 | CTSD, GSN, MIF, PKM, TIMP1, TFRC | NONE |
| 2483 | CLU, DPP4, GDF15, GSN, MIF, PKM | NONE |
| 2484 | CLU, DPP4, GDF15, GSN, MIF, TIMP1 | NONE |
| 2485 | CLU, DPP4, GDF15, GSN, MIF, TFRC | NONE |
| 2486 | CLU, DPP4, GDF15, GSN, PKM, TIMP1 | NONE |
| 2487 | CLU, DPP4, GDF15, GSN, PKM, TFRC | NONE |
| 2488 | CLU, DPP4, GDF15, GSN, TIMP1, TFRC | NONE |
| 2489 | CLU, DPP4, GDF15, MIF, PKM, TIMP1 | NONE |
| 2490 | CLU, DPP4, GDF15, MIF, PKM, TFRC | NONE |
| 2491 | CLU, DPP4, GDF15, MIF, TIMP1, TFRC | NONE |
| 2492 | CLU, DPP4, GDF15, PKM, TIMP1, TFRC | NONE |
| 2493 | CLU, DPP4, GSN, MIF, PKM, TIMP1 | NONE |
| 2494 | CLU, DPP4, GSN, MIF, PKM, TFRC | NONE |
| 2495 | CLU, DPP4, GSN, MIF, TIMP1, TFRC | NONE |
| 2496 | CLU, DPP4, GSN, PKM, TIMP1, TFRC | NONE |
| 2497 | CLU, DPP4, MIF, PKM, TIMP1, TFRC | NONE |
| 2498 | CLU, GDF15, GSN, MIF, PKM, TIMP1 | NONE |
| 2499 | CLU, GDF15, GSN, MIF, PKM, TFRC | NONE |
| 2500 | CLU, GDF15, GSN, MIF, TIMP1, TFRC | NONE |
| 2501 | CLU, GDF15, GSN, PKM, TIMP1, TFRC | NONE |
| 2502 | CLU, GDF15, MIF, PKM, TIMP1, TFRC | NONE |
| 2503 | CLU, GSN, MIF, PKM, TIMP1, TFRC | NONE |
| 2504 | DPP4, GDF15, GSN, MIF, PKM, TIMP1 | NONE |
| 2505 | DPP4, GDF15, GSN, MIF, PKM, TFRC | NONE |
| 2506 | DPP4, GDF15, GSN, MIF, TIMP1, TFRC | NONE |
| 2507 | DPP4, GDF15, GSN, PKM, TIMP1, TFRC | NONE |
| 2508 | DPP4, GDF15, MIF, PKM, TIMP1, TFRC | NONE |
| 2509 | DPP4, GSN, MIF, PKM, TIMP1, TFRC | NONE |
| 2510 | GDF15, GSN, MIF, PKM, TIMP1, TFRC | NONE |
| 2511 | SERPINA1, SERPINA3, CTSD, CLU | Age |
| 2512 | SERPINA1, SERPINA3, CTSD, DPP4 | Age |
| 2513 | SERPINA1, SERPINA3, CTSD, GDF15 | Age |
| 2514 | SERPINA1, SERPINA3, CTSD, GSN | Age |
| 2515 | SERPINA1, SERPINA3, CTSD, MIF | Age |
| 2516 | SERPINA1, SERPINA3, CTSD, PKM | Age |
| 2517 | SERPINA1, SERPINA3, CTSD, TIMP1 | Age |
| 2518 | SERPINA1, SERPINA3, CTSD, TFRC | Age |
| 2519 | SERPINA1, SERPINA3, CLU, DPP4 | Age |
| 2520 | SERPINA1, SERPINA3, CLU, GDF15 | Age |
| 2521 | SERPINA1, SERPINA3, CLU, GSN | Age |
| 2522 | SERPINA1, SERPINA3, CLU, MIF | Age |
| 2523 | SERPINA1, SERPINA3, CLU, PKM | Age |
| 2524 | SERPINA1, SERPINA3, CLU, TIMP1 | Age |
| 2525 | SERPINA1, SERPINA3, CLU, TFRC | Age |
| 2526 | SERPINA1, SERPINA3, DPP4, GDF15 | Age |
| 2527 | SERPINA1, SERPINA3, DPP4, GSN | Age |
| 2528 | SERPINA1, SERPINA3, DPP4, MIF | Age |
| 2529 | SERPINA1, SERPINA3, DPP4, PKM | Age |
| 2530 | SERPINA1, SERPINA3, DPP4, TIMP1 | Age |
| 2531 | SERPINA1, SERPINA3, DPP4, TFRC | Age |
| 2532 | SERPINA1, SERPINA3, GDF15, GSN | Age |
| 2533 | SERPINA1, SERPINA3, GDF15, MIF | Age |
| 2534 | SERPINA1, SERPINA3, GDF15, PKM | Age |
| 2535 | SERPINA1, SERPINA3, GDF15, TIMP1 | Age |
| 2536 | SERPINA1, SERPINA3, GDF15, TFRC | Age |
| 2537 | SERPINA1, SERPINA3, GSN, MIF | Age |
| 2538 | SERPINA1, SERPINA3, GSN, PKM | Age |
| 2539 | SERPINA1, SERPINA3, GSN, TIMP1 | Age |
| 2540 | SERPINA1, SERPINA3, GSN, TFRC | Age |
| 2541 | SERPINA1, SERPINA3, MIF, PKM | Age |
| 2542 | SERPINA1, SERPINA3, MIF, TIMP1 | Age |
| 2543 | SERPINA1, SERPINA3, MIF, TFRC | Age |
| 2544 | SERPINA1, SERPINA3, PKM, TIMP1 | Age |
| 2545 | SERPINA1, SERPINA3, PKM, TFRC | Age |
| 2546 | SERPINA1, SERPINA3, TIMP1, TFRC | Age |
| 2547 | SERPINA1, CTSD, CLU, DPP4 | Age |
| 2548 | SERPINA1, CTSD, CLU, GDF15 | Age |
| 2549 | SERPINA1, CTSD, CLU, GSN | Age |
| 2550 | SERPINA1, CTSD, CLU, MIF | Age |
| 2551 | SERPINA1, CTSD, CLU, PKM | Age |
| 2552 | SERPINA1, CTSD, CLU, TIMP1 | Age |
| 2553 | SERPINA1, CTSD, CLU, TFRC | Age |
| 2554 | SERPINA1, CTSD, DPP4, GDF15 | Age |
| 2555 | SERPINA1, CTSD, DPP4, GSN | Age |
| 2556 | SERPINA1, CTSD, DPP4, MIF | Age |
| 2557 | SERPINA1, CTSD, DPP4, PKM | Age |
| 2558 | SERPINA1, CTSD, DPP4, TIMP1 | Age |
| 2559 | SERPINA1, CTSD, DPP4, TFRC | Age |
| 2560 | SERPINA1, CTSD, GDF15, GSN | Age |
| 2561 | SERPINA1, CTSD, GDF15, MIF | Age |
| 2562 | SERPINA1, CTSD, GDF15, PKM | Age |
| 2563 | SERPINA1, CTSD, GDF15, TIMP1 | Age |
| 2564 | SERPINA1, CTSD, GDF15, TFRC | Age |
| 2565 | SERPINA1, CTSD, GSN, MIF | Age |
| 2566 | SERPINA1, CTSD, GSN, PKM | Age |
| 2567 | SERPINA1, CTSD, GSN, TIMP1 | Age |
| 2568 | SERPINA1, CTSD, GSN, TFRC | Age |
| 2569 | SERPINA1, CTSD, MIF, PKM | Age |
| 2570 | SERPINA1, CTSD, MIF, TIMP1 | Age |
| 2571 | SERPINA1, CTSD, MIF, TFRC | Age |
| 2572 | SERPINA1, CTSD, PKM, TIMP1 | Age |
| 2573 | SERPINA1, CTSD, PKM, TFRC | Age |
| 2574 | SERPINA1, CTSD, TIMP1, TFRC | Age |
| 2575 | SERPINA1, CLU, DPP4, GDF15 | Age |
| 2576 | SERPINA1, CLU, DPP4, GSN | Age |
| 2577 | SERPINA1, CLU, DPP4, MIF | Age |
| 2578 | SERPINA1, CLU, DPP4, PKM | Age |
| 2579 | SERPINA1, CLU, DPP4, TIMP1 | Age |
| 2580 | SERPINA1, CLU, DPP4, TFRC | Age |
| 2581 | SERPINA1, CLU, GDF15, GSN | Age |
| 2582 | SERPINA1, CLU, GDF15, MIF | Age |
| 2583 | SERPINA1, CLU, GDF15, PKM | Age |
| 2584 | SERPINA1, CLU, GDF15, TIMP1 | Age |
| 2585 | SERPINA1, CLU, GDF15, TFRC | Age |
| 2586 | SERPINA1, CLU, GSN, MIF | Age |
| 2587 | SERPINA1, CLU, GSN, PKM | Age |
| 2588 | SERPINA1, CLU, GSN, TIMP1 | Age |
| 2589 | SERPINA1, CLU, GSN, TFRC | Age |
| 2590 | SERPINA1, CLU, MIF, PKM | Age |
| 2591 | SERPINA1, CLU, MIF, TIMP1 | Age |
| 2592 | SERPINA1, CLU, MIF, TFRC | Age |
| 2593 | SERPINA1, CLU, PKM, TIMP1 | Age |
| 2594 | SERPINA1, CLU, PKM, TFRC | Age |
| 2595 | SERPINA1, CLU, TIMP1, TFRC | Age |
| 2596 | SERPINA1, DPP4, GDF15, GSN | Age |
| 2597 | SERPINA1, DPP4, GDF15, MIF | Age |
| 2598 | SERPINA1, DPP4, GDF15, PKM | Age |
| 2599 | SERPINA1, DPP4, GDF15, TIMP1 | Age |
| 2600 | SERPINA1, DPP4, GDF15, TFRC | Age |
| 2601 | SERPINA1, DPP4, GSN, MIF | Age |
| 2602 | SERPINA1, DPP4, GSN, PKM | Age |
| 2603 | SERPINA1, DPP4, GSN, TIMP1 | Age |
| 2604 | SERPINA1, DPP4, GSN, TFRC | Age |
| 2605 | SERPINA1, DPP4, MIF, PKM | Age |
| 2606 | SERPINA1, DPP4, MIF, TIMP1 | Age |
| 2607 | SERPINA1, DPP4, MIF, TFRC | Age |
| 2608 | SERPINA1, DPP4, PKM, TIMP1 | Age |
| 2609 | SERPINA1, DPP4, PKM, TFRC | Age |
| 2610 | SERPINA1, DPP4, TIMP1, TFRC | Age |
| 2611 | SERPINA1, GDF15, GSN, MIF | Age |
| 2612 | SERPINA1, GDF15, GSN, PKM | Age |
| 2613 | SERPINA1, GDF15, GSN, TIMP1 | Age |
| 2614 | SERPINA1, GDF15, GSN, TFRC | Age |
| 2615 | SERPINA1, GDF15, MIF, PKM | Age |
| 2616 | SERPINA1, GDF15, MIF, TIMP1 | Age |
| 2617 | SERPINA1, GDF15, MIF, TFRC | Age |
| 2618 | SERPINA1, GDF15, PKM, TIMP1 | Age |
| 2619 | SERPINA1, GDF15, PKM, TFRC | Age |
| 2620 | SERPINA1, GDF15, TIMP1, TFRC | Age |
| 2621 | SERPINA1, GSN, MIF, PKM | Age |
| 2622 | SERPINA1, GSN, MIF, TIMP1 | Age |
| 2623 | SERPINA1, GSN, MIF, TFRC | Age |
| 2624 | SERPINA1, GSN, PKM, TIMP1 | Age |
| 2625 | SERPINA1, GSN, PKM, TFRC | Age |
| 2626 | SERPINA1, GSN, TIMP1, TFRC | Age |
| 2627 | SERPINA1, MIF, PKM, TIMP1 | Age |
| 2628 | SERPINA1, MIF, PKM, TFRC | Age |
| 2629 | SERPINA1, MIF, TIMP1, TFRC | Age |
| 2630 | SERPINA1, PKM, TIMP1, TFRC | Age |
| 2631 | SERPINA3, CTSD, CLU, DPP4 | Age |
| 2632 | SERPINA3, CTSD, CLU, GDF15 | Age |
| 2633 | SERPINA3, CTSD, CLU, GSN | Age |
| 2634 | SERPINA3, CTSD, CLU, MIF | Age |
| 2635 | SERPINA3, CTSD, CLU, PKM | Age |
| 2636 | SERPINA3, CTSD, CLU, TIMP1 | Age |
| 2637 | SERPINA3, CTSD, CLU, TFRC | Age |
| 2638 | SERPINA3, CTSD, DPP4, GDF15 | Age |
| 2639 | SERPINA3, CTSD, DPP4, GSN | Age |
| 2640 | SERPINA3, CTSD, DPP4, MIF | Age |
| 2641 | SERPINA3, CTSD, DPP4, PKM | Age |
| 2642 | SERPINA3, CTSD, DPP4, TIMP1 | Age |
| 2643 | SERPINA3, CTSD, DPP4, TFRC | Age |
| 2644 | SERPINA3, CTSD, GDF15, GSN | Age |
| 2645 | SERPINA3, CTSD, GDF15, MIF | Age |
| 2646 | SERPINA3, CTSD, GDF15, PKM | Age |
| 2647 | SERPINA3, CTSD, GDF15, TIMP1 | Age |
| 2648 | SERPINA3, CTSD, GDF15, TFRC | Age |
| 2649 | SERPINA3, CTSD, GSN, MIF | Age |
| 2650 | SERPINA3, CTSD, GSN, PKM | Age |
| 2651 | SERPINA3, CTSD, GSN, TIMP1 | Age |
| 2652 | SERPINA3, CTSD, GSN, TFRC | Age |
| 2653 | SERPINA3, CTSD, MIF, PKM | Age |
| 2654 | SERPINA3, CTSD, MIF, TIMP1 | Age |
| 2655 | SERPINA3, CTSD, MIF, TFRC | Age |
| 2656 | SERPINA3, CTSD, PKM, TIMP1 | Age |
| 2657 | SERPINA3, CTSD, PKM, TFRC | Age |
| 2658 | SERPINA3, CTSD, TIMP1, TFRC | Age |
| 2659 | SERPINA3, CLU, DPP4, GDF15 | Age |
| 2660 | SERPINA3, CLU, DPP4, GSN | Age |
| 2661 | SERPINA3, CLU, DPP4, MIF | Age |
| 2662 | SERPINA3, CLU, DPP4, PKM | Age |
| 2663 | SERPINA3, CLU, DPP4, TIMP1 | Age |
| 2664 | SERPINA3, CLU, DPP4, TFRC | Age |
| 2665 | SERPINA3, CLU, GDF15, GSN | Age |
| 2666 | SERPINA3, CLU, GDF15, MIF | Age |
| 2667 | SERPINA3, CLU, GDF15, PKM | Age |
| 2668 | SERPINA3, CLU, GDF15, TIMP1 | Age |
| 2669 | SERPINA3, CLU, GDF15, TFRC | Age |
| 2670 | SERPINA3, CLU, GSN, MIF | Age |
| 2671 | SERPINA3, CLU, GSN, PKM | Age |
| 2672 | SERPINA3, CLU, GSN, TIMP1 | Age |
| 2673 | SERPINA3, CLU, GSN, TFRC | Age |
| 2674 | SERPINA3, CLU, MIF, PKM | Age |
| 2675 | SERPINA3, CLU, MIF, TIMP1 | Age |
| 2676 | SERPINA3, CLU, MIF, TFRC | Age |
| 2677 | SERPINA3, CLU, PKM, TIMP1 | Age |
| 2678 | SERPINA3, CLU, PKM, TFRC | Age |
| 2679 | SERPINA3, CLU, TIMP1, TFRC | Age |
| 2680 | SERPINA3, DPP4, GDF15, GSN | Age |
| 2681 | SERPINA3, DPP4, GDF15, MIF | Age |
| 2682 | SERPINA3, DPP4, GDF15, PKM | Age |
| 2683 | SERPINA3, DPP4, GDF15, TIMP1 | Age |
| 2684 | SERPINA3, DPP4, GDF15, TFRC | Age |
| 2685 | SERPINA3, DPP4, GSN, MIF | Age |
| 2686 | SERPINA3, DPP4, GSN, PKM | Age |
| 2687 | SERPINA3, DPP4, GSN, TIMP1 | Age |
| 2688 | SERPINA3, DPP4, GSN, TFRC | Age |
| 2689 | SERPINA3, DPP4, MIF, PKM | Age |
| 2690 | SERPINA3, DPP4, MIF, TIMP1 | Age |
| 2691 | SERPINA3, DPP4, MIF, TFRC | Age |
| 2692 | SERPINA3, DPP4, PKM, TIMP1 | Age |
| 2693 | SERPINA3, DPP4, PKM, TFRC | Age |
| 2694 | SERPINA3, DPP4, TIMP1, TFRC | Age |
| 2695 | SERPINA3, GDF15, GSN, MIF | Age |
| 2696 | SERPINA3, GDF15, GSN, PKM | Age |
| 2697 | SERPINA3, GDF15, GSN, TIMP1 | Age |
| 2698 | SERPINA3, GDF15, GSN, TFRC | Age |
| 2699 | SERPINA3, GDF15, MIF, PKM | Age |
| 2700 | SERPINA3, GDF15, MIF, TIMP1 | Age |
| 2701 | SERPINA3, GDF15, MIF, TFRC | Age |
| 2702 | SERPINA3, GDF15, PKM, TIMP1 | Age |
| 2703 | SERPINA3, GDF15, PKM, TFRC | Age |
| 2704 | SERPINA3, GDF15, TIMP1, TFRC | Age |
| 2705 | SERPINA3, GSN, MIF, PKM | Age |
| 2706 | SERPINA3, GSN, MIF, TIMP1 | Age |
| 2707 | SERPINA3, GSN, MIF, TFRC | Age |
| 2708 | SERPINA3, GSN, PKM, TIMP1 | Age |
| 2709 | SERPINA3, GSN, PKM, TFRC | Age |
| 2710 | SERPINA3, GSN, TIMP1, TFRC | Age |
| 2711 | SERPINA3, MIF, PKM, TIMP1 | Age |
| 2712 | SERPINA3, MIF, PKM, TFRC | Age |
| 2713 | SERPINA3, MIF, TIMP1, TFRC | Age |
| 2714 | SERPINA3, PKM, TIMP1, TFRC | Age |
| 2715 | CTSD, CLU, DPP4, GDF15 | Age |
| 2716 | CTSD, CLU, DPP4, GSN | Age |
| 2717 | CTSD, CLU, DPP4, MIF | Age |
| 2718 | CTSD, CLU, DPP4, PKM | Age |
| 2719 | CTSD, CLU, DPP4, TIMP1 | Age |
| 2720 | CTSD, CLU, DPP4, TFRC | Age |
| 2721 | CTSD, CLU, GDF15, GSN | Age |
| 2722 | CTSD, CLU, GDF15, MIF | Age |
| 2723 | CTSD, CLU, GDF15, PKM | Age |
| 2724 | CTSD, CLU, GDF15, TIMP1 | Age |
| 2725 | CTSD, CLU, GDF15, TFRC | Age |
| 2726 | CTSD, CLU, GSN, MIF | Age |
| 2727 | CTSD, CLU, GSN, PKM | Age |
| 2728 | CTSD, CLU, GSN, TIMP1 | Age |
| 2729 | CTSD, CLU, GSN, TFRC | Age |
| 2730 | CTSD, CLU, MIF, PKM | Age |
| 2731 | CTSD, CLU, MIF, TIMP1 | Age |
| 2732 | CTSD, CLU, MIF, TFRC | Age |
| 2733 | CTSD, CLU, PKM, TIMP1 | Age |
| 2734 | CTSD, CLU, PKM, TFRC | Age |
| 2735 | CTSD, CLU, TIMP1, TFRC | Age |
| 2736 | CTSD, DPP4, GDF15, GSN | Age |
| 2737 | CTSD, DPP4, GDF15, MIF | Age |
| 2738 | CTSD, DPP4, GDF15, PKM | Age |
| 2739 | CTSD, DPP4, GDF15, TIMP1 | Age |
| 2740 | CTSD, DPP4, GDF15, TFRC | Age |
| 2741 | CTSD, DPP4, GSN, MIF | Age |
| 2742 | CTSD, DPP4, GSN, PKM | Age |
| 2743 | CTSD, DPP4, GSN, TIMP1 | Age |
| 2744 | CTSD, DPP4, GSN, TFRC | Age |
| 2745 | CTSD, DPP4, MIF, PKM | Age |
| 2746 | CTSD, DPP4, MIF, TIMP1 | Age |
| 2747 | CTSD, DPP4, MIF, TFRC | Age |
| 2748 | CTSD, DPP4, PKM, TIMP1 | Age |
| 2749 | CTSD, DPP4, PKM, TFRC | Age |
| 2750 | CTSD, DPP4, TIMP1, TFRC | Age |
| 2751 | CTSD, GDF15, GSN, MIF | Age |
| 2752 | CTSD, GDF15, GSN, PKM | Age |
| 2753 | CTSD, GDF15, GSN, TIMP1 | Age |
| 2754 | CTSD, GDF15, GSN, TFRC | Age |
| 2755 | CTSD, GDF15, MIF, PKM | Age |
| 2756 | CTSD, GDF15, MIF, TIMP1 | Age |
| 2757 | CTSD, GDF15, MIF, TFRC | Age |
| 2758 | CTSD, GDF15, PKM, TIMP1 | Age |
| 2759 | CTSD, GDF15, PKM, TFRC | Age |
| 2760 | CTSD, GDF15, TIMP1, TFRC | Age |
| 2761 | CTSD, GSN, MIF, PKM | Age |
| 2762 | CTSD, GSN, MIF, TIMP1 | Age |
| 2763 | CTSD, GSN, MIF, TFRC | Age |
| 2764 | CTSD, GSN, PKM, TIMP1 | Age |
| 2765 | CTSD, GSN, PKM, TFRC | Age |
| 2766 | CTSD, GSN, TIMP1, TFRC | Age |
| 2767 | CTSD, MIF, PKM, TIMP1 | Age |
| 2768 | CTSD, MIF, PKM, TFRC | Age |
| 2769 | CTSD, MIF, TIMP1, TFRC | Age |
| 2770 | CTSD, PKM, TIMP1, TFRC | Age |
| 2771 | CLU, DPP4, GDF15, GSN | Age |
| 2772 | CLU, DPP4, GDF15, MIF | Age |
| 2773 | CLU, DPP4, GDF15, PKM | Age |
| 2774 | CLU, DPP4, GDF15, TIMP1 | Age |
| 2775 | CLU, DPP4, GDF15, TFRC | Age |
| 2776 | CLU, DPP4, GSN, MIF | Age |
| 2777 | CLU, DPP4, GSN, PKM | Age |
| 2778 | CLU, DPP4, GSN, TIMP1 | Age |
| 2779 | CLU, DPP4, GSN, TFRC | Age |
| 2780 | CLU, DPP4, MIF, PKM | Age |
| 2781 | CLU, DPP4, MIF, TIMP1 | Age |
| 2782 | CLU, DPP4, MIF, TFRC | Age |
| 2783 | CLU, DPP4, PKM, TIMP1 | Age |
| 2784 | CLU, DPP4, PKM, TFRC | Age |
| 2785 | CLU, DPP4, TIMP1, TFRC | Age |
| 2786 | CLU, GDF15, GSN, MIF | Age |
| 2787 | CLU, GDF15, GSN, PKM | Age |
| 2788 | CLU, GDF15, GSN, TIMP1 | Age |
| 2789 | CLU, GDF15, GSN, TFRC | Age |
| 2790 | CLU, GDF15, MIF, PKM | Age |
| 2791 | CLU, GDF15, MIF, TIMP1 | Age |
| 2792 | CLU, GDF15, MIF, TFRC | Age |
| 2793 | CLU, GDF15, PKM, TIMP1 | Age |
| 2794 | CLU, GDF15, PKM, TFRC | Age |
| 2795 | CLU, GDF15, TIMP1, TFRC | Age |
| 2796 | CLU, GSN, MIF, PKM | Age |
| 2797 | CLU, GSN, MIF, TIMP1 | Age |
| 2798 | CLU, GSN, MIF, TFRC | Age |
| 2799 | CLU, GSN, PKM, TIMP1 | Age |
| 2800 | CLU, GSN, PKM, TFRC | Age |
| 2801 | CLU, GSN, TIMP1, TFRC | Age |
| 2802 | CLU, MIF, PKM, TIMP1 | Age |
| 2803 | CLU, MIF, PKM, TFRC | Age |
| 2804 | CLU, MIF, TIMP1, TFRC | Age |
| 2805 | CLU, PKM, TIMP1, TFRC | Age |
| 2806 | DPP4, GDF15, GSN, MIF | Age |
| 2807 | DPP4, GDF15, GSN, PKM | Age |
| 2808 | DPP4, GDF15, GSN, TIMP1 | Age |
| 2809 | DPP4, GDF15, GSN, TFRC | Age |
| 2810 | DPP4, GDF15, MIF, PKM | Age |
| 2811 | DPP4, GDF15, MIF, TIMP1 | Age |
| 2812 | DPP4, GDF15, MIF, TFRC | Age |
| 2813 | DPP4, GDF15, PKM, TIMP1 | Age |
| 2814 | DPP4, GDF15, PKM, TFRC | Age |
| 2815 | DPP4, GDF15, TIMP1, TFRC | Age |
| 2816 | DPP4, GSN, MIF, PKM | Age |
| 2817 | DPP4, GSN, MIF, TIMP1 | Age |
| 2818 | DPP4, GSN, MIF, TFRC | Age |
| 2819 | DPP4, GSN, PKM, TIMP1 | Age |
| 2820 | DPP4, GSN, PKM, TFRC | Age |
| 2821 | DPP4, GSN, TIMP1, TFRC | Age |
| 2822 | DPP4, MIF, PKM, TIMP1 | Age |
| 2823 | DPP4, MIF, PKM, TFRC | Age |
| 2824 | DPP4, MIF, TIMP1, TFRC | Age |
| 2825 | DPP4, PKM, TIMP1, TFRC | Age |
| 2826 | GDF15, GSN, MIF, PKM | Age |
| 2827 | GDF15, GSN, MIF, TIMP1 | Age |
| 2828 | GDF15, GSN, MIF, TFRC | Age |
| 2829 | GDF15, GSN, PKM, TIMP1 | Age |
| 2830 | GDF15, GSN, PKM, TFRC | Age |
| 2831 | GDF15, GSN, TIMP1, TFRC | Age |
| 2832 | GDF15, MIF, PKM, TIMP1 | Age |
| 2833 | GDF15, MIF, PKM, TFRC | Age |
| 2834 | GDF15, MIF, TIMP1, TFRC | Age |
| 2835 | GDF15, PKM, TIMP1, TFRC | Age |
| 2836 | GSN, MIF, PKM, TIMP1 | Age |
| 2837 | GSN, MIF, PKM, TFRC | Age |
| 2838 | GSN, MIF, TIMP1, TFRC | Age |
| 2839 | GSN, PKM, TIMP1, TFRC | Age |
| 2840 | MIF, PKM, TIMP1, TFRC | Age |
| 2841 | SERPINA1, SERPINA3, CTSD, CLU, DPP4 | NONE |
| 2842 | SERPINA1, SERPINA3, CTSD, CLU, GDF15 | NONE |
| 2843 | SERPINA1, SERPINA3, CTSD, CLU, GSN | NONE |
| 2844 | SERPINA1, SERPINA3, CTSD, CLU, MIF | NONE |
| 2845 | SERPINA1, SERPINA3, CTSD, CLU, PKM | NONE |
| 2846 | SERPINA1, SERPINA3, CTSD, CLU, TIMP1 | NONE |
| 2847 | SERPINA1, SERPINA3, CTSD, CLU, TFRC | NONE |
| 2848 | SERPINA1, SERPINA3, CTSD, DPP4, GDF15 | NONE |
| 2849 | SERPINA1, SERPINA3, CTSD, DPP4, GSN | NONE |
| 2850 | SERPINA1, SERPINA3, CTSD, DPP4, MIF | NONE |
| 2851 | SERPINA1, SERPINA3, CTSD, DPP4, PKM | NONE |
| 2852 | SERPINA1, SERPINA3, CTSD, DPP4, TIMP1 | NONE |
| 2853 | SERPINA1, SERPINA3, CTSD, DPP4, TFRC | NONE |
| 2854 | SERPINA1, SERPINA3, CTSD, GDF15, GSN | NONE |
| 2855 | SERPINA1, SERPINA3, CTSD, GDF15, MIF | NONE |
| 2856 | SERPINA1, SERPINA3, CTSD, GDF15, PKM | NONE |
| 2857 | SERPINA1, SERPINA3, CTSD, GDF15, TIMP1 | NONE |
| 2858 | SERPINA1, SERPINA3, CTSD, GDF15, TFRC | NONE |
| 2859 | SERPINA1, SERPINA3, CTSD, GSN, MIF | NONE |
| 2860 | SERPINA1, SERPINA3, CTSD, GSN, PKM | NONE |
| 2861 | SERPINA1, SERPINA3, CTSD, GSN, TIMP1 | NONE |
| 2862 | SERPINA1, SERPINA3, CTSD, GSN, TFRC | NONE |
| 2863 | SERPINA1, SERPINA3, CTSD, MIF, PKM | NONE |
| 2864 | SERPINA1, SERPINA3, CTSD, MIF, TIMP1 | NONE |
| 2865 | SERPINA1, SERPINA3, CTSD, MIF, TFRC | NONE |
| 2866 | SERPINA1, SERPINA3, CTSD, PKM, TIMP1 | NONE |
| 2867 | SERPINA1, SERPINA3, CTSD, PKM, TFRC | NONE |
| 2868 | SERPINA1, SERPINA3, CTSD, TIMP1, TFRC | NONE |
| 2869 | SERPINA1, SERPINA3, CLU, DPP4, GDF15 | NONE |
| 2870 | SERPINA1, SERPINA3, CLU, DPP4, GSN | NONE |
| 2871 | SERPINA1, SERPINA3, CLU, DPP4, MIF | NONE |
| 2872 | SERPINA1, SERPINA3, CLU, DPP4, PKM | NONE |
| 2873 | SERPINA1, SERPINA3, CLU, DPP4, TIMP1 | NONE |
| 2874 | SERPINA1, SERPINA3, CLU, DPP4, TFRC | NONE |
| 2875 | SERPINA1, SERPINA3, CLU, GDF15, GSN | NONE |
| 2876 | SERPINA1, SERPINA3, CLU, GDF15, MIF | NONE |
| 2877 | SERPINA1, SERPINA3, CLU, GDF15, PKM | NONE |
| 2878 | SERPINA1, SERPINA3, CLU, GDF15, TIMP1 | NONE |
| 2879 | SERPINA1, SERPINA3, CLU, GDF15, TFRC | NONE |
| 2880 | SERPINA1, SERPINA3, CLU, GSN, MIF | NONE |
| 2881 | SERPINA1, SERPINA3, CLU, GSN, PKM | NONE |
| 2882 | SERPINA1, SERPINA3, CLU, GSN, TIMP1 | NONE |
| 2883 | SERPINA1, SERPINA3, CLU, GSN, TFRC | NONE |
| 2884 | SERPINA1, SERPINA3, CLU, MIF, PKM | NONE |
| 2885 | SERPINA1, SERPINA3, CLU, MIF, TIMP1 | NONE |
| 2886 | SERPINA1, SERPINA3, CLU, MIF, TFRC | NONE |
| 2887 | SERPINA1, SERPINA3, CLU, PKM, TIMP1 | NONE |
| 2888 | SERPINA1, SERPINA3, CLU, PKM, TFRC | NONE |
| 2889 | SERPINA1, SERPINA3, CLU, TIMP1, TFRC | NONE |
| 2890 | SERPINA1, SERPINA3, DPP4, GDF15, GSN | NONE |
| 2891 | SERPINA1, SERPINA3, DPP4, GDF15, MIF | NONE |
| 2892 | SERPINA1, SERPINA3, DPP4, GDF15, PKM | NONE |
| 2893 | SERPINA1, SERPINA3, DPP4, GDF15, TIMP1 | NONE |
| 2894 | SERPINA1, SERPINA3, DPP4, GDF15, TFRC | NONE |
| 2895 | SERPINA1, SERPINA3, DPP4, GSN, MIF | NONE |
| 2896 | SERPINA1, SERPINA3, DPP4, GSN, PKM | NONE |
| 2897 | SERPINA1, SERPINA3, DPP4, GSN, TIMP1 | NONE |
| 2898 | SERPINA1, SERPINA3, DPP4, GSN, TFRC | NONE |
| 2899 | SERPINA1, SERPINA3, DPP4, MIF, PKM | NONE |
| 2900 | SERPINA1, SERPINA3, DPP4, MIF, TIMP1 | NONE |
| 2901 | SERPINA1, SERPINA3, DPP4, MIF, TFRC | NONE |
| 2902 | SERPINA1, SERPINA3, DPP4, PKM, TIMP1 | NONE |
| 2903 | SERPINA1, SERPINA3, DPP4, PKM, TFRC | NONE |
| 2904 | SERPINA1, SERPINA3, DPP4, TIMP1, TFRC | NONE |
| 2905 | SERPINA1, SERPINA3, GDF15, GSN, MIF | NONE |
| 2906 | SERPINA1, SERPINA3, GDF15, GSN, PKM | NONE |
| 2907 | SERPINA1, SERPINA3, GDF15, GSN, TIMP1 | NONE |
| 2908 | SERPINA1, SERPINA3, GDF15, GSN, TFRC | NONE |
| 2909 | SERPINA1, SERPINA3, GDF15, MIF, PKM | NONE |
| 2910 | SERPINA1, SERPINA3, GDF15, MIF, TIMP1 | NONE |
| 2911 | SERPINA1, SERPINA3, GDF15, MIF, TFRC | NONE |
| 2912 | SERPINA1, SERPINA3, GDF15, PKM, TIMP1 | NONE |
| 2913 | SERPINA1, SERPINA3, GDF15, PKM, TFRC | NONE |
| 2914 | SERPINA1, SERPINA3, GDF15, TIMP1, TFRC | NONE |
| 2915 | SERPINA1, SERPINA3, GSN, MIF, PKM | NONE |
| 2916 | SERPINA1, SERPINA3, GSN, MIF, TIMP1 | NONE |
| 2917 | SERPINA1, SERPINA3, GSN, MIF, TFRC | NONE |
| 2918 | SERPINA1, SERPINA3, GSN, PKM, TIMP1 | NONE |
| 2919 | SERPINA1, SERPINA3, GSN, PKM, TFRC | NONE |
| 2920 | SERPINA1, SERPINA3, GSN, TIMP1, TFRC | NONE |
| 2921 | SERPINA1, SERPINA3, MIF, PKM, TIMP1 | NONE |
| 2922 | SERPINA1, SERPINA3, MIF, PKM, TFRC | NONE |
| 2923 | SERPINA1, SERPINA3, MIF, TIMP1, TFRC | NONE |
| 2924 | SERPINA1, SERPINA3, PKM, TIMP1, TFRC | NONE |
| 2925 | SERPINA1, CTSD, CLU, DPP4, GDF15 | NONE |
| 2926 | SERPINA1, CTSD, CLU, DPP4, GSN | NONE |
| 2927 | SERPINA1, CTSD, CLU, DPP4, MIF | NONE |
| 2928 | SERPINA1, CTSD, CLU, DPP4, PKM | NONE |
| 2929 | SERPINA1, CTSD, CLU, DPP4, TIMP1 | NONE |
| 2930 | SERPINA1, CTSD, CLU, DPP4, TFRC | NONE |
| 2931 | SERPINA1, CTSD, CLU, GDF15, GSN | NONE |
| 2932 | SERPINA1, CTSD, CLU, GDF15, MIF | NONE |
| 2933 | SERPINA1, CTSD, CLU, GDF15, PKM | NONE |
| 2934 | SERPINA1, CTSD, CLU, GDF15, TIMP1 | NONE |
| 2935 | SERPINA1, CTSD, CLU, GDF15, TFRC | NONE |
| 2936 | SERPINA1, CTSD, CLU, GSN, MIF | NONE |
| 2937 | SERPINA1, CTSD, CLU, GSN, PKM | NONE |
| 2938 | SERPINA1, CTSD, CLU, GSN, TIMP1 | NONE |
| 2939 | SERPINA1, CTSD, CLU, GSN, TFRC | NONE |
| 2940 | SERPINA1, CTSD, CLU, MIF, PKM | NONE |
| 2941 | SERPINA1, CTSD, CLU, MIF, TIMP1 | NONE |
| 2942 | SERPINA1, CTSD, CLU, MIF, TFRC | NONE |
| 2943 | SERPINA1, CTSD, CLU, PKM, TIMP1 | NONE |
| 2944 | SERPINA1, CTSD, CLU, PKM, TFRC | NONE |
| 2945 | SERPINA1, CTSD, CLU, TIMP1, TFRC | NONE |
| 2946 | SERPINA1, CTSD, DPP4, GDF15, GSN | NONE |
| 2947 | SERPINA1, CTSD, DPP4, GDF15, MIF | NONE |
| 2948 | SERPINA1, CTSD, DPP4, GDF15, PKM | NONE |
| 2949 | SERPINA1, CTSD, DPP4, GDF15, TIMP1 | NONE |
| 2950 | SERPINA1, CTSD, DPP4, GDF15, TFRC | NONE |
| 2951 | SERPINA1, CTSD, DPP4, GSN, MIF | NONE |
| 2952 | SERPINA1, CTSD, DPP4, GSN, PKM | NONE |
| 2953 | SERPINA1, CTSD, DPP4, GSN, TIMP1 | NONE |
| 2954 | SERPINA1, CTSD, DPP4, GSN, TFRC | NONE |
| 2955 | SERPINA1, CTSD, DPP4, MIF, PKM | NONE |
| 2956 | SERPINA1, CTSD, DPP4, MIF, TIMP1 | NONE |
| 2957 | SERPINA1, CTSD, DPP4, MIF, TFRC | NONE |
| 2958 | SERPINA1, CTSD, DPP4, PKM, TIMP1 | NONE |
| 2959 | SERPINA1, CTSD, DPP4, PKM, TFRC | NONE |
| 2960 | SERPINA1, CTSD, DPP4, TIMP1, TFRC | NONE |
| 2961 | SERPINA1, CTSD, GDF15, GSN, MIF | NONE |
| 2962 | SERPINA1, CTSD, GDF15, GSN, PKM | NONE |
| 2963 | SERPINA1, CTSD, GDF15, GSN, TIMP1 | NONE |
| 2964 | SERPINA1, CTSD, GDF15, GSN, TFRC | NONE |
| 2965 | SERPINA1, CTSD, GDF15, MIF, PKM | NONE |
| 2966 | SERPINA1, CTSD, GDF15, MIF, TIMP1 | NONE |
| 2967 | SERPINA1, CTSD, GDF15, MIF, TFRC | NONE |
| 2968 | SERPINA1, CTSD, GDF15, PKM, TIMP1 | NONE |
| 2969 | SERPINA1, CTSD, GDF15, PKM, TFRC | NONE |
| 2970 | SERPINA1, CTSD, GDF15, TIMP1, TFRC | NONE |
| 2971 | SERPINA1, CTSD, GSN, MIF, PKM | NONE |
| 2972 | SERPINA1, CTSD, GSN, MIF, TIMP1 | NONE |
| 2973 | SERPINA1, CTSD, GSN, MIF, TFRC | NONE |
| 2974 | SERPINA1, CTSD, GSN, PKM, TIMP1 | NONE |
| 2975 | SERPINA1, CTSD, GSN, PKM, TFRC | NONE |
| 2976 | SERPINA1, CTSD, GSN, TIMP1, TFRC | NONE |
| 2977 | SERPINA1, CTSD, MIF, PKM, TIMP1 | NONE |
| 2978 | SERPINA1, CTSD, MIF, PKM, TFRC | NONE |
| 2979 | SERPINA1, CTSD, MIF, TIMP1, TFRC | NONE |
| 2980 | SERPINA1, CTSD, PKM, TIMP1, TFRC | NONE |
| 2981 | SERPINA1, CLU, DPP4, GDF15, GSN | NONE |
| 2982 | SERPINA1, CLU, DPP4, GDF15, MIF | NONE |
| 2983 | SERPINA1, CLU, DPP4, GDF15, PKM | NONE |
| 2984 | SERPINA1, CLU, DPP4, GDF15, TIMP1 | NONE |
| 2985 | SERPINA1, CLU, DPP4, GDF15, TFRC | NONE |
| 2986 | SERPINA1, CLU, DPP4, GSN, MIF | NONE |
| 2987 | SERPINA1, CLU, DPP4, GSN, PKM | NONE |
| 2988 | SERPINA1, CLU, DPP4, GSN, TIMP1 | NONE |
| 2989 | SERPINA1, CLU, DPP4, GSN, TFRC | NONE |
| 2990 | SERPINA1, CLU, DPP4, MIF, PKM | NONE |
| 2991 | SERPINA1, CLU, DPP4, MIF, TIMP1 | NONE |
| 2992 | SERPINA1, CLU, DPP4, MIF, TFRC | NONE |
| 2993 | SERPINA1, CLU, DPP4, PKM, TIMP1 | NONE |
| 2994 | SERPINA1, CLU, DPP4, PKM, TFRC | NONE |
| 2995 | SERPINA1, CLU, DPP4, TIMP1, TFRC | NONE |
| 2996 | SERPINA1, CLU, GDF15, GSN, MIF | NONE |
| 2997 | SERPINA1, CLU, GDF15, GSN, PKM | NONE |
| 2998 | SERPINA1, CLU, GDF15, GSN, TIMP1 | NONE |
| 2999 | SERPINA1, CLU, GDF15, GSN, TFRC | NONE |
| 3000 | SERPINA1, CLU, GDF15, MIF, PKM | NONE |
| 3001 | SERPINA1, CLU, GDF15, MIF, TIMP1 | NONE |
| 3002 | SERPINA1, CLU, GDF15, MIF, TFRC | NONE |
| 3003 | SERPINA1, CLU, GDF15, PKM, TIMP1 | NONE |
| 3004 | SERPINA1, CLU, GDF15, PKM, TFRC | NONE |
| 3005 | SERPINA1, CLU, GDF15, TIMP1, TFRC | NONE |
| 3006 | SERPINA1, CLU, GSN, MIF, PKM | NONE |
| 3007 | SERPINA1, CLU, GSN, MIF, TIMP1 | NONE |
| 3008 | SERPINA1, CLU, GSN, MIF, TFRC | NONE |
| 3009 | SERPINA1, CLU, GSN, PKM, TIMP1 | NONE |
| 3010 | SERPINA1, CLU, GSN, PKM, TFRC | NONE |
| 3011 | SERPINA1, CLU, GSN, TIMP1, TFRC | NONE |
| 3012 | SERPINA1, CLU, MIF, PKM, TIMP1 | NONE |
| 3013 | SERPINA1, CLU, MIF, PKM, TFRC | NONE |
| 3014 | SERPINA1, CLU, MIF, TIMP1, TFRC | NONE |
| 3015 | SERPINA1, CLU, PKM, TIMP1, TFRC | NONE |
| 3016 | SERPINA1, DPP4, GDF15, GSN, MIF | NONE |
| 3017 | SERPINA1, DPP4, GDF15, GSN, PKM | NONE |
| 3018 | SERPINA1, DPP4, GDF15, GSN, TIMP1 | NONE |
| 3019 | SERPINA1, DPP4, GDF15, GSN, TFRC | NONE |
| 3020 | SERPINA1, DPP4, GDF15, MIF, PKM | NONE |
| 3021 | SERPINA1, DPP4, GDF15, MIF, TIMP1 | NONE |
| 3022 | SERPINA1, DPP4, GDF15, MIF, TFRC | NONE |
| 3023 | SERPINA1, DPP4, GDF15, PKM, TIMP1 | NONE |
| 3024 | SERPINA1, DPP4, GDF15, PKM, TFRC | NONE |
| 3025 | SERPINA1, DPP4, GDF15, TIMP1, TFRC | NONE |
| 3026 | SERPINA1, DPP4, GSN, MIF, PKM | NONE |
| 3027 | SERPINA1, DPP4, GSN, MIF, TIMP1 | NONE |
| 3028 | SERPINA1, DPP4, GSN, MIF, TFRC | NONE |
| 3029 | SERPINA1, DPP4, GSN, PKM, TIMP1 | NONE |
| 3030 | SERPINA1, DPP4, GSN, PKM, TFRC | NONE |
| 3031 | SERPINA1, DPP4, GSN, TIMP1, TFRC | NONE |
| 3032 | SERPINA1, DPP4, MIF, PKM, TIMP1 | NONE |
| 3033 | SERPINA1, DPP4, MIF, PKM, TFRC | NONE |
| 3034 | SERPINA1, DPP4, MIF, TIMP1, TFRC | NONE |
| 3035 | SERPINA1, DPP4, PKM, TIMP1, TFRC | NONE |
| 3036 | SERPINA1, GDF15, GSN, MIF, PKM | NONE |
| 3037 | SERPINA1, GDF15, GSN, MIF, TIMP1 | NONE |
| 3038 | SERPINA1, GDF15, GSN, MIF, TFRC | NONE |
| 3039 | SERPINA1, GDF15, GSN, PKM, TIMP1 | NONE |
| 3040 | SERPINA1, GDF15, GSN, PKM, TFRC | NONE |
| 3041 | SERPINA1, GDF15, GSN, TIMP1, TFRC | NONE |
| 3042 | SERPINA1, GDF15, MIF, PKM, TIMP1 | NONE |
| 3043 | SERPINA1, GDF15, MIF, PKM, TFRC | NONE |
| 3044 | SERPINA1, GDF15, MIF, TIMP1, TFRC | NONE |
| 3045 | SERPINA1, GDF15, PKM, TIMP1, TFRC | NONE |
| 3046 | SERPINA1, GSN, MIF, PKM, TIMP1 | NONE |
| 3047 | SERPINA1, GSN, MIF, PKM, TFRC | NONE |
| 3048 | SERPINA1, GSN, MIF, TIMP1, TFRC | NONE |
| 3049 | SERPINA1, GSN, PKM, TIMP1, TFRC | NONE |
| 3050 | SERPINA1, MIF, PKM, TIMP1, TFRC | NONE |
| 3051 | SERPINA3, CTSD, CLU, DPP4, GDF15 | NONE |
| 3052 | SERPINA3, CTSD, CLU, DPP4, GSN | NONE |
| 3053 | SERPINA3, CTSD, CLU, DPP4, MIF | NONE |
| 3054 | SERPINA3, CTSD, CLU, DPP4, PKM | NONE |
| 3055 | SERPINA3, CTSD, CLU, DPP4, TIMP1 | NONE |
| 3056 | SERPINA3, CTSD, CLU, DPP4, TFRC | NONE |
| 3057 | SERPINA3, CTSD, CLU, GDF15, GSN | NONE |
| 3058 | SERPINA3, CTSD, CLU, GDF15, MIF | NONE |
| 3059 | SERPINA3, CTSD, CLU, GDF15, PKM | NONE |
| 3060 | SERPINA3, CTSD, CLU, GDF15, TIMP1 | NONE |
| 3061 | SERPINA3, CTSD, CLU, GDF15, TFRC | NONE |
| 3062 | SERPINA3, CTSD, CLU, GSN, MIF | NONE |
| 3063 | SERPINA3, CTSD, CLU, GSN, PKM | NONE |
| 3064 | SERPINA3, CTSD, CLU, GSN, TIMP1 | NONE |
| 3065 | SERPINA3, CTSD, CLU, GSN, TFRC | NONE |
| 3066 | SERPINA3, CTSD, CLU, MIF, PKM | NONE |
| 3067 | SERPINA3, CTSD, CLU, MIF, TIMP1 | NONE |
| 3068 | SERPINA3, CTSD, CLU, MIF, TFRC | NONE |
| 3069 | SERPINA3, CTSD, CLU, PKM, TIMP1 | NONE |
| 3070 | SERPINA3, CTSD, CLU, PKM, TFRC | NONE |
| 3071 | SERPINA3, CTSD, CLU, TIMP1, TFRC | NONE |
| 3072 | SERPINA3, CTSD, DPP4, GDF15, GSN | NONE |
| 3073 | SERPINA3, CTSD, DPP4, GDF15, MIF | NONE |
| 3074 | SERPINA3, CTSD, DPP4, GDF15, PKM | NONE |
| 3075 | SERPINA3, CTSD, DPP4, GDF15, TIMP1 | NONE |
| 3076 | SERPINA3, CTSD, DPP4, GDF15, TFRC | NONE |
| 3077 | SERPINA3, CTSD, DPP4, GSN, MIF | NONE |
| 3078 | SERPINA3, CTSD, DPP4, GSN, PKM | NONE |
| 3079 | SERPINA3, CTSD, DPP4, GSN, TIMP1 | NONE |
| 3080 | SERPINA3, CTSD, DPP4, GSN, TFRC | NONE |
| 3081 | SERPINA3, CTSD, DPP4, MIF, PKM | NONE |
| 3082 | SERPINA3, CTSD, DPP4, MIF, TIMP1 | NONE |
| 3083 | SERPINA3, CTSD, DPP4, MIF, TFRC | NONE |
| 3084 | SERPINA3, CTSD, DPP4, PKM, TIMP1 | NONE |
| 3085 | SERPINA3, CTSD, DPP4, PKM, TFRC | NONE |
| 3086 | SERPINA3, CTSD, DPP4, TIMP1, TFRC | NONE |
| 3087 | SERPINA3, CTSD, GDF15, GSN, MIF | NONE |
| 3088 | SERPINA3, CTSD, GDF15, GSN, PKM | NONE |
| 3089 | SERPINA3, CTSD, GDF15, GSN, TIMP1 | NONE |
| 3090 | SERPINA3, CTSD, GDF15, GSN, TFRC | NONE |
| 3091 | SERPINA3, CTSD, GDF15, MIF, PKM | NONE |
| 3092 | SERPINA3, CTSD, GDF15, MIF, TIMP1 | NONE |
| 3093 | SERPINA3, CTSD, GDF15, MIF, TFRC | NONE |
| 3094 | SERPINA3, CTSD, GDF15, PKM, TIMP1 | NONE |
| 3095 | SERPINA3, CTSD, GDF15, PKM, TFRC | NONE |
| 3096 | SERPINA3, CTSD, GDF15, TIMP1, TFRC | NONE |
| 3097 | SERPINA3, CTSD, GSN, MIF, PKM | NONE |
| 3098 | SERPINA3, CTSD, GSN, MIF, TIMP1 | NONE |
| 3099 | SERPINA3, CTSD, GSN, MIF, TFRC | NONE |
| 3100 | SERPINA3, CTSD, GSN, PKM, TIMP1 | NONE |
| 3101 | SERPINA3, CTSD, GSN, PKM, TFRC | NONE |
| 3102 | SERPINA3, CTSD, GSN, TIMP1, TFRC | NONE |
| 3103 | SERPINA3, CTSD, MIF, PKM, TIMP1 | NONE |
| 3104 | SERPINA3, CTSD, MIF, PKM, TFRC | NONE |
| 3105 | SERPINA3, CTSD, MIF, TIMP1, TFRC | NONE |
| 3106 | SERPINA3, CTSD, PKM, TIMP1, TFRC | NONE |
| 3107 | SERPINA3, CLU, DPP4, GDF15, GSN | NONE |
| 3108 | SERPINA3, CLU, DPP4, GDF15, MIF | NONE |
| 3109 | SERPINA3, CLU, DPP4, GDF15, PKM | NONE |
| 3110 | SERPINA3, CLU, DPP4, GDF15, TIMP1 | NONE |
| 3111 | SERPINA3, CLU, DPP4, GDF15, TFRC | NONE |
| 3112 | SERPINA3, CLU, DPP4, GSN, MIF | NONE |
| 3113 | SERPINA3, CLU, DPP4, GSN, PKM | NONE |
| 3114 | SERPINA3, CLU, DPP4, GSN, TIMP1 | NONE |
| 3115 | SERPINA3, CLU, DPP4, GSN, TFRC | NONE |
| 3116 | SERPINA3, CLU, DPP4, MIF, PKM | NONE |
| 3117 | SERPINA3, CLU, DPP4, MIF, TIMP1 | NONE |
| 3118 | SERPINA3, CLU, DPP4, MIF, TFRC | NONE |
| 3119 | SERPINA3, CLU, DPP4, PKM, TIMP1 | NONE |
| 3120 | SERPINA3, CLU, DPP4, PKM, TFRC | NONE |
| 3121 | SERPINA3, CLU, DPP4, TIMP1, TFRC | NONE |
| 3122 | SERPINA3, CLU, GDF15, GSN, MIF | NONE |
| 3123 | SERPINA3, CLU, GDF15, GSN, PKM | NONE |
| 3124 | SERPINA3, CLU, GDF15, GSN, TIMP1 | NONE |
| 3125 | SERPINA3, CLU, GDF15, GSN, TFRC | NONE |
| 3126 | SERPINA3, CLU, GDF15, MIF, PKM | NONE |
| 3127 | SERPINA3, CLU, GDF15, MIF, TIMP1 | NONE |
| 3128 | SERPINA3, CLU, GDF15, MIF, TFRC | NONE |
| 3129 | SERPINA3, CLU, GDF15, PKM, TIMP1 | NONE |
| 3130 | SERPINA3, CLU, GDF15, PKM, TFRC | NONE |
| 3131 | SERPINA3, CLU, GDF15, TIMP1, TFRC | NONE |
| 3132 | SERPINA3, CLU, GSN, MIF, PKM | NONE |
| 3133 | SERPINA3, CLU, GSN, MIF, TIMP1 | NONE |
| 3134 | SERPINA3, CLU, GSN, MIF, TFRC | NONE |
| 3135 | SERPINA3, CLU, GSN, PKM, TIMP1 | NONE |
| 3136 | SERPINA3, CLU, GSN, PKM, TFRC | NONE |
| 3137 | SERPINA3, CLU, GSN, TIMP1, TFRC | NONE |
| 3138 | SERPINA3, CLU, MIF, PKM, TIMP1 | NONE |
| 3139 | SERPINA3, CLU, MIF, PKM, TFRC | NONE |
| 3140 | SERPINA3, CLU, MIF, TIMP1, TFRC | NONE |
| 3141 | SERPINA3, CLU, PKM, TIMP1, TFRC | NONE |
| 3142 | SERPINA3, DPP4, GDF15, GSN, MIF | NONE |
| 3143 | SERPINA3, DPP4, GDF15, GSN, PKM | NONE |
| 3144 | SERPINA3, DPP4, GDF15, GSN, TIMP1 | NONE |
| 3145 | SERPINA3, DPP4, GDF15, GSN, TFRC | NONE |
| 3146 | SERPINA3, DPP4, GDF15, MIF, PKM | NONE |
| 3147 | SERPINA3, DPP4, GDF15, MIF, TIMP1 | NONE |
| 3148 | SERPINA3, DPP4, GDF15, MIF, TFRC | NONE |
| 3149 | SERPINA3, DPP4, GDF15, PKM, TIMP1 | NONE |
| 3150 | SERPINA3, DPP4, GDF15, PKM, TFRC | NONE |
| 3151 | SERPINA3, DPP4, GDF15, TIMP1, TFRC | NONE |
| 3152 | SERPINA3, DPP4, GSN, MIF, PKM | NONE |
| 3153 | SERPINA3, DPP4, GSN, MIF, TIMP1 | NONE |
| 3154 | SERPINA3, DPP4, GSN, MIF, TFRC | NONE |
| 3155 | SERPINA3, DPP4, GSN, PKM, TIMP1 | NONE |
| 3156 | SERPINA3, DPP4, GSN, PKM, TFRC | NONE |
| 3157 | SERPINA3, DPP4, GSN, TIMP1, TFRC | NONE |
| 3158 | SERPINA3, DPP4, MIF, PKM, TIMP1 | NONE |
| 3159 | SERPINA3, DPP4, MIF, PKM, TFRC | NONE |
| 3160 | SERPINA3, DPP4, MIF, TIMP1, TFRC | NONE |
| 3161 | SERPINA3, DPP4, PKM, TIMP1, TFRC | NONE |
| 3162 | SERPINA3, GDF15, GSN, MIF, PKM | NONE |
| 3163 | SERPINA3, GDF15, GSN, MIF, TIMP1 | NONE |
| 3164 | SERPINA3, GDF15, GSN, MIF, TFRC | NONE |
| 3165 | SERPINA3, GDF15, GSN, PKM, TIMP1 | NONE |
| 3166 | SERPINA3, GDF15, GSN, PKM, TFRC | NONE |
| 3167 | SERPINA3, GDF15, GSN, TIMP1, TFRC | NONE |
| 3168 | SERPINA3, GDF15, MIF, PKM, TIMP1 | NONE |
| 3169 | SERPINA3, GDF15, MIF, PKM, TFRC | NONE |
| 3170 | SERPINA3, GDF15, MIF, TIMP1, TFRC | NONE |
| 3171 | SERPINA3, GDF15, PKM, TIMP1, TFRC | NONE |
| 3172 | SERPINA3, GSN, MIF, PKM, TIMP1 | NONE |
| 3173 | SERPINA3, GSN, MIF, PKM, TFRC | NONE |
| 3174 | SERPINA3, GSN, MIF, TIMP1, TFRC | NONE |
| 3175 | SERPINA3, GSN, PKM, TIMP1, TFRC | NONE |
| 3176 | SERPINA3, MIF, PKM, TIMP1, TFRC | NONE |
| 3177 | CTSD, CLU, DPP4, GDF15, GSN | NONE |
| 3178 | CTSD, CLU, DPP4, GDF15, MIF | NONE |
| 3179 | CTSD, CLU, DPP4, GDF15, PKM | NONE |
| 3180 | CTSD, CLU, DPP4, GDF15, TIMP1 | NONE |
| 3181 | CTSD, CLU, DPP4, GDF15, TFRC | NONE |
| 3182 | CTSD, CLU, DPP4, GSN, MIF | NONE |
| 3183 | CTSD, CLU, DPP4, GSN, PKM | NONE |
| 3184 | CTSD, CLU, DPP4, GSN, TIMP1 | NONE |
| 3185 | CTSD, CLU, DPP4, GSN, TFRC | NONE |
| 3186 | CTSD, CLU, DPP4, MIF, PKM | NONE |
| 3187 | CTSD, CLU, DPP4, MIF, TIMP1 | NONE |
| 3188 | CTSD, CLU, DPP4, MIF, TFRC | NONE |
| 3189 | CTSD, CLU, DPP4, PKM, TIMP1 | NONE |
| 3190 | CTSD, CLU, DPP4, PKM, TFRC | NONE |
| 3191 | CTSD, CLU, DPP4, TIMP1, TFRC | NONE |
| 3192 | CTSD, CLU, GDF15, GSN, MIF | NONE |
| 3193 | CTSD, CLU, GDF15, GSN, PKM | NONE |
| 3194 | CTSD, CLU, GDF15, GSN, TIMP1 | NONE |
| 3195 | CTSD, CLU, GDF15, GSN, TFRC | NONE |
| 3196 | CTSD, CLU, GDF15, MIF, PKM | NONE |
| 3197 | CTSD, CLU, GDF15, MIF, TIMP1 | NONE |
| 3198 | CTSD, CLU, GDF15, MIF, TFRC | NONE |
| 3199 | CTSD, CLU, GDF15, PKM, TIMP1 | NONE |
| 3200 | CTSD, CLU, GDF15, PKM, TFRC | NONE |
| 3201 | CTSD, CLU, GDF15, TIMP1, TFRC | NONE |
| 3202 | CTSD, CLU, GSN, MIF, PKM | NONE |
| 3203 | CTSD, CLU, GSN, MIF, TIMP1 | NONE |
| 3204 | CTSD, CLU, GSN, MIF, TFRC | NONE |
| 3205 | CTSD, CLU, GSN, PKM, TIMP1 | NONE |
| 3206 | CTSD, CLU, GSN, PKM, TFRC | NONE |
| 3207 | CTSD, CLU, GSN, TIMP1, TFRC | NONE |
| 3208 | CTSD, CLU, MIF, PKM, TIMP1 | NONE |
| 3209 | CTSD, CLU, MIF, PKM, TFRC | NONE |
| 3210 | CTSD, CLU, MIF, TIMP1, TFRC | NONE |
| 3211 | CTSD, CLU, PKM, TIMP1, TFRC | NONE |
| 3212 | CTSD, DPP4, GDF15, GSN, MIF | NONE |
| 3213 | CTSD, DPP4, GDF15, GSN, PKM | NONE |
| 3214 | CTSD, DPP4, GDF15, GSN, TIMP1 | NONE |
| 3215 | CTSD, DPP4, GDF15, GSN, TFRC | NONE |
| 3216 | CTSD, DPP4, GDF15, MIF, PKM | NONE |
| 3217 | CTSD, DPP4, GDF15, MIF, TIMP1 | NONE |
| 3218 | CTSD, DPP4, GDF15, MIF, TFRC | NONE |
| 3219 | CTSD, DPP4, GDF15, PKM, TIMP1 | NONE |
| 3220 | CTSD, DPP4, GDF15, PKM, TFRC | NONE |
| 3221 | CTSD, DPP4, GDF15, TIMP1, TFRC | NONE |
| 3222 | CTSD, DPP4, GSN, MIF, PKM | NONE |
| 3223 | CTSD, DPP4, GSN, MIF, TIMP1 | NONE |
| 3224 | CTSD, DPP4, GSN, MIF, TFRC | NONE |
| 3225 | CTSD, DPP4, GSN, PKM, TIMP1 | NONE |
| 3226 | CTSD, DPP4, GSN, PKM, TFRC | NONE |
| 3227 | CTSD, DPP4, GSN, TIMP1, TFRC | NONE |
| 3228 | CTSD, DPP4, MIF, PKM, TIMP1 | NONE |
| 3229 | CTSD, DPP4, MIF, PKM, TFRC | NONE |
| 3230 | CTSD, DPP4, MIF, TIMP1, TFRC | NONE |
| 3231 | CTSD, DPP4, PKM, TIMP1, TFRC | NONE |
| 3232 | CTSD, GDF15, GSN, MIF, PKM | NONE |
| 3233 | CTSD, GDF15, GSN, MIF, TIMP1 | NONE |
| 3234 | CTSD, GDF15, GSN, MIF, TFRC | NONE |
| 3235 | CTSD, GDF15, GSN, PKM, TIMP1 | NONE |
| 3236 | CTSD, GDF15, GSN, PKM, TFRC | NONE |
| 3237 | CTSD, GDF15, GSN, TIMP1, TFRC | NONE |
| 3238 | CTSD, GDF15, MIF, PKM, TIMP1 | NONE |
| 3239 | CTSD, GDF15, MIF, PKM, TFRC | NONE |
| 3240 | CTSD, GDF15, MIF, TIMP1, TFRC | NONE |
| 3241 | CTSD, GDF15, PKM, TIMP1, TFRC | NONE |
| 3242 | CTSD, GSN, MIF, PKM, TIMP1 | NONE |
| 3243 | CTSD, GSN, MIF, PKM, TFRC | NONE |
| 3244 | CTSD, GSN, MIF, TIMP1, TFRC | NONE |
| 3245 | CTSD, GSN, PKM, TIMP1, TFRC | NONE |
| 3246 | CTSD, MIF, PKM, TIMP1, TFRC | NONE |
| 3247 | CLU, DPP4, GDF15, GSN, MIF | NONE |
| 3248 | CLU, DPP4, GDF15, GSN, PKM | NONE |
| 3249 | CLU, DPP4, GDF15, GSN, TIMP1 | NONE |
| 3250 | CLU, DPP4, GDF15, GSN, TFRC | NONE |
| 3251 | CLU, DPP4, GDF15, MIF, PKM | NONE |
| 3252 | CLU, DPP4, GDF15, MIF, TIMP1 | NONE |
| 3253 | CLU, DPP4, GDF15, MIF, TFRC | NONE |
| 3254 | CLU, DPP4, GDF15, PKM, TIMP1 | NONE |
| 3255 | CLU, DPP4, GDF15, PKM, TFRC | NONE |
| 3256 | CLU, DPP4, GDF15, TIMP1, TFRC | NONE |
| 3257 | CLU, DPP4, GSN, MIF, PKM | NONE |
| 3258 | CLU, DPP4, GSN, MIF, TIMP1 | NONE |
| 3259 | CLU, DPP4, GSN, MIF, TFRC | NONE |
| 3260 | CLU, DPP4, GSN, PKM, TIMP1 | NONE |
| 3261 | CLU, DPP4, GSN, PKM, TFRC | NONE |
| 3262 | CLU, DPP4, GSN, TIMP1, TFRC | NONE |
| 3263 | CLU, DPP4, MIF, PKM, TIMP1 | NONE |
| 3264 | CLU, DPP4, MIF, PKM, TFRC | NONE |
| 3265 | CLU, DPP4, MIF, TIMP1, TFRC | NONE |
| 3266 | CLU, DPP4, PKM, TIMP1, TFRC | NONE |
| 3267 | CLU, GDF15, GSN, MIF, PKM | NONE |
| 3268 | CLU, GDF15, GSN, MIF, TIMP1 | NONE |
| 3269 | CLU, GDF15, GSN, MIF, TFRC | NONE |
| 3270 | CLU, GDF15, GSN, PKM, TIMP1 | NONE |
| 3271 | CLU, GDF15, GSN, PKM, TFRC | NONE |
| 3272 | CLU, GDF15, GSN, TIMP1, TFRC | NONE |
| 3273 | CLU, GDF15, MIF, PKM, TIMP1 | NONE |
| 3274 | CLU, GDF15, MIF, PKM, TFRC | NONE |
| 3275 | CLU, GDF15, MIF, TIMP1, TFRC | NONE |
| 3276 | CLU, GDF15, PKM, TIMP1, TFRC | NONE |
| 3277 | CLU, GSN, MIF, PKM, TIMP1 | NONE |
| 3278 | CLU, GSN, MIF, PKM, TFRC | NONE |
| 3279 | CLU, GSN, MIF, TIMP1, TFRC | NONE |
| 3280 | CLU, GSN, PKM, TIMP1, TFRC | NONE |
| 3281 | CLU, MIF, PKM, TIMP1, TFRC | NONE |
| 3282 | DPP4, GDF15, GSN, MIF, PKM | NONE |
| 3283 | DPP4, GDF15, GSN, MIF, TIMP1 | NONE |
| 3284 | DPP4, GDF15, GSN, MIF, TFRC | NONE |
| 3285 | DPP4, GDF15, GSN, PKM, TIMP1 | NONE |
| 3286 | DPP4, GDF15, GSN, PKM, TFRC | NONE |
| 3287 | DPP4, GDF15, GSN, TIMP1, TFRC | NONE |
| 3288 | DPP4, GDF15, MIF, PKM, TIMP1 | NONE |
| 3289 | DPP4, GDF15, MIF, PKM, TFRC | NONE |
| 3290 | DPP4, GDF15, MIF, TIMP1, TFRC | NONE |
| 3291 | DPP4, GDF15, PKM, TIMP1, TFRC | NONE |
| 3292 | DPP4, GSN, MIF, PKM, TIMP1 | NONE |
| 3293 | DPP4, GSN, MIF, PKM, TFRC | NONE |
| 3294 | DPP4, GSN, MIF, TIMP1, TFRC | NONE |
| 3295 | DPP4, GSN, PKM, TIMP1, TFRC | NONE |
| 3296 | DPP4, MIF, PKM, TIMP1, TFRC | NONE |
| 3297 | GDF15, GSN, MIF, PKM, TIMP1 | NONE |
| 3298 | GDF15, GSN, MIF, PKM, TFRC | NONE |
| 3299 | GDF15, GSN, MIF, TIMP1, TFRC | NONE |
| 3300 | GDF15, GSN, PKM, TIMP1, TFRC | NONE |
| 3301 | GDF15, MIF, PKM, TIMP1, TFRC | NONE |
| 3302 | GSN, MIF, PKM, TIMP1, TFRC | NONE |
| 3303 | SERPINA1, SERPINA3, CTSD | Age |
| 3304 | SERPINA1, SERPINA3, CLU | Age |
| 3305 | SERPINA1, SERPINA3, DPP4 | Age |
| 3306 | SERPINA1, SERPINA3, GDF15 | Age |
| 3307 | SERPINA1, SERPINA3, GSN | Age |
| 3308 | SERPINA1, SERPINA3, MIF | Age |
| 3309 | SERPINA1, SERPINA3, PKM | Age |
| 3310 | SERPINA1, SERPINA3, TIMP1 | Age |
| 3311 | SERPINA1, SERPINA3, TFRC | Age |
| 3312 | SERPINA1, CTSD, CLU | Age |
| 3313 | SERPINA1, CTSD, DPP4 | Age |
| 3314 | SERPINA1, CTSD, GDF15 | Age |
| 3315 | SERPINA1, CTSD, GSN | Age |
| 3316 | SERPINA1, CTSD, MIF | Age |
| 3317 | SERPINA1, CTSD, PKM | Age |
| 3318 | SERPINA1, CTSD, TIMP1 | Age |
| 3319 | SERPINA1, CTSD, TFRC | Age |
| 3320 | SERPINA1, CLU, DPP4 | Age |
| 3321 | SERPINA1, CLU, GDF15 | Age |
| 3322 | SERPINA1, CLU, GSN | Age |
| 3323 | SERPINA1, CLU, MIF | Age |
| 3324 | SERPINA1, CLU, PKM | Age |
| 3325 | SERPINA1, CLU, TIMP1 | Age |
| 3326 | SERPINA1, CLU, TFRC | Age |
| 3327 | SERPINA1, DPP4, GDF15 | Age |
| 3328 | SERPINA1, DPP4, GSN | Age |
| 3329 | SERPINA1, DPP4, MIF | Age |
| 3330 | SERPINA1, DPP4, PKM | Age |
| 3331 | SERPINA1, DPP4, TIMP1 | Age |
| 3332 | SERPINA1, DPP4, TFRC | Age |
| 3333 | SERPINA1, GDF15, GSN | Age |
| 3334 | SERPINA1, GDF15, MIF | Age |
| 3335 | SERPINA1, GDF15, PKM | Age |
| 3336 | SERPINA1, GDF15, TIMP1 | Age |
| 3337 | SERPINA1, GDF15, TFRC | Age |
| 3338 | SERPINA1, GSN, MIF | Age |
| 3339 | SERPINA1, GSN, PKM | Age |
| 3340 | SERPINA1, GSN, TIMP1 | Age |
| 3341 | SERPINA1, GSN, TFRC | Age |
| 3342 | SERPINA1, MIF, PKM | Age |
| 3343 | SERPINA1, MIF, TIMP1 | Age |
| 3344 | SERPINA1, MIF, TFRC | Age |
| 3345 | SERPINA1, PKM, TIMP1 | Age |
| 3346 | SERPINA1, PKM, TFRC | Age |
| 3347 | SERPINA1, TIMP1, TFRC | Age |
| 3348 | SERPINA3, CTSD, CLU | Age |
| 3349 | SERPINA3, CTSD, DPP4 | Age |
| 3350 | SERPINA3, CTSD, GDF15 | Age |
| 3351 | SERPINA3, CTSD, GSN | Age |
| 3352 | SERPINA3, CTSD, MIF | Age |
| 3353 | SERPINA3, CTSD, PKM | Age |
| 3354 | SERPINA3, CTSD, TIMP1 | Age |
| 3355 | SERPINA3, CTSD, TFRC | Age |
| 3356 | SERPINA3, CLU, DPP4 | Age |
| 3357 | SERPINA3, CLU, GDF15 | Age |
| 3358 | SERPINA3, CLU, GSN | Age |
| 3359 | SERPINA3, CLU, MIF | Age |
| 3360 | SERPINA3, CLU, PKM | Age |
| 3361 | SERPINA3, CLU, TIMP1 | Age |
| 3362 | SERPINA3, CLU, TFRC | Age |
| 3363 | SERPINA3, DPP4, GDF15 | Age |
| 3364 | SERPINA3, DPP4, GSN | Age |
| 3365 | SERPINA3, DPP4, MIF | Age |
| 3366 | SERPINA3, DPP4, PKM | Age |
| 3367 | SERPINA3, DPP4, TIMP1 | Age |
| 3368 | SERPINA3, DPP4, TFRC | Age |
| 3369 | SERPINA3, GDF15, GSN | Age |
| 3370 | SERPINA3, GDF15, MIF | Age |
| 3371 | SERPINA3, GDF15, PKM | Age |
| 3372 | SERPINA3, GDF15, TIMP1 | Age |
| 3373 | SERPINA3, GDF15, TFRC | Age |
| 3374 | SERPINA3, GSN, MIF | Age |
| 3375 | SERPINA3, GSN, PKM | Age |
| 3376 | SERPINA3, GSN, TIMP1 | Age |
| 3377 | SERPINA3, GSN, TFRC | Age |
| 3378 | SERPINA3, MIF, PKM | Age |
| 3379 | SERPINA3, MIF, TIMP1 | Age |
| 3380 | SERPINA3, MIF, TFRC | Age |
| 3381 | SERPINA3, PKM, TIMP1 | Age |
| 3382 | SERPINA3, PKM, TFRC | Age |
| 3383 | SERPINA3, TIMP1, TFRC | Age |
| 3384 | CTSD, CLU, DPP4 | Age |
| 3385 | CTSD, CLU, GDF15 | Age |
| 3386 | CTSD, CLU, GSN | Age |
| 3387 | CTSD, CLU, MIF | Age |
| 3388 | CTSD, CLU, PKM | Age |
| 3389 | CTSD, CLU, TIMP1 | Age |
| 3390 | CTSD, CLU, TFRC | Age |
| 3391 | CTSD, DPP4, GDF15 | Age |
| 3392 | CTSD, DPP4, GSN | Age |
| 3393 | CTSD, DPP4, MIF | Age |
| 3394 | CTSD, DPP4, PKM | Age |
| 3395 | CTSD, DPP4, TIMP1 | Age |
| 3396 | CTSD, DPP4, TFRC | Age |
| 3397 | CTSD, GDF15, GSN | Age |
| 3398 | CTSD, GDF15, MIF | Age |
| 3399 | CTSD, GDF15, PKM | Age |
| 3400 | CTSD, GDF15, TIMP1 | Age |
| 3401 | CTSD, GDF15, TFRC | Age |
| 3402 | CTSD, GSN, MIF | Age |
| 3403 | CTSD, GSN, PKM | Age |
| 3404 | CTSD, GSN, TIMP1 | Age |
| 3405 | CTSD, GSN, TFRC | Age |
| 3406 | CTSD, MIF, PKM | Age |
| 3407 | CTSD, MIF, TIMP1 | Age |
| 3408 | CTSD, MIF, TFRC | Age |
| 3409 | CTSD, PKM, TIMP1 | Age |
| 3410 | CTSD, PKM, TFRC | Age |
| 3411 | CTSD, TIMP1, TFRC | Age |
| 3412 | CLU, DPP4, GDF15 | Age |
| 3413 | CLU, DPP4, GSN | Age |
| 3414 | CLU, DPP4, MIF | Age |
| 3415 | CLU, DPP4, PKM | Age |
| 3416 | CLU, DPP4, TIMP1 | Age |
| 3417 | CLU, DPP4, TFRC | Age |
| 3418 | CLU, GDF15, GSN | Age |
| 3419 | CLU, GDF15, MIF | Age |
| 3420 | CLU, GDF15, PKM | Age |
| 3421 | CLU, GDF15, TIMP1 | Age |
| 3422 | CLU, GDF15, TFRC | Age |
| 3423 | CLU, GSN, MIF | Age |
| 3424 | CLU, GSN, PKM | Age |
| 3425 | CLU, GSN, TIMP1 | Age |
| 3426 | CLU, GSN, TFRC | Age |
| 3427 | CLU, MIF, PKM | Age |
| 3428 | CLU, MIF, TIMP1 | Age |
| 3429 | CLU, MIF, TFRC | Age |
| 3430 | CLU, PKM, TIMP1 | Age |
| 3431 | CLU, PKM, TFRC | Age |
| 3432 | CLU, TIMP1, TFRC | Age |
| 3433 | DPP4, GDF15, GSN | Age |
| 3434 | DPP4, GDF15, MIF | Age |
| 3435 | DPP4, GDF15, PKM | Age |
| 3436 | DPP4, GDF15, TIMP1 | Age |
| 3437 | DPP4, GDF15, TFRC | Age |
| 3438 | DPP4, GSN, MIF | Age |
| 3439 | DPP4, GSN, PKM | Age |
| 3440 | DPP4, GSN, TIMP1 | Age |
| 3441 | DPP4, GSN, TFRC | Age |
| 3442 | DPP4, MIF, PKM | Age |
| 3443 | DPP4, MIF, TIMP1 | Age |
| 3444 | DPP4, MIF, TFRC | Age |
| 3445 | DPP4, PKM, TIMP1 | Age |
| 3446 | DPP4, PKM, TFRC | Age |
| 3447 | DPP4, TIMP1, TFRC | Age |
| 3448 | GDF15, GSN, MIF | Age |
| 3449 | GDF15, GSN, PKM | Age |
| 3450 | GDF15, GSN, TIMP1 | Age |
| 3451 | GDF15, GSN, TFRC | Age |
| 3452 | GDF15, MIF, PKM | Age |
| 3453 | GDF15, MIF, TIMP1 | Age |
| 3454 | GDF15, MIF, TFRC | Age |
| 3455 | GDF15, PKM, TIMP1 | Age |
| 3456 | GDF15, PKM, TFRC | Age |
| 3457 | GDF15, TIMP1, TFRC | Age |
| 3458 | GSN, MIF, PKM | Age |
| 3459 | GSN, MIF, TIMP1 | Age |
| 3460 | GSN, MIF, TFRC | Age |
| 3461 | GSN, PKM, TIMP1 | Age |
| 3462 | GSN, PKM, TFRC | Age |
| 3463 | GSN, TIMP1, TFRC | Age |
| 3464 | MIF, PKM, TIMP1 | Age |
| 3465 | MIF, PKM, TFRC | Age |
| 3466 | MIF, TIMP1, TFRC | Age |
| 3467 | PKM, TIMP1, TFRC | Age |
| 3468 | SERPINA1, SERPINA3, CTSD, CLU | NONE |
| 3469 | SERPINA1, SERPINA3, CTSD, DPP4 | NONE |
| 3470 | SERPINA1, SERPINA3, CTSD, GDF15 | NONE |
| 3471 | SERPINA1, SERPINA3, CTSD, GSN | NONE |
| 3472 | SERPINA1, SERPINA3, CTSD, MIF | NONE |
| 3473 | SERPINA1, SERPINA3, CTSD, PKM | NONE |
| 3474 | SERPINA1, SERPINA3, CTSD, TIMP1 | NONE |
| 3475 | SERPINA1, SERPINA3, CTSD, TFRC | NONE |
| 3476 | SERPINA1, SERPINA3, CLU, DPP4 | NONE |
| 3477 | SERPINA1, SERPINA3, CLU, GDF15 | NONE |
| 3478 | SERPINA1, SERPINA3, CLU, GSN | NONE |
| 3479 | SERPINA1, SERPINA3, CLU, MIF | NONE |
| 3480 | SERPINA1, SERPINA3, CLU, PKM | NONE |
| 3481 | SERPINA1, SERPINA3, CLU, TIMP1 | NONE |
| 3482 | SERPINA1, SERPINA3, CLU, TFRC | NONE |
| 3483 | SERPINA1, SERPINA3, DPP4, GDF15 | NONE |
| 3484 | SERPINA1, SERPINA3, DPP4, GSN | NONE |
| 3485 | SERPINA1, SERPINA3, DPP4, MIF | NONE |
| 3486 | SERPINA1, SERPINA3, DPP4, PKM | NONE |
| 3487 | SERPINA1, SERPINA3, DPP4, TIMP1 | NONE |
| 3488 | SERPINA1, SERPINA3, DPP4, TFRC | NONE |
| 3489 | SERPINA1, SERPINA3, GDF15, GSN | NONE |
| 3490 | SERPINA1, SERPINA3, GDF15, MIF | NONE |
| 3491 | SERPINA1, SERPINA3, GDF15, PKM | NONE |
| 3492 | SERPINA1, SERPINA3, GDF15, TIMP1 | NONE |
| 3493 | SERPINA1, SERPINA3, GDF15, TFRC | NONE |
| 3494 | SERPINA1, SERPINA3, GSN, MIF | NONE |
| 3495 | SERPINA1, SERPINA3, GSN, PKM | NONE |
| 3496 | SERPINA1, SERPINA3, GSN, TIMP1 | NONE |
| 3497 | SERPINA1, SERPINA3, GSN, TFRC | NONE |
| 3498 | SERPINA1, SERPINA3, MIF, PKM | NONE |
| 3499 | SERPINA1, SERPINA3, MIF, TIMP1 | NONE |
| 3500 | SERPINA1, SERPINA3, MIF, TFRC | NONE |
| 3501 | SERPINA1, SERPINA3, PKM, TIMP1 | NONE |
| 3502 | SERPINA1, SERPINA3, PKM, TFRC | NONE |
| 3503 | SERPINA1, SERPINA3, TIMP1, TFRC | NONE |
| 3504 | SERPINA1, CTSD, CLU, DPP4 | NONE |
| 3505 | SERPINA1, CTSD, CLU, GDF15 | NONE |
| 3506 | SERPINA1, CTSD, CLU, GSN | NONE |
| 3507 | SERPINA1, CTSD, CLU, MIF | NONE |
| 3508 | SERPINA1, CTSD, CLU, PKM | NONE |
| 3509 | SERPINA1, CTSD, CLU, TIMP1 | NONE |
| 3510 | SERPINA1, CTSD, CLU, TFRC | NONE |
| 3511 | SERPINA1, CTSD, DPP4, GDF15 | NONE |
| 3512 | SERPINA1, CTSD, DPP4, GSN | NONE |
| 3513 | SERPINA1, CTSD, DPP4, MIF | NONE |
| 3514 | SERPINA1, CTSD, DPP4, PKM | NONE |
| 3515 | SERPINA1, CTSD, DPP4, TIMP1 | NONE |
| 3516 | SERPINA1, CTSD, DPP4, TFRC | NONE |
| 3517 | SERPINA1, CTSD, GDF15, GSN | NONE |
| 3518 | SERPINA1, CTSD, GDF15, MIF | NONE |
| 3519 | SERPINA1, CTSD, GDF15, PKM | NONE |
| 3520 | SERPINA1, CTSD, GDF15, TIMP1 | NONE |
| 3521 | SERPINA1, CTSD, GDF15, TFRC | NONE |
| 3522 | SERPINA1, CTSD, GSN, MIF | NONE |
| 3523 | SERPINA1, CTSD, GSN, PKM | NONE |
| 3524 | SERPINA1, CTSD, GSN, TIMP1 | NONE |
| 3525 | SERPINA1, CTSD, GSN, TFRC | NONE |
| 3526 | SERPINA1, CTSD, MIF, PKM | NONE |
| 3527 | SERPINA1, CTSD, MIF, TIMP1 | NONE |
| 3528 | SERPINA1, CTSD, MIF, TFRC | NONE |
| 3529 | SERPINA1, CTSD, PKM, TIMP1 | NONE |
| 3530 | SERPINA1, CTSD, PKM, TFRC | NONE |
| 3531 | SERPINA1, CTSD, TIMP1, TFRC | NONE |
| 3532 | SERPINA1, CLU, DPP4, GDF15 | NONE |
| 3533 | SERPINA1, CLU, DPP4, GSN | NONE |
| 3534 | SERPINA1, CLU, DPP4, MIF | NONE |
| 3535 | SERPINA1, CLU, DPP4, PKM | NONE |
| 3536 | SERPINA1, CLU, DPP4, TIMP1 | NONE |
| 3537 | SERPINA1, CLU, DPP4, TFRC | NONE |
| 3538 | SERPINA1, CLU, GDF15, GSN | NONE |
| 3539 | SERPINA1, CLU, GDF15, MIF | NONE |
| 3540 | SERPINA1, CLU, GDF15, PKM | NONE |
| 3541 | SERPINA1, CLU, GDF15, TIMP1 | NONE |
| 3542 | SERPINA1, CLU, GDF15, TFRC | NONE |
| 3543 | SERPINA1, CLU, GSN, MIF | NONE |
| 3544 | SERPINA1, CLU, GSN, PKM | NONE |
| 3545 | SERPINA1, CLU, GSN, TIMP1 | NONE |
| 3546 | SERPINA1, CLU, GSN, TFRC | NONE |
| 3547 | SERPINA1, CLU, MIF, PKM | NONE |
| 3548 | SERPINA1, CLU, MIF, TIMP1 | NONE |
| 3549 | SERPINA1, CLU, MIF, TFRC | NONE |
| 3550 | SERPINA1, CLU, PKM, TIMP1 | NONE |
| 3551 | SERPINA1, CLU, PKM, TFRC | NONE |
| 3552 | SERPINA1, CLU, TIMP1, TFRC | NONE |
| 3553 | SERPINA1, DPP4, GDF15, GSN | NONE |
| 3554 | SERPINA1, DPP4, GDF15, MIF | NONE |
| 3555 | SERPINA1, DPP4, GDF15, PKM | NONE |
| 3556 | SERPINA1, DPP4, GDF15, TIMP1 | NONE |
| 3557 | SERPINA1, DPP4, GDF15, TFRC | NONE |
| 3558 | SERPINA1, DPP4, GSN, MIF | NONE |
| 3559 | SERPINA1, DPP4, GSN, PKM | NONE |
| 3560 | SERPINA1, DPP4, GSN, TIMP1 | NONE |
| 3561 | SERPINA1, DPP4, GSN, TFRC | NONE |
| 3562 | SERPINA1, DPP4, MIF, PKM | NONE |
| 3563 | SERPINA1, DPP4, MIF, TIMP1 | NONE |
| 3564 | SERPINA1, DPP4, MIF, TFRC | NONE |
| 3565 | SERPINA1, DPP4, PKM, TIMP1 | NONE |
| 3566 | SERPINA1, DPP4, PKM, TFRC | NONE |
| 3567 | SERPINA1, DPP4, TIMP1, TFRC | NONE |
| 3568 | SERPINA1, GDF15, GSN, MIF | NONE |
| 3569 | SERPINA1, GDF15, GSN, PKM | NONE |
| 3570 | SERPINA1, GDF15, GSN, TIMP1 | NONE |
| 3571 | SERPINA1, GDF15, GSN, TFRC | NONE |
| 3572 | SERPINA1, GDF15, MIF, PKM | NONE |
| 3573 | SERPINA1, GDF15, MIF, TIMP1 | NONE |
| 3574 | SERPINA1, GDF15, MIF, TFRC | NONE |
| 3575 | SERPINA1, GDF15, PKM, TIMP1 | NONE |
| 3576 | SERPINA1, GDF15, PKM, TFRC | NONE |
| 3577 | SERPINA1, GDF15, TIMP1, TFRC | NONE |
| 3578 | SERPINA1, GSN, MIF, PKM | NONE |
| 3579 | SERPINA1, GSN, MIF, TIMP1 | NONE |
| 3580 | SERPINA1, GSN, MIF, TFRC | NONE |
| 3581 | SERPINA1, GSN, PKM, TIMP1 | NONE |
| 3582 | SERPINA1, GSN, PKM, TFRC | NONE |
| 3583 | SERPINA1, GSN, TIMP1, TFRC | NONE |
| 3584 | SERPINA1, MIF, PKM, TIMP1 | NONE |
| 3585 | SERPINA1, MIF, PKM, TFRC | NONE |
| 3586 | SERPINA1, MIF, TIMP1, TFRC | NONE |
| 3587 | SERPINA1, PKM, TIMP1, TFRC | NONE |
| 3588 | SERPINA3, CTSD, CLU, DPP4 | NONE |
| 3589 | SERPINA3, CTSD, CLU, GDF15 | NONE |
| 3590 | SERPINA3, CTSD, CLU, GSN | NONE |
| 3591 | SERPINA3, CTSD, CLU, MIF | NONE |
| 3592 | SERPINA3, CTSD, CLU, PKM | NONE |
| 3593 | SERPINA3, CTSD, CLU, TIMP1 | NONE |
| 3594 | SERPINA3, CTSD, CLU, TFRC | NONE |
| 3595 | SERPINA3, CTSD, DPP4, GDF15 | NONE |
| 3596 | SERPINA3, CTSD, DPP4, GSN | NONE |
| 3597 | SERPINA3, CTSD, DPP4, MIF | NONE |
| 3598 | SERPINA3, CTSD, DPP4, PKM | NONE |
| 3599 | SERPINA3, CTSD, DPP4, TIMP1 | NONE |
| 3600 | SERPINA3, CTSD, DPP4, TFRC | NONE |
| 3601 | SERPINA3, CTSD, GDF15, GSN | NONE |
| 3602 | SERPINA3, CTSD, GDF15, MIF | NONE |
| 3603 | SERPINA3, CTSD, GDF15, PKM | NONE |
| 3604 | SERPINA3, CTSD, GDF15, TIMP1 | NONE |
| 3605 | SERPINA3, CTSD, GDF15, TFRC | NONE |
| 3606 | SERPINA3, CTSD, GSN, MIF | NONE |
| 3607 | SERPINA3, CTSD, GSN, PKM | NONE |
| 3608 | SERPINA3, CTSD, GSN, TIMP1 | NONE |
| 3609 | SERPINA3, CTSD, GSN, TFRC | NONE |
| 3610 | SERPINA3, CTSD, MIF, PKM | NONE |
| 3611 | SERPINA3, CTSD, MIF, TIMP1 | NONE |
| 3612 | SERPINA3, CTSD, MIF, TFRC | NONE |
| 3613 | SERPINA3, CTSD, PKM, TIMP1 | NONE |
| 3614 | SERPINA3, CTSD, PKM, TFRC | NONE |
| 3615 | SERPINA3, CTSD, TIMP1, TFRC | NONE |
| 3616 | SERPINA3, CLU, DPP4, GDF15 | NONE |
| 3617 | SERPINA3, CLU, DPP4, GSN | NONE |
| 3618 | SERPINA3, CLU, DPP4, MIF | NONE |
| 3619 | SERPINA3, CLU, DPP4, PKM | NONE |
| 3620 | SERPINA3, CLU, DPP4, TIMP1 | NONE |
| 3621 | SERPINA3, CLU, DPP4, TFRC | NONE |
| 3622 | SERPINA3, CLU, GDF15, GSN | NONE |
| 3623 | SERPINA3, CLU, GDF15, MIF | NONE |
| 3624 | SERPINA3, CLU, GDF15, PKM | NONE |
| 3625 | SERPINA3, CLU, GDF15, TIMP1 | NONE |
| 3626 | SERPINA3, CLU, GDF15, TFRC | NONE |
| 3627 | SERPINA3, CLU, GSN, MIF | NONE |
| 3628 | SERPINA3, CLU, GSN, PKM | NONE |
| 3629 | SERPINA3, CLU, GSN, TIMP1 | NONE |
| 3630 | SERPINA3, CLU, GSN, TFRC | NONE |
| 3631 | SERPINA3, CLU, MIF, PKM | NONE |
| 3632 | SERPINA3, CLU, MIF, TIMP1 | NONE |
| 3633 | SERPINA3, CLU, MIF, TFRC | NONE |
| 3634 | SERPINA3, CLU, PKM, TIMP1 | NONE |
| 3635 | SERPINA3, CLU, PKM, TFRC | NONE |
| 3636 | SERPINA3, CLU, TIMP1, TFRC | NONE |
| 3637 | SERPINA3, DPP4, GDF15, GSN | NONE |
| 3638 | SERPINA3, DPP4, GDF15, MIF | NONE |
| 3639 | SERPINA3, DPP4, GDF15, PKM | NONE |
| 3640 | SERPINA3, DPP4, GDF15, TIMP1 | NONE |
| 3641 | SERPINA3, DPP4, GDF15, TFRC | NONE |
| 3642 | SERPINA3, DPP4, GSN, MIF | NONE |
| 3643 | SERPINA3, DPP4, GSN, PKM | NONE |
| 3644 | SERPINA3, DPP4, GSN, TIMP1 | NONE |
| 3645 | SERPINA3, DPP4, GSN, TFRC | NONE |
| 3646 | SERPINA3, DPP4, MIF, PKM | NONE |
| 3647 | SERPINA3, DPP4, MIF, TIMP1 | NONE |
| 3648 | SERPINA3, DPP4, MIF, TFRC | NONE |
| 3649 | SERPINA3, DPP4, PKM, TIMP1 | NONE |
| 3650 | SERPINA3, DPP4, PKM, TFRC | NONE |
| 3651 | SERPINA3, DPP4, TIMP1, TFRC | NONE |
| 3652 | SERPINA3, GDF15, GSN, MIF | NONE |
| 3653 | SERPINA3, GDF15, GSN, PKM | NONE |
| 3654 | SERPINA3, GDF15, GSN, TIMP1 | NONE |
| 3655 | SERPINA3, GDF15, GSN, TFRC | NONE |
| 3656 | SERPINA3, GDF15, MIF, PKM | NONE |
| 3657 | SERPINA3, GDF15, MIF, TIMP1 | NONE |
| 3658 | SERPINA3, GDF15, MIF, TFRC | NONE |
| 3659 | SERPINA3, GDF15, PKM, TIMP1 | NONE |
| 3660 | SERPINA3, GDF15, PKM, TFRC | NONE |
| 3661 | SERPINA3, GDF15, TIMP1, TFRC | NONE |
| 3662 | SERPINA3, GSN, MIF, PKM | NONE |
| 3663 | SERPINA3, GSN, MIF, TIMP1 | NONE |
| 3664 | SERPINA3, GSN, MIF, TFRC | NONE |
| 3665 | SERPINA3, GSN, PKM, TIMP1 | NONE |
| 3666 | SERPINA3, GSN, PKM, TFRC | NONE |
| 3667 | SERPINA3, GSN, TIMP1, TFRC | NONE |
| 3668 | SERPINA3, MIF, PKM, TIMP1 | NONE |
| 3669 | SERPINA3, MIF, PKM, TFRC | NONE |
| 3670 | SERPINA3, MIF, TIMP1, TFRC | NONE |
| 3671 | SERPINA3, PKM, TIMP1, TFRC | NONE |
| 3672 | CTSD, CLU, DPP4, GDF15 | NONE |
| 3673 | CTSD, CLU, DPP4, GSN | NONE |
| 3674 | CTSD, CLU, DPP4, MIF | NONE |
| 3675 | CTSD, CLU, DPP4, PKM | NONE |
| 3676 | CTSD, CLU, DPP4, TIMP1 | NONE |
| 3677 | CTSD, CLU, DPP4, TFRC | NONE |
| 3678 | CTSD, CLU, GDF15, GSN | NONE |
| 3679 | CTSD, CLU, GDF15, MIF | NONE |
| 3680 | CTSD, CLU, GDF15, PKM | NONE |
| 3681 | CTSD, CLU, GDF15, TIMP1 | NONE |
| 3682 | CTSD, CLU, GDF15, TFRC | NONE |
| 3683 | CTSD, CLU, GSN, MIF | NONE |
| 3684 | CTSD, CLU, GSN, PKM | NONE |
| 3685 | CTSD, CLU, GSN, TIMP1 | NONE |
| 3686 | CTSD, CLU, GSN, TFRC | NONE |
| 3687 | CTSD, CLU, MIF, PKM | NONE |
| 3688 | CTSD, CLU, MIF, TIMP1 | NONE |
| 3689 | CTSD, CLU, MIF, TFRC | NONE |
| 3690 | CTSD, CLU, PKM, TIMP1 | NONE |
| 3691 | CTSD, CLU, PKM, TFRC | NONE |
| 3692 | CTSD, CLU, TIMP1, TFRC | NONE |
| 3693 | CTSD, DPP4, GDF15, GSN | NONE |
| 3694 | CTSD, DPP4, GDF15, MIF | NONE |
| 3695 | CTSD, DPP4, GDF15, PKM | NONE |
| 3696 | CTSD, DPP4, GDF15, TIMP1 | NONE |
| 3697 | CTSD, DPP4, GDF15, TFRC | NONE |
| 3698 | CTSD, DPP4, GSN, MIF | NONE |
| 3699 | CTSD, DPP4, GSN, PKM | NONE |
| 3700 | CTSD, DPP4, GSN, TIMP1 | NONE |
| 3701 | CTSD, DPP4, GSN, TFRC | NONE |
| 3702 | CTSD, DPP4, MIF, PKM | NONE |
| 3703 | CTSD, DPP4, MIF, TIMP1 | NONE |
| 3704 | CTSD, DPP4, MIF, TFRC | NONE |
| 3705 | CTSD, DPP4, PKM, TIMP1 | NONE |
| 3706 | CTSD, DPP4, PKM, TFRC | NONE |
| 3707 | CTSD, DPP4, TIMP1, TFRC | NONE |
| 3708 | CTSD, GDF15, GSN, MIF | NONE |
| 3709 | CTSD, GDF15, GSN, PKM | NONE |
| 3710 | CTSD, GDF15, GSN, TIMP1 | NONE |
| 3711 | CTSD, GDF15, GSN, TFRC | NONE |
| 3712 | CTSD, GDF15, MIF, PKM | NONE |
| 3713 | CTSD, GDF15, MIF, TIMP1 | NONE |
| 3714 | CTSD, GDF15, MIF, TFRC | NONE |
| 3715 | CTSD, GDF15, PKM, TIMP1 | NONE |
| 3716 | CTSD, GDF15, PKM, TFRC | NONE |
| 3717 | CTSD, GDF15, TIMP1, TFRC | NONE |
| 3718 | CTSD, GSN, MIF, PKM | NONE |
| 3719 | CTSD, GSN, MIF, TIMP1 | NONE |
| 3720 | CTSD, GSN, MIF, TFRC | NONE |
| 3721 | CTSD, GSN, PKM, TIMP1 | NONE |
| 3722 | CTSD, GSN, PKM, TFRC | NONE |
| 3723 | CTSD, GSN, TIMP1, TFRC | NONE |
| 3724 | CTSD, MIF, PKM, TIMP1 | NONE |
| 3725 | CTSD, MIF, PKM, TFRC | NONE |
| 3726 | CTSD, MIF, TIMP1, TFRC | NONE |
| 3727 | CTSD, PKM, TIMP1, TFRC | NONE |
| 3728 | CLU, DPP4, GDF15, GSN | NONE |
| 3729 | CLU, DPP4, GDF15, MIF | NONE |
| 3730 | CLU, DPP4, GDF15, PKM | NONE |
| 3731 | CLU, DPP4, GDF15, TIMP1 | NONE |
| 3732 | CLU, DPP4, GDF15, TFRC | NONE |
| 3733 | CLU, DPP4, GSN, MIF | NONE |
| 3734 | CLU, DPP4, GSN, PKM | NONE |
| 3735 | CLU, DPP4, GSN, TIMP1 | NONE |
| 3736 | CLU, DPP4, GSN, TFRC | NONE |
| 3737 | CLU, DPP4, MIF, PKM | NONE |
| 3738 | CLU, DPP4, MIF, TIMP1 | NONE |
| 3739 | CLU, DPP4, MIF, TFRC | NONE |
| 3740 | CLU, DPP4, PKM, TIMP1 | NONE |
| 3741 | CLU, DPP4, PKM, TFRC | NONE |
| 3742 | CLU, DPP4, TIMP1, TFRC | NONE |
| 3743 | CLU, GDF15, GSN, MIF | NONE |
| 3744 | CLU, GDF15, GSN, PKM | NONE |
| 3745 | CLU, GDF15, GSN, TIMP1 | NONE |
| 3746 | CLU, GDF15, GSN, TFRC | NONE |
| 3747 | CLU, GDF15, MIF, PKM | NONE |
| 3748 | CLU, GDF15, MIF, TIMP1 | NONE |
| 3749 | CLU, GDF15, MIF, TFRC | NONE |
| 3750 | CLU, GDF15, PKM, TIMP1 | NONE |
| 3751 | CLU, GDF15, PKM, TFRC | NONE |
| 3752 | CLU, GDF15, TIMP1, TFRC | NONE |
| 3753 | CLU, GSN, MIF, PKM | NONE |
| 3754 | CLU, GSN, MIF, TIMP1 | NONE |
| 3755 | CLU, GSN, MIF, TFRC | NONE |
| 3756 | CLU, GSN, PKM, TIMP1 | NONE |
| 3757 | CLU, GSN, PKM, TFRC | NONE |
| 3758 | CLU, GSN, TIMP1, TFRC | NONE |
| 3759 | CLU, MIF, PKM, TIMP1 | NONE |
| 3760 | CLU, MIF, PKM, TFRC | NONE |
| 3761 | CLU, MIF, TIMP1, TFRC | NONE |
| 3762 | CLU, PKM, TIMP1, TFRC | NONE |
| 3763 | DPP4, GDF15, GSN, MIF | NONE |
| 3764 | DPP4, GDF15, GSN, PKM | NONE |
| 3765 | DPP4, GDF15, GSN, TIMP1 | NONE |
| 3766 | DPP4, GDF15, GSN, TFRC | NONE |
| 3767 | DPP4, GDF15, MIF, PKM | NONE |
| 3768 | DPP4, GDF15, MIF, TIMP1 | NONE |
| 3769 | DPP4, GDF15, MIF, TFRC | NONE |
| 3770 | DPP4, GDF15, PKM, TIMP1 | NONE |
| 3771 | DPP4, GDF15, PKM, TFRC | NONE |
| 3772 | DPP4, GDF15, TIMP1, TFRC | NONE |
| 3773 | DPP4, GSN, MIF, PKM | NONE |
| 3774 | DPP4, GSN, MIF, TIMP1 | NONE |
| 3775 | DPP4, GSN, MIF, TFRC | NONE |
| 3776 | DPP4, GSN, PKM, TIMP1 | NONE |
| 3777 | DPP4, GSN, PKM, TFRC | NONE |
| 3778 | DPP4, GSN, TIMP1, TFRC | NONE |
| 3779 | DPP4, MIF, PKM, TIMP1 | NONE |
| 3780 | DPP4, MIF, PKM, TFRC | NONE |
| 3781 | DPP4, MIF, TIMP1, TFRC | NONE |
| 3782 | DPP4, PKM, TIMP1, TFRC | NONE |
| 3783 | GDF15, GSN, MIF, PKM | NONE |
| 3784 | GDF15, GSN, MIF, TIMP1 | NONE |
| 3785 | GDF15, GSN, MIF, TFRC | NONE |
| 3786 | GDF15, GSN, PKM, TIMP1 | NONE |
| 3787 | GDF15, GSN, PKM, TFRC | NONE |
| 3788 | GDF15, GSN, TIMP1, TFRC | NONE |
| 3789 | GDF15, MIF, PKM, TIMP1 | NONE |
| 3790 | GDF15, MIF, PKM, TFRC | NONE |
| 3791 | GDF15, MIF, TIMP1, TFRC | NONE |
| 3792 | GDF15, PKM, TIMP1, TFRC | NONE |
| 3793 | GSN, MIF, PKM, TIMP1 | NONE |
| 3794 | GSN, MIF, PKM, TFRC | NONE |
| 3795 | GSN, MIF, TIMP1, TFRC | NONE |
| 3796 | GSN, PKM, TIMP1, TFRC | NONE |
| 3797 | MIF, PKM, TIMP1, TFRC | NONE |
| 3798 | SERPINA1, SERPINA3 | Age |
| 3799 | SERPINA1, CTSD | Age |
| 3800 | SERPINA1, CLU | Age |
| 3801 | SERPINA1, DPP4 | Age |
| 3802 | SERPINA1, GDF15 | Age |
| 3803 | SERPINA1, GSN | Age |
| 3804 | SERPINA1, MIF | Age |
| 3805 | SERPINA1, PKM | Age |
| 3806 | SERPINA1, TIMP1 | Age |
| 3807 | SERPINA1, TFRC | Age |
| 3808 | SERPINA3, CTSD | Age |
| 3809 | SERPINA3, CLU | Age |
| 3810 | SERPINA3, DPP4 | Age |
| 3811 | SERPINA3, GDF15 | Age |
| 3812 | SERPINA3, GSN | Age |
| 3813 | SERPINA3, MIF | Age |
| 3814 | SERPINA3, PKM | Age |
| 3815 | SERPINA3, TIMP1 | Age |
| 3816 | SERPINA3, TFRC | Age |
| 3817 | CTSD, CLU | Age |
| 3818 | CTSD, DPP4 | Age |
| 3819 | CTSD, GDF15 | Age |
| 3820 | CTSD, GSN | Age |
| 3821 | CTSD, MIF | Age |
| 3822 | CTSD, PKM | Age |
| 3823 | CTSD, TIMP1 | Age |
| 3824 | CTSD, TFRC | Age |
| 3825 | CLU, DPP4 | Age |
| 3826 | CLU, GDF15 | Age |
| 3827 | CLU, GSN | Age |
| 3828 | CLU, MIF | Age |
| 3829 | CLU, PKM | Age |
| 3830 | CLU, TIMP1 | Age |
| 3831 | CLU, TFRC | Age |
| 3832 | DPP4, GDF15 | Age |
| 3833 | DPP4, GSN | Age |
| 3834 | DPP4, MIF | Age |
| 3835 | DPP4, PKM | Age |
| 3836 | DPP4, TIMP1 | Age |
| 3837 | DPP4, TFRC | Age |
| 3838 | GDF15, GSN | Age |
| 3839 | GDF15, MIF | Age |
| 3840 | GDF15, PKM | Age |
| 3841 | GDF15, TIMP1 | Age |
| 3842 | GDF15, TFRC | Age |
| 3843 | GSN, MIF | Age |
| 3844 | GSN, PKM | Age |
| 3845 | GSN, TIMP1 | Age |
| 3846 | GSN, TFRC | Age |
| 3847 | MIF, PKM | Age |
| 3848 | MIF, TIMP1 | Age |
| 3849 | MIF, TFRC | Age |
| 3850 | PKM, TIMP1 | Age |
| 3851 | PKM, TFRC | Age |
| 3852 | TIMP1, TFRC | Age |
| 3853 | SERPINA1, SERPINA3, CTSD | NONE |
| 3854 | SERPINA1, SERPINA3, CLU | NONE |
| 3855 | SERPINA1, SERPINA3, DPP4 | NONE |
| 3856 | SERPINA1, SERPINA3, GDF15 | NONE |
| 3857 | SERPINA1, SERPINA3, GSN | NONE |
| 3858 | SERPINA1, SERPINA3, MIF | NONE |
| 3859 | SERPINA1, SERPINA3, PKM | NONE |
| 3860 | SERPINA1, SERPINA3, TIMP1 | NONE |
| 3861 | SERPINA1, SERPINA3, TFRC | NONE |
| 3862 | SERPINA1, CTSD, CLU | NONE |
| 3863 | SERPINA1, CTSD, DPP4 | NONE |
| 3864 | SERPINA1, CTSD, GDF15 | NONE |
| 3865 | SERPINA1, CTSD, GSN | NONE |
| 3866 | SERPINA1, CTSD, MIF | NONE |
| 3867 | SERPINA1, CTSD, PKM | NONE |
| 3868 | SERPINA1, CTSD, TIMP1 | NONE |
| 3869 | SERPINA1, CTSD, TFRC | NONE |
| 3870 | SERPINA1, CLU, DPP4 | NONE |
| 3871 | SERPINA1, CLU, GDF15 | NONE |
| 3872 | SERPINA1, CLU, GSN | NONE |
| 3873 | SERPINA1, CLU, MIF | NONE |
| 3874 | SERPINA1, CLU, PKM | NONE |
| 3875 | SERPINA1, CLU, TIMP1 | NONE |
| 3876 | SERPINA1, CLU, TFRC | NONE |
| 3877 | SERPINA1, DPP4, GDF15 | NONE |
| 3878 | SERPINA1, DPP4, GSN | NONE |
| 3879 | SERPINA1, DPP4, MIF | NONE |
| 3880 | SERPINA1, DPP4, PKM | NONE |
| 3881 | SERPINA1, DPP4, TIMP1 | NONE |
| 3882 | SERPINA1, DPP4, TFRC | NONE |
| 3883 | SERPINA1, GDF15, GSN | NONE |
| 3884 | SERPINA1, GDF15, MIF | NONE |
| 3885 | SERPINA1, GDF15, PKM | NONE |
| 3886 | SERPINA1, GDF15, TIMP1 | NONE |
| 3887 | SERPINA1, GDF15, TFRC | NONE |
| 3888 | SERPINA1, GSN, MIF | NONE |
| 3889 | SERPINA1, GSN, PKM | NONE |
| 3890 | SERPINA1, GSN, TIMP1 | NONE |
| 3891 | SERPINA1, GSN, TFRC | NONE |
| 3892 | SERPINA1, MIF, PKM | NONE |
| 3893 | SERPINA1, MIF, TIMP1 | NONE |
| 3894 | SERPINA1, MIF, TFRC | NONE |
| 3895 | SERPINA1, PKM, TIMP1 | NONE |
| 3896 | SERPINA1, PKM, TFRC | NONE |
| 3897 | SERPINA1, TIMP1, TFRC | NONE |
| 3898 | SERPINA3, CTSD, CLU | NONE |
| 3899 | SERPINA3, CTSD, DPP4 | NONE |
| 3900 | SERPINA3, CTSD, GDF15 | NONE |
| 3901 | SERPINA3, CTSD, GSN | NONE |
| 3902 | SERPINA3, CTSD, MIF | NONE |
| 3903 | SERPINA3, CTSD, PKM | NONE |
| 3904 | SERPINA3, CTSD, TIMP1 | NONE |
| 3905 | SERPINA3, CTSD, TFRC | NONE |
| 3906 | SERPINA3, CLU, DPP4 | NONE |
| 3907 | SERPINA3, CLU, GDF15 | NONE |
| 3908 | SERPINA3, CLU, GSN | NONE |
| 3909 | SERPINA3, CLU, MIF | NONE |
| 3910 | SERPINA3, CLU, PKM | NONE |
| 3911 | SERPINA3, CLU, TIMP1 | NONE |
| 3912 | SERPINA3, CLU, TFRC | NONE |
| 3913 | SERPINA3, DPP4, GDF15 | NONE |
| 3914 | SERPINA3, DPP4, GSN | NONE |
| 3915 | SERPINA3, DPP4, MIF | NONE |
| 3916 | SERPINA3, DPP4, PKM | NONE |
| 3917 | SERPINA3, DPP4, TIMP1 | NONE |
| 3918 | SERPINA3, DPP4, TFRC | NONE |
| 3919 | SERPINA3, GDF15, GSN | NONE |
| 3920 | SERPINA3, GDF15, MIF | NONE |
| 3921 | SERPINA3, GDF15, PKM | NONE |
| 3922 | SERPINA3, GDF15, TIMP1 | NONE |
| 3923 | SERPINA3, GDF15, TFRC | NONE |
| 3924 | SERPINA3, GSN, MIF | NONE |
| 3925 | SERPINA3, GSN, PKM | NONE |
| 3926 | SERPINA3, GSN, TIMP1 | NONE |
| 3927 | SERPINA3, GSN, TFRC | NONE |
| 3928 | SERPINA3, MIF, PKM | NONE |
| 3929 | SERPINA3, MIF, TIMP1 | NONE |
| 3930 | SERPINA3, MIF, TFRC | NONE |
| 3931 | SERPINA3, PKM, TIMP1 | NONE |
| 3932 | SERPINA3, PKM, TFRC | NONE |
| 3933 | SERPINA3, TIMP1, TFRC | NONE |
| 3934 | CTSD, CLU, DPP4 | NONE |
| 3935 | CTSD, CLU, GDF15 | NONE |
| 3936 | CTSD, CLU, GSN | NONE |
| 3937 | CTSD, CLU, MIF | NONE |
| 3938 | CTSD, CLU, PKM | NONE |
| 3939 | CTSD, CLU, TIMP1 | NONE |
| 3940 | CTSD, CLU, TFRC | NONE |
| 3941 | CTSD, DPP4, GDF15 | NONE |
| 3942 | CTSD, DPP4, GSN | NONE |
| 3943 | CTSD, DPP4, MIF | NONE |
| 3944 | CTSD, DPP4, PKM | NONE |
| 3945 | CTSD, DPP4, TIMP1 | NONE |
| 3946 | CTSD, DPP4, TFRC | NONE |
| 3947 | CTSD, GDF15, GSN | NONE |
| 3948 | CTSD, GDF15, MIF | NONE |
| 3949 | CTSD, GDF15, PKM | NONE |
| 3950 | CTSD, GDF15, TIMP1 | NONE |
| 3951 | CTSD, GDF15, TFRC | NONE |
| 3952 | CTSD, GSN, MIF | NONE |
| 3953 | CTSD, GSN, PKM | NONE |
| 3954 | CTSD, GSN, TIMP1 | NONE |
| 3955 | CTSD, GSN, TFRC | NONE |
| 3956 | CTSD, MIF, PKM | NONE |
| 3957 | CTSD, MIF, TIMP1 | NONE |
| 3958 | CTSD, MIF, TFRC | NONE |
| 3959 | CTSD, PKM, TIMP1 | NONE |
| 3960 | CTSD, PKM, TFRC | NONE |
| 3961 | CTSD, TIMP1, TFRC | NONE |
| 3962 | CLU, DPP4, GDF15 | NONE |
| 3963 | CLU, DPP4, GSN | NONE |
| 3964 | CLU, DPP4, MIF | NONE |
| 3965 | CLU, DPP4, PKM | NONE |
| 3966 | CLU, DPP4, TIMP1 | NONE |
| 3967 | CLU, DPP4, TFRC | NONE |
| 3968 | CLU, GDF15, GSN | NONE |
| 3969 | CLU, GDF15, MIF | NONE |
| 3970 | CLU, GDF15, PKM | NONE |
| 3971 | CLU, GDF15, TIMP1 | NONE |
| 3972 | CLU, GDF15, TFRC | NONE |
| 3973 | CLU, GSN, MIF | NONE |
| 3974 | CLU, GSN, PKM | NONE |
| 3975 | CLU, GSN, TIMP1 | NONE |
| 3976 | CLU, GSN, TFRC | NONE |
| 3977 | CLU, MIF, PKM | NONE |
| 3978 | CLU, MIF, TIMP1 | NONE |
| 3979 | CLU, MIF, TFRC | NONE |
| 3980 | CLU, PKM, TIMP1 | NONE |
| 3981 | CLU, PKM, TFRC | NONE |
| 3982 | CLU, TIMP1, TFRC | NONE |
| 3983 | DPP4, GDF15, GSN | NONE |
| 3984 | DPP4, GDF15, MIF | NONE |
| 3985 | DPP4, GDF15, PKM | NONE |
| 3986 | DPP4, GDF15, TIMP1 | NONE |
| 3987 | DPP4, GDF15, TFRC | NONE |
| 3988 | DPP4, GSN, MIF | NONE |
| 3989 | DPP4, GSN, PKM | NONE |
| 3990 | DPP4, GSN, TIMP1 | NONE |
| 3991 | DPP4, GSN, TFRC | NONE |
| 3992 | DPP4, MIF, PKM | NONE |
| 3993 | DPP4, MIF, TIMP1 | NONE |
| 3994 | DPP4, MIF, TFRC | NONE |
| 3995 | DPP4, PKM, TIMP1 | NONE |
| 3996 | DPP4, PKM, TFRC | NONE |
| 3997 | DPP4, TIMP1, TFRC | NONE |
| 3998 | GDF15, GSN, MIF | NONE |
| 3999 | GDF15, GSN, PKM | NONE |
| 4000 | GDF15, GSN, TIMP1 | NONE |
| 4001 | GDF15, GSN, TFRC | NONE |
| 4002 | GDF15, MIF, PKM | NONE |
| 4003 | GDF15, MIF, TIMP1 | NONE |
| 4004 | GDF15, MIF, TFRC | NONE |
| 4005 | GDF15, PKM, TIMP1 | NONE |
| 4006 | GDF15, PKM, TFRC | NONE |
| 4007 | GDF15, TIMP1, TFRC | NONE |
| 4008 | GSN, MIF, PKM | NONE |
| 4009 | GSN, MIF, TIMP1 | NONE |
| 4010 | GSN, MIF, TFRC | NONE |
| 4011 | GSN, PKM, TIMP1 | NONE |
| 4012 | GSN, PKM, TFRC | NONE |
| 4013 | GSN, TIMP1, TFRC | NONE |
| 4014 | MIF, PKM, TIMP1 | NONE |
| 4015 | MIF, PKM, TFRC | NONE |
| 4016 | MIF, TIMP1, TFRC | NONE |
| 4017 | PKM, TIMP1, TFRC | NONE |

Additional exemplary AA panels consistent with the disclosure herein are listed in Table 6. Also disclosed are panels comprising the markers listed in entries of Table 6. In some cases, the panels listed in Table 6 can be used as alternatives to panels listed in Table 5 above. Table 6 also includes the Area Under Curve values “AUC”, sensitivity “Sens” and specificity “Spec” values corresponding to each panel.

TABLE 6
AA biomarker panel constituents

| | | | | | Sens/ |
|---|---|---|---|---|---|
| Ref | AA Protein Biomarker | Demographics | Features | AUC | Spec |
| 1 | ORM1, SERPINA1, SERPINA3, CTSD, CEA, | Age | 15 | 65 | 38/80 |
| | CLU, C9, GDF15, GSN, MIF, PKM, SAA, TFRC, | | | | |
| | TIMP1 | | | | |
| 2 | ORM1, SERPINA1, SERPINA3, | Age | 14 | 65 | 38/80 |
| | CTSD, CLU, C9, GDF15, GSN, MIF, PKM, SAA, | | | | |
| | TFRC, TIMP1 | | | | |
| 3 | ORM1, SERPINA1, | Age | 13 | 65 | 38/80 |
| | CTSD, CLU, C9, GDF15, GSN, MIF, PKM, SAA, | | | | |
| | TFRC, TIMP1 | | | | |
| 4 | ORM1, SERPINA1, | Age | 8 | 62 | 35/80 |
| | CTSD, DPP4, PKM, SAA, TIMP1 | | | | |
| 5 | SERPINA1, SERPINA3, | Age and Gender | 13 | 65 | 38/80 |
| | CTSD, CEA, CLU, C9, GDF15, GSN, | | | | |
| | MIF, PKM, TFRC | | | | |
| 6 | SERPINA1, SERPINA3, CTSD, CEA, CLU, GSN | Age and Gender | 13 | 65 | 38/80 |
| | MIF, PKM, SAA, TFRC, TIMP1 | | | | |
| 7 | SERPINA1, CTSD, CEA, CLU, DPP4, GSN, | Age and Gender | 13 | 66 | 39/80 |
| | MIF, PKM, SAA, TFRC, TIMP1 | | | | |
| 8 | SERPINA1, | Age | 10 | 63 | 38/80 |
| | CTSD, CLU, C9, DPP4, GSN, MIF, SAA, TIMP1 | | | | |
| 9 | SERPINA1, CTSD, CLU, C9, DPP4, MIF, | Age | 9 | 66 | 39/80 |
| | PKM, TIMP1 | | | | |
| 10 | SERPINA1, CTSD, C9, DPP4, GSN, MIF, | Age | 9 | 66 | 40/80 |
| | PKM, TIMP1 | | | | |
| 11 | SERPINA3, CTSD, CLU, DPP4, GSN, | Age and Gender | 12 | 66 | 39/80 |
| | MIF, PKM, SAA, TFRC, TIMP1 | | | | |
| 12 | SERPINA3, CTSD, DPP4, MIF, PKM, TFRC | Age | 7 | 66 | 39/80 |
| 13 | CTSD, GDF15, MIF, PKM | Age | 6 | 65 | 38/80 |
| 14 | CEA, C9, DPP4, GSN, MIF, PKM, SAA, TFRC | Age and Gender | 10 | 64 | 40/80 |

### Health Assessment Assays

The biomarker panels, methods, compositions, and kits described herein provide assays for at least one of advanced colorectal adenoma and CRC based on detection or measurement of biomarkers in a biological sample obtained from a subject. The biological sample preferably is a blood sample drawn from an artery or vein of an individual. The blood sample can be a whole blood sample, a plasma sample, or a serum sample. The disclosure provided herein detects at least one of advanced colorectal adenoma and CRC from a sample such as a blood sample with a sensitivity and a specificity that renders the outcome of the test reliable enough to be medically actionable. Health assessment methods, systems, kits and panels herein have at least one of a sensitivity of at least 40%, at least 50%, at least 60%, at least 70% and specificity of at least 70%. Such CRC related methods can have at least one of a sensitivity of 70% or greater and specificity of at least 70% based on measurement of 15 or fewer biomarkers in the biological sample. In some cases, a method provided herein detects at least one of advanced colorectal adenoma and CRC. Such method can have at least one of a sensitivity at least 40% for AA detection and at least 70% for CRC detection and specificity at least 70% based on measurement of no more than 4 biomarkers, 5 biomarkers, 6 biomarkers, 7, biomarkers, 8 biomarkers, 9 biomarkers, 10 biomarkers, 11, biomarkers, 12 biomarkers, 13 biomarkers, 14 biomarkers, or 15 biomarkers. Some preferred embodiments allow one to assess colorectal cancer using a biomarker panel of at least 8 markers. Some preferred embodiments allow one to assess advanced adenoma using a panel of at least 4 biomarkers. Some biomarker panels allow one to assess both colorectal cancer and advanced adenoma using a combined panel of 11, 12, 13, 14, 15, 16, 17, or more than 17 biomarkers.

In some cases the biomarker panels, methods, compositions, and kits described herein are useful to screen for individuals at elevated risk for CRC or advanced adenoma. In some cases, a positive detection of at least one of an advanced colorectal adenoma and CRC based upon a method described herein is used to identify patients for whom to recommend an additional diagnostic method. For example, in some cases where a method herein yields a positive result, such method is used to alert a caregiver to perform an additional test such as a colonoscopy, a sigmoidoscopy, an independent cancer assay, or a stool cancer assay.

The biomarker panels, methods, compositions, and kits described herein are also useful as a quality control metric for a colonoscopy, sigmoidoscopy, or colon tissue biopsy. For example, a positive detection of at least one of an advanced colorectal adenoma and CRC based upon a method described herein can be used to validate a result of a colonoscopy, sigmoidoscopy, or colon tissue biopsy. For example, in some cases wherein a colonoscopy, sigmoidoscopy, or colon tissue biopsy yielded a negative result, but a method described herein yielded a positive result, such method can be used to alert a caregiver to perform another colonoscopy, sigmoidoscopy, or colon tissue biopsy, or to initiate a treatment regimen such as administration of a pharmaceutical composition. The treatment regimen may include one or more other procedures as described herein.

Some methods provided herein comprise (a) obtaining a biological sample from a subject; (b) measuring a panel of biomarkers in the biological sample of the subject; (c) detecting a presence or absence of at least one of advanced colorectal adenoma and CRC in the subject based upon the measuring; and (d) either (i) treating the at least one of advanced colorectal adenoma CRC and in the subject based upon the detecting, or (ii) recommending to the subject a colonoscopy, sigmoidoscopy, or colorectal tissue biopsy based upon the results of the detecting. For the purposes of one or more methods described herein, “treating” comprises providing a written report to the subject or to a caretaker of the subject which includes a recommendation to initiate a treatment for the CRC. For the purposes of one or more methods described herein, “recommending to the subject a colonoscopy” comprises providing a written report to the subject or to a caretaker of the subject which includes a recommendation that the subject undergo a colonoscopy, sigmoidoscopy, or tissue biopsy to confirm an assessment of the CRC. In some cases, the colonoscopy, sigmoidoscopy, or tissue biopsy can be used to remove the at least one of advanced colorectal adenoma and CRC, thereby treating the at least one of advanced colorectal adenoma and CRC.

Exemplary methods optionally comprise (a) obtaining data comprising a measurement of a biomarker panel in a biological sample obtained from a subject, (b) generating a subject-specific profile of the biomarker panel based upon the measurement data, (c) comparing the subject-specific profile of the biomarker panel to a reference profile of the biomarker panel; and (d) determining a likelihood of at least one of advanced colorectal adenoma and colorectal cancer based upon (c).

Exemplary methods optionally comprise (a) measuring a biomarker panel in a biological sample obtained from the subject; (b) detecting a presence or absence of colorectal cancer and/or advanced colorectal adenoma in the subject based upon the measuring; and (c) treating the colorectal cancer in the subject based upon the detecting.

Exemplary methods optionally comprise (a) obtaining data comprising a measurement of a biomarker panel in a biological sample obtained from a subject, (b) generating a subject-specific profile of the biomarker panel based upon the measurement data, (c) comparing the subject-specific profile of the biomarker panel to a reference profile of the biomarker panel; and (d) determining a likelihood of at least one of advanced colorectal adenoma and colorectal cancer based upon (c). Some methods provided herein comprise (a) measuring a biomarker panel in a biological sample obtained from the subject; (b) detecting a presence or absence of colorectal cancer and/or advanced colorectal adenoma in the subject based upon the measuring; and (c) recommending to the subject at least one of a colonoscopy, sigmoidoscopy, and tissue biopsy in the subject based upon the detecting. Exemplary methods optionally comprise diagnosis of colorectal cancer or monitoring colorectal cancer, so as to establish a prognosis for the subject. The levels of one or a combination of the proteins listed can over time be linked to differential outcomes for cancer patients, possibly depending on the treatment chosen. Exemplary methods optionally comprise monitoring the progression of cancer in a subject by comparing the accumulation levels of one or more biomarkers in a sample from a subject to the accumulation levels of the one or more biomarkers in a sample obtained from the subject at a subsequent point in time, wherein a difference in the expression of said one or more biomarkers diagnoses or aids in the diagnosis of the progression of the cancer in the subject. Some exemplary methods comprise monitoring the effectiveness of a treatment. In some cases, a method for monitoring the effectiveness of a treatment comprises comparing the accumulation levels of one or more biomarkers in a sample from a subject prior to providing at least a portion of a treatment to the accumulation levels of said one or more biomarkers in a sample obtained from the subject after the subject has received at least a portion of the treatment, and wherein a difference in the accumulation levels of said one or more biomarker diagnoses or aids in the diagnosis of the efficacy of the treatment.

Monitoring of the subject can be performed for a duration of more than about 3 months, about 6 months, about 9 months, about 12 months, about 15 months, about 18 months, about 21 months, or about 24 months. For example, at least one of monitoring of the health status of the subject and effectiveness of an administrated treatment can be performed for one or more of the durations described above. In some cases, at least one of testing and treatment of the subject can be repeated after one or more durations described above. For example, the subject may be retested at about 3 months, about 6 months, about 9 months, about 12 months, about 15 months, about 18 months, about 21 months, or about 24 months.

In some cases, exemplary methods include recommending one or more of chemotherapy, radiation, immunotherapy, administration of a biologic therapeutic agent, polypectomy, partial colectomy, low anterior resection or abdominoperineal resection and colostomy. In some cases, exemplary methods can include recommending administrating to the subject one or more of leucovorin, 5-FU, oxaliplatin (Eloxatin®), irinotecan (Camptosar®), capecitabine (Xeloda®), Cetuximab, Panitumumab, Regorafenib (Stivarga®), trifluridine and tipiracil (Lonsurf®). In some cases, exemplary methods can include recommending administrating to the subject one or more of FOLFOX: leucovorin, 5-FU, and oxaliplatin (Eloxatin®); FOLFIRI: leucovorin, 5-FU, and irinotecan (Camptosar®); CapeOX: capecitabine (Xeloda®) and oxaliplatin; and FOLFOXIRI: leucovorin, 5-FU, oxaliplatin, and irinotecan. In some cases, exemplary methods can include recommending administrating to the subject one or more of a drug that targets VEGF (e.g., bevacizumab (Avastin®), ziv-aflibercept (Zaltrap®), ramucirumab (Cyramza®), and a drug that targets EGFR (e.g., cetuximab (Erbitux®), panitumumab (Vectibix®)). For example, exemplary methods can include providing a written report, such as to a subject or a caretaker of the subject, which includes a recommendation for the subject to undergo one or more of the regimens described herein, including one or more of chemotherapy, radiation, immunotherapy, administration of a biologic therapeutic agent, polypectomy, partial colectomy, low anterior resection or abdominoperineal resection and colostomy.

### Biomarker Measurement

Biomarkers are measured through a number of approaches consistent with the disclosure herein. In many cases biomarkers are measured through an immunological interaction, such as that which occurs in an ELISA assay through which proteins or protein fragments in a blood sample from an individual are bound to specific antibodies, and the extent of binding is quantified as a measure of protein abundance in the sample. ELISA assays capable of measuring biomarker panels as disclosed herein are contemplated as embodiments of the present disclosure as kits.

Alternately or in combination, biomarkers are measured through mass spectrometric methods such as MS, MS/MS, MALDI-TOF or other mass spectrometric approaches as appropriate. Often, the MS approach quantifies a fragment of a biomarker rather than the full-length protein. However, such approaches are sufficient to determine the protein level of the biomarker to an accuracy sufficient for a colorectal health assessment as disclosed herein.

Some details of panel performance is dependent upon assay approach, such that some panels perform slightly better using an immunological or a mass spectrometric approach. However, it is observed that in many cases panel performance is largely independent of assay method, such that a panel that performs slightly better using an immunological assay is nonetheless informative as to an individual's colorectal health status when assayed using mass spectrometric analysis, or vice versa.

Once an expression level for a biomarker panel is determined, a colorectal health assessment is available for the individual from which the sample is obtained. A number of approaches are available to one of skill in the art to generate or come to a colorectal health assessment from an individual's biomarker panel expression level.

Some assessments rely upon comparison of an individual's biomarker panel level to a reference level, such as a reference biomarker panel level from an individual known or independently verified to be in good colorectal health, or from an individual known or independently verified to be in poor colorectal health, such as is the case for an individual having colorectal cancer or at least one advanced adenoma. Alternately or in combination an individual's biomarker panel level is compared to a reference level constructed from a plurality of individuals of common known colorectal health status. In some cases the reference is an average of known panel levels from a plurality of individuals, or alternately is a range defined by the range of panel levels observed in the reference individuals. A range reference panel level is in some cases a weighted range, such that outlier values among the individuals having a common colorectal health status are given lower predictive value than panel levels that are common to a plurality or majority or all of the panel levels.

In more complex assessment approaches, an individual's biomarker panel level is compared to a reference level constructed from a larger number of individuals of common known colorectal health status, such as at least 10, at least 50, at least 100, at least 500, at least 1000 or more individuals. Often, the reference individuals are evenly distributed in health status between positive and negative for a colorectal health status such as positive and negative for colorectal cancer, or positive and negative for advanced adenoma. Assessment comprises in some cases iterative or simultaneous comparison of an individual's biomarker panel level to a plurality of references of known health status.

Alternately or in combination, a plurality of known reference biomarker panel levels are used to train a computational assessment algorithm such as a machine learning model such that a single comparison between an individual's biomarker panel level and a reference provides an outcome that integrates or aggregates information from a large number of individuals of common known colorectal health status, such as at least 10, at least 50, at least 100, at least 500, at least 1000 or more individuals. Generation of such a reference often facilitates much faster assessment of an individual's colorectal health status, or assessment using much less computational power.

A reference is generated from a plurality of reference individual biomarker levels through any of a number of computational approaches known to one of skill in the art. Machine learning models are readily constructed, for example, using any number of statistical programming languages such as R, scripting languages such as Python and associated machine learning packages, data mining software such as Weka or Java, Mathematica, Matlab or SAS.

An individual's biomarker panel level is compared to a reference as generated above or otherwise by one of skill in the art, and an output assessment is generated. A number of output assessments are consistent with the disclosure herein. Output assessments comprise a single assessment, often narrowed by a sensitivity, specificity or sensitivity and specificity parameter, indicating a colorectal health status assessment. Alternately or in combination, additional parameters are provided, such as an odds ratio indicative of the relative increase in chance of suffering from a colorectal health issue in light of the individual's biomarker panel level or biomarker panel level assessment.

Results are variously provided to the individual or to a health care professional or other professional. Results are optionally accompanied by a heath recommendation, such as a recommendation to confirm or independently assess a colorectal health status assessment, for example using a stool sample assay or an invasive approach such as a colonoscopy, sigmoidoscopy or other supplemental assay for colorectal health.

A recommendation optionally includes information relevant to a treatment regimen, such as information indicating that a treatment regimen such as a polypectomy, radiotherapy, chemotherapy, antibody therapy, biosimilar treatment or other treatment regimen, such as information indicative of success or efficacy of the regimen. Efficacy of a regimen is assessed in some cases by comparison of an individual's biomarker panel level at a first time point, optionally prior to a treatment and a later second time point, optionally subsequent to a treatment instance. Biomarker panel levels are compared to one another, each to a reference, or otherwise assessed so as to determine whether a treatment regimen demonstrates efficacy such that it should be continued, increased, replaced with an alternate regimen or discontinued because of its success in addressing the colorectal health issue such as colorectal cancer or advanced adenoma. Some assessments rely upon comparison of an individual's biomarker panel level at multiple time points, such as at least one time point prior to a treatment and at least one time point following a treatment. Biomarker panel levels are compared one to another or to at least one reference biomarker panel level or both to one another and to at least one reference biomarker panel level.

### Biomarker Panel Assessment

Some methods described herein comprise comparing the amount of each of the at least two biomarkers in the biological sample to a reference amount of each of the at least two biomarkers. Some methods herein comprise comparing the profile of the biomarker panel in a subject to a reference profile of the biomarker panel. The reference amount is in some cases an amount of the biomarker in a control subject. The reference profile of the biomarker panel is in some cases a biomarker profile of a control subject. The control subject is in some cases a subject having a known diagnosis. For example, the control subject can be a negative control subject. The negative control subject can be a subject that does not have advanced colorectal adenoma. The negative control subject can be a subject that does not have CRC. The negative control subject can be a subject that does not have a colon polyp. For other example, the control subject can be a positive control subject. The positive control subject can be a subject having a confirmed diagnosis of advanced colorectal adenoma. The positive control subject can be a subject having a confirmed diagnosis of CRC. The positive control subject can be a subject having a confirmed diagnosis of any stage of CRC (for example, Stage 0, Stage I, Stage II, Stage IIA, Stage IIB, Stage IIC, Stage III, Stage IIIA, Stage IIIB, Stage IIIC, Stage IV, Stage IVA, or Stage IVB). The reference amount can be a predetermined level of the biomarker, wherein the predetermined level is set based upon a measured amount of the biomarker in a control subject.

Some reference biomarker panel levels comprises average values for a number of individuals having a common condition status, such as 10 individuals free of CRC or AA, or 10 individuals of a known stage of CRC or a known AA status. Alternately, in some cases references comprise a set of protein accumulation levels, and age in some embodiments, that correspond to a set of individuals of known CRC or AA status. In these cases, levels are not averaged; rather, a patient's levels are compared to each set of accumulation levels of each standard or reference individual in the set, and a determination is made if the patient's accumulation levels do not differ significantly from those of at least one reference set. In some cases the reference set comprises individuals of known cancer-free status, while in some cases the reference set comprises individuals of known CRC or AA stage status, such as Stage 0, Stage I, Stage II, Stage 11A, Stage IIB, Stage IIC, Stage III, Stage 111A, Stage IIIB, Stage IIIC, Stage IV, Stage IVA, or Stage IVB. In some cases a patient is categorized as having a condition if the patient's panel accumulation levels match or do not differ significantly from those of a reference. In some cases a patient is categorized as not having a condition if a patient's panel accumulation levels differ significantly from those of a reference.

In some cases, comparing comprises determining a difference between an amount of the biomarker in the biological sample obtained from the subject and the reference amount of the biomarker. The method comprises, in some cases, detecting a presence or absence of at least one of advanced colorectal adenoma and CRC based upon a deviation (for example, measured difference) of the amount of at least one of the measured biomarkers in the biological sample obtained from the subject as compared to a reference amount of the at least one measured biomarkers. In some cases, the method comprises detecting a presence of at least one of advanced colorectal adenoma and CRC if the deviation of the amount of the at least one measured biomarker from the biological sample obtained from the subject as compared to a positive reference value (for example, an amount of the measured biomarker from a positive control subject) is low. In other cases, the method comprises detecting a presence of at least one of advanced colorectal adenoma and CRC if the deviation of the amount of the at least one measured biomarker from the biological sample obtained from the subject as compared to a negative reference value (for example, measured from a negative control subject) is high. In some cases, the method comprises detecting an absence of at least one of advanced colorectal adenoma and CRC if the deviation of the amount of the at least one measured biomarker from the biological sample obtained from the subject as compared to a positive reference value (for example, measured from a positive control subject) is high. In some examples, the method comprises detecting an absence of at least one of advanced colorectal adenoma and CRC if the deviation of the amount of the at least one measured biomarker from the biological sample obtained from the subject as compared to a negative reference value (for example, measured from a negative control subject) is low. In some cases, detection of a presence or absence of at least one of advanced colorectal adenoma and CRC can be based upon a clinical outcome score produced by an algorithm described herein. In some cases, the method comprises detection of a presence or absence of colorectal cancer based upon a classifier that divides a feature space into feature values that are predictive of the presence of colorectal cancer and feature values that are predictive of the absence of colorectal cancer. In some cases, the method comprises classifying a subject's colorectal cancer status as “undetermined” (e.g., “no call”) in order to reduce false positives and/or false negatives. In some cases, patients with an undetermined colorectal cancer status are retested at a later point. The algorithm can be used for assessing the deviation between an amount of a measured biomarker in the biological sample obtained from the subject and a reference amount of the biomarker.

In some cases, a classifier is used to determine the colorectal cancer status of a subject. For example, given N measurements as inputs into the classifier (e.g., the biomarkers comprising proteins and the age of the subject), the subject can be represented as a point in an N-dimensional space wherein each axis is a measurement. In some cases, the classifier defines an N-1)-dimensional shape that divides the N-dimensional space into two or more categories. In some cases, the two categories are a subject with cancer and a subject without cancer. In some cases there are three categories. In some cases the categories are a subject with cancer, a subject without cancer, and a no-call region where the cancer status of the subject cannot be reliably determined. In some cases, the classifier allows ‘shifting’ cutoffs for particular proteins. For example, consider a classifier defined by the boundary y=1/x, where x and y are both greater than zero, and each of the two axes is the accumulation level of a protein indicative of cancer status. In such a case, all the subjects whose protein accumulation levels fall beneath the boundary (e.g., [0, 0], [2, 0.3], etc.) are classified as not having the condition, whereas any subject whose protein accumulation levels lie above the boundary are classified as having the condition. If the x-axis protein has a value of 1, then in this example the y-axis protein must be more than one to result in a cancer diagnosis. However, if the x-axis protein has a value of 10, then the y-axis protein need only have a value more than 0.1 to result in a cancer diagnosis. This example can be extrapolated to an N-dimensional shape using an (N-1)-dimensional shape as the classifier.

The intrinsic performance of a particular classification model depends on the distributions and separation of model scores for the two classes. With the rare exception of perfect class separation, most classification models make mistakes because of class overlap across the range of classifier scores. For example, such an overlap may occur near the middle of the score range where the probability of being in one class or the other is close to 50%.

Within such an overlap region, it is sometimes advantageous to add a third class to the final set of classification calls. The third class optionally indicates the uncertainty of a call in this score region. This is implemented, for example, by defining an indeterminate region of classification scores. Samples with scores in this region are given an “indeterminate” or “no call” test result. Samples with scores above or below this region would be given standard positive or negative test results depending on their positions relative to the test cutoff. In some cases, the “no call” rate, or the frequency with which samples fall into the “no call” region, is about 1%, about 2%, about 3%, about 4%, about 5%, about 10%, about 15%, or about 20%. In particular, the “no call” rate can be about 10%. The benefit of adding an indeterminate region to a classification model is that classification performance can improve for samples outside of the indeterminate region, i.e. mistakes are less likely for the remaining positive and negative tests. However, if the indeterminate range is too large, there may be too many indeterminate results, and the value of the test may be put into question.

### Classifier Construction

Reference classifiers are readily constructed by one of skill in the art using any number of available technologies. Reference classifiers are, for example, generated by assaying panel levels for a plurality of samples, such as blood sample, obtained from individuals of known colorectal health status. As many as 1000 samples or more, comprising samples obtained from individuals known or later confirmed to have colorectal cancer or known or later confirmed not to have colorectal cancer, as assayed as to their biomarker panel levels. Age, a non-protein biomarker constituent of some panels, is also recorded for each individual at the time of sample collection.

In some cases, the biomarker panel levels for each sample are used individually as a reference panel level for comparison so as to classify an individual's biomarker panel level as indicative of a healthy colorectal health status or a colorectal health issue warranting further investigation. A panel level to be classified is compared to the positive and the negative biomarker panel levels, and the outcome as judged by, for example, the number samples of each category from which the testing individual's panel level does not differ significantly.

Alternately, a classifier is assembled from the collection of biomarker panel levels. Classifier assembly is well known to those of skill in the art. Machine learning models, in particular, are useful in assembling a classifier from a set of panel levels obtained from samples of known colorectal health status. Machine learning models are readily constructed, for example, using any number of statistical programming languages such as R, scripting languages such as Python and associated machine learning packages, data mining software such as Weka or Java, Mathematica, Matlab or SAS.

### Implementation of Classifiers in Colorectal Health Assessment

In practicing any of the methods described herein, comparing optionally comprises determining a difference between a biomarker profile of a subject to a reference biomarker profile. The method can, for example, comprise detecting a presence or absence of at least one of advanced colorectal adenoma and CRC based upon a deviation (for example, measured difference) of the biomarker profile of the subject as compared to a reference biomarker profile. For example, some methods comprise detecting a presence of at least one of advanced colorectal adenoma and CRC if the deviation of the biomarker profile of the subject as compared to a positive reference biomarker profile (for example, a biomarker profile based upon measurements of panel biomarkers from a positive control subject) is low. As an additional example, some methods comprise detecting a presence of at least one of advanced colorectal adenoma and CRC if the deviation of the biomarker profile of the subject as compared to a negative reference biomarker profile (for example, a biomarker profile based upon measurements of panel biomarkers from a negative control subject) is high. In some cases, the method comprises detecting an absence of at least one of advanced colorectal adenoma and CRC if the deviation of the biomarker profile of the subject as compared to a positive reference biomarker profile is high. In some examples, the method comprises detecting an absence of at least one of advanced colorectal adenoma and CRC if the deviation of the biomarker profile of the subject as compared to a negative reference biomarker profile is low. In some cases, detection of a presence or absence of at least one of advanced colorectal adenoma and CRC can be based upon a clinical outcome score produced by an algorithm described herein. The algorithm can be used for assessing the deviation between the biomarker profile of the subject to a reference biomarker profile.

Some methods comprise detecting a presence or absence of an advanced colorectal adenoma in the subject in some cases. The advanced colorectal adenoma can be a colorectal advanced colorectal adenoma. The methods described herein are be used to detect a presence or absence of an advanced colorectal adenoma of any size, such as an advanced adenoma having a dimension that is greater than 1 cm. The methods described herein are used to detect a presence or absence of an advanced colorectal adenoma of villous, serrated, sessile or non-pedunculated character.

In some cases, a diagnostic method provided herein comprises measuring a biomarker panel comprising at least five biomarkers in the biological sample, wherein the at least three biomarkers comprise AACT, CATD, CEA, CO3, CO9, MIF, PSGL, and SEPR. In some cases, the method comprises providing a positive diagnosis of advanced colorectal adenoma if a deviation in the panel level of a panel comprising AACT, CATD, CEA, CO3, CO9, MIF, PSGL, and SEPR in the biological sample obtained from the subject as compared to a positive reference value is low. In some cases, the method comprises providing a positive diagnosis of advanced colorectal adenoma if a deviation in the panel level of a panel comprising AACT, CATD, CEA, CO3, CO9, MIF, PSGL, and SEPR in the biological sample obtained from the subject as compared to a negative reference value is high. In some cases, the method comprises providing a positive diagnosis of advanced colorectal adenoma if a deviation in the panel level of a panel comprising AACT, CATD, CEA, CO3, CO9, MIF, PSGL, and SEPR in the biological sample obtained from the subject as compared to a positive reference value is high. In some cases, the method comprises providing a positive diagnosis of advanced colorectal adenoma if a deviation in the panel level of a panel comprising AACT, CATD, CEA, CO3, CO9, MIF, PSGL, and SEPR in the biological sample obtained from the subject as compared to a negative reference value is low.

Methods, compositions, kits and systems disclosed herein detect advanced colorectal adenoma with a sensitivity of at least 40%, 41%, 42%, 43%, 44%, 45%, 46%, 47%, 48%, 49%, 40%, 51%, 52%, 53%, 54%, 55%, 56%, 57%, 58%, 59%, 60%, 61%, 62%, 63%, 64%, 65%, 70%, 75%, 80% or greater that 80%.

In some cases, a panel comprises a ratio of a level of a first biomarker to a level of a second biomarker. Accordingly, in some cases, a diagnostic method provided herein comprises determining a ratio of a level of the first biomarker to a level of the second biomarker in the biological sample obtained from the subject. In some cases, the method comprises providing a positive diagnosis of CRC if a deviation in the ratio of the first biomarker to the second biomarker in the biological sample obtained from the subject as compared to a positive reference value is low. In some cases, the method comprises providing a positive diagnosis of CRC if a deviation in the ratio of the first biomarker to the second biomarker in the biological sample obtained from the subject as compared to a negative reference value is high. In some cases, the method comprises providing a positive diagnosis of if a deviation in the ratio of the first biomarker to the second biomarker in the biological sample obtained from the subject as compared to a positive reference value is high. In some cases, the method comprises providing a positive diagnosis of CRC if a deviation in the ratio of the first biomarker to the second biomarker in the biological sample obtained from the subject as compared to a negative reference value is low.

In some cases, a panel comprises a ratio of a level of a first biomarker to a level of a second biomarker. Accordingly, in some cases, a diagnostic method provided herein comprises determining a ratio of a level of the first biomarker to a level of the second biomarker in the biological sample obtained from the subject. In some cases, the method comprises providing a positive diagnosis of AA if a deviation in the ratio of the first biomarker to the second biomarker in the biological sample obtained from the subject as compared to a positive reference value is low. In some cases, the method comprises providing a positive diagnosis of AA if a deviation in the ratio of the first biomarker to the second biomarker in the biological sample obtained from the subject as compared to a negative reference value is high. In some cases, the method comprises providing a positive diagnosis of if a deviation in the ratio of the first biomarker to the second biomarker in the biological sample obtained from the subject as compared to a positive reference value is high. In some cases, the method comprises providing a positive diagnosis of AA if a deviation in the ratio of the first biomarker to the second biomarker in the biological sample obtained from the subject as compared to a negative reference value is low.

Diagnostic methods described herein for detection of CRC in a subject detects CRC with a sensitivity greater than 75%, greater than 80%, greater than 85%, greater than 90%, greater than 95%, greater than 96%, greater than 97%, greater than 98%, greater than 99%, or about 100%. Such diagnostic methods detect CRC with a sensitivity that is between about 70%-100%, between about 80%-100%, or between about 90-100%. Such diagnostic methods detect CRC with a specificity greater than 70%, greater than 75%, greater than 80%, greater than 85%, greater than 90%, greater than 95%, greater than 96%, greater than 97%, greater than 98%, greater than 99%, or about 100%. Such diagnostic methods detect CRC with a specificity that is between about 50%400%, between about 60%400%, between about 70%400%, between about 80%-100%, or between about 90-100%. In particular embodiments, such diagnostic methods detect CRC with a sensitivity and a specificity that is 50% or greater, 60% or greater, 70% or greater, 75% or greater, 80% or greater, 85% or greater, 90% or greater. In particular embodiments, such diagnostic methods detect CRC with a sensitivity and a specificity that is between about 50%400%, between about 60%400%, between about 70%400%, between about 80%-100%, or between about 90-100%.

The overall performance of a classifier is assessed in some cases via the AUC of the ROC as reported herein. An ROC considers the performance of the classifier at all possible model score cutoff points. However, when a classification decision needs to be made (e.g., is this patient sick or healthy?), a cutoff point is used to define the two groups. Classification scores at or above the cutoff point are assessed as positive (or sick) while points below are assessed as negative (or healthy) in various embodiments.

For some classification models disclosed herein, a classification score cutoff point is established by selecting the point of maximum accuracy on the validation ROC. The point of maximum accuracy on an ROC is the cutoff point or points for which the total number of correct classification calls is maximized. Here, the positive and negative classification calls are weighted equally. In cases where multiple maximum accuracy points are present on a given ROC, the point with the associated maximum sensitivity is selected in some cases.

### Algorithm-Based Methods

Methods, compositions, kits, and systems described herein utilize an algorithm-based diagnostic assay for predicting a presence or absence of at least one of: advanced colorectal adenoma and CRC in a subject. Expression level of one or more protein biomarker, and optionally one or more subject characteristics, such as, for example, age, weight, gender, medical history, risk factors, or family history are used alone or arranged into functional subsets to calculate a quantitative score that is used to predict the likelihood of a presence or absence of at least one of advanced colorectal adenoma and CRC. Although lead embodiments herein focus upon biomarker panels that are predominantly protein or polypeptide panels, the measurements of any of the biomarker panels may comprise protein and non-protein components such as RNA, DNA, organic metabolites, or inorganic molecules or metabolites (e.g. iron, magnesium, selenium, calcium, or others).

The algorithm-based assay and associated information provided by the practice of any of the methods described herein can facilitate optimal treatment decision-making in subjects. For example, such a clinical tool can enable a physician or caretaker to identify patients who have a low likelihood of having an advanced colorectal adenoma or carcinoma and therefore would not need treatment, or increased monitoring for advanced colorectal adenoma or CRC, or who have a high likelihood of having an advanced colorectal adenoma or CRC and therefore would need treatment or increased monitoring of said advanced colorectal adenoma or CRC.

A quantitative score is determined by the application of a specific algorithm in some cases. The algorithm used to calculate the quantitative score in the methods disclosed herein may group the expression level values of a biomarker or groups of biomarkers. The formation of a particular group of biomarkers, in addition, can facilitate the mathematical weighting of the contribution of various expression levels of biomarker or biomarker subsets (for example classifier) to the quantitative score. Described herein are exemplary algorithms for calculating the quantitative scores.

Exemplary biomarkers and, when applicable their human amino acid sequences, are listed in Tables 1 and in panels in Tables 3-4. Biomarkers may comprise full length molecules of the polypeptide sequences of Table 1, as well as uniquely identifiable fragments of the polypeptide sequences of Table 1. Markers can be but do not need to be full length to be informative. In many cases, so long as a fragment is uniquely identifiable as being derived from or representing a polypeptide of Table 1, it is informative for purposes herein.

### Exemplary Subjects

Biological samples are collected from a number of eligible subjects, such as subjects who want to determine their likelihood of having at least one of advanced colorectal adenoma and CRC. The subject is in some cases healthy and asymptomatic. The subject's age is not constrained. For example, the subject is between the ages of 0 to about 30 years, about 20 to about 50 years, or about 40 or older. In various cases, the subject is healthy, asymptomatic and between the ages of 0-30 years, 20-50 years, or 40 or older. The subject is at least 30 years of age, at least 40 years of age, or at least 50 years of age. The subject is less than 50 years of age, less than 40 years of age, or less than 30 years of age. In various examples, the subject is healthy and asymptomatic. In various examples, the subject has no family history of at least one of: CRC, adenoma, and polyps. In various examples, the subject has not had a colonoscopy, sigmoidoscopy, or colon tissue biopsy. In various examples, the subject is healthy and asymptomatic and has not received a colonoscopy, sigmoidoscopy, or colon tissue biopsy. In some cases, the subject has not received a colonoscopy, sigmoidoscopy, or colon tissue biopsy and has one or more of: a symptom of CRC, a family history of CRC, and a risk factor for CRC. In some cases, a biological sample can be obtained from a subject during routine examination, or to establish baseline levels of the biomarkers. In some cases, a subject has no symptoms for colorectal carcinoma, has no family history for colorectal carcinoma, has no recognized risk factors for colorectal carcinoma.

In some cases, a subject presents at least one of: a symptom for colorectal carcinoma, a family history for colorectal carcinoma, and a recognized risk factor for colorectal carcinoma. In some cases, a subject is identified through screening assays (for example, fecal occult blood testing or sigmoidoscopy) or rectal digital exam or rigid or flexible colonoscopy or CT scan or other x-ray techniques as being at high risk for or having CRC. For example, one or more methods described herein are applied to a subject undergoing treatment for CRC, to determine the effectiveness of the therapy or treatment they are receiving.

### Exemplary Biological Samples

Biological samples in some exemplary embodiments are circulating blood samples or are samples obtained from the vein or artery of an individual. Samples are optionally processed, so as to isolate plasma, circulating free proteins, or a whole protein fraction from the blood sample. Samples are often treated to facilitate storage or to allow shipment at room temperature, although in preferred embodiments samples are shipped frozen, for example with or on dry ice, to preserve the samples for analysis at a processing center separate from a phlebotomist's office.

As a representative sample collection protocol, blood samples for serum, EDTA plasma, citrate plasma and buffy-coats are collected with light tournique from an antecubital vein using endotoxin-, deoxyribonuclease (DNAse-) and ribonuclease (RNAse-) free collection and handling equipment, collection tubes and storage vials from Becton-Dickinson, Franklin Lakes, N.J., USA and Almeco A/S, Esbj erg, Denmark. The blood samples are centrifuged at 3,000×G for 10 mins at 21° C. and serum and plasma are immediately separated from the red cell and buffy-coat layers. Contamination by white cells and platelets is reduced by leaving 0.5 cm of untouched serum or plasma above the buffy-coat, which is separately transferred for freezing. All separated samples are marked with unique barcodes for storage identification, which is performed using the FreezerWorks®, Seattle, Wash., USA tracking system. Separated samples are frozen at −80° C. under continuous electronic surveillance. The entire procedure is completed within 2 hours of initial sample draw.

Additional biological samples include one or more of, but are not limited to: urine, stool, tears, whole blood, serum, plasma, blood constituent, bone marrow, tissue, cells, organs, saliva, cheek swab, lymph fluid, cerebrospinal fluid, lesion exudates and other fluids produced by the body. The biological sample is in some cases a solid biological sample, for example, a tissue biopsy. The biopsy can be fixed, paraffin embedded, or fresh. In many embodiments herein, a preferred sample is a blood sample drawn from a vein or artery of an individual, or a processed product thereof.

Biological samples are optionally processed using any approach known in the art or otherwise described herein to facilitate measurement of one or more biomarkers as described herein. Sample preparation operations comprise, for example, extraction and/or isolation of intracellular material from a cell or tissue such as the extraction of nucleic acids, protein, or other macromolecules. Sample preparation which can be used with the methods of disclosure include but are not limited to, centrifugation, affinity chromatography, magnetic separation, immunoassay, nucleic acid assay, receptor-based assay, cytometric assay, colorimetric assay, enzymatic assay, electrophoretic assay, electrochemical assay, spectroscopic assay, chromatographic assay, microscopic assay, topographic assay, calorimetric assay, radioisotope assay, protein synthesis assay, histological assay, culture assay, and combinations thereof.

Sample preparation optionally includes dilution by an appropriate solvent and amount to ensure the appropriate range of concentration level is detected by a given assay.

Accessing the nucleic acids and macromolecules from the intercellular space of the sample is performed by either physical, chemical methods, or a combination of both. In some applications of the methods, following the isolation of the crude extract, it will often be desirable to separate the nucleic acids, proteins, cell membrane particles, and the like. In some applications of the methods it will be desirable to keep the nucleic acids with its proteins, and cell membrane particles.

In some applications of the methods provided herein, nucleic acids and proteins are extracted from a biological sample prior to analysis using methods of the disclosure. Extraction is accomplished, for example through use of detergent lysates, sonication, or vortexing using glass beads.

Molecules can be isolated using any technique suitable in the art including, but not limited to, techniques using gradient centrifugation (for example, cesium chloride gradients, sucrose gradients, glucose gradients, or other gradients), centrifugation protocols, boiling, purification kits, and the use of liquid extraction with agent extraction methods such as methods using Trizol or DNAzol.

Some samples are partially prepared at a separate location prior to being sent for analysis. For example, a phlebotomist draws a blood sample at a clinic or hospital. The sample can be partially processed, for example, by placing in anticoagulant-treated tubes and centrifuging to produce plasma. The partially processed sample, such as the plasma, is then shipped (e.g., mailed on ice or in preservative at room temperature) to a separate facility where any of the methods disclosed herein can be performed to determine a biomarker panel level and/or CRC or advanced adenoma health status.

Samples are prepared according to standard biological sample preparation depending on the desired detection method. For example, for mass spectrometry detection, biological samples obtained from a patient may be centrifuged, filtered, processed by immunoaffinity column, separated into fractions, partially digested, and combinations thereof. Various fractions may be resuspended in appropriate carrier such as buffer or other type of loading solution for detection and analysis, including LCMS loading buffer.

### Biomarker Assessment

The present disclosure provides for methods for measuring one or more biomarker panels in biological samples. Any suitable method can be used to detect one or more of the biomarkers of any of the panels described herein.

In some cases, only values falling within specific ranges are reported. For example, assayed protein concentrations or other biomarker levels below a given cutoff indicate a failed assay in some cases, while assayed protein concentrations or other biomarker levels above a threshold may indicate a suspect or inaccurate reading.

Useful analyte capture agents used in practice of methods described herein include but are not limited to antibodies, such as crude serum containing antibodies, purified antibodies, monoclonal antibodies, polyclonal antibodies, synthetic antibodies, antibody fragments (for example, Fab fragments); antibody interacting agents, such as protein A, carbohydrate binding proteins, and other interactants; protein interactants (for example avidin and its derivatives); peptides; and small chemical entities, such as enzyme substrates, cofactors, metal ions/chelates, aptamers, and haptens. Antibodies may be modified or chemically treated to optimize binding to targets or solid surfaces (for example biochips and columns).

Biomarkers are measured in some cases in a biological sample using an immunoassay. Some immunoassays use antibodies that specifically or informatively bind to or recognize an antigen (for example site on a protein or peptide, biomarker target). Some immunoassays include the steps of contacting the biological sample using the antibody and allowing the antibody to form a complex of with the antigen in the sample, washing the sample and detecting the antibody-antigen complex with a detection reagent. Antibodies that recognize the biomarkers may be commercially available. An antibody that recognizes the biomarkers can be generated by known methods of antibody production.

Immunoassays include indirect assays, wherein, for example, a second, labeled antibody can be used to detect bound marker-specific antibody. Exemplary detectable labels include magnetic beads (for example, DYNABEADS™), fluorescent dyes, radiolabels, enzymes (for example, horseradish peroxide, alkaline phosphatase and others commonly used), and calorimetric labels such as colloidal gold or colored glass or plastic beads. The biomarker in the sample can be measured using a competition or inhibition assay wherein, for example, a monoclonal antibody which binds to a distinct epitope of the marker is incubated simultaneously with the mixture.

The conditions to detect an antigen using an immunoassay are dependent on the particular antibody used. Also, the incubation time can depend upon the assay format, marker, volume of solution, concentrations and the like. Immunoassays can be carried out at room temperature, although they can be conducted over a range of temperatures, such as from about 0 degrees to about 40 degrees Celsius depending on the antibody used.

There are various types of immunoassay known in the art that as a starting basis can be used to tailor the assay for the detection of the biomarkers of the present disclosure. Useful assays can include, for example, an enzyme immune assay (EIA) such as enzyme-linked immunosorbent assay (ELISA). For example, if an antigen can be bound to a solid support or surface, it can be detected by reacting it with a specific antibody and the antibody can be quantitated by reacting it with either a secondary antibody or by incorporating a label directly into the primary antibody. Alternatively, an antibody can be bound to a solid surface and the antigen added. A second antibody that recognizes a distinct epitope on the antigen can then be added and detected. Such assay can be referred to as a ‘sandwich assay’ and can be used to avoid problems of high background or non-specific reactions. These types of assays can be sensitive and reproducible enough to measure low concentrations of antigens in a biological sample.

Immunoassays are used to determine presence or absence of a marker in a sample as well as the quantity of a marker in a sample. Methods for measuring the amount of, or presence of, antibody-marker complex include but are not limited to, fluorescence, luminescence, chemiluminescence, absorbance, reflectance, transmittance, birefringence or refractive index (for example, surface plasmon resonance, ellipsometry, a resonant mirror method, a grating coupler waveguide method or interferometry). Such reagents can be used with optical detection methods, such as various forms of microscopy, imaging methods and non-imaging methods. Electrochemical methods can include voltammetry and amperometry methods. Radio frequency methods can include multipolar resonance spectroscopy.

Measurement of biomarkers optionally involves use of an antibody. Antibodies that specifically bind to any of the biomarkers described herein can be prepared using standard methods known in the art. For example polyclonal antibodies can be produced by injecting an antigen into a mammal, such as a mouse, rat, rabbit, goat, sheep, or horse for large quantities of antibody. Blood isolated from these animals can contain polyclonal antibodies—multiple antibodies that bind to the same antigen. Alternatively, polyclonal antibodies can be produced by injecting the antigen into chickens for generation of polyclonal antibodies in egg yolk. In addition, antibodies can be made to specifically recognize modified forms for the biomarkers such as a phosphorylated form of the biomarker, for example, they can recognize a tyrosine or a serine after phosphorylation, but not in the absence of phosphate. In this way antibodies can be used to determine the phosphorylation state of a particular biomarker.

Antibodies are obtained commercially or produced using well-established methods. To obtain antibodies specific for a single epitope of an antigen, antibody-secreting lymphocytes are isolated from the animal and immortalized by fusing them with a cancer cell line. The fused cells are referred to as hybridomas, and can continually grow and secrete antibody in culture. Single hybridoma cells are isolated by dilution cloning to generate cell clones that all produce the same antibody; these antibodies can be referred to as monoclonal antibodies.

Polyclonal and monoclonal antibodies can be purified in several ways. For example, one can isolate an antibody using antigen-affinity chromatography which can be couple to bacterial proteins such as Protein A, Protein G, Protein L or the recombinant fusion protein, Protein A/G followed by detection of via UV light at 280 nm absorbance of the eluate fractions to determine which fractions contain the antibody. Protein A/G can bind to all subclasses of human IgG, making it useful for purifying polyclonal or monoclonal IgG antibodies whose subclasses have not been determined. In addition, Protein A/G can bind to IgA, IgE, IgM and (in some cases to a lesser extent) IgD. Protein A/G can bind to all subclasses of mouse IgG but in some cases does not bind mouse IgA, IgM or serum albumin. This feature can allow Protein A/G to be used for purification and detection of mouse monoclonal IgG antibodies, without interference from IgA, IgM and serum albumin.

Antibodies are derived from different classes or isotypes of molecules such as, for example, IgA, IgA IgD, IgE, IgM and IgG. The IgA can be designed for secretion in the bodily fluids while others, like the IgM are designed to be expressed on the cell surface. The antibody can be an IgG antibody. In some cases, IgG comprises two subunits including two “heavy” chains and two “light” chains. These can be assembled in a symmetrical structure and each IgG can have two identical antigen recognition domains. The antigen recognition domain can be a combination of amino acids from both the heavy and light chains. The molecule can be roughly shaped like a “Y” and the arms/tips of the molecule comprise the antigen-recognizing regions or Fab (fragment, antigen binding) region, while the stem of Fc (Fragment, crystallizable) region is not necessarily involved in recognition and can be fairly constant. The constant region can be identical in all antibodies of the same isotype, but can differ in antibodies of different isotypes.

It is also possible to use an antibody to detect a protein after fractionation by western blotting. Western blotting is used in some cases for the detection and/or measurement of protein or polypeptide biomarkers.

Some detection methods can employ flow cytometry. Flow cytometry can be a laser based, biophysical technology that can be used for biomarker detection, quantification (cell counting) and cell isolation. This technology can be used in the diagnosis of health disorders, especially blood cancers. In general, flow cytometry can comprise suspending single cells in a stream of fluid. A beam of light (usually laser light) of a single wavelength can be directed onto the stream of liquid, and the scatter light caused by a passing cell can be detected by an electronic detection apparatus. A flow cytometry methodology useful in one or more methods described herein can include Fluorescence-activated cell sorting (FACS). FACS can use florescent-labeled antibodies to detect antigens on cell of interest. This additional feature of antibody labeling use in FACS can enable simultaneous multiparametric analysis and quantification based upon the specific light scattering and fluorescent characteristics of each cell florescent-labeled cell and it provides physical separation of the population of cells of interest as well as traditional flow cytometry does.

A wide range of fluorophores can be used as labels in flow cytometry. Fluorophores can be typically attached to an antibody that recognizes a target feature on or in the cell. Examples of suitable fluorescent labels include, but are not limited to: fluorescein (FITC), 5, 6-carboxymethyl fluorescein, Texas red, nitrobenz-2-oxa-1,3-diazol-4-yl (NBD), and the cyanine dyes Cy3, Cy3.5, Cy5, Cy5.5 and Cy7. Other Fluorescent labels such as Alexa Fluor® dyes, DNA content dye such as DAPI, and Hoechst dyes are well known in the art and can be easily obtained from a variety of commercial sources. Each fluorophore can have a characteristic peak excitation and emission wavelength, and the emission spectra often overlap. The absorption and emission maxima, respectively, for these fluors can be: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm). The fluorescent labels can be obtained from a variety of commercial sources. Quantum dots can be used in place of traditional fluorophores. Other methods that can be used for detecting include isotope labeled antibodies, such as lanthanide isotopes.

Immunoassays optionally comprise immunohistochemistry. Immunohistochemistry is used to detect expression of the claimed biomarkers in a tissue sample. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody can be used in conjunction with a labeled secondary antibody, comprising antisera, polyclonal antisera or a monoclonal antibody specific for the primary antibody. Immunohistochemistry protocols are well known in the art and protocols and antibodies are commercially available. Alternatively, one raises an antibody to the biomarkers or modified versions of the biomarker or binding partners as disclosure herein that would be useful for determining the expression levels of the proteins in a tissue sample.

Some measurement of biomarkers comprises use of a biochip. Biochips can be used to screen a large number of macromolecules. Biochips can be designed with immobilized nucleic acid molecules, full-length proteins, antibodies, affibodies (small molecules engineered to mimic monoclonal antibodies), aptamers (nucleic acid-based ligands) or chemical compounds. A chip could be designed to detect multiple macromolecule types on one chip. For example, a chip could be designed to detect nucleic acid molecules, proteins and metabolites on one chip. The biochip can be used to and designed to simultaneously analyze a panel biomarker in a single sample, producing a subjects profile for these biomarkers. The use of the biochip allows for the multiple analyses to be performed reducing the overall processing time and the amount of sample required.

Protein microarray can be a particular type of biochip which can be used with the present disclosure. In some cases, the chip comprises a support surface such as a glass slide, nitrocellulose membrane, bead, or microtitre plate, to which an array of capture proteins can be bound in an arrayed format onto a solid surface. Protein array detection methods can give a high signal and a low background. Detection probe molecules, typically labeled with a fluorescent dye, can be added to the array. Any reaction between the probe and the immobilized protein can result in emission of a detectable signal. Such protein microarrays can be rapid, automated, and offer high sensitivity of protein biomarker read-outs for diagnostic tests. However, it would be immediately appreciated to those skilled in the art that there are a variety of detection methods that can be used with this technology. Exemplary microarrays include analytical microarrays (also known as capture arrays), functional protein microarrays (also known as target protein arrays) and reverse phase protein microarray (RPA).

Analytical protein microarrays can be constructed using a library of antibodies, aptamers or affibodies. The array can be probed with a complex protein solution such as a blood, serum or a cell lysate that function by capturing protein molecules they specifically bind to. Analysis of the resulting binding reactions using various detection systems can provide information about expression levels of particular proteins in the sample as well as measurements of binding affinities and specificities. This type of protein microarray can be especially useful in comparing protein expression in different samples. Functional protein microarrays can be constructed by immobilizing large numbers of purified full-length functional proteins or protein domains and can be used to identify protein-protein, protein-DNA, protein-RNA, protein-phospholipid, and protein-small molecule interactions, to assay enzymatic activity and to detect antibodies and demonstrate their specificity. These protein microarray biochips can be used to study the biochemical activities of the entire proteome in a sample.

One or more biomarkers can be measured using reverse phase protein microarray (RPA). Reverse phase protein microarray can be constructed from tissue and cell lysates that can be arrayed onto the microarray and probed with antibodies against the target protein of interest. These antibodies can be detected with chemiluminescent, fluorescent or colorimetric assays. In addition to the protein in the lysate, reference control peptides can be printed on the slides to allow for protein quantification. RPAs allow for the determination of the presence of altered proteins or other agents that may be the result of disease and present in a diseased cell.

One or more biomarkers can be measured using mass spectroscopy (alternatively referred to as mass spectrometry). Mass spectrometry (MS) can refer to an analytical technique that measures the mass-to-charge ratio of charged particles. It can be primarily used for determining the elemental composition of a sample or molecule, and for elucidating the chemical structures of molecules, such as peptides and other chemical compounds. MS works by ionizing chemical compounds to generate charged molecules or molecule fragments and measuring their mass-to-charge ratios MS instruments typically consist of three modules (1) an ion source, which can convert gas phase sample molecules into ions (or, in the case of electrospray ionization, move ions that exist in solution into the gas phase) (2) a mass analyzer, which sorts the ions by their masses by applying electromagnetic fields and (3) detector, which measures the value of an indicator quantity and thus provides data for calculating the abundances of each ion present.

Suitable mass spectrometry methods to be used with the present disclosure include but are not limited to, one or more of electrospray ionization mass spectrometry (ESI-MS), ESI-MS/MS, ESI-MS/(MS)ₙ, matrix-assisted laser desorption ionization time-of-flight mass spectrometry (MALDI-TOF-MS), surface-enhanced laser desorption/ionization time-of-flight mass spectrometry (SELDI-TOF-MS), tandem liquid chromatography-mass spectrometry (LC-MS/MS) mass spectrometry, desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS), atmospheric pressure photoionization mass spectrometry (APPI-MS), APPI-MS/MS, and APPI-(MS)ₙ, quadrupole mass spectrometry, Fourier transform mass spectrometry (FTMS), and ion trap mass spectrometry, where n can be an integer greater than zero.

LC-MS can be commonly used to resolve the components of a complex mixture. LC-MS method generally involves protease digestion and denaturation (usually involving a protease, such as trypsin and a denaturant such as, urea to denature tertiary structure and iodoacetamide to cap cysteine residues) followed by LC-MS with peptide mass fingerprinting or LC-MS/MS (tandem MS) to derive sequence of individual peptides. LC-MS/MS can be used for proteomic analysis of complex samples where peptide masses may overlap even with a high-resolution mass spectrometer. Samples of complex biological fluids like human serum may be first separated on an SDS-PAGE gel or HPLC-SCX and then run in LC-MS/MS allowing for the identification of over 1000 proteins.

While multiple mass spectrometric approaches are compatible with the methods of the disclosure as provided herein, in some applications it is desired to quantify proteins in biological samples from a selected subset of proteins of interest. One such MS technique that is compatible with the present disclosure is Multiple Reaction Monitoring Mass Spectrometry (MRM-MS), or alternatively referred to as Selected Reaction Monitoring Mass Spectrometry (SRM-MS).

The MRM-MS technique involves a triple quadrupole (QQQ) mass spectrometer to select a positively charged ion from the peptide of interest, fragment the positively charged ion and then measure the abundance of a selected positively charged fragment ion. This measurement is commonly referred to as a transition and/or transition ion.

Alternately or in combination, a sample prepared for MS analysis is supplemented with at least one labeled protein or polypeptide, such that the labeled protein or polypeptide migrates with or near a protein or fragment in a sample. In some cases a heavy-isotope labeled protein or fragment is introduced into a sample, such that the labeled protein or fragment migrates near but not identically to an unlabeled, native version of the protein in the sample. With an understanding of the position of the labeled protein and the impact of its labeling on MS migration, one can readily identify the corresponding native protein in the sample. In some cases a panel of labeled proteins or protein fragments are adopted, so that a panel of proteins is readily assayed from MS data but, concurrently, untargeted data of a broad range of proteins or fragments is also obtained.

In some applications the MRM-MS is coupled with High-Pressure Liquid Chromatography (HPLC) and more recently Ultra High-Pressure Liquid Chromatography (UHPLC). In other applications MRM-MS can be coupled with UHPLC with a QQQ mass spectrometer to make the desired LC-MS transition measurements for all of the peptides and proteins of interest.

In some applications the utilization of a quadrupole time-of-flight (qTOF) mass spectrometer, time-of-flight time-of-flight (TOF-TOF) mass spectrometer, Orbitrap mass spectrometer, quadrupole Orbitrap mass spectrometer or any Quadrupolar Ion Trap mass spectrometer can be used to select for a positively charged ion from one or more peptides of interest. The fragmented, positively charged ions can then be measured to determine the abundance of a positively charged ion for the quantitation of the peptide or protein of interest.

In some applications the utilization of a time-of-flight (TOF), quadrupole time-of-flight (qTOF) mass spectrometer, time-of-flight time-of-flight (TOF-TOF) mass spectrometer, Orbitrap mass spectrometer or quadrupole Orbitrap mass spectrometer is used to measure the mass and abundance of a positively charged peptide ion from the protein of interest without fragmentation for quantitation. In this application, the accuracy of the analyte mass measurement can be used as selection criteria of the assay. An isotopically labeled internal standard of a known composition and concentration can be used as part of the mass spectrometric quantitation methodology.

In some applications, time-of-flight (TOF), quadrupole time-of-flight (qTOF) mass spectrometer, time-of-flight time-of-flight (TOF-TOF) mass spectrometer, Orbitrap mass spectrometer or quadrupole Orbitrap mass spectrometer is used to measure the mass and abundance of a protein of interest for quantitation. In this application, the accuracy of the analyte mass measurement can be used as selection criteria of the assay. Optionally this application can use proteolytic digestion of the protein prior to analysis by mass spectrometry. An isotopically labeled internal standard of a known composition and concentration can be used as part of the mass spectrometric quantitation methodology.

In some applications, various ionization techniques can be coupled to the mass spectrometers provide herein to generate the desired information. Non-limiting exemplary ionization techniques that are used with the present disclosure include but are not limited to Matrix Assisted Laser Desorption Ionization (MALDI), Desorption Electrospray Ionization (DESI), Direct Assisted Real Time (DART), Surface Assisted Laser Desorption Ionization (SALDI), or Electrospray Ionization (ESI).

In some applications, HPLC and UHPLC can be coupled to a mass spectrometer a number of other peptide and protein separation techniques can be performed prior to mass spectrometric analysis. Some exemplary separation techniques which can be used for separation of the desired analyte (for example, peptide or protein) from the matrix background include but are not limited to Reverse Phase Liquid Chromatography (RP-LC) of proteins or peptides, offline Liquid Chromatography (LC) prior to MALDI, 1 dimensional gel separation, 2-dimensional gel separation, Strong Cation Exchange (SCX) chromatography, Strong Anion Exchange (SAX) chromatography, Weak Cation Exchange (WCX), and Weak Anion Exchange (WAX). One or more of the above techniques can be used prior to mass spectrometric analysis.

One or more biomarkers can be measured using a microarray. Differential gene expression can also be identified, or confirmed using the microarray technique. Thus, the expression profile biomarkers can be measured in either fresh or fixed tissue, using microarray technology. In this method, polynucleotide sequences of interest (including cDNAs and oligonucleotides) can be plated, or arrayed, on a microchip substrate. The arrayed sequences can be then hybridized with specific DNA probes from cells or tissues of interest. The source of mRNA can be total RNA isolated from a biological sample, and corresponding normal tissues or cell lines may be used to determine differential expression.

One or more biomarkers can be measured by sequencing. Differential gene expression can also be identified, or confirmed using the sequencing technique. Thus, the expression profile biomarkers can be measured in either fresh or fixed sample, using sequencing technology. In this method, polynucleotide sequences of interest (including cDNAs and oligonucleotides) can used as templates to synthesize sequencing libraries. The libraries can be sequenced, and the reads mapped to an appropriate reference. The source of mRNA can be total RNA isolated from a biological sample, and corresponding normal tissues or cell lines may be used to determine differential expression. Exemplary sequencing techniques can include, for example emulsion PCR (pyrosequencing from Roche 454, semiconductor sequencing from Ion Torrent, SOLiD sequencing by ligation from Life Technologies, sequencing by synthesis from Intelligent Biosystems), bridge amplification on a flow cell (e.g. Solexa/111umina), isothermal amplification by Wildfire technology (Life Technologies) or rolonies/nanoballs generated by rolling circle amplification (Complete Genomics, Intelligent Biosystems, Polonator). Sequencing technologies like Heliscope (Helicos), SMRT technology (Pacific Biosciences) or nanopore sequencing (Oxford Nanopore) allow direct sequencing of single molecules without prior clonal amplification may be suitable sequencing platforms. Sequencing may be performed with or without target enrichment. In some cases, polynucleotides from a sample are amplified by any suitable means prior to and/or during sequencing.

PCR amplified inserts of cDNA clones can be applied to a substrate in a dense array. Preferably at least 10,000 nucleotide sequences can be applied to the substrate. The microarrayed genes, immobilized on the microchip at 10,000 elements each, can be suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the microarray chip can be scanned by a device such as, confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA can be hybridized pair-wise to the array. The relative abundance of the transcripts from the two sources corresponding to each specified gene can be thus determined simultaneously. Microarray analysis can be performed by commercially available equipment, following manufacturer's protocols.

One or more biomarkers can be measured using qRT-PCR, which can be used to compare mRNA levels in different sample populations, in normal and tumor tissues, with or without drug treatment, to characterize patterns of gene expression, to discriminate between closely related mRNAs, and to analyze RNA structure. The first step in gene expression profiling by RT-PCR can be extracting RNA from a biological sample followed by the reverse transcription of the RNA template into cDNA and amplification by a PCR reaction. The reverse transcription reaction step can be generally primed using specific primers, random hexamers, or oligo-dT primers, depending on the goal of expression profiling. Reverse transcriptases can be avilo myeloblastosis virus reverse transcriptase (AMV-RT) and/or Moloney murine leukemia virus reverse transcriptase (MLV-RT).

Although the PCR step can use a variety of thermostable DNA-dependent DNA polymerases, it typically employs the Taq DNA polymerase, which can have a 5′-3′ nuclease activity but lacks a 3′-5′ proofreading endonuclease activity. Thus, TaqMan™ PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers can be used to generate an amplicon typical of a PCR reaction. A third oligonucleotide, or probe, can be designed to detect nucleotide sequence located between the two PCR primers. The probe can be non-extendible by Taq DNA polymerase enzyme, and can be labeled with a reporter fluorescent dye and a quencher fluorescent dye. Any laser-induced emission from the reporter dye can be quenched by the quenching dye when the two dyes are located close together as they are on the probe. During the amplification reaction, the Taq DNA polymerase enzyme can cleave the probe in a template-dependent manner. The resultant probe fragments can disassociate in solution, and signal from the released reporter dye can be freed from the quenching effect of the second fluorophore. One molecule of reporter dye can be liberated for each new molecule synthesized, and detection of the unquenched reporter dye can provide basis for quantitative interpretation of the data.

TaqMan™ RT-PCR can be performed using commercially available equipment, such as, for example, ABI PRISM 7700 Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 Sequence Detection System™. The system comprises a thermocycler, laser, charge-coupled device (CCD), camera and computer. The system includes software for running the instrument and for analyzing the data. 5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant can be the threshold cycle (Ct).

To minimize errors and the effect of sample-to-sample variation, RT-PCR can be performed using an internal standard. An internal standard can be expressed at a constant level among different tissues, and can be unaffected by the experimental treatment. RNAs most frequently used to normalize patterns of gene expression are mRNAs for the housekeeping genes glyceraldehyde-3-phosphate-dehydrogenase (GAPDH) and Beta-Actin.

A more recent variation of the RT-PCR technique can include the real time quantitative PCR, which can measure PCR product accumulation through a dual-labeled fluorogenic probe (i.e., TaqMan™ probe). Real time PCR can be compatible both with quantitative competitive PCR, where internal competitor for each target sequence can be used for normalization, and with quantitative comparative PCR using a normalization gene contained within the sample, or a housekeeping gene for RT-PCR. For further details see, for example Held et al., Genome Research 6:986-994 (1996).

### Normalization of Data

Measurement data used in the methods, systems, kits and compositions disclosed herein are optionally normalized. Normalization refers to a process to correct for example, differences in the amount of genes or protein levels assayed and variability in the quality of the template used, to remove unwanted sources of systematic variation measurements involved in the processing and detection of genes or protein expression. Other sources of systematic variation are attributable to laboratory processing conditions.

In some instances, normalization methods are used for the normalization of laboratory processing conditions. Non-limiting examples of normalization of laboratory processing that may be used with methods of the disclosure include but are not limited to: accounting for systematic differences between the instruments, reagents, and equipment used during the data generation process, and/or the date and time or lapse of time in the data collection.

Assays can provide for normalization by incorporating the expression of certain normalizing standard genes or proteins, which do not significantly differ in expression levels under the relevant conditions, that is to say they are known to have a stabilized and consistent expression level in that particular sample type. Suitable normalization genes and proteins that can be used with the present disclosure include housekeeping genes. (See, for example, E. Eisenberg, et al., Trends in Genetics 19(7):362-365 (2003). In some applications, the normalizing biomarkers (genes and proteins), also referred to as reference genes, known not to exhibit meaningfully different expression levels in subjects with advanced colorectal adenoma or CRC as compared to control subjects without advanced colorectal adenoma or CRC. In some applications, it may be useful to add a stable isotope labeled standards which can be used and represent an entity with known properties for use in data normalization. In other applications, a standard, fixed sample can be measured with each analytical batch to account for instrument and day-to-day measurement variability.

### Clinical Outcome Score

Machine learning algorithms for sub-selecting discriminating biomarkers and optionally subject characteristics, and for building classification models, are used in some methods and systems herein to determine clinical outcome scores. These algorithms include, but are not limited to, elastic networks, random forests, support vector machines, and logistic regression. These algorithms can aid in selection of important biomarker features and transform the underlying measurements into a score or probability relating to, for example, clinical outcome, disease risk, disease likelihood, presence or absence of disease, treatment response, and/or classification of disease status.

A clinical outcome score is determined by comparing a level of at least two biomarkers in the biological sample obtained from the subject to a reference level of the at least two biomarkers. Alternately or in combination, a clinical outcome score is determined by comparing a subject-specific profile of a biomarker panel to a reference profile of the biomarker panel. Often, a reference level or reference profile represents a known diagnosis. For example, a reference level or reference profile represents a positive diagnosis of advanced colorectal adenoma. A reference level or reference profile can represent a positive diagnosis of CRC. As another example, a reference level or reference profile represents a negative diagnosis of advanced colorectal adenoma. Similarly, a reference level or reference profile can represent a negative diagnosis of CRC

In some cases, an increase in a score indicates an increased likelihood of one or more of: a poor clinical outcome, good clinical outcome, high risk of disease, low risk of disease, complete response, partial response, stable disease, non-response, and recommended treatments for disease management. In some cases, a decrease in the quantitative score indicates an increased likelihood of one or more of: a poor clinical outcome, good clinical outcome, high risk of disease, low risk of disease, complete response, partial response, stable disease, non-response, and recommended treatments for disease management.

A similar biomarker profile from a patient to a reference profile often indicates an increased likelihood of one or more of: a poor clinical outcome, good clinical outcome, high risk of disease, low risk of disease, complete response, partial response, stable disease, non-response, and recommended treatments for disease management. In some applications, a dissimilar biomarker profile from a patient to a reference profile indicates one or more of: an increased likelihood of a poor clinical outcome, good clinical outcome, high risk of disease, low risk of disease, complete response, partial response, stable disease, non-response, and recommended treatments for disease management.

An increase in one or more biomarker threshold values often indicates an increased likelihood of one or more of: a poor clinical outcome, good clinical outcome, high risk of disease, low risk of disease, complete response, partial response, stable disease, non-response, and recommended treatments for disease management. In some applications, a decrease in one or more biomarker threshold values indicates an increased likelihood of one or more of: a poor clinical outcome, good clinical outcome, high risk of disease, low risk of disease, complete response, partial response, stable disease, non-response, and recommended treatments for disease management.

An increase in at least one of a quantitative score, one or more biomarker thresholds, a similar biomarker profile values indicates an increased likelihood of one or more of: a poor clinical outcome, good clinical outcome, high risk of disease, low risk of disease, complete response, partial response, stable disease, non-response, and recommended treatments for disease management. Similarly, a decrease in at least one of a quantitative score, one or more biomarker thresholds, a similar biomarker profile values or combinations thereof indicates an increased likelihood of one or more of: a poor clinical outcome, good clinical outcome, high risk of disease, low risk of disease, complete response, partial response, stable disease, non-response, and recommended treatments for disease management.

A clinical outcome score is optionally updated based on additional information derived during treatment. Such updates often comprise the addition of other biomarkers. Such biomarkers include additional proteins, metabolite accumulation levels, physical characteristics of the subject (e.g., age, race, weight, demographic history), medical history of the subject (e.g., family history of advanced colorectal adenoma, prior quantitative score of the protein panels). Such updates can comprise an adjustment of the test sensitivity. Such updates can comprise an adjustment of the test sensitivity. Such updates can comprise an adjustment of the test thresholds. Such updates can comprise an adjustment of the predicted clinical outcomes.

For example, in some cases a patient at risk of advanced colorectal adenoma is tested using a panel as disclosed herein. The patient may be categorized as having or being likely to have, advanced colorectal adenoma. In some cases, the thresholds of a protein panel disclosed herein will be updated based on additional biomarkers, such as age of the patient. For example, a patient over the age of 60 is more likely than a patient under 60 to have advanced colorectal adenoma. Therefore, the positive predictive value of the protein panel can be higher in the population over 60 than the population under 60. In some cases, the threshold for proteins in the protein panel can be altered based on an additional biomarker (e.g., age) to reflect this, such as by lowering the threshold in a population over 60 compared to a population under 60. A patient's personal threshold may be updated based on previous test results. For example, a patient may have an indeterminate or positive clinical outcome score. Such a patient may have additional tests recommended. Such a patient may have a colonoscopy recommended. Such additional tests and colonoscopies can come back negative, and the persistence of an indeterminate or positive clinical outcome score can lead to the patient's thresholds being updated to reflect their persistent indeterminate or positive clinical outcome score.

In some cases, the specificity and sensitivity of the test is adjusted based on an additional biomarker. For example, the protein panels disclosed herein may have different sensitivities or specificities in populations of individuals with a given genetic or racial background. In some cases, based on an additional biomarker, the clinical outcome score may be adjusted to reflect a changing sensitivity or specificity of the test.

### Treatment and Diagnostic Regimens

Provided herein are treatment and diagnostic regimens for implementing any of the methods described herein for detecting a presence or absence of advanced colorectal adenoma and treatment of the same.

Provided herein are methods for detecting a presence or absence of colorectal cancer. Methods disclosed herein can comprise performing a test for colorectal cancer, performing a colonoscopy, during which detected colorectal cancers are surgically excised or otherwise removed, and performing the test for colorectal cancer a second time at a later date. The second test can be positive and a second colonoscopy can be performed. In some cases, the second colonoscopy can include searching for and monitoring sessile colorectal cancers. In some cases, the second colonoscopy can include searching for and surgically removing sessile colorectal cancers. In some cases the second test for colorectal cancer can be positive and an additional treatment regimen can be recommended. In some cases, the second test for colorectal cancer can be negative and no additional testing can be recommended. In some cases, the second test for advanced colorectal adenoma can be negative and more frequent testing can be recommended for a given period of time.

A number of treatment regimens are contemplated herein, such as chemotherapy, radiation, immunotherapy, administration of a biologic therapeutic agent, and surgical intervention. A treatment regimen can be performed in response to a positive result, for example positive for colorectal cancer. The treatment regimen can be performed in response to a positive result for advanced colorectal adenoma. Surgical intervention can include, for example, polypectomy to remove a detected polyp. In some cases, surgical intervention can include partial colectomy to remove a part of the colon. In some cases, surgical intervention can include low anterior resection or abdominoperineal resection and colostomy. In some cases, a treatment regimen can include administrating to the subject one or more of leucovorin, 5-FU, oxaliplatin (Eloxatin®), irinotecan (Camptosar®), capecitabine (Xeloda®), Cetuximab, Panitumumab, Regorafenib (Stivarga®), trifluridine and tipiracil (Lonsurf®). In some cases, a treatment regimen can include administrating to the subject one or more of FOLFOX: leucovorin, 5-FU, and oxaliplatin (Eloxatin®); FOLFIRI: leucovorin, 5-FU, and irinotecan (Camptosar®); CapeOX: capecitabine (Xeloda®) and oxaliplatin; and FOLFOXIRI: leucovorin, 5-FU, oxaliplatin, and irinotecan. In some cases, a treatment regimen can include administrating to the subject one or more of a drug that targets VEGF (e.g., bevacizumab (Avastin®), ziv-aflibercept (Zaltrap®), ramucirumab (Cyramza®), and a drug that targets EGFR (e.g., cetuximab (Erbitux®), panitumumab (Vectibix®)).

One or more treatment regimens as described herein can be administered alone or in combination with one another. For example, a treatment regimen can include removal of malignant tissue in combination with one or more of radiation therapy, immunotherapy and chemotherapy. In some cases, more than one treatment regimen may be administered. In some cases, a treatment regimen may be repeated. For example, a subject may be monitored, such as after one or more periods described herein, after a first treatment regimen and a follow up treatment regimen may be administered if appropriate.

In some cases, a positive clinical outcome score can lead to the recommendation of a drug therapeutic regimen. For example, a positive clinical outcome score can result in the recommendation that a Wnt pathway inhibitor be administered to the subject. After the Wnt pathway inhibitor is administered, a second test for advanced colorectal adenoma can be administered to the subject. A negative or less severe clinical outcome score can indicate that the treatment is effective. A second positive or more severe clinical outcome score can indicate that the treatment is not effective.

### Computer Systems

Provided herein are computer systems for implementing any of the methods described herein for detecting a presence or absence of at least one of advanced colorectal adenoma and CRC. Also provided herein are computer systems for detecting a presence or absence of CRC. Computer systems disclosed herein comprises a memory unit. The memory unit can be configured to receive data comprising measurement of a biomarker panel from a biological sample of a subject. The biomarker panel can be any biomarker panel described herein. For example, the biomarker panel can comprise at least two biomarkers selected from the group comprising C9, CEA, DPP4, MIF, ORM1, PKM, SAA, and TFRC, and also including individual age and gender. Optionally, the biomarker panel includes CLU, CTSD, DPP4, GDF15, GSN, MIF, PKM, SERPINA1, SERPINA3, TFRC, and TIMP1, and in some cases includes age as an additional biomarker. In some cases a biomarker panel is selected from Table 3, or is selected from Table 4, or is selected from Table 5, or is selected from Table 6, or is a combination of biomarkers of at least two of Table 3, Table 4, Table 5 and Table 6.

Computer systems disclosed herein comprise computer executable code for performing at least one of: generating a subject-specific profile of a biomarker panel described herein based upon the measurement data, comparing the subject-specific profile of the biomarker panel to a reference profile of the biomarker panel, and determining a likelihood of advanced colorectal adenoma in the subject. Computer systems disclosed herein comprises computer executable code for performing at least one of: generating a subject-specific profile of a biomarker panel described herein based upon the measurement data, comparing the subject-specific profile of the biomarker panel to a reference profile of the biomarker panel, and determining a likelihood of CRC in the subject.

Additionally, provided herein are computer systems for implementing any of the methods described herein for detecting a presence or absence of at least one of advanced colorectal adenoma and CRC. For example, provided herein are computer systems for detecting a presence or absence of advanced colorectal adenoma. Also provided herein are computer systems for detecting a presence or absence of CRC. Computer systems disclosed herein comprises a memory unit. The memory unit can be configured to receive data comprising measurement of a biomarker panel from a biological sample of a subject. The biomarker panel can be any biomarker panel described herein. For example, the biomarker panel can comprise at least two biomarkers selected from the group comprising C9, CEA, DPP4, MIF, ORM1, PKM, SAA, and TFRC, and also including individual age and gender, or at least two biomarkers selected from the group comprising CLU, CTSD, DPP4, GDF15, GSN, MIF, PKM, SERPINA1, SERPINA3, TFRC, and TIMP1, and obtaining the age of the individual, or a biomarker panel of at least one of Table 3, Table 4, Table 5 and Table 6, such as a combination of biomarkers of at least two of Table 3, Table 4, Table 5 and Table 6.

Computer systems disclosed herein optionally comprise computer executable code for performing at least one of: generating a subject-specific profile of a biomarker panel described herein based upon the measurement data, comparing the subject-specific profile of the biomarker panel to a reference profile of the biomarker panel, and determining a likelihood of advanced colorectal adenoma in the subject. Computer systems disclosed herein optionally comprise computer executable code for performing at least one of: generating a subject-specific profile of a biomarker panel described herein based upon the measurement data, comparing the subject-specific profile of the biomarker panel to a reference profile of the biomarker panel, and determining a likelihood of CRC in the subject.

Computer systems described herein optionally comprise computer-executable code for performing any of the algorithms described herein. The computer system can further comprise computer-executable code for providing a report communicating the presence or absence of the at least one of advanced colorectal adenoma and CRC, for recommending a colonoscopy, sigmoidoscopy, or colorectal tissue biopsy, and/or for recommending a treatment. In some embodiments, the computer system executes instructions contained in a computer-readable medium.

In some embodiments, the processor is associated with one or more controllers, calculation units, and/or other units of a computer system, or implanted in firmware. In some embodiments, one or more steps of the method are implemented in hardware. In some embodiments, one or more steps of the method are implemented in software. Software routines may be stored in any computer readable memory unit such as flash memory, RAM, ROM, magnetic disk, laser disk, or other storage medium as described herein or known in the art. Software may be communicated to a computing device by any known communication method including, for example, over a communication channel such as a telephone line, the internet, a wireless connection, or by a transportable medium, such as a computer readable disk, flash drive, etc. The one or more steps of the methods described herein may be implemented as various operations, tools, blocks, modules and techniques which, in turn, may be implemented in firmware, hardware, software, or any combination of firmware, hardware, and software. When implemented in hardware, some or all of the blocks, operations, techniques, etc. may be implemented in, for example, an application specific integrated circuit (ASIC), custom integrated circuit (IC), field programmable logic array (FPGA), or programmable logic array (PLA).

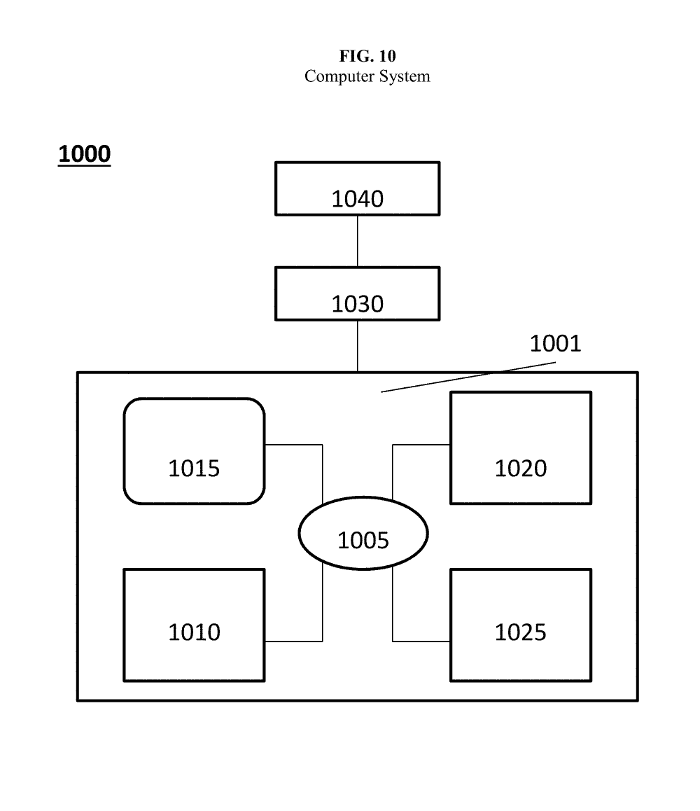

FIG. 10 depicts an exemplary computer system 1000 adapted to implement a method described herein. The system 1000 includes a central computer server 1001 that is programmed to implement exemplary methods described herein. The server 1001 includes a central processing unit (CPU, also “processor”) 1005 which can be a single core processor, a multi core processor, or plurality of processors for parallel processing. The server 1001 also includes memory 1010 (for example random access memory, read-only memory, flash memory); electronic storage unit 1015 (for example hard disk); communications interface 1020 (for example network adaptor) for communicating with one or more other systems; and peripheral devices 1025 which may include cache, other memory, data storage, and/or electronic display adaptors. The memory 1010, storage unit 1015, interface 1020, and peripheral devices 1025 are in communication with the processor 1005 through a communications bus (solid lines), such as a motherboard. The storage unit 1015 can be a data storage unit for storing data. The server 1001 is operatively coupled to a computer network (“network”) 1030 with the aid of the communications interface 1020. The network 1030 can be the Internet, an intranet and/or an extranet, an intranet and/or extranet that is in communication with the Internet, a telecommunication or data network. The network 1030 in some cases, with the aid of the server 1001, can implement a peer-to-peer network, which may enable devices coupled to the server 1001 to behave as a client or a server.

The storage unit 1015 can store files, such as subject reports, and/or communications with the caregiver, sequencing data, data about individuals, or any aspect of data associated with the disclosure herein.

The server can communicate with one or more remote computer systems through the network 1030. The one or more remote computer systems may be, for example, personal computers, laptops, tablets, telephones, Smart phones, or personal digital assistants.

In some situations the system 1000 includes a single server 1001. In other situations, the system includes multiple servers in communication with one another through an intranet, extranet and/or the Internet.

The server 1001 can be adapted to store measurement data, patient information from the subject, such as, for example, polymorphisms, mutations, medical history, family history, demographic data and/or other information of potential relevance. Such information can be stored on the storage unit 1015 or the server 1001 and such data can be transmitted through a network.

Methods as described herein are in some cases implemented by way of machine (or computer processor) executable code (or software) stored on an electronic storage location of the server 1001, such as, for example, on the memory 1010, or electronic storage unit 1015. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010. Alternatively, the code can be executed on a second computer system 1040.

Aspects of the systems and methods provided herein, such as the server 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (for example, read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

The results of detection of a presence or absence of at least one of an advanced colorectal adenoma and CRC, generating a subject report, and/or communicating the report to a caregiver can be presented to a user with the aid of a user interface, such as a graphical user interface.

A computer system may be used to implement one or more steps of a method described herein, including, for example, sample collection, sample processing, measurement of an amount of one or more proteins described herein to produce measurement data, determination of a ratio of a protein to another protein to produce measurement data, comparing measurement data to a reference amount, generating a subject-specific profile of a biomarker panel, comparing the subject-specific profile to a reference profile, receiving medical history, receiving medical records, receiving and storing measurement data obtained by one or more methods described herein, analyzing said measurement data to determine a presence or absence of at least one of an advanced colorectal adenoma and CRC (for example, by performing an algorithm described herein), generating a report, and reporting results to a receiver.

A client-server and/or relational database architecture can be used in any of the methods described herein. In general, a client-server architecture is a network architecture in which each computer or process on the network is either a client or a server. Server computers can be powerful computers dedicated to managing disk drives (file servers), printers (print servers), or network traffic (network servers). Client computers can include PCs (personal computers) or workstations on which users run applications, as well as example output devices as disclosed herein. Client computers can rely on server computers for resources, such as files, devices, and even processing power. The server computer handles all of the database functionality. The client computer can have software that handles front-end data management and receive data input from users.

After performing a calculation, a processor can provide the output, such as from a calculation, back to, for example, the input device or storage unit, to another storage unit of the same or different computer system, or to an output device. Output from the processor can be displayed by a data display, for example, a display screen (for example, a monitor or a screen on a digital device), a print-out, a data signal (for example, a packet), a graphical user interface (for example, a webpage), an alarm (for example, a flashing light or a sound), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In some embodiments, an output device is the same device as the input device. Example output devices include, but are not limited to, a telephone, a wireless telephone, a mobile phone, a PDA, a flash memory drive, a light source, a sound generator, a fax machine, a computer, a computer monitor, a printer, an iPod, and a webpage. The user station may be in communication with a printer or a display monitor to output the information processed by the server. Such displays, output devices, and user stations can be used to provide an alert to the subject or to a caregiver thereof.

Data relating to the present disclosure can be transmitted over a network or connections for reception and/or review by a receiver. The receiver can be but is not limited to the subject to whom the report pertains; or to a caregiver thereof, for example, a health care provider, manager, other healthcare professional, or other caretaker; a person or entity that performed and/or ordered the genotyping analysis; a genetic counselor. The receiver can also be a local or remote system for storing such reports (for example servers or other systems of a “cloud computing” architecture). In one embodiment, a computer-readable medium includes a medium suitable for transmission of a result of an analysis of a biological sample.

### Kits

The present disclosure also provides kits. In some cases, a kit described herein comprises one or more compositions, reagents, and/or device components for measuring and/or detecting one or more biomarkers described herein. A kit as described herein can further comprise instructions for practicing any of the methods provided herein. The kits can further comprise reagents to enable the detection of biomarker by various assays types such as antibody binding florescence assay, ELISA assay, immunoassay, protein chip or microarray, mass spectrometry, immunohistochemistry, flow cytometry, or high content cell screening. Kits can also comprise a computer readable medium comprising computer executable code for implementing a method described herein.

In some embodiments, a kit provided herein comprises antibodies to the biomarkers described elsewhere in the disclosure. A kit may comprise at least two antibodies that are each reactive against a biomarkers selected from the group consisting of C9, CEA, CLU, CTSD, DPP4, GDF15, GSN, MIF, ORM1, PKM, SAA, SERPINA1, SERPINA3, TFRC, and TIMP1. A kit may comprise antibodies to detect proteins of a panel of Table 3, and optionally a form for indicating age and optionally gender. In some cases, a kit provided herein comprises antibodies to C9, CEA, DPP4, MIF, ORM1, PKM, SAA, and TFRC. In other cases, a kit provided herein comprises antibodies to CLU, CTSD, DPP4, GDF15, GSN, MIF, PKM, SERPINA1, SERPINA3, TFRC, and TIMP1. A kit may comprise antibodies to detect proteins of a panel of Table 5, and optionally a form for indicating age and optionally gender.

In some embodiments, kits described herein include a packaging material. As used herein, the term “packaging material” can refer to a physical structure housing the components of the kit. The packaging material can maintain sterility of the kit components, and can be made of material commonly used for such purposes (for example, paper, corrugated fiber, glass, plastic, foil, ampules, etc.). Kits can also include a buffering agent, a preservative, or a protein/nucleic acid stabilizing agent. Kits can include components for obtaining a biological sample from a patient. Non-limiting examples of such components can be gloves, hypodermic needles or syringes, tubing, tubes or vessels to hold the biological sample, sterilization components (e.g. isopropyl alcohol wipes or sterile gauze), and/or cooling material (e.g., freezer pack, dry ice, or ice).

In some cases, kits disclosed herein are used in accordance of any of the disclosed methods.

### Incorporation of Indeterminate Classification Calls (NoC Method)

The intrinsic performance of a particular classification model depends on the distributions and separation of model scores for the two classes. With the rare exception of perfect class separation, most classification models make mistakes because of class overlap across the range of classifier scores. For example, such an overlap may occur near the middle of the score range where the probability of being in one class or the other is close to 50%.

Within such an overlap region, it may be advantageous to add a third class to the final set of classification calls; the third class would indicate the uncertainty of a call in this score region. This could be implemented, for example, by defining an indeterminate region of classification scores. Samples with scores in this region would be given an “indeterminate” or “no call” test result. Samples with scores above or below this region would be given standard positive or negative test results depending on their positions relative to the test cutoff. The benefit of adding an indeterminate region to a classification model is that classification performance can improve for samples outside of the indeterminate region, i.e. mistakes are less likely for the remaining positive and negative tests. However, if the indeterminate range is too large, there may be too many indeterminate results, and the value of the test may be put into question.

In some analyses, referred to here as NoC (“No Call”), the effect of using an indeterminate region with the classification models was investigated. In one of these analyses, the percentage of samples targeted to receive a “no call” result was set to 10%. To determine the optimal score range for the indeterminate region (NoC region) with 10% of the samples, the specificity was maximized at a sensitivity of >=90% as follows: All possible contiguous sets of 10% of samples were determined across the classifier scores range. For each set, the associated set of 10% of samples were marked as no calls. These samples were removed from the analysis set and the ROC curve was generated from the remaining 90% of the samples. The maximum specificity at >=90% sensitivity was then determined and used as the evaluation score for the NoC region in question. After all NoC regions were evaluated in this manner, the region with the highest specificity score given a criterion minimum sensitivity score was selected as the optimal NoC region. The score range defining this NOC region was taken from the upper and lower classification scores of the associated 10% no call samples.

### Characteristics of Panels Disclosed Herein Relative to Other Biomarker Panels

Panels disclosed herein substantially outperform individual markers or randomly generated panels. Although at least some members of the panels herein are implicated in cancer, the panels herein far outperform panels derived randomly from any art teachings. This is illustrated by examination of panel performance as compared to individual members, randomly generated panels, and in light of the unpredictability of individual markers for any individual health assessment.

Panels were constructed from an original candidate pool of 187 potential biomarkers selected from the literature. Using a 274 member age and gender matched discovery sample set, targeted mass spectroscopy was used to identify 31 biomarkers from the original set that co-vary with health status of the 274 members of the discovery sample set. This 31 member set is not a random selection of the 187 member original candidate pool, and the 31 member set was not selected from the original 187 member candidate pool based upon any teaching in the art. Nonetheless, the 31 member panel may serve in some cases as a proxy for markers that one may identify in related art.

The curated set of 31 biomarkers was further narrowed to identify sets of proteins. A set of 27 of the original 31 biomarkers was used to run 4,507 samples to generate a set of new classifiers. Two of the 27 biomarkers were considered poor quality because they had concerns over reagent strength, resulting in a set of 25 biomarkers of which 15 were included in the classifier build effort. A brute force method was used to evaluate the performance of millions of classifiers that were part of the build effort, and the effect of this on a discovery set of proteins.

The 25 member set was tested against a separate age and gender matched 300 member sample set to come to CRC panels as disclosed herein, such as the 8 member panel comprising C9, CEA, DPP4, MIF, ORM1, PKM, SAA, and TFRC. This and similar panels were selected from an original 187 member candidate pool. The panel is come to through repeated analysis of independently derived samples.

Biomarker panels herein perform substantially better than any random selection of biomarkers individually implicated in cancer generally, such as those of the 187 member candidate pool. That is, if one of skill in the art were to start with a list of biomarkers available in the literature and randomly assemble, or even assemble in light of teachings available to one of skill in the art, a biomarker panel to use to assay for a colorectal health issue such as colorectal cancer or advanced adenoma in an individual, one does not come to a biomarker as disclosed herein. Biomarker panels disclosed herein substantially outperform randomly selected panels and panels selected in light of the art.

Biomarker panels herein perform substantially better than any individual constituent marker individually implicated in cancer generally, such as those of the 187 member candidate pool. Some individual biomarkers indicate CRC or advanced adenoma, but with a sensitivity and a specificity that is far below that of the biomarker panels as disclosed herein. Use of individual biomarkers, or combinations of biomarkers not recited or readily apparent to one of skill in the art from the disclosure herein, is not contemplated pursuant to this disclosure.

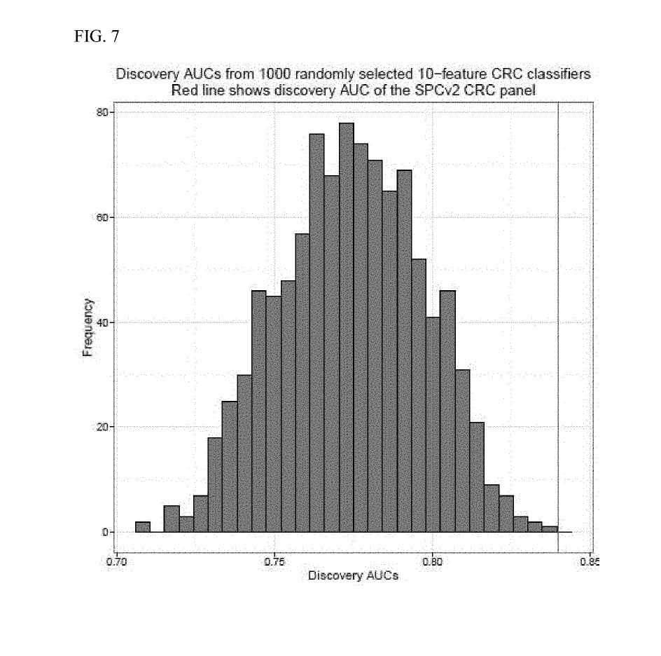

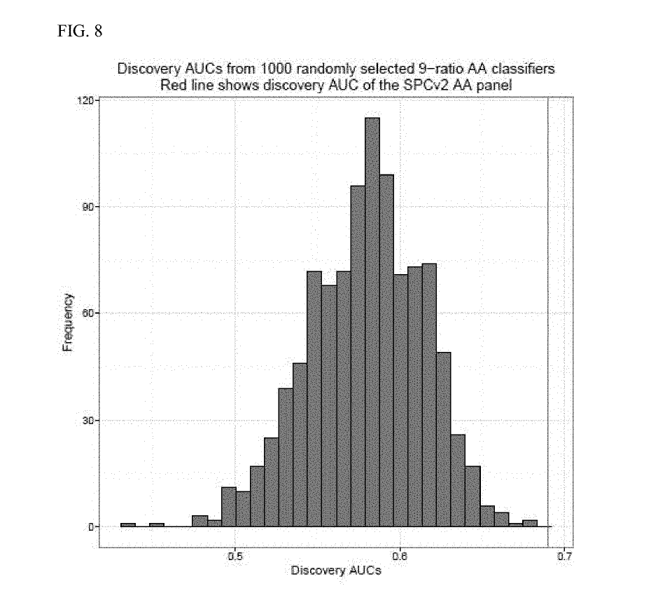

Aggregation of protein markers alone does not accomplish the level of performance of the panels disclosed herein. In illustration of this assertion, random panels were generated from a targeted enriched set of 25 markers, and their performance is compared to that of the panels herein (see FIGS. 7-8). The enriched 25 member set is already expected to yield panels that perform much better than those generated from the unenriched parent 187 marker set. It is observed that the panels herein, as shown, substantially outperform panels generated at random from an already enriched set of protein markers. These random panels do not represent panels that one would come to from the art, as they are already enriched from the 187 member list as mentioned in the art as being relevant to cancer detection.

Biomarker panels herein yield results that are more reliable, more sensitive and more specific than simply the collection of their individual constituents. That is, in some cases individual biomarkers are detected at levels that are individually not informative with a degree of sensitivity and specificity to be medically relevant, but the level of the biomarker panel nonetheless provides a colorectal health assessment with a degree of confidence that is medically actionable. In some cases no individual biomarker of the panel is present at a level that is individually indicative of a health issue warranting follow-up, but the biomarker panel as a whole, assessed as indicated herein, provides an assessment that is indicative of a health issue warranting follow-up.

Biomarkers herein yield results that are in some cases qualitatively different from those of their constituent biomarkers. That is, in some cases one or more individual biomarkers of the panel are present at a level that is individually indicative of a colorectal health status that is contradictory to the health status indicated by the level of the panel as a whole, including the contradictory biomarker. In such cases, it is often found that independent health assessment, for example by colonoscopy or by stool sample analysis, supports the panel assessment rather than the health status assessment provided by the contradictory individual marker.

Reference is made to Table 7. In that table, one sees data for the use of a CRC panel in the determination of patient CRC risk. One observes that the CRC biomarker panels provide predictions that are inconsistent with the predictions that result from looking at constituent biomarker levels in isolation. Shaded cells highlight situations where the same measurement, in different patient samples, corresponds to different patient CRC status calls.

The protein CEA, and the marker of age are shaded in Table 7 below, in instances where a single measurement level contributed to diverging conclusions in consecutive samples. CEA is known to correspond with cancer status in a number of cancer conditions. However, as demonstrated in the table below, panels as disclosed herein provide a level of accuracy that surpasses that of any individual marker constituent, such that an aberrant signal from a single marker can nonetheless lead to a correct overall panel health status call.

If one were to use CEA in isolation, then one would expect the first and second entries in Table 7 to have a common health status call. However, using the panel analysis as disclosed herein, one comes to a result that is qualitatively different from the result expected by examination of an individual panel biomarker in isolation. This data as presented in Table 7, below, highlights the fact that the panels herein are not simply quantitatively better but are also in some cases qualitatively different from their individual biomarker constituents.

Accordingly, biomarker panels disclosed herein are understood to perform better than a random collection of candidate markers as taught by the literature. Biomarker panels disclosed herein are also understood to perform better statistically, and in some cases qualitatively differently, than do their individual biomarker constituents, such that a health assessment from the biomarker panel as a whole is either more accurate or in some cases provides a result that is qualitatively different from that of one or more individual biomarker constituents.

### Additional In Vitro Analyses

The disclosure herein makes reference to methods comprising obtaining samples from individuals and analyzing said samples form the presence or level of accumulation of circulating proteins or polypeptides. In alternate embodiments, methods are performed on in vitro samples, independent of the sample source. In these embodiments, similar or identical panels, detection steps and analyses are performed, but these embodiments do not recite drawing blood from an individual. Rather, samples, independent of origin, are obtained in a laboratory or other experimental setting, and are subject to analysis so as to obtain panel information for downstream analysis as disclosed herein. In these embodiments, samples may ultimately have arisen from human patients, but the sample source is not recited in any associated claim, such that the claims do not recite acting on a human patient. Instead, the claims recite performing analyses upon in vitro samples obtained in a lab.

### Additional Reference to Figures

The disclosure herein is delineated throughout the specification and claims appended herewith, supported by the figures. Referring to the figures in more detail, one observes the following.

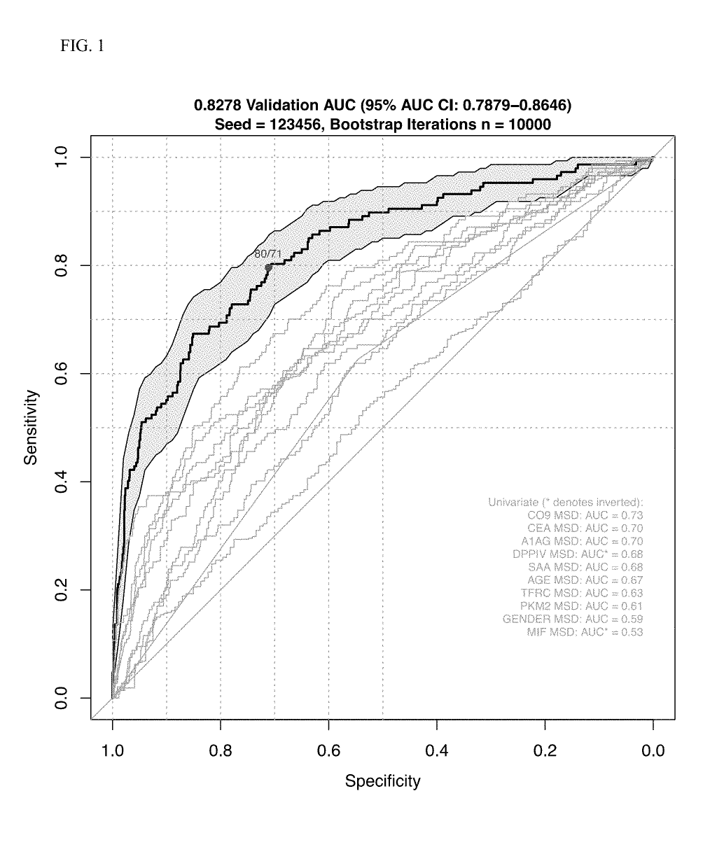

At FIG. 1, one sees an AUC plot for a lead CRC panel. The panel exhibits an 0.8278 Validation AUC (95% AUC confidence interval of 0.7879-0.8646), with a seed of 123456 and 10,000 Bootstrap iterations. Depicted on the plot are the panel's performance of 80% sensitivity at 71% specificity. In repeated panel tests, the panel classified 59 of 75 class I/II CRC blood samples correctly, for a sensitivity of 0.79, and classified 58 of 73 class III/IV samples correctly for a sensitivity of 0.81, with a Fisher's test P-value of 0.839.

Individual panel constituents are also depicted on the AUC plot. It is observed that the panel substantially outperformed individual members, with individual panel constituents exhibiting AUC values as follows: CO9 0.73; CEA 0.70; A1AG 0.70; DPP4 0.68; SAA 0.68; AGE 0.67; TFRC 0.63; PKM2 0.61; Gender 0.59; MIF 0.53.

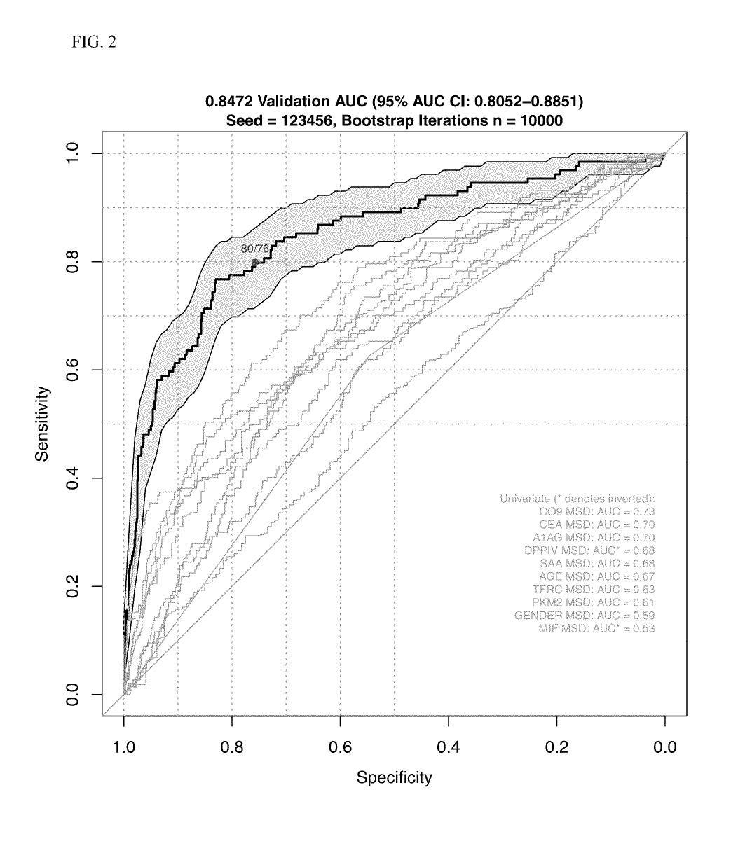

At FIG. 2, one sees an AUC plot for the lead CRC panel of FIG. 1 with a 15% NoC. The panel exhibits an 0.8472 Validation AUC (95% AUC confidence interval of 0.8052-0.8851), with a seed of 123456 and 10,000 Bootstrap iterations. Depicted on the plot are the panel's performance of 80% sensitivity at 76% specificity. In repeated panel tests, the panel classified 50 of 63 class I/II CRC blood samples correctly, for a sensitivity of 0.79, and classified 53 of 66 class III/IV samples correctly for a sensitivity of 0.80, with a Fisher's test P-value of 0.839. The AUC plot was 0.85, with Val NoC of 12.3% (HERE: validation NoC?)

Individual panel constituents are also depicted on the AUC plot. It is observed that the panel substantially outperformed individual members, with individual panel constituents exhibiting AUC values as follows: CO9 0.73; CEA 0.70; A1AG 0.70; DPP4 0.68; SAA 0.68; AGE 0.67; TFRC 0.63; PKM2 0.61; Gender 0.59; MIF 0.53.

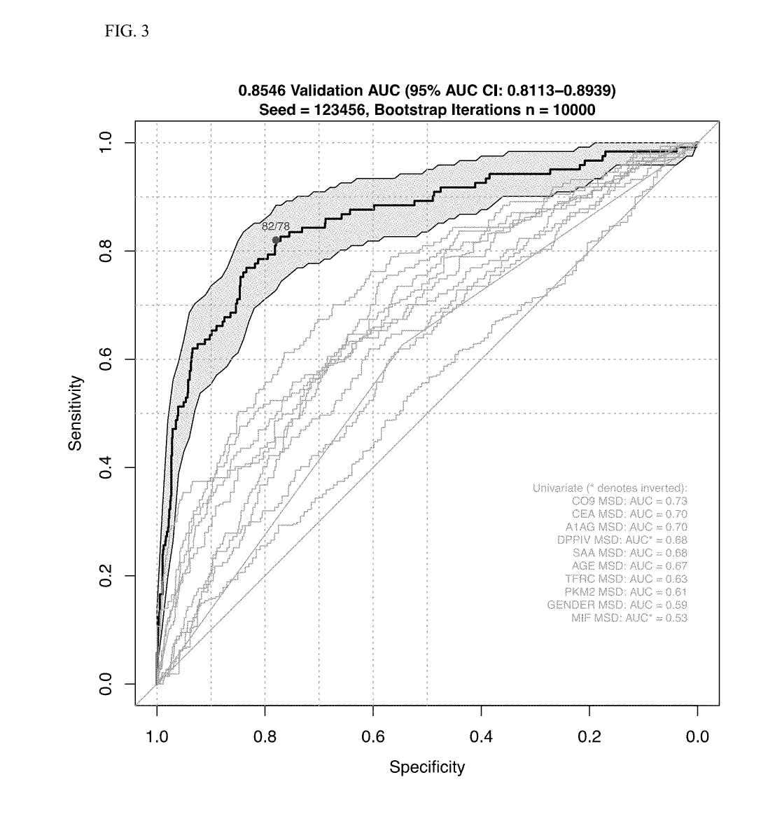

At FIG. 3, one sees an AUC plot for the lead CRC panel of FIG. 1 with a 20% NoC. The panel exhibits an 0.8546 Validation AUC (95% AUC confidence interval of 0.8113-0.8939), with a seed of 123456 and 10,000 Bootstrap iterations. Depicted on the plot are the panel's performance of 82% sensitivity at 78% specificity. In repeated panel tests, the panel classified 45 of 57 class I/II CRC blood samples correctly, for a sensitivity of 0.79, and classified 54 of 73 class III/IV samples correctly for a sensitivity of 0.74, with a Fisher's test P-value of 0.485. The AUC plot was 0.85, with a Val NoC of 18.2%.

Individual panel constituents are also depicted on the AUC plot. It is observed that the panel substantially outperformed individual members, with individual panel constituents exhibiting AUC values as follows: CO9 0.73; CEA 0.70; A1AG 0.70; DPP4 0.68; SAA 0.68; AGE 0.67; TFRC 0.63; PKM2 0.61; Gender 0.59; MIF 0.53.

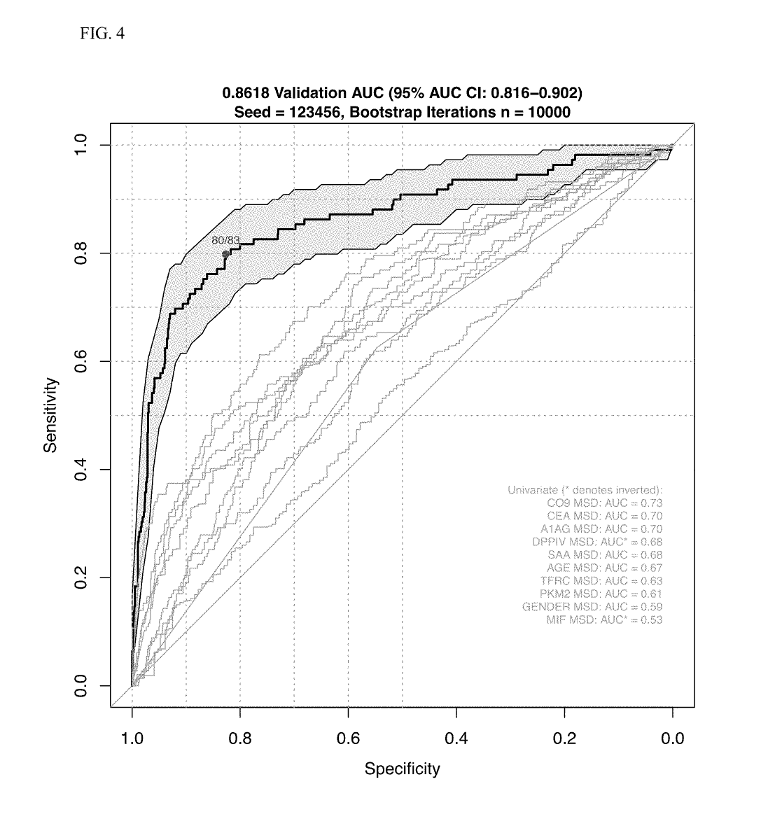

At FIG. 4, one sees an AUC plot for a lead CRC panel of FIG. 1 with a 25% NoC. The panel exhibits an 0.8618 Validation AUC (95% AUC confidence interval of 0.816-0.902), with a seed of 123456 and 10,000 Bootstrap iterations. Depicted on the plot are the panel's performance of 80% sensitivity at 83% specificity. In repeated panel tests, the panel classified 36 of 48 class I/II CRC blood samples correctly, for a sensitivity of 0.75, and classified 51 of 61 class III/IV samples correctly for a sensitivity of 0.84, with a Fisher's test P-value of 0.338. The AUC plot was 0.86 with a Val NoC of 23.2%.

Individual panel constituents are also depicted on the AUC plot. It is observed that the panel substantially outperformed individual members, with individual panel constituents exhibiting AUC values as follows: CO9 0.73; CEA 0.70; A1AG 0.70; DPP4 0.68; SAA 0.68; AGE 0.67; TFRC 0.63; PKM2 0.61; Gender 0.59; MIF 0.53.

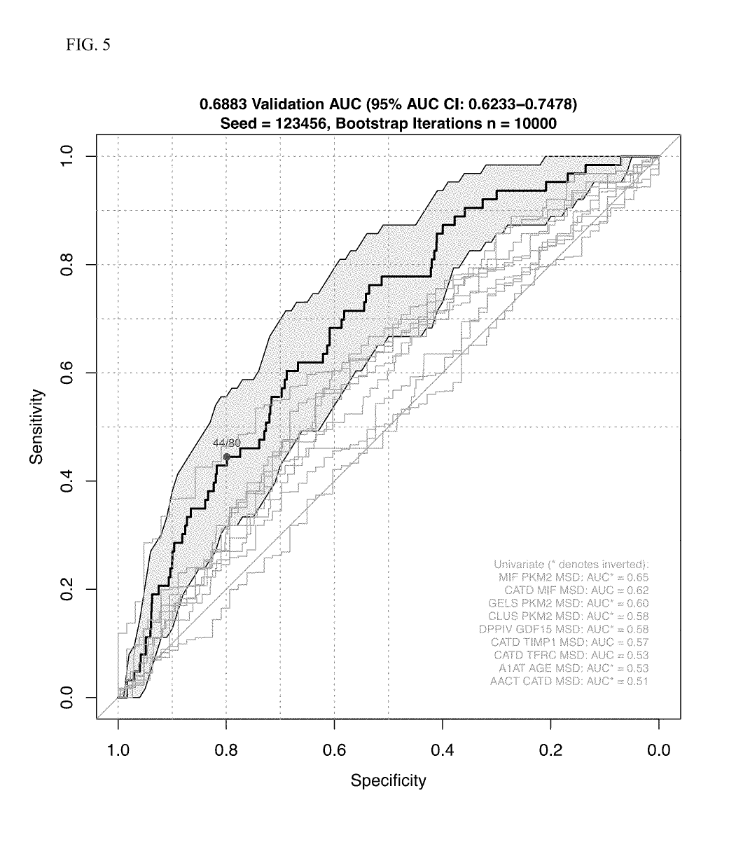

At FIG. 5, one sees an AUC plot for a lead AA panel. The panel exhibits an 0.6883 Validation AUC (95% AUC confidence interval of 0.6233-0.7478), with a seed of 123456 and 10,000 Bootstrap iterations. Depicted on the plot are the panel's performance of 44% sensitivity at 80% specificity with an AUC of 0.69.

Individual panel constituents are also depicted on the AUC plot. It is observed that the panel substantially outperformed individual members, with individual panel constituents exhibiting AUC values as follows: MIF PKM2 0.73; CATD MIF 0.70; GELS PKM2 0.70; CLUS PKM2 0.68; DPP4 GDF15 0.68; CATD TIMP1 0.67; CATD TFRC 0.63; A1AT AGE 0.61; AACT CATD 0.59.

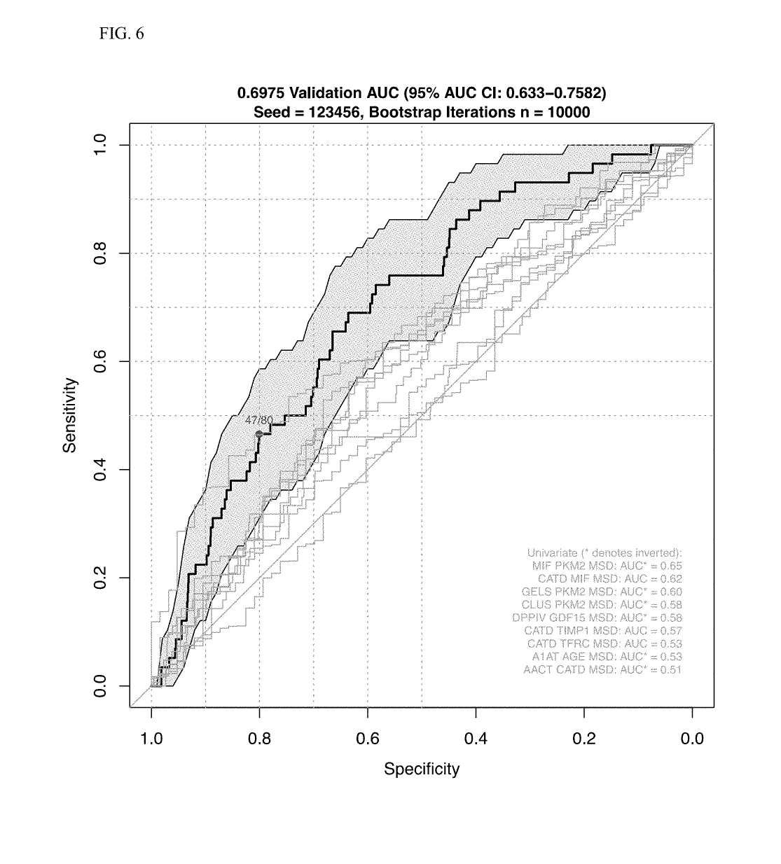

At FIG. 6, one sees an AUC plot for a lead AA panel. The panel exhibits an 0.6975 Validation AUC (95% AUC confidence interval of 0.633-0.7582), with a seed of 123456 and 10,000 Bootstrap iterations. Depicted on the plot are the panel's performance of 47% sensitivity at 80% specificity with an AUC of 0.69 and a Val NoC 8.5%.

Individual panel constituents are also depicted on the AUC plot. It is observed that the panel substantially outperformed individual members, with individual panel constituents exhibiting AUC values as follows: MIF PKM2 0.65; CATD MIF 0.62; GELS PKM2 0.60; CLUS PKM2 0.58; DPP4 GDF15 0.58; CATD TIMP1 0.57; CATD TFRC 0.53; A1AT AGE 0.53; AACT CATD 0.51.

At FIG. 7, one sees an analysis of 1000 randomly selected 10-feature CRC classifiers. Classifiers were selected from the 25-member precursor set of markers relevant to CRC and AA. The Y axis indicates frequency while the X-axis indicates the discovery AUC. The height of each column indicates the frequency by which a randomly selected panel from the set of 25 enriched biomarkers exhibited the indicated AUC. The thin line at far right indicates the discovery AUC exhibited by lead CRC panels as disclosed herein. These results indicate that the lead panels disclosed herein substantially outperform randomly selected panels, even when selected from a marker set substantially enriched for relevance in CRC detection.

At FIG. 8, one sees an analysis of 1000 randomly selected 9-feature AA classifiers. Classifiers were selected from the 25-member precursor set of markers relevant to CRC and AA, with marker values mathematically combined into 9 separate features as in the lead AA classifiers. The Y axis indicates frequency while the X-axis indicates the discovery AUC. The height of each column indicates the frequency by which a randomly selected panel from the set of 25 enriched biomarkers exhibited the indicated AUC. The thin line at far right indicates the discovery AUC exhibited by the lead AA panels as disclosed herein. These results indicate that the lead panels disclosed herein substantially outperform randomly selected panels, even when selected from a marker set substantially enriched for relevance in AA detection.

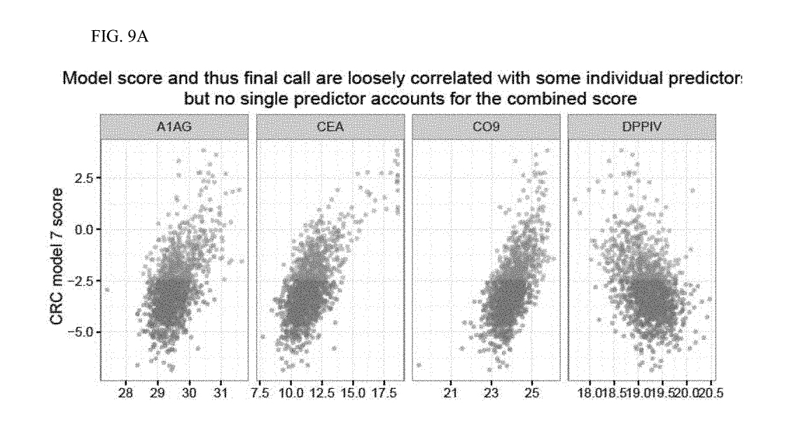

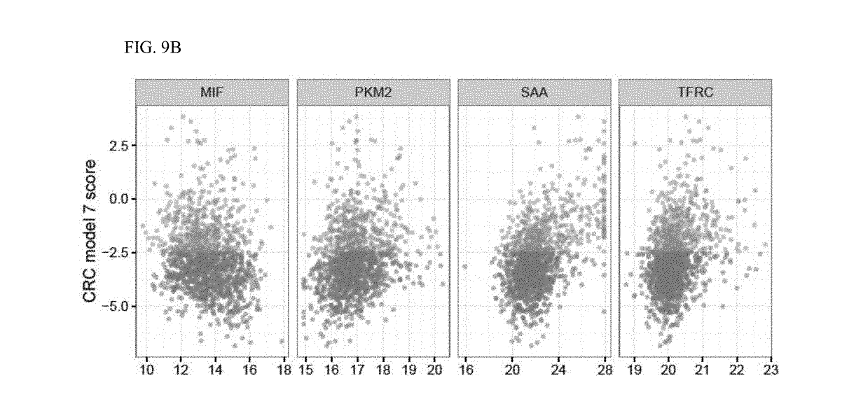

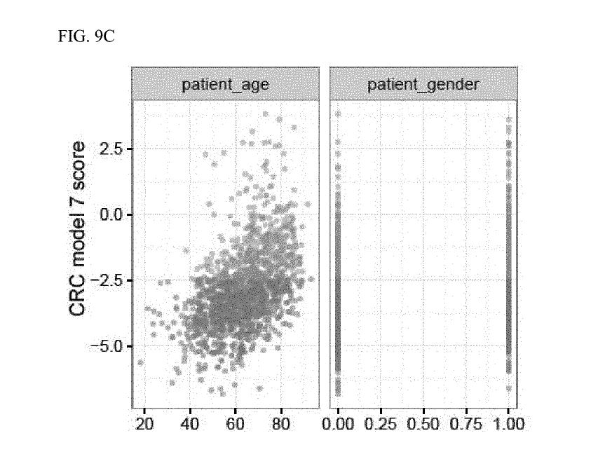

At FIGS. 9A-9C, one sees graphs of CRC model score against individual marker log 2 concentration (9A, 9B) or age in years or gender (9C). Marker identity is indicated at the top of each panel. Model score is indicated on the Y-axis. Each point is mapped to its concentration and to the score of the model on which it was a member. The border between a positive and a negative call is at −2.5 on the y-axis. Points below Y=−2.5 are shaded more darkly than are points above Y=−2.5.

One sees from FIGS. 9A-9C that individual CRC markers show varying degrees of correlations to overall panel prediction. For A1AG, CEA, CO9, PKM2, SAA, TRFC and age, there is a noticeable positive correlation between concentration or amount and a disease call. For MIF and DPPIV, the correlation is negative.

However, one also sees that the correlations are weak, and that there is no clear concentration or accumulation level that definitively predicts overall CRC model disease call. For example, referring to A1AG at FIG. 9A, one sees a general positive correlation between concentration and a positive CRC model call. However, there are a very large number of exceptions. A concentration of between 29 and 30, in particular, one sees a large number of points that do not follow the general correlation. That is, at a concentration of 29 there are a number of points that nonetheless correspond to CRC positive models, while at a concentration of 30 there are a number of points that nonetheless correspond to CRC negative models.

Similarly, looking at TFRC accumulation levels, one observes a generally positive correlation between concentration and CRC model positive prediction. However, even at the highest concentration, one sees samples for which a high TFRG concentration mapped to a negative panel result.

At FIG. 10, one sees a computer system consistent with the methods and panels disclosed herein.

### Reference Art and Definitions

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present disclosure can employ, unless otherwise indicated, conventional techniques of immunology, biochemistry, chemistry, molecular biology, microbiology, cell biology, genomics and recombinant DNA, which are within the skill of the art. See, for example, Sambrook, Fritsch and Maniatis, MOLECULAR CLONING: A LABORATORY MANUAL, 4th edition (2012); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), CULTURE OF ANIMAL CELLS: A MANUAL OF BASIC TECHNIQUE AND SPECIALIZED APPLICATIONS, 6th Edition (R. I. Freshney, ed. (2010), and Lange, et. al., Molecular Systems Biology Vol. 4:Article 222 (2008), which are hereby incorporated by reference.

Some colorectal health assays comprising panels are described, for example, in U.S. Patent Application Publication No. US2016/0299144, published Oct. 13, 2016, which is hereby incorporated by reference in its entirety.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement, and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

The terms “panel”, “biomarker panel”, “protein panel”, “classifier model”, and “model” are used interchangeably herein to refer to a set of biomarkers, wherein the set of biomarkers comprises at least two biomarkers. Exemplary biomarkers are proteins or polypeptide fragments of proteins that are uniquely or confidently mapped to particular proteins. However, additional biomarkers are also contemplated, for example age or gender of the individual providing a sample. The biomarker panel is often predictive and/or informative of a subject's health status, disease, or condition.

The “level” of a biomarker panel refers to the absolute and relative levels of the panel's constituent markers and the relative pattern of the panel's constituent biomarkers.

The terms “colorectal cancer” and “CRC” are used interchangeably herein. The term “colorectal cancer status”, “CRC status” can refer to the status of the disease in subject. Examples of types of CRC statuses include, but are not limited to, the subject's risk of cancer, including colorectal carcinoma, the presence or absence of disease (for example, polyp or adenocarcinoma), the stage of disease in a patient (for example, carcinoma), and the effectiveness of treatment of disease.

The term “mass spectrometer” can refer to a gas phase ion spectrometer that measures a parameter that can be translated into mass-to-charge (m/z) ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” can refer to the use of a mass spectrometer to detect gas phase ions.

The term “tandem mass spectrometer” can refer to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that can be capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector-magnetic sector mass spectrometers, and combinations thereof.

The term “biochip” can refer to a solid substrate having a generally planar surface to which an adsorbent is attached. In some cases, a surface of the biochip comprises a plurality of addressable locations, each of which location may have the adsorbent bound there. Biochips can be adapted to engage a probe interface, and therefore, function as probes. Protein biochips are adapted for the capture of polypeptides and can be comprise surfaces having chromatographic or biospecific adsorbents attached thereto at addressable locations. Microarray chips are generally used for DNA and RNA gene expression detection.

The term “biomarker” and “marker” are used interchangeably herein, and can refer to a polypeptide, gene, nucleic acid (for example, DNA and/or RNA) which is differentially present in a sample taken from a subject having a disease for which a diagnosis is desired (for example, CRC), or to other data obtained from the subject with or without sample acquisition, such as patient age information or patient gender information, as compared to a comparable sample or comparable data taken from control subject that does not have the disease (for example, a person with a negative diagnosis or undetectable CRC, normal or healthy subject, or, for example, from the same individual at a different time point). Common biomarkers herein include proteins, or protein fragments that are uniquely or confidently mapped to a particular protein (or, in cases such as SAA, above, a pair or group of closely related proteins), transition ion of an amino acid sequence, or one or more modifications of a protein such as phosphorylation, glycosylation or other post-translational or co-translational modification. In addition, a protein biomarker can be a binding partner of a protein, protein fragment, or transition ion of an amino acid sequence.

The terms “polypeptide,” “peptide” and “protein” are often used interchangeably herein in reference to a polymer of amino acid residues. A protein, generally, refers to a full-length polypeptide as translated from a coding open reading frame, or as processed to its mature form, while a polypeptide or peptide informally refers to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

An “immunoassay” is an assay that uses an antibody to specifically bind an antigen (for example, a marker). The immunoassay can be characterized by the use of specific binding properties of a particular antibody to isolate, target, and/or quantify the antigen.

The term “antibody” can refer to a polypeptide ligand substantially encoded by an immunoglobulin gene or immunoglobulin genes, or fragments thereof, which specifically binds and recognizes an epitope. Antibodies exist, for example, as intact immunoglobulins or as a number of well-characterized fragments produced by digestion with various peptidases. This includes, for example, Fab″ and F(ab)″2 fragments. As used herein, the term “antibody” also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody can refer to that portion of an immunoglobulin heavy chain that comprises one or more heavy chain constant region domains, but does not include the heavy chain variable region.

The term “tumor” can refer to a solid or fluid-filled lesion or structure that may be formed by cancerous or non-cancerous cells, such as cells exhibiting aberrant cell growth or division. The terms “mass” and “nodule” are often used synonymously with “tumor”. Tumors include malignant tumors or benign tumors. An example of a malignant tumor can be a carcinoma which is known to comprise transformed cells.

The term “binding partners” can refer to pairs of molecules, typically pairs of biomolecules that exhibit specific binding. Protein-protein interactions can occur between two or more proteins, when bound together they often to carry out their biological function. Interactions between proteins are important for the majority of biological functions. For example, signals from the exterior of a cell are mediated via ligand receptor proteins to the inside of that cell by protein-protein interactions of the signaling molecules. For example, molecular binding partners include, without limitation, receptor and ligand, antibody and antigen, biotin and avidin, and others.

The term “control reference” can refer to a known or determined amount of a biomarker associated with a known condition that can be used to compare to an amount of the biomarker associated with an unknown condition. A control reference can also refer to a steady-state molecule which can be used to calibrate or normalize values of a non-steady state molecule. A control reference value can be a calculated value from a combination of factors or a combination of a range of factors, such as a combination of biomarker concentrations or a combination of ranges of concentrations.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be cancer. The cancer can be CRC (CRC). In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An “ex vivo” assay is not performed on a subject. Rather, it is performed upon a sample separate from a subject. An example of an ‘ex vivo’ assay performed on a sample is an ‘in vitro’ assay.

The term “in vitro” is used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

The term specificity, or true negative rate, can refer to a test's ability to exclude a condition correctly. For example, in a diagnostic test, the specificity of a test is the proportion of patients known not to have the disease, who will test negative for it. In some cases, this is calculated by determining the proportion of true negatives (i.e. patients who test negative who do not have the disease) to the total number of healthy individuals in the population (i.e., the sum of patients who test negative and do not have the disease and patients who test positive and do not have the disease).

The term sensitivity, or true positive rate, can refer to a test's ability to identify a condition correctly. For example, in a diagnostic test, the sensitivity of a test is the proportion of patients known to have the disease, who will test positive for it. In some cases, this is calculated by determining the proportion of true positives (i.e. patients who test positive who have the disease) to the total number of individuals in the population with the condition (i.e., the sum of patients who test positive and have the condition and patients who test negative and have the condition).

The quantitative relationship between sensitivity and specificity can change as different diagnostic cut-offs are chosen. This variation can be represented using ROC curves. The x-axis of a ROC curve shows the false-positive rate of an assay, which can be calculated as (1−specificity). The y-axis of a ROC curve reports the sensitivity for an assay. This allows one to easily determine a sensitivity of an assay for a given specificity, and vice versa.

As used herein, the term ‘about’ a number refers to that number plus or minus 10% of that number. The term ‘about’ a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

### Numbered Embodiments

The following embodiments recite nonlimiting permutations of combinations of features disclosed herein. Other permutations of combinations of features are also contemplated.

1. A method of assessing a colorectal health risk status in an individual, comprising steps of obtaining a circulating blood sample from said individual; and obtaining a biomarker panel level for a biomarker panel indicated in at least one of table 3 and table 5, and assessing colorectal health risk status. 2. A method of analyzing a biological sample, comprising: obtaining protein levels in said biological sample for each protein of a biomarker panel comprising C9, CEA, DPP4, MIF, ORM1, PKM, SAA, and TFRC to determine a panel information for said biomarker panel; comparing said panel information to a reference panel information, wherein said reference panel information corresponds to a known colorectal cancer status; and categorizing said biological sample as having a positive colorectal cancer risk status if said panel information does not differ significantly from said reference panel information, wherein said biological sample is derived from a circulating blood sample. 3. The method of embodiment 2, wherein said biomarker panel further comprises at least one of an individual age and an individual gender. 4. The method of embodiment 2, wherein said known colorectal cancer status comprises at least one of early CRC and advanced CRC. 5. The method of embodiment 2, wherein said known colorectal cancer status comprises at least one of Stage 0 CRC, stage I CRC, Stage II CRC, stage III CRC, and stage IV CRC. 6. The method of embodiment 2, wherein said biomarker panel comprises no more than 15 proteins. 7. The method of embodiment 2, wherein said biomarker panel comprises no more than 8 proteins. 8. The method of embodiment 2, wherein said categorizing has a sensitivity of at least 80% and a specificity of at least 71%. 9. The method of embodiment 2, further comprising performing a treatment regimen in response to said categorizing. 10. The method of embodiment 9, wherein said treatment regimen comprises at least one of chemotherapy, radiation, immunotherapy, administration of a biologic therapeutic agent, polypectomy, partial colectomy, low anterior resection or abdominoperineal resection and colostomy. 11. The method of embodiment 2, further comprising transmitting a report of results of said categorizing to a health practitioner. 12. The method of embodiment 11, wherein said report indicates a sensitivity of at least 80%. 13. The method of embodiment 11, wherein said report indicates a specificity of at least 71%. 14. The method of embodiment 11, wherein said report indicates a recommendation for a treatment regimen comprising at least one of chemotherapy, radiation, immunotherapy, administration of a biologic therapeutic agent, polypectomy, partial colectomy, low anterior resection or abdominoperineal resection and colostomy. 15. The method of embodiment 11, wherein said report indicates a recommendation for a colonoscopy. 16. The method of embodiment 11, wherein said report indicates a recommendation for undergoing an independent cancer assay. 17. The method of embodiment 11, wherein said report indicates a recommendation for undergoing a stool cancer assay. 18. The method of embodiment 2, further comprising performing a stool cancer assay in response to said categorizing. 19. The method of embodiment 2, further comprising continued monitoring for a period of 3 months or greater. 20. The method of embodiment 2, further comprising continued monitoring for a period of between 3 months and 24 months. 21. The method of embodiment 2, wherein said obtaining said protein levels comprises subjecting said biological sample to a mass spectrometric analysis. 22. The method of embodiment 2, wherein said obtaining said protein levels comprises subjecting said biological sample to an immunoassay analysis. 23. A method of analyzing a biological sample, comprising: obtaining protein levels in said biological sample for each protein of a biomarker panel comprising CLU, CTSD, DPP4, GDF15, GSN, MIF, PKM, SERPINA1, SERPINA3, TFRC, and TIMP1 to determine a panel information for said biomarker panel; comparing said panel information to a reference panel information, wherein said reference panel information corresponds to a known advanced adenoma status; and categorizing said blood sample as having a positive advanced adenoma risk status if said panel information does not differ significantly from said reference panel information, wherein said biological sample is derived from a circulating blood sample. 24. The method of embodiment 23, wherein said biomarker panel further comprises at least one of an individual age and an individual gender. 25. The method of embodiment 23, wherein said biomarker panel comprises no more than 15 proteins. 26. The method of embodiment 23, wherein said biomarker panel comprises no more than 8 proteins. 27. The method of embodiment 23, wherein said categorizing has a sensitivity of at least 44% and a specificity of at least 80%. 28. The method of embodiment 23, further comprising performing a treatment regimen in response to said categorizing. 29. The method of embodiment 28, wherein said treatment regimen comprises at least one of chemotherapy, radiation, immunotherapy, administration of a biologic therapeutic agent, polypectomy, partial colectomy, low anterior resection or abdominoperineal resection and colostomy. 30. The method of embodiment 23, comprising transmitting a report of results of said categorizing to a health practitioner. 31. The method of embodiment 30, wherein said report indicates a sensitivity of at least 44%. 32. The method of embodiment 30, wherein said report indicates a specificity of at least 80%. 33. The method of embodiment 30, wherein said report indicates a recommendation for a treatment regimen comprising at least one of chemotherapy, radiation, immunotherapy, administration of a biologic therapeutic agent, polypectomy, partial colectomy, low anterior resection or abdominoperineal resection and colostomy. 34. The method of embodiment 30, wherein said report indicates a recommendation for a colonoscopy. 35. The method of embodiment 30, wherein said report indicates a recommendation for undergoing an independent cancer assay. 36. The method of embodiment 30, wherein said report indicates a recommendation for undergoing a stool cancer assay. 37. The method of embodiment 23, further comprising performing a stool cancer assay. 38. The method of embodiment 23, further comprising continued monitoring for a period of 3 months or greater. 39. The method of embodiment 23, further comprising continued monitoring for a period of between 3 months and 24 months. 40. The method of embodiment 23, wherein obtaining said protein levels comprises subjecting said biological sample to a mass spectrometric analysis. 41. The method of embodiment 23, wherein said obtaining said protein levels comprises subjecting said biological sample to an immunoassay analysis. 42. A method of analyzing data generated in vitro, comprising: storing, by a processor, a panel information corresponding to a biological sample, wherein said panel information comprises protein levels for each protein of a biomarker panel comprising C9, CEA, DPP4, MIF, ORM1, PKM, SAA, and TFRC; comparing, by said processor, said panel information to a reference panel information, wherein said reference panel information corresponds to a known colorectal cancer status; and categorizing, by said processor, said panel information as having a positive colorectal cancer risk status if said panel information does not differ significantly from said reference panel information. 43. The method of embodiment 42, wherein said biomarker panel further comprises at least one of an individual age and an individual gender. 44. The method of embodiment 42, wherein said known colorectal cancer status comprises at least one of early CRC and advanced CRC. 45. The method of embodiment 42, wherein said known colorectal cancer status comprises at least one of Stage 0 CRC, stage I CRC, Stage II CRC, stage III CRC, and stage IV CRC. 46. The method of embodiment 42, wherein said biomarker panel comprises no more than 15 proteins. 47. The method of embodiment 42, wherein said biomarker panel comprises no more than 8 proteins. 48. The method of embodiment 42, wherein said categorizing has a sensitivity of at least 80% and a specificity of at least 71%. 49. The method of embodiment 42, wherein said processor is further configured to generate a report indicating said positive colorectal cancer risk status. 50. The method of embodiment 49, wherein said report further indicates recommendation for a treatment regimen in response to said categorizing. 51. The method of embodiment 49, wherein said treatment regimen comprises at least one of chemotherapy, radiation, immunotherapy, administration of a biologic therapeutic agent, polypectomy, partial colectomy, low anterior resection or abdominoperineal resection and colostomy. 52. The method of embodiment 49, wherein said report indicates a sensitivity of at least 80%. 53. The method of embodiment 49, wherein said report indicates a specificity of at least 71%. 54. The method of embodiment 49, wherein said report indicates recommendation for a colonoscopy. 55. The method of embodiment 49, wherein said report indicates recommendation for undergoing an independent cancer assay. 56. The method of embodiment 49, wherein said report indicates recommendation for undergoing a stool cancer assay. 57. A method of analyzing data generated in vitro, comprising: storing a panel information comprising protein levels for each protein of a biomarker panel comprising CLU, CTSD, DPP4, GDF15, GSN, MIF, PKM, SERPINA1, SERPINA3, TFRC, and TIMP1; comparing said panel information to a reference panel information, wherein said reference panel information corresponds to a known advanced adenoma status; and categorizing said panel information as having a positive advance adenoma risk status if said panel information does not differ significantly from said reference panel information. 58. The method of embodiment 57, wherein said biomarker panel further comprises at least one of an individual age and an individual gender. 59. The method of embodiment 57, wherein said biomarker panel comprises no more than 15 proteins. 60. The method of embodiment 57, wherein said biomarker panel comprises no more than 8 proteins. 61. The method of embodiment 57, wherein said categorizing has a sensitivity of at least 44% and a specificity of at least 80%. 62. The method of embodiment 57, further comprising generating a report indicating said positive advanced adenoma status. 63. The method of embodiment 62, wherein said report further indicates recommendation for a treatment regimen in response to said categorizing. 64. The method of embodiment 63, wherein said treatment regimen comprises at least one of chemotherapy, radiation, immunotherapy, administration of a biologic therapeutic agent, polypectomy, partial colectomy, low anterior resection or abdominoperineal resection and colostomy. 65. The method of embodiment 62, wherein said report indicates a sensitivity of at least 44%. 66. The method of embodiment 62, wherein said report indicates a specificity of at least 80%. 67. The method of embodiment 62, wherein said report indicates recommendation for a colonoscopy. 68. The method of embodiment 62, wherein said report indicates recommendation for undergoing an independent cancer assay. 69. The method of embodiment 62, wherein said report indicates recommendation for undergoing a stool cancer assay. 70. A computer system for analyzing data generated in vitro, comprising: (a) a memory unit for receiving a panel information comprising measurement of protein levels of each protein in a biomarker panel from a biological sample, wherein the biomarker panel comprises C9, CEA, DPP4, MIF, ORM1, PKM, SAA, and TFRC; (b) computer-executable instructions for comparing said panel information to a reference panel information, wherein said reference panel information corresponds to a known colorectal cancer status; and (c) computer-executable instructions for categorizing said panel information as having a positive colorectal cancer status if said panel information does not differ significantly from said reference panel information. 71. The computer system of embodiment 70, further comprising computer-executable instructions to generate a report of said positive colorectal cancer status. 72. The computer system of embodiment 70, wherein said biomarker panel further comprises at least one of an individual age and an individual gender. 73. The computer system of embodiment 70, wherein said known colorectal cancer status comprises at least one of early CRC and advanced CRC. 74. The computer system of embodiment 70, wherein said known colorectal cancer status comprises at least one of Stage 0 CRC, stage I CRC, Stage II CRC, stage III CRC, and stage IV CRC. 75. The computer system of embodiment 70, wherein said biomarker panel comprises no more than 15 proteins. 76. The computer system of embodiment 70, wherein said biomarker panel comprises no more than 8 proteins. 77. The computer system of embodiment 70, wherein said categorizing has a sensitivity of at least 80% and a specificity of at least 71%. 78. The computer system of embodiment 70, further comprising generating a report indicating said positive colorectal cancer risk status. 79. The computer system of embodiment 78, wherein said report further indicates recommendation for a treatment regimen in response to said categorizing. 80. The computer system of embodiment 79, wherein said treatment regimen comprises at least one of chemotherapy, radiation, immunotherapy, administration of a biologic therapeutic agent, polypectomy, partial colectomy, low anterior resection or abdominoperineal resection and colostomy. 81. The computer system of embodiment 78, wherein said report indicates a sensitivity of at least 80%. 82. The computer system of embodiment 78, wherein said report indicates a specificity of at least 71%. 83. The computer system of embodiment 78, wherein said report indicates recommendation for a colonoscopy. 84. The computer system of embodiment 78, wherein said report indicates recommendation for undergoing an independent cancer assay. 85. The computer system of embodiment 79, wherein said report indicates recommendation for undergoing a stool cancer assay. 86. The computer system of embodiment 70, further comprising a user interface configured to communicate or display said report to a user. 87. A computer system for analyzing data generated in vitro: (a) a memory unit for receiving a panel information comprising measurement of protein levels of each protein in a biomarker panel from a biological sample, wherein said biomarker panel comprises CLU, CTSD, DPP4, GDF15, GSN, MIF, PKM, SERPINA1, SERPINA3, TFRC, and TIMP1; (b) computer-executable instructions for comparing said panel information to a reference panel information, wherein said reference panel information corresponds to a known advanced adenoma status; and (c) computer-executable instructions for categorizing said panel information as having a positive advanced adenoma status if said panel information does not differ significantly from said reference panel information. 88. The computer system of embodiment 87, wherein said biomarker panel further comprises at least one of an individual age and an individual gender. 89. The computer system of embodiment 87, wherein said biomarker panel comprises no more than 15 proteins. 90. The computer system of embodiment 87, wherein biomarker panel comprises no more than 8 proteins. 91. The computer system of embodiment 87, wherein said categorizing has a sensitivity of at least 80% and a specificity of at least 71%. 92. The computer system of embodiment 87, further comprising computer-executable instructions to generate a report of said positive advanced adenoma status. 93. The computer system of embodiment 92, wherein said report further indicates recommendation for a treatment regimen in response to said categorizing. 94. The computer system of embodiment 93, wherein said treatment regimen comprises at least one of chemotherapy, radiation, immunotherapy, administration of a biologic therapeutic agent, polypectomy, partial colectomy, low anterior resection or abdominoperineal resection and colostomy. 95. The computer system of embodiment 92, wherein said report indicates a sensitivity of at least 44%. 96. The computer system of embodiment 92, wherein said report indicates a specificity of at least 80%. 97. The computer system of embodiment 92, wherein said report indicates recommendation for a colonoscopy. 98. The computer system of embodiment 92, wherein said report indicates recommendation for undergoing an independent cancer assay. 99. The computer system of embodiment 92, wherein said report indicates recommendation for undergoing a stool cancer assay. 100. A method of assessing colorectal health of an individual, comprising: obtaining a circulating blood sample from said individual; and detecting protein levels for each member of a list of proteins in said sample, said list of proteins comprising C9, CEA, ORM1 and DPP4. 101. The method of embodiment 100, further comprising diagnosing said individual as having a colorectal cancer status when said protein levels from said individual do not differ significantly from a reference panel information set corresponding to a known colorectal cancer risk status. 102. The method of embodiment 101, further comprising performing colonoscopy on said individual. 103. The method of embodiment 101, wherein said known colorectal cancer status comprises at least one of early CRC and advanced CRC. 104. The method of embodiment 101, wherein said known colorectal cancer status comprises at least one of Stage 0 CRC, stage I CRC, Stage II CRC, stage III CRC, and stage IV CRC. 105. The method of embodiment 101, further performing a treatment regimen upon said individual. 106. The method of embodiment 105, wherein said treatment regimen comprises a polypectomy. 107. The method of embodiment 105, wherein said treatment regimen comprises radiation. 108. The method of embodiment 105, wherein said treatment regimen comprises chemotherapy. 109. The method of embodiment 100, wherein said list of proteins further comprises at least one of SAA, TFRC, PKM and MIF. 110. The method of embodiment 100, wherein said list of proteins further comprises at least two of SAA, TFRC, PKM and MIF. 111. The method of embodiment 100, wherein said list of proteins further comprises each OF SAA, TFRC, PKM and MIF. 112. The method of embodiment 100, further comprising obtaining at least one of an age and a gender of said individual. 113. The method of embodiment 100, further comprising transmitting a report to a health practitioner of results of said detecting. 114. The method of embodiment 113, wherein said report indicates recommendation for a colonoscopy for said individual. 115. The method of embodiment 113, wherein said report indicates recommendation for a polypectomy for said individual. 116. The method of embodiment 113, wherein said report indicates recommendation for radiation for said individual. 117. The method of embodiment 113, wherein said report indicates recommendation for chemotherapy for said individual. 118. The method of embodiment 113, wherein said report indicates recommendation for undergoing an independent cancer assay. 119. The method of embodiment 113, wherein said report indicates recommendation for undergoing a stool cancer assay. 120. The method of embodiment 100, wherein said list of proteins comprises no more than 15 proteins. 121. The method of embodiment 100, wherein said list of proteins comprises no more than 8 proteins. 122. A method of assessing colorectal health of an individual, comprising: obtaining a circulating blood sample from said individual; and detecting protein levels for each member of a list of proteins in said sample, said list of proteins comprising ORM and MIF; and obtaining an age of said individual. 123. The method of embodiment 122, further comprising diagnosing said individual as having a colorectal cancer status when said protein levels from said individual do not differ significantly from a reference panel information set corresponding to a known colorectal cancer risk status. 124. The method of embodiment 123, further comprising performing colonoscopy on said individual. 125. The method of embodiment 123, wherein said known colorectal cancer status comprises at least one of early CRC and advanced CRC. 126. The method of embodiment 123, wherein said known colorectal cancer status comprises at least one of Stage 0 CRC, stage I CRC, Stage II CRC, stage III CRC, and stage IV CRC. 127. The method of embodiment 123, further performing a treatment regimen upon said individual. 128. The method of embodiment 127, wherein said treatment regimen comprises polypectomy. 129. The method of embodiment 127, wherein said treatment regimen comprises radiation. 130. The method of embodiment 127, wherein said treatment regimen comprises chemotherapy. 131. The method of embodiment 122, wherein said list of proteins further comprises at least one of SAA, CEA, DPP4, PKM and C9. 132. The method of embodiment 122, wherein said list of proteins further comprises at least two of SAA, CEA, DPP4, PKM and C9. 133. The method of embodiment 122, wherein said list of proteins further comprises at least three of SAA, CEA, DPP4, PKM and C9. 134. The method of embodiment 122, wherein said list of proteins further comprises each of SAA, CEA, DPP4, PKM and C9. 135. The method of embodiment 122, further comprising obtaining a gender of said individual. 136. The method of embodiment 122, further comprising transmitting a report to a health practitioner of results of said detecting. 137. The method of embodiment 136, wherein said report indicates recommendation for a colonoscopy for said individual. 138. The method of embodiment 136, wherein said report indicates recommendation for a polypectomy for said individual. 139. The method of embodiment 136, wherein said report indicates recommendation for radiation for said individual. 140. The method of embodiment 136, wherein said report indicates recommendation for chemotherapy for said individual. 141. The method of embodiment 136, wherein said report indicates recommendation for undergoing an independent cancer assay. 142. The method of embodiment 136, wherein said report indicates recommendation for undergoing a stool cancer assay. 143. The method of embodiment 122, wherein said list of proteins comprises no more than 15 proteins. 144. The method of embodiment 122, wherein said list of proteins comprises no more than 8 proteins. 145. A method of assessing colorectal health of an individual, comprising: obtaining a circulating blood sample from said individual; and detecting protein levels for each member of a list of proteins in the sample, said list of proteins comprising MIF, PKM, CTSD, GELS and CLUS. 146. The method of embodiment 145, further comprising diagnosing said individual as having an advanced adenoma status when said protein levels from said individual do not differ significantly from a reference panel information set corresponding to a known advanced adenoma risk status. 147. The method of embodiment 146, further comprising performing colonoscopy on said individual. 148. The method of embodiment 146, further performing a treatment regimen upon said individual. 149. The method of embodiment 148, wherein said treatment regimen comprises polypectomy. 150. The method of embodiment 148, wherein said treatment regimen comprises radiation. 151. The method of embodiment 148, wherein said treatment regimen comprises chemotherapy. 152. The method of embodiment 145, wherein said list of proteins further comprises at least one of DPP4, GDF15, TIMP1, TFRC and A1AT. 153. The method of embodiment 145, wherein said list of proteins further comprises at least two of DPP4, GDF15, TIMP1, TFRC and A1AT. 154. The method of embodiment 145, wherein said list of proteins further comprises at least three of DPP4, GDF15, TIMP1, TFRC and A1AT. 155. The method of embodiment 145, wherein said list of proteins further comprises each of DPP4, GDF15, TIMP1, TFRC and A1AT. 156. The method of embodiment 145, further comprising obtaining a gender of said individual. 157. The method of embodiment 145, further comprising transmitting a report to a health practitioner of results of said detecting. 158. The method of embodiment 157, wherein said report indicates recommendation for a colonoscopy for said individual. 159. The method of embodiment 157, wherein said report indicates recommendation for a polypectomy for said individual. 160. The method of embodiment 157, wherein said report indicates recommendation for radiation for said individual. 161. The method of embodiment 157, wherein said report indicates recommendation for chemotherapy for said individual. 162. The method of embodiment 157, wherein said report indicates recommendation for undergoing an independent cancer assay. 163. The method of embodiment 157, wherein said report indicates recommendation for undergoing a stool cancer assay. 164. The method of embodiment 145, wherein said list of proteins comprises no more than 15 proteins. 165. The method of embodiment 145, wherein said list of proteins comprises no more than 8 proteins. 166. A method of assessing colorectal health of an individual, comprising: obtaining a circulating blood sample from said individual; detecting protein levels for each member of a list of proteins in sample, said list of proteins comprising PKM, MIF and CTSD; and obtaining an age of said individual. 167. The method of embodiment 166, further comprising diagnosing said individual as having an advanced adenoma status when said protein levels from said individual do not differ significantly from a reference panel information set corresponding to a known advanced adenoma risk status. 168. The method of embodiment 167, further comprising performing colonoscopy on said individual. 169. The method of embodiment 167, further performing a treatment regimen upon said individual. 170. The method of embodiment 169, wherein said treatment regimen comprises polypectomy. 171. The method of embodiment 169, wherein said treatment regimen comprises radiation. 172. The method of embodiment 169, wherein said treatment regimen comprises chemotherapy. 173. The method of embodiment 166, wherein said list of proteins further comprises at least one of SERPINA1, GSN and TIMP1. 174. The method of embodiment 173, wherein said list of proteins further comprises at least one of CLU, TFCR, DPP4, SERPINA3 and GDF15. 175. The method of embodiment 166, further comprising obtaining a gender of said individual. 176. The method of embodiment 166, further comprising transmitting a report to a health practitioner of results of said detecting. 177. The method of embodiment 176, wherein said report indicates recommendation for a colonoscopy for said individual. 178. The method of embodiment 176, wherein said report indicates recommendation for a polypectomy for said individual. 179. The method of embodiment 176, wherein said report indicates recommendation for radiation for said individual. 180. The method of embodiment 176, wherein said report indicates recommendation for chemotherapy for said individual. 181. The method of embodiment 176, wherein said report indicates recommendation for undergoing an independent cancer assay. 182. The method of embodiment 176, wherein said report indicates recommendation for undergoing a stool cancer assay. 183. The method of embodiment 166, wherein said list of proteins comprises no more than 15 proteins. 184. The method of embodiment 166, wherein said list of proteins comprises no more than 8 proteins. 185. A method of assessing colorectal health of an individual, comprising: obtaining a circulating blood sample from said individual; detecting protein levels for each member of a list of proteins in sample, said list of proteins comprising DPPIV, CO9 and CEA. 186. The method of embodiment 185, further comprising diagnosing said individual as having a colorectal cancer status when said protein levels from said individual do not differ significantly from a reference panel information set corresponding to a known colorectal cancer risk status. 187. The method of embodiment 185 or 186, further comprising performing colonoscopy on said individual. 188. The method of any one of embodiments 185 to 187, further performing a treatment regimen upon said individual. 189. The method of embodiment 188, wherein said treatment regimen comprises polypectomy. 190. The method of embodiment 188, wherein said treatment regimen comprises radiation. 191. The method of embodiment 188, wherein said treatment regimen comprises chemotherapy. 192. The method of embodiment 185, wherein said list of proteins further comprises at least one of ORM1, MIF, PKM2, SAA, and TFRC. 193. The method of embodiment 185, wherein said list of proteins further comprises ORM1, MIF, PKM2, SAA, and TFRC. 194. The method of embodiment 185, comprising obtaining age information for said individual. 195. The method of embodiment 185, comprising obtaining gender information for said individual. 196. The method of embodiment 185, comprising obtaining age information and gender information for said individual. 197. The method of any one of embodiments 185 to 196, further comprising transmitting a report to a health practitioner of results of said detecting. 198. The method of any one of embodiments 195 to 197, further comprising diagnosing said individual as having a colorectal cancer status when said protein levels, age and gender from said individual as a whole do not differ significantly from a reference panel information set corresponding to a known colorectal cancer risk status. 199. The method of embodiment 185, wherein said report indicates recommendation for a colonoscopy for said individual. 200. The method of embodiment 197, wherein said report indicates recommendation for a polypectomy for said individual. 201. The method of embodiment 197, wherein said report indicates recommendation for radiation for said individual. 202. The method of embodiment 197, wherein said report indicates recommendation for chemotherapy for said individual. 203. The method of embodiment 197, wherein said report indicates recommendation for undergoing an independent cancer assay. 204. The method embodiment 197, wherein said report indicates recommendation for undergoing a stool cancer assay. 205. The method of any one of embodiments 185 to 204, wherein said list of proteins comprises no more than 15 proteins. 206. The method of embodiment 185, wherein said list of proteins comprises no more than 8 proteins. 207. 208. A method of assessing colorectal health of an individual, comprising: obtaining a circulating blood sample from said individual; detecting protein levels for each member of a list of proteins in sample, said list of proteins comprising CATD, TFRC and TIMP1. 209. The method of embodiment 208, further comprising diagnosing said individual as having an advanced adenoma status when said protein levels from said individual do not differ significantly from a reference panel information set corresponding to a known advanced adenoma risk status. 210. The method of embodiment 208 or 209, further comprising performing colonoscopy on said individual. 211. The method of any one of embodiments 208 to 210, further performing a treatment regimen upon said individual. 212. The method of embodiment 211, wherein said treatment regimen comprises polypectomy. 213. The method of embodiment 211, wherein said treatment regimen comprises radiation. 214. The method of embodiment 211, wherein said treatment regimen comprises chemotherapy. 215. The method of embodiment 208, wherein said list of proteins further comprises at least one of MIF, CLUS, PKM2, DPPIV, GDF15, GELS, A1AT and AACT. 216. The method of embodiment 208, wherein said list of proteins further comprises MIF, CLUS, PKM2, DPPIV, GDF15, GELS, A1AT and AACT. 217. The method of embodiment 208, comprising obtaining age information for said individual. 218. The method of embodiment 208, comprising obtaining gender information for said individual. 219. The method of embodiment 208, comprising obtaining age information and gender information for said individual. 220. The method of any one of embodiments 208 to 219, further comprising transmitting a report to a health practitioner of results of said detecting. 221. The method of any one of embodiments 208 to 219, further comprising diagnosing said individual as having an advanced adenoma status when said protein levels and age from said individual as a whole do not differ significantly from a reference panel information set corresponding to a known advanced adenoma risk status. 222. The method of embodiment 220, wherein said report indicates recommendation for a colonoscopy for said individual. 223. The method of embodiment 220, wherein said report indicates recommendation for a polypectomy for said individual. 224. The method of embodiment 220, wherein said report indicates recommendation for radiation for said individual. 225. The method of embodiment 220, wherein said report indicates recommendation for chemotherapy for said individual. 226. The method of embodiment 220, wherein said report indicates recommendation for undergoing an independent cancer assay. 227. The method of embodiment 220, wherein said report indicates recommendation for undergoing a stool cancer assay. 228. The method of any one of embodiments 208 to 227, wherein said list of proteins comprises no more than 15 proteins. 229. The method of any one of embodiments 208 to 227, wherein said list of proteins comprises no more than 10 proteins.

Further understanding of the disclosure herein is gained through reference to the following embodiments.

### EXAMPLES

### Example 1

A patient at risk of colorectal cancer is tested using a panel as disclosed herein. A blood sample is taken from the patient. The blood sample is mailed to a facility, where plasma is prepared and protein accumulation levels are measured using antibody florescence binding assay to detect members of a panel comprising C9, CEA, DPP4, MIF, ORM1, PKM, SAA, TFRC and also factoring in the patient's gender and age. The patient's panel results are compared to panel results of known status, and the patient is categorized with an at least 81% sensitivity, and an at least 78% specificity as having colon cancer. A colonoscopy is recommended and evidence of colorectal cancer is detected in the individual.

### Example 2

The patient of Example 1 is prescribed a treatment regimen comprising a surgical intervention. A blood sample is taken from the patient prior to surgical intervention and protein accumulation levels are measured for a panel comprising C9, CEA, DPP4, MIF, ORM1, PKM, SAA, TFRC and also factoring in the patient's gender and age. The patient's panel results are compared to panel results of known status, and the patient is categorized with an 81% sensitivity, a 78% specificity, and a 31% positive predictive value as having colon cancer.

A blood sample is taken from the patient subsequent to surgical intervention and protein accumulation levels are measured for a panel comprising C9, CEA, DPP4, MIF, ORM1, PKM, SAA, TFRC and also factoring in the patient's gender and age. The patient's panel results are compared to panel results of known status, and the patient is patient is categorized with an 81% sensitivity, and a 78% specificity as having colon cancer.

### Example 3

The patient of Example 1 is prescribed a treatment regimen comprising a chemotherapeutic intervention comprising 5-FU administration. A blood sample is taken from the patient prior to chemotherapeutic intervention and protein accumulation levels are measured for a panel comprising C9, CEA, DPP4, MIF, ORM1, PKM, SAA, TFRC and also factoring in the patient's gender and age. The patient's panel results are compared to panel results of known status, and the patient is patient is categorized with an 81% sensitivity, and a 78% specificity as having colon cancer.

A blood sample is taken from the patient at weekly intervals during chemotherapy treatment and protein accumulation levels are measured for a panel comprising C9, CEA, DPP4, MIF, ORM1, PKM, SAA, TFRC and also factoring in the patient's gender and age. The patient's panel results are compared to panel results of known status. The patient's panel results over time indicate that the cancer has responded to the chemotherapy treatment and that the colorectal cancer is no longer detectable by completion of the treatment regimen.

### Example 4

The patient of Example 1 is prescribed a treatment regimen comprising a chemotherapeutic intervention comprising oral capecitabine administration. A blood sample is taken from the patient prior to chemotherapeutic intervention and protein accumulation levels are measured for a panel comprising C9, CEA, DPP4, MIF, ORM1, PKM, SAA, TFRC and also factoring in the patient's gender and age. The patient's panel results are compared to panel results of known status, and the patient is patient is categorized with an 81% sensitivity, and a 78% specificity as having colon cancer.

A blood sample is taken from the patient at weekly intervals during chemotherapy treatment and protein accumulation levels are measured for a panel comprising C9, CEA, DPP4, MIF, ORM1, PKM, SAA, TFRC and also factoring in the patient's gender and age. The patient's panel results over time indicate that the cancer has responded to the chemotherapy treatment and that the colorectal cancer is no longer detectable by completion of the treatment regimen.

### Example 5

The patient of Example 1 is prescribed a treatment regimen comprising a chemotherapeutic intervention comprising oral oxaliplatin administration. A blood sample is taken from the patient prior to chemotherapeutic intervention and protein accumulation levels are measured for a panel comprising C9, CEA, DPP4, MIF, ORM1, PKM, SAA, TFRC and also factoring in the patient's gender and age. The patient's panel results are compared to panel results of known status, and the patient is patient is categorized with an 81% sensitivity, and a 78% specificity as having colon cancer.

A blood sample is taken from the patient at weekly intervals during chemotherapy treatment and protein accumulation levels are measured for a panel comprising C9, CEA, DPP4, MIF, ORM1, PKM, SAA, TFRC and also factoring in the patient's gender and age. The patient's panel results are compared to panel results of known status. The patient's panel results over time indicate that the cancer has responded to the chemotherapy treatment and that the colorectal cancer is no longer detectable by completion of the treatment regimen.

### Example 6

The patient of Example 1 is prescribed a treatment regimen comprising a chemotherapeutic intervention comprising oral oxaliplatin administration in combination with bevacizumab. A blood sample is taken from the patient prior to chemotherapeutic intervention and protein accumulation levels are measured for a panel comprising C9, CEA, DPP4, MIF, ORM1, PKM, SAA, TFRC and also factoring in the patient's gender and age. The patient's panel results are compared to panel results of known status, and the patient is patient is categorized with an 81% sensitivity, and a 78% specificity as having colon cancer.

A blood sample is taken from the patient at weekly intervals during chemotherapy treatment and protein accumulation levels are measured for a panel comprising C9, CEA, DPP4, MIF, ORM1, PKM, SAA, TFRC and also factoring in the patient's gender and age. The patient's panel results are compared to panel results of known status. The patient's panel results over time indicate that the cancer has responded to the chemotherapy treatment and that the colorectal cancer is no longer detectable by completion of the treatment regimen.

### Example 7

A patient at risk of colorectal cancer is tested using a panel as disclosed herein. A blood sample is taken from the patient and protein accumulation levels are measured using reagents in an ELISA kit to detect members of a panel comprising C9, CEA, DPP4, MIF, ORM1, PKM, SAA, TFRC and also factoring in the patient's gender and age. The patient's panel results are compared to panel results of known status, and the patient is patient is categorized with an 81% sensitivity, and a 78% specificity as having colon cancer. A colonoscopy is recommended and evidence of colorectal cancer is detected in the individual.

### Example 8

A patient at risk of colorectal cancer is tested using a panel as disclosed herein. A blood sample is taken from the patient and protein accumulation levels are measured using mass spectrometry to detect members of a panel comprising C9, CEA, DPP4, MIF, ORM1, PKM, SAA, TFRC and also factoring in the patient's gender and age. The patient's panel results are compared to panel results of known status, and the patient is categorized with an 81% sensitivity, and a 78% specificity as having colon cancer. A colonoscopy is recommended and evidence of colorectal cancer is detected in the individual.

### Example 9

1000 patients at risk of colorectal cancer are tested using a panel as disclosed herein. A blood sample is taken from the patient and protein accumulation levels are measured to detect members of a panel comprising C9, CEA, DPP4, MIF, ORM1, PKM, SAA, TFRC and also factoring in the patient's gender and age. The patients' panel results are compared to panel results of known status, and the patients are categorized with an 81% sensitivity, and a 78% specificity into a colon cancer category. A colonoscopy is recommended for patients categorized as positive. Of the patients categorized as having colon cancer, 80% are independently confirmed to have colon cancer. Of the patients categorized as not having colon cancer, 20% are later found to have colon cancer through an independent follow up test, confirmed via a colonoscopy.

### Example 10

A patient at risk of advanced adenoma is tested using a panel as disclosed herein. A blood sample is taken from the patient. The blood sample is mailed to a facility, where plasma is prepared and protein accumulation levels are measured using an antibody florescence binding assay to detect members of a panel comprising SERPINA1, SERPINA3, CTSD, CLU, DPP4, GDF15, GSN, MIF, PKM, TIMP1, TFRC, and patient age is also considered. The patient's panel results are compared to panel results of known status, and the patient is categorized as being at risk of advanced adenoma.

### Example 11—Clinical Utility of Noninvasive, Accurate Colorectal Health Assay

A recalcitrant patient demonstrated symptoms of CRC but refused a colonoscopy. The patient's primary care physician ordered a SimpliPro colorectal health assessment test. The results indicated that the patient was at a high risk for CRC and for AA. The patient consulted with family and was convinced to schedule a colonoscopy. The colonoscopy revealed polyps and an early stage cancerous mass, all of which were removed during the procedure. A follow-up colorectal health assessment indicated that the patient is cancer free. The patient's early stage cancerous mass would likely have progressed to advanced disease with a high probability of death without the colonoscopy and concurrent polypectomy.

This Example demonstrates the benefit to the public of offering a noninvasive colorectal health assay that is both sensitive and specific, and is easily complied with. This example demonstrates that the reluctance to undergo a colonoscopy is common, and that it can have severe health consequences if it results in an early stage cancer not being detected when it is relatively easily treated.

### Example 12—Clinical Utility of Noninvasive, Accurate Colorectal Health Assay

A recalcitrant patient demonstrated symptoms of CRC but delayed a colonoscopy for over 6 months. The patient's primary care physician ordered a SimpliPro colorectal health assessment test. The results indicated that the patient was at a high risk for CRC and for AA. The patient scheduled a colonoscopy. During the procedure, a 6 cm malignant mass was identified and removed. A follow-up colorectal health assessment indicated that the patient is cancer free. The patient's early stage cancerous mass would likely have progressed to advanced disease with a high probability of death without the colonoscopy and concurrent polypectomy.

This Example demonstrates the benefit to the public of offering a noninvasive colorectal health assay that is both sensitive and specific, and is easily complied with. This example demonstrates that the reluctance to undergo a colonoscopy is common, and that it can have severe health consequences if it results in an early stage cancer not being detected when it is relatively easily treated.

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of assessing colorectal health of an individual, comprising:
obtaining a circulating blood sample from said individual;
detecting protein levels for each member of a list of proteins in sample, said list of proteins comprising DPPIV, CO9, TFRC, ORM1, MIF, PKM, SAA, and CEA; and
obtaining age information and gender information for said individual.

2. (canceled)

3. The method of claim 1, further comprising performing colonoscopy on said individual.

4. The method of claim 1, further comprising performing a treatment regimen upon said individual.

5. The method of claim 4, wherein said treatment regimen comprises polypectomy.

6. The method of claim 4, wherein said treatment regimen comprises radiation.

7. The method of claim 4, wherein said treatment regimen comprises chemotherapy.

8. (canceled)

9. (canceled)

10. (canceled)

11. (canceled)

12. (canceled)

13. (canceled)

14. (canceled)

15. (canceled)

16. (canceled)

17. (canceled)

18. The method of claim 1, further comprising transmitting a report to a health practitioner of results of said detecting.

19. The method of claim 18, wherein said report indicates recommendation for a colonoscopy for said individual.

20. The method of claim 18, wherein said report indicates recommendation for a polypectomy for said individual.

21. The method of claim 18, wherein said report indicates recommendation for radiation therapy for said individual.

22. The method of claim 18, wherein said report indicates recommendation for chemotherapy for said individual.

23. The method of claim 18, wherein said report indicates recommendation for undergoing an independent cancer assay.

24. The method claim 18, wherein said report indicates recommendation for undergoing a stool cancer assay.

25. The method of claim 1, wherein said list of proteins comprises no more than 15 proteins.

26. The method of claim 1, wherein said list of proteins comprises no more than 8 proteins.


SEQUENCE LISTING (16 sequences)

SEQ ID NO: 1 - protein, 201 aa, Homo sapiens
MALSWVLTVLSLLPLLEAQIPLCANLVPVPITNATLDQITGKWFYIASAFRNEEYNKSVQEIQATFFYFTPNKTEDTIFLREYQTRQDQCIYNTTYLNVQRENGTISRYVGGQEHFAHLLILRDTKTYMLAFDVNDEKNWGLSVYADKPETTKEQLGEFYEALDCLRIPKSDVVYTDWKKDKCEPLEKQHEKERKQEEGES

SEQ ID NO: 2 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 3 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 4 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 5 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 6 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 7 - protein, 559 aa, Homo sapiens
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

SEQ ID NO: 8 - protein, 765 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 9 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 10 - protein, 115 aa, Homo sapiens
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 11 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 12 - protein, 122 aa, Homo sapiens
MKLLTGLVFCSLVLGVSSRSFFSFLGEAFDGARDMWRAYSDMREANYIGSDKYFHARGNYDAAKRGPGGVWAAEAISDARENIQRFFGHGAEDSLADQAANEWGRSGKDPNHFRPAGLPEKY

SEQ ID NO: 13 - protein, 122 aa, Homo sapiens
MKLLTGLVFCSLVLSVSSRSFFSFLGEAFDGARDMWRAYSDMREANYIGSDKYFHARGNYDAAKRGPGGAWAAEVISNARENIQRLTGRGAEDSLADQAANKWGRSGRDPNHFRPAGLPEKY

SEQ ID NO: 14 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 15 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 16 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI